(19) United States
(12) Patent Application Publication
(10) Pub. No.: US 2018/0179267 A1
(43) Pub. Date: Jun. 28, 2018

(54) ANTI-STAPHYLOCOCCUS AUREUS ANTIBODY COMBINATION PREPARATION

(71) Applicant: ARSANIS BIOSCIENCES GMBH, Vienna (AT)

(72) Inventors: Eszter NAGY, Vienna (AT); Adriana BADARAU, Vienna (AT); Harald ROUHA, Wien (AT); Lukas STULIK, Vienna (AT); Zehra VISRAM, Vienna (AT)

(21) Appl. No.: 15/567,345

(22) PCT Filed: Apr. 14, 2016
(86) PCT No.: PCT/EP2016/058240

§ 371 Date: Oct. 17, 2017

(30) Foreign Application Priority Data
Apr. 17, 2015 (EP) 15164000.0

(51) Int. Cl. C07K 16/12; A61P 31/04

(52) U.S. Cl. CPC C07K 16/1271; A61K 2039/507; C07K 2317/33; A61P 31/04

(57) ABSTRACT

An anti-Staphylococcus aureus antibody combination preparation comprising a) a toxin cross-neutralizing antibody comprising at least one polyspecific binding site that binds to alpha-toxin (HIa) and at least one of the bi-component toxins selected from the group consisting of HIg AB, HIg CB, LukSF, LukED, Luk S-HIg B, LukSD, HIg A-LukD, HIg A-LukF, Luk EF, LukE-HIg B, HIg C-LukD and HIg C-LukF; and b) an anti-Luk GH antibody; and/or c) an OPK antibody which recognizes a S. aureus surface protein thereby inducing OPK, specifically an anti-Ig-binding protein (IGBP) antibody comprising at least one CDR binding site recognizing any of the S. aureus Ig G binding domains of Protein A or Sbi.

The invention refers to a combination of isolated antibodies directed against Staphylococcus aureus targeting alpha-toxin, leukocidins, and optionally an anti-Ig-binding protein (IGBP) and/or S. aureus surface proteins, with specific characteristics.

### BACKGROUND OF THE INVENTION

Staphylococcus aureus is a highly versatile opportunistic pathogen with numerous virulence mechanisms and complex pathogenesis. It is most often a harmless colonizer and present in 25-30% of individuals in the anterior nares, skin, gut and throat. When this “peaceful” co-existence is disturbed, S. aureus can become a powerful pathogen and can cause infection practically in all tissues, most commonly skin and soft tissue infections, pneumonia, bacteremia and sepsis (Lowy, 1998). In hospital settings S. aureus is one of most common causes of wound infection, catheter-, prosthetic device and ventilator-associated infections. In spite of repeated exposures to S. aureus and mild infections that do induce antibody response, this acquired immunity does not seem to be protective against disease in most individuals when they become vulnerable. S. aureus is a pyogenic bacterium and induces pronounced inflammatory responses. It expresses multiple virulence factors that disarm the innate defense system, most notably it produces powerful cytotoxins that cause local tissue damage and attack innate immune cells, such as granulocytes (polymorphonuclear leukocytes, PMNs) that are recruited to the site of infection (Rigby, 2012; Vandenesh, 2012; Spaan, 2013; Alonzo, 2013; Alonzo, 2014). The dead PMNs evoke further inflammation by activating another type of phagocytic cells, macrophages to remove the “carcasses”. This process is disarmed again by cytotoxins that kill not only PMNs but also macrophages. S. aureus produces an arsenal of leukotoxic molecules that eliminate innate immune cells. The different S. aureus strains can produce up to five bi-component leukocidins that without exception use immune receptors to find their target cells. LukSF (also called Panton Valentine Leukocidin, PVL) and HIgCB (gamma-hemolysin CB) use the complement receptors C5aR and C5L2 (Spaan, 2013; Spaan, 2014). LukGH (also called LukAB) targets phagocytic cells via another complement receptor CR3 formed by CD11b and CD18, expressed by all human professional phagocytic cells (Dumont, 2013). LukED and HIgAB share phagocytic cell targeting receptors CXCR1 and CXCR2, while they also bind to additional receptors, CCR5 and CCR2, respectively (Reyes-Robles, 2013; Spaan, 2014).

This high level of redundancy serves the bacterium very well. The different receptor specificities of the leukotoxins ensure that phagocytic cells with different subtypes and activation state can be all targeted. All S. aureus isolates produce gamma-hemolysins (HIgAB, HgCB) and LukGH, approximately 40-60% also process the lukED genes on their chromosomes, while lukSF (pvI) is carried by phages and expressed by approximately 5-10% of clinical isolates.

Previous attempts to counteract S. aureus disease pathogenesis with vaccines, polyclonal serum therapy or anti-staphylococcal monoclonal antibodies all failed to demonstrate clinical efficacy (Oleksiewicz, 2012; Jansen, 2013). All these approaches relied on antibodies targeting surface expressed molecules (adhesins and transport proteins) and aimed at inducing opsonophagocytic uptake and killing of S. aureus. In the light of recent research uncovering the powerful role of leukocidins, it is plausible that these antibodies were insufficient to promote bacterial elimination because the effector cells, the phagocytes were disarmed and the host could not benefit from more surface binding antibodies. The presence of high level of immunoglobulins targeting the S. aureus surface in both healthy and diseased people (Dryla, 2005) suggest that the lack of protection from repeated S. aureus infections is not due to absence of this type of antibodies. Seroepidemiology studies suggested that neutralizing antibodies against certain toxins are positively correlated with better clinical outcome (Fritz, 2012; Adhikari, 2012). Therefore, supplementing the antibody repertoire with monoclonal antibodies neutralizing the leukocidins offers great therapeutic options.

In addition to leukocidins, alpha-toxin (alpha-hemolysin or HIa) that targets epithelial and endothelial cells, also induces inflammation, and although it does not directly lyse PMNs and macrophages, it can negatively affect viability of these cells and also those of undifferentiated immune cells.

WO2014/187746A2 describes a highly potent LukGH neutralizing human mAb generated with heterodimers, but not with LukG or LukH monomers. LukGH (also called LukAB) is a powerful leucocidin that is the most different among the five leukocidins based on lower sequence homology (˜30-40%) and formation of heterodimer in solution (DuMont, 2014; Badarau, 2015). Uniquely among the leukocidins, LukGH displays significant sequence variations among clinical isolates.

WO2013/156534A1 describes a cross-neutralizing antibody comprising at least one polyspecific binding site that binds to alpha-toxin and at least one of the bi-component toxins of Staphylococcus aureus.

Rouha (2015) describes the use of a unique human monoclonal antibody cross-reacting with four of the five leukocidins and alpha-hemolysin.

Besides cytolytic toxins, another powerful virulence mechanism is employed by S. aureus that leads to evasion of innate immune defense. S. aureus expresses two IgG binding proteins, Staphylococcal Surface Protein A (Spa or Protein A) and Staphylococcal binder of IgG (Sbi) are multifunctional virulence factors that interact with several human proteins, and act mainly as immune evasion molecules (Falugi, 2013; Smith, 2011). By binding to the Fc portion of immunoglobulins, SpA and Sbi protect Staphylococcus aureus from phagocytosis.

Given the complex pathogenesis of S. aureus, there is a need to develop an improved antibody preparation that is able to inactivate several exotoxins, which would significantly increase the potency of anti-S. aureus therapy.

### SUMMARY OF THE INVENTION

It is the objective of the present invention to provide for toxin-neutralizing antibodies in an antibody preparation with broad cross-neutralizing potency.

The object is solved by the subject of the present invention.

According to the invention, there is provided an anti-Staphylococcus aureus antibody combination preparation comprising

a) a toxin cross-neutralizing antibody comprising at least one polyspecific binding site that binds to alpha-toxin (HIa) and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and

b) an anti-LukGH antibody, specifically or preferably and in particular, an anti-LukGH antibody comprising at least one binding site that specifically binds to the LukGH complex or any of the LukG or LukH as individual targets; and/or

c) an OPK antibody which recognizes a S. aureus surface protein thereby inducing OPK, specifically or preferably and in particular, an anti-Ig-binding protein (IGBP) antibody comprising at least one CDR binding site recognizing any of the S. aureus IgG binding domains of Protein A or Sbi.

Specifically, the antibody combination preparation as described herein comprises

a) a toxin cross-neutralizing antibody comprising at least one polyspecific binding site that binds to alpha-toxin (HIa) and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and

b) an anti-LukGH antibody; and/or

c) an antibody specifically recognizing one or more S. aureus IgG binding domains of SpA or Sbi or an IGBP; and/or

d) an antibody specifically recognizing any S. aureus surface protein to bind an antibody thereby inducing OPK (herein referred to as OPK antibody).

Specifically, the toxin cross-neutralizing antibody has a cross-specificity to bind HIa and at least two or three of the bi-component leukotoxins.

Specifically, the toxin cross-neutralizing antibody has a cross-specificity to bind HIa and at least one of the F-components and/or at least one of the S-components of the bi-component toxins, preferably at least two or three different components of the bi-component toxins,

preferably wherein an F-component is selected from the group consisting of HIgB, LukF and LukD, or any F-component of the cognate and non-cognate pairs of F and S components of gamma-hemolysins, PVL toxins and PVL-like toxins, preferably HIgAB, HIgCB, LukSF, LukED, LukS-HIgB, LukSD, HIgA-LukD, HIgA-LukF, LukEF, LukE-HIgB, HIgC-LukD or HIgC-LukF; and

preferably wherein an S-component is selected from the group consisting of HIgA, HIgC, LukE, and LukS, or any S-component of the cognate and non-cognate pairs of F and S components of gamma-hemolysins, PVL toxins and PVL-like toxins, preferably HIgAB, HIgCB, LukSF, LukED, LukS-HIgB, LukSD, HIgA-LukD, HIgA-LukF, LukEF, LukE-HIgB, HIgC-LukD or HIgC-LukF.

Specifically, the S-component targeted by the antibody as described herein is any one, two, three or four of HIgA, HIgC, LukE, and LukS.

Specifically, the toxin cross-neutralizing antibody has a cross-specificity to bind HIa and at least one of the F-components of the bi-component toxins, preferably at least two or three thereof, preferably wherein the F-components are selected from the group consisting of HIgB, LukF and LukD, or any F-component of the cognate and non-cognate pairs of F and S components of gamma-hemolysins, PVL toxins and PVL-like toxins, preferably HIgAB, HIgCB, LukSF, LukED, LukS-HIgB, LukSD, HIgA-LukD, HIgA-LukF, LukEF, LukE-HIgB, HIgC-LukD or HIgC-LukF.

Specifically, the F-component targeted by the antibody as described herein is any one, two or three of HIgB, LukF and LukD.

Specifically, the toxin cross-neutralizing antibody has a cross-specificity to bind HIa and at least one of HIgAB, HIgCB, LukSF, and LukED, preferably at least two, three or each of the HIgAB, HIgCB, LukSF, and LukED.

According to a specific aspect, the toxin cross-neutralizing antibody inhibits the binding of one or more of the toxins to phosphocholine or phosphatidylcholine, in particular the phosphatidylcholine of mammalian cell membranes.

According to a specific aspect, the toxin cross-neutralizing antibody exhibits in vitro neutralization potency in a cell-based assay with an IC50 of less than 100:1 mAb:toxin ratio (mol/mol), preferably less than 50:1, preferably less than 25:1, preferably less than 10:1, more preferably less than 1:1.

According to a further specific aspect, the toxin cross-neutralizing antibody neutralizes the targeted toxins in animals, including both, human and non-human animals, and inhibits S. aureus pathogenesis in vivo, preferably any models of pneumonia, bacteremia, sepsis, abscess, skin infection, peritonitis, catheter and prothetic devices related infection and osteomyelitis.

Specifically, the toxin cross-neutralizing antibody comprises three complementarity determining regions (CDR1 to CDR3) of the antibody heavy chain variable region (VH) and three complementarity determining regions (CDR4 to CDR6) of the antibody light chain variable region (VL).

Specifically, the toxin cross-neutralizing antibody comprises at least three complementarity determining regions (CDR1 to CDR3) of the antibody heavy chain variable region (VH) of any of the antibodies shown in Table 1 (FIG. 1), or functionally active CDR variants of any of the foregoing.

Specifically, the toxin cross-neutralizing antibody comprises three complementarity determining regions (CDR1 to CDR3) of the antibody heavy chain variable region (VH) of any of the antibodies listed in Table 1, or functionally active CDR variants of any of the foregoing; and three complementarity determining regions (CDR4 to CDR6) of the antibody light chain variable region (VL) of any of the antibodies listed in Table 1, or functionally active CDR variants of any of the foregoing.

Specifically, the toxin cross-neutralizing antibody comprises six complementarity determining regions (CDR1 to CDR6) of any of the antibodies listed in Table 1, or functionally active CDR variants of any of the foregoing.

Specifically, the toxin cross-neutralizing antibody comprises at least CDR1, CDR2, and CDR3 of VH, wherein

A) the antibody comprises
a) a CDR1 comprising or consisting of the amino acid sequence SEQ ID 1; and
b) a CDR2 comprising or consisting of the amino acid sequence SEQ ID 2; and
c) a CDR3 comprising or consisting of the amino acid sequence SEQ ID 3;
i.e. herein referred to as toxin cross-neutralizing antibody of embodiment VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 1;
b) the parent CDR2 consists of the amino acid sequence SEQ ID 2; and
c) the parent CDR3 consists of the amino acid sequence SEQ ID 3;
i.e. herein referred to as toxin cross-neutralizing antibody of embodiment VH-B.

Specifically, the toxin cross-neutralizing antibody comprising such functionally active CDR variant (the toxin cross-neutralizing antibody of embodiment VH-B above) is characterized by any of the following amino acid residues:
a) in VH CDR1 at position 5, the amino acid residue selected from the group consisting of S, A, D, E, F, G, H, I, K, L, M, N, Q, R, T V, W and Y, preferentially any of H, R and W;
b) in VH CDR1 at position 7, the amino acid residue selected from the group consisting of M, H, K, Q, R and W, preferentially any of K, R or W;
c) in VH CDR2 at position 3, the amino acid residue is selected from the group consisting of D and R;
d) in VH CDR2 at position 7, the amino acid residue selected from the group consisting of S, A, D, E, F, H, K, M, N, Q, R, T, W and Y, preferentially any of D, H, K, N or Q, and more preferentially is Q;
e) in VH CDR2 at position 9, the amino acid residue selected from the group consisting of Y, F, K, L, Q and R, and preferentially is R;
f) in VH CDR3 at position 5, the amino acid residue selected from the group consisting of G, A, D, F, H, I, M, N, R, S, T, V and Y, preferentially any of D, F, H, I, M, N, R, T, V or Y;
g) in VH CDR3 at position 6, the amino acid residue selected from the group consisting of H, E, Q and S, preferentially any of E or Q;
h) in VH CDR3 at position 7, the amino acid residue selected from the group consisting of G, A, D, E, H, I, M, N, Q, S, T, V and W, and preferentially is W; and/or
i) in VH CDR3 at position 8, the amino acid residue selected from the group consisting of V, A, D, E, G, I, K, L, M, Q, R, S and T, preferentially any of M or R.

Specifically, the toxin cross-neutralizing antibody comprises a functionally active CDR variant of a parent antibody, wherein the parent antibody is e.g. the toxin cross-neutralizing antibody of embodiment VH-A or VH-B above, in particular any of the antibodies listed in Table 1, which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

Specifically, the toxin cross-neutralizing antibody of embodiment VH-B above comprises at least one functionally active CDR variant which is any of
a) a CDR1 sequence selected from the group consisting of SEQ ID 4, and

SEQ ID 5; or
b) a CDR2 sequence selected from the group consisting of SEQ ID 6, SEQ ID 7, SEQ ID 8, SEQ ID 9, and SEQ ID 10; or
c) a CDR3 sequence selected from the group consisting of SEQ ID 11, and SEQ ID 12.

Specifically, the toxin cross-neutralizing antibody of embodiment VH-B above is selected from the group consisting of

a) an antibody comprising
a. the CDR1 sequence SEQ ID 1; and
b. the CDR2 sequence SEQ ID 6; and
c. the CDR3 sequence SEQ ID 11;

b) an antibody comprising
a. the CDR1 sequence SEQ ID 4; and
b. the CDR2 sequence SEQ ID 7; and
c. the CDR3 sequence SEQ ID 3;

c) an antibody comprising
a. the CDR1 sequence SEQ ID 1; and
b. the CDR2 sequence SEQ ID 8; and
c. the CDR3 sequence SEQ ID 3;

d) an antibody comprising
a. the CDR1 sequence SEQ ID 1; and
b. the CDR2 sequence SEQ ID 2; and
c. the CDR3 sequence SEQ ID 12;

e) an antibody comprising
a. the CDR1 sequence SEQ ID 5; and
b. the CDR2 sequence SEQ ID 9; and
c. the CDR3 sequence SEQ ID 3; and

f) an antibody comprising
a. the CDR1 sequence SEQ ID 5; and
b. the CDR2 sequence SEQ ID 10; and
c. the CDR3 sequence SEQ ID 3;

Specifically, the toxin cross-neutralizing antibody comprises any of the VH amino acid sequence as depicted in FIG. 2, in particular FIG. 2a.

Specifically, the toxin cross-neutralizing antibody comprises a VH amino acid sequence selected from the group consisting of SEQ ID 20-31, preferably comprising an antibody heavy chain (HC) amino acid sequence selected from the group consisting of SEQ ID 40-51, or any of the amino acid sequences SEQ ID 40-51 with a deletion of the C-terminal amino acid.

According to a specific aspect, each of the HC sequences may be terminally extended or deleted in the constant region, e.g. a deletion of one or more or the C-terminal amino acids.

Specifically, each of the HC sequences that comprises an C-terminal Lysine residue is preferably employed with a deletion of such C-terminal Lysine residue.

Specifically, SEQ ID 40-51 show the HC sequences which is N-terminally extended by a signal sequence. It is understood that the specific antibody comprises such HC amino acid sequence with or without the respective signal sequence, or with alternative signal or leader sequences.

While the toxin cross-neutralizing antibody may be provided as an antibody comprising a binding site determined by CDR sequences of the VH sequence only, e.g. a VH antibody or a heavy chain antibody, according to a specific aspect, the binding site may be further determined by CDR sequences of the antibody light chain variable region (VL), preferably which comprises any of the CDR4 to CDR6 sequences as listed in Table 1, or functionally active CDR variants thereof.

Specifically, the toxin cross-neutralizing antibody of embodiment VH-A or VH-B above further comprises at least three complementarity determining regions (CDR4 to CDR6) of the VL, preferably wherein

A) the antibody comprises
a) a CDR4 comprising or consisting of the amino acid sequence SEQ ID 32; and
b) a CDR5 comprising or consisting of the amino acid sequence SEQ ID 33; and
c) a CDR6 comprising or consisting of the amino acid sequence SEQ ID 34;
i.e. herein referred to as toxin cross-neutralizing antibody of embodiment VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 32;
b) the parent CDR5 consists of the amino acid sequence SEQ ID 33;
c) the parent CDR6 consists of the amino acid sequence SEQ ID 34;
i.e. herein referred to as toxin cross-neutralizing antibody of embodiment VL-B.

Specifically, the toxin cross-neutralizing antibody comprising such functionally active CDR variant (the toxin cross-neutralizing antibody of embodiment VL-B above) is characterized by any of the following amino acid residues:
a) in VL CDR4 at position 7, the amino acid residue selected from the group consisting of S, A, E, F, G, K, L, M, N, Q, R, W and Y, preferentially any of L, M, R or W, and more preferentially is R;
b) in VL CDR5 at position 1, the amino acid residue selected from the group consisting of A and G;
c) in VL CDR5 at position 3, the amino acid residue selected from the group consisting of S, A, D, G, H, I, K, L, N, Q, R, T, V and W;
d) in VL CDR5 at position 4, the amino acid residue selected from the group consisting of S, D, E, H, I, K, M, N, Q, R, T and V, preferentially any of K, N, Q and R;
e) in VL CDR6 at position 3, the amino acid residue selected from the group consisting of G, A, D, E, F, H, I, K, L, N, Q, R, S, T, V, W and Y;
f) in VL CDR6 at position 4, the amino acid residue selected from the group consisting of Y, D, F, H, M, R and W;
g) in VL CDR6 at position 5, the amino acid residue selected from the group consisting of V, A, D, E, F, G, H, I, K, L, M, N, Q, R, S, T, and W; and/or
h) in VL CDR6 at position 6, the amino acid residue selected from the group consisting of F and W.

Specifically, the toxin cross-neutralizing antibody comprises a functionally active CDR variant of a parent antibody, wherein the parent antibody is e.g. the toxin cross-neutralizing antibody of embodiment VL-A or VL-B above, in particular any of the antibodies listed in Table 1, which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

Specifically, the toxin cross-neutralizing antibody comprises a VL amino acid sequence SEQ ID 39 or an antibody light chain (LC) amino acid SEQ ID 52.

According to a specific embodiment, the toxin cross-neutralizing antibody comprises at least one polyspecific binding site that binds to alpha-toxin (HIa) and at least one of the bi-component toxins of S. aureus, which antibody is a functionally active variant antibody of a parent antibody that comprises a polyspecific binding site of the VH amino acid sequence SEQ ID 20, and the VL amino acid sequence SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a K_{D} of less than 10⁻⁸M, preferably less than 10⁻⁹M.

Specifically, such functionally active variant antibody comprises
a) in VH CDR1 at position 5, the amino acid residue selected from the group consisting of S, A, D, E, F, G, H, I, K, L, M, N, Q, R, T V, W and Y, preferentially any of H, R and W;
b) in VH CDR1 at position 7, the amino acid residue selected from the group consisting of M, H, K, Q, R and W, preferentially any of K, R or W;
c) in VH CDR2 at position 3, the amino acid residue selected from the group consisting of D and R;
d) in VH CDR2 at position 7, the amino acid residue selected from the group consisting of S, A, D, E, F, H, K, M, N, Q, R, T, W and Y, preferentially any of D, H, K, N or Q, and more preferentially is Q;
e) in VH CDR2 at position 9, the amino acid residue selected from the group consisting of Y, F, K, L, Q and R, and preferentially is R;
f) in VH CDR3 at position 5, the amino acid residue selected from the group consisting of G, A, D, F, H, I, M, N, R, S, T, V and Y, preferentially any of D, F, H, I, M, N, R, T, V or Y;
g) in VH CDR3 at position 6, the amino acid residue selected from the group consisting of H, E, Q and S, preferentially any of E or Q;
h) in VH CDR3 at position 7, the amino acid residue selected from the group consisting of G, A, D, E, H, I, M, N, Q, S, T, V and W, and preferentially is W; and/or
i) in VH CDR3 at position 8, the amino acid residue selected from the group consisting of V, A, D, E, G, I, K, L, M, Q, R, S and T, preferentially any of M or R.

Specifically, such functionally active variant antibody comprises
a) in VL CDR4 at position 7, the amino acid residue selected from the group consisting of S, A, E, F, G, K, L, M, N, Q, R, W and Y, preferentially any of L, M, R or W, and more preferentially is R;
b) in VL CDR5 at position 1, the amino acid residue selected from the group consisting of A and G;
c) in VL CDR5 at position 3, the amino acid residue selected from the group consisting of S, A, D, G, H, I, K, L, N, Q, R, T, V and W;
d) in VL CDR5 at position 4, the amino acid residue selected from the group consisting of S, D, E, H, I, K, M, N, Q, R, T and V, preferentially any of K, N, Q and R;
e) in VL CDR6 at position 3, the amino acid residue selected from the group consisting of G, A, D, E, F, H, I, K, L, N, Q, R, S, T, V, W and Y;
f) in VL CDR6 at position 4, the amino acid residue selected from the group consisting of Y, D, F, H, M, R and W;
g) in VL CDR6 at position 5, the amino acid residue selected from the group consisting of V, A, D, E, F, G, H, I, K, L, M, N, Q, R, S, T, and W; and/or
h) in VL CDR6 at position 6, the amino acid residue selected from the group consisting of F and W.

According to a specific embodiment, the anti-LukGH antibody comprises an antibody heavy chain variable region (VH) comprising the CDR1 to CDR3 sequences of any antibody listed in Table 2 (Table 2 is herein understood as any of the Tables 2 of FIG. 1), or functionally active CDR variants thereof, and an antibody light chain variable region (VL) comprising the CDR4 to CDR6 sequences of any antibody listed in Table 2, or functionally active CDR variants thereof.

According to a specific aspect, the anti-LukGH antibody comprises any of the CDR1 to CDR3 sequences as listed in Table 2, specifically the CDR1 to CDR3 sequences of any of the antibodies listed in Table 2, more specifically the VH CDR1 to CDR3, and the VL CDR4 to CDR6 sequences of any of the antibodies listed in Table 2, or functionally active CDR variants of any of the foregoing.

Specifically, the anti-LukGH antibody is selected from the group consisting of group members i) to viii), each being either embodiment A or B, herein referred to as anti-LukGH antibody of embodiments VH-A or VH-B, wherein

i)

A) the antibody comprises
a) a CDR1 comprising or consisting of the amino acid sequence SEQ ID 86 or SEQ ID 99; and
b) a CDR2 comprising or consisting of the amino acid sequence SEQ ID 88; and
c) a CDR3 comprising or consisting of the amino acid sequence SEQ ID 90;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 86 or SEQ ID 99;
b) the parent CDR2 consists of the amino acid sequence SEQ ID 88;
c) the parent CDR3 consists of the amino acid sequence SEQ ID 90;
i.e. herein referred to as anti-LukGH antibody of one of the embodiments VH-B;

ii)

A) the antibody comprises
a) a CDR1 comprising or consisting of any of the amino acid sequences SEQ ID 110, SEQ ID 120, or SEQ ID 122; and
b) a CDR2 comprising or consisting of any of the amino acid sequences SEQ ID 112, SEQ ID 121, SEQ ID 123, or SEQ ID 124; and
c) a CDR3 comprising or consisting of the amino acid sequence SEQ ID 114;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 110, SEQ ID 120, or SEQ ID 122;
b) the parent CDR2 consists of the amino acid sequence SEQ ID 112, SEQ ID 121, SEQ ID 123, or SEQ ID 124;
c) the parent CDR3 consists of the amino acid sequence SEQ ID 114;
i.e. herein referred to as anti-LukGH antibody of one of the embodiments VH-B;

iii)

A) the antibody comprises
a) a CDR1 comprising or consisting any of the amino acid sequences SEQ ID 131, SEQ ID 139, SEQ ID 141, SEQ ID 143, SEQ ID 145, SEQ ID 147, or SEQ ID 148; and
b) a CDR2 comprising or consisting of any of the amino acid sequences SEQ ID 133, SEQ ID 140, SEQ ID 142, SEQ ID 144, SEQ ID 146, SEQ ID 149, or SEQ ID 150; and
c) a CDR3 comprising or consisting of the amino acid sequence SEQ ID 135;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 131, SEQ ID 139, SEQ ID 141, SEQ ID 143, SEQ ID 145, SEQ ID 147, or SEQ ID 148;
b) the parent CDR2 consists of the amino acid sequence SEQ ID 133, SEQ ID 140, SEQ ID 142, SEQ ID 144, SEQ ID 146, SEQ ID 149, or SEQ ID 150;
c) the parent CDR3 consists of the amino acid sequence SEQ ID 135;
i.e. herein referred to as anti-LukGH antibody of one of the embodiments VH-B;

iv)

A) the antibody comprises
a) a CDR1 comprising or consisting of any of the amino acid sequences SEQ ID 155, SEQ ID 161, SEQ ID 163, SEQ ID 165, SEQ ID 167, or SEQ ID 169; and
b) a CDR2 comprising or consisting of any of the amino acid sequences SEQ ID 156, SEQ ID 162, SEQ ID 168, or SEQ ID 88; and
c) a CDR3 comprising or consisting of the amino acid sequence SEQ ID 157;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 155, SEQ ID 161, SEQ ID 163, SEQ ID 165, SEQ ID 167, or SEQ ID 169;
b) the parent CDR2 consists of the amino acid sequence SEQ ID 156, SEQ ID 162, SEQ ID 168, or SEQ ID 88;
c) the parent CDR3 consists of the amino acid sequence SEQ ID 157;
i.e. herein referred to as anti-LukGH antibody of one of the embodiments VH-B;

v)

A) the antibody comprises
a) a CDR1 comprising or consisting of any of the amino acid sequences SEQ ID 171, SEQ ID 181, SEQ ID 183, or SEQ ID 185; and
b) a CDR2 comprising or consisting of any of the amino acid sequences SEQ ID 172, SEQ ID 182, SEQ ID 184, or SEQ ID 186; and
c) a CDR3 comprising or consisting of the amino acid sequence SEQ ID 173;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 171, SEQ ID 181, SEQ ID 183, or SEQ ID 185;
b) the parent CDR2 consists of the amino acid sequence SEQ ID 172, SEQ ID 182, SEQ ID 184, or SEQ ID 186;
c) the parent CDR3 consists of the amino acid sequence SEQ ID 173;
i.e. herein referred to as anti-LukGH antibody of one of the embodiments VH-B;

vi)

A) the antibody comprises
a) a CDR1 comprising or consisting of any of the amino acid sequences SEQ ID 188, SEQ ID 194, SEQ ID 196, SEQ ID 122, SEQ ID 198, SEQ ID 203, or SEQ ID 204; and
b) a CDR2 comprising or consisting of any of the amino acid sequences SEQ ID 189, SEQ ID 193, SEQ ID 195, SEQ ID 197, SEQ ID 186, SEQ ID 199, or SEQ ID 205; and
c) a CDR3 comprising or consisting of the amino acid sequence SEQ ID 190;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 188, SEQ ID 194, SEQ ID 196, SEQ ID 122, SEQ ID 198, SEQ ID 203, or SEQ ID 204;
b) the parent CDR2 consists of the amino acid sequence SEQ ID 189, SEQ ID 193, SEQ ID 195, SEQ ID 197, SEQ ID 186, SEQ ID 199, or SEQ ID 205;
c) the parent CDR3 consists of the amino acid sequence SEQ ID 190;
i.e. herein referred to as anti-LukGH antibody of one of the embodiments VH-B;

vii)

A) the antibody comprises
a) a CDR1 comprising or consisting of the amino acid sequence SEQ ID 209; and
b) a CDR2 comprising or consisting of the amino acid sequence SEQ ID 210; and
c) a CDR3 comprising or consisting of the amino acid sequence SEQ ID 211;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 209;
b) the parent CDR2 consists of the amino acid sequence SEQ ID 210;
c) the parent CDR3 consists of the amino acid sequence SEQ ID 211;
i.e. herein referred to as anti-LukGH antibody of one of the embodiments VH-B;

and viii)

A) the antibody comprises
a) a CDR1 comprising or consisting of the amino acid sequence SEQ ID 218; and
b) a CDR2 comprising or consisting of the amino acid sequence SEQ ID 219; and
c) a CDR3 comprising or consisting of the amino acid sequence SEQ ID 221;
i.e. herein referred to as anti-LukGH antibody of one of the embodiments VH-A;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 218;
b) the parent CDR2 consists of the amino acid sequence SEQ ID 219;
c) the parent CDR3 consists of the amino acid sequence SEQ ID 221;
i.e. herein referred to as anti-LukGH antibody of one of the embodiments VH-B.

Specifically, the anti-LukGH antibody of group member iv) above, such as including e.g.

iv)

A) the antibody comprising
a) a CDR1 comprising or consisting of any of the amino acid sequences SEQ ID 155, SEQ ID 161, SEQ ID 163, SEQ ID 165, SEQ ID 167, or SEQ ID 169; and
b) a CDR2 comprising or consisting of any of the amino acid sequences SEQ ID 156, SEQ ID 162, SEQ ID 168, or SEQ ID 88; and
c) a CDR3 comprising or consisting of the amino acid sequence SEQ ID 157;

i.e. herein referred to as anti-LukGH antibody of embodiment VH-A;

or

B) the antibody which is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 155, SEQ ID 161, SEQ ID 163, SEQ ID 165, SEQ ID 167, or SEQ ID 169;
b) the parent CDR2 consists of the amino acid sequence SEQ ID 156, SEQ ID 162, SEQ ID 168, or SEQ ID 88;
c) the parent CDR3 consists of the amino acid sequence SEQ ID 157;

i.e. herein referred to as anti-LukGH antibody of embodiment VH-B;

is an antibody of embodiment VH-B or a functionally active variant thereof, characterized by any of the following amino acid residues:

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H; and/or

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y.

Specifically, the anti-LukGH antibody or the functionally active variant thereof comprises a VH amino acid sequence selected from any of the VH sequences as depicted in FIG. 2, in particular FIG. 2b, Group 4, or an antibody heavy chain (HC) amino acid sequence selected from the group consisting of SEQ ID 241, SEQ ID 243, SEQ ID 245, which may be used in the combination preparation as such, or used as parent antibody to produce functionally active variants.

Specifically, the anti-LukGH antibody comprises a functionally active CDR variant of a parent antibody, wherein the parent antibody is e.g. the anti-LukGH antibody of one of the embodiments VH-A or VH-B above, in particular any of the antibodies listed in Table 2 (any of Groups 1-8), which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

Specifically, the anti-LukGH antibody is selected from the group consisting of

a) an antibody comprising
a. the CDR1 sequence SEQ ID 122; and
b. the CDR2 sequence SEQ ID 123; and
c. the CDR3 sequence SEQ ID 114;

b) an antibody comprising
a. the CDR1 sequence SEQ ID 131; and
b. the CDR2 sequence SEQ ID 133; and
c. the CDR3 sequence SEQ ID 135;

c) an antibody comprising
a. the CDR1 sequence SEQ ID 167; and
b. the CDR2 sequence SEQ ID 168; and
c. the CDR3 sequence SEQ ID 157;

d) an antibody comprising
a. the CDR1 sequence SEQ ID 188; and
b. the CDR2 sequence SEQ ID 189; and
c. the CDR3 sequence SEQ ID 190; and

e) an antibody comprising
a. the CDR1 sequence SEQ ID 198; and
b. the CDR2 sequence SEQ ID 199; and
c. the CDR3 sequence SEQ ID 190.

Specifically, the anti-LukGH antibody comprises any of
a) a VH amino acid sequence selected from any of the VH sequences as depicted in FIG. 2, in particular FIG. 2b;
b) an antibody heavy chain (HC) amino acid sequence selected from the group consisting of SEQ ID 231, SEQ ID 233, SEQ ID 235, SEQ ID 237, SEQ ID 239, SEQ ID 241, SEQ ID 243, SEQ ID 245, SEQ ID 247, SEQ ID 249, SEQ ID 251, SEQ ID 253, and SEQ ID 255; or
c) an antibody heavy chain (HC) amino acid sequence selected from the group consisting of SEQ ID 231, SEQ ID 233, SEQ ID 235, SEQ ID 237, SEQ ID 239, SEQ ID 241, SEQ ID 243, SEQ ID 245, SEQ ID 247, SEQ ID 249, SEQ ID 251, SEQ ID 253, and SEQ ID 255, which is further comprising a deletion of the C-terminal amino acid and/or a Q1E point mutation, if the first amino acid of the VH sequence is a Q.

While the anti-LukGH antibody may be provided as an antibody comprising a binding site determined by CDR sequences of the VH sequence only, e.g. a VH antibody or a heavy chain antibody, according to a specific aspect, the binding site may be further determined by CDR sequences of the antibody light chain variable region (VL), preferably which comprises any of the CDR4 to CDR6 sequences as listed in Table 2 (any of Groups 1-8, or VH and VL within the same Group of any of Groups 1-8), or functionally active CDR variants thereof.

Specifically, the anti-LukGH antibody of one of the embodiments VH-A or VH-B above further comprises at least three complementarity determining regions (CDR4 to CDR6) of the VL, preferably wherein the anti-LukGH antibody is selected from the group consisting of group members i) to viii), each being either embodiment A or B, herein referred to as anti-LukGH antibody of embodiments VL-A or VL-B, wherein

i)

A) the antibody comprises
a) a CDR4 comprising or consisting of the amino acid sequence SEQ ID 93 or SEQ ID 103; and
b) a CDR5 comprising or consisting of any of the amino acid sequences SEQ ID 95, SEQ ID 100, or SEQ ID 105; and
c) a CDR6 comprising or consisting of any of the amino acid sequences SEQ ID 97, SEQ ID 101, SEQ ID 107, or SEQ ID 108;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 93 or SEQ ID 103;
b) the parent CDR5 consists of the amino acid sequence SEQ ID 95, SEQ ID 100, or SEQ ID 105;
c) the parent CDR6 consists of the amino acid sequence SEQ ID 97, SEQ ID 101, SEQ ID 107, or SEQ ID 108;
i.e. herein referred to as anti-LukGH antibody of one of the embodiments VL-B;

ii)

A) the antibody comprises
a) a CDR4 comprising or consisting of the amino acid sequence SEQ ID 116; and
b) a CDR5 comprising or consisting of the amino acid sequence SEQ ID 117 or SEQ ID 125; and
c) a CDR6 comprising or consisting of any of the amino acid sequences SEQ ID 119, SEQ ID 126, SEQ ID 127, or SEQ ID 129;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 116;
b) the parent CDR5 consists of the amino acid sequence SEQ ID 117 or SEQ ID 125;
c) the parent CDR6 consists of the amino acid sequence SEQ ID 119, SEQ ID 126, SEQ ID 127, or SEQ ID 129;
i.e. herein referred to as anti-LukGH antibody of one of the embodiments VL-B;

iii)

A) the antibody comprises
a) a CDR4 comprising or consisting of any of the amino acid sequences SEQ ID 137, SEQ ID 151, or SEQ ID 103; and
b) a CDR5 comprising or consisting of the amino acid sequence SEQ ID 105; and
c) a CDR6 comprising or consisting of any of the amino acid sequences SEQ ID 138, SEQ ID 152, SEQ ID 153, or SEQ ID 154;
i.e. herein referred to as anti-LukGH antibody of one of the embodiments VL-A;
or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 137, SEQ ID 151, or SEQ ID 103; or
b) the parent CDR5 consists of the amino acid sequence SEQ ID 105; or
c) the parent CDR6 consists of the amino acid sequence SEQ ID 138, SEQ ID 152, SEQ ID 153, or SEQ ID 154;
i.e. herein referred to as anti-LukGH antibody of one of the embodiments VL-B;

iv)

A) the antibody comprises
a) a CDR4 comprising or consisting of the amino acid sequence SEQ ID 159 or SEQ ID 116; and
b) a CDR5 comprising or consisting of the amino acid sequence SEQ ID 125; and
c) a CDR6 comprising or consisting of the amino acid sequence SEQ ID 160 or SEQ ID 170;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 159 or SEQ ID 116; or
b) the parent CDR5 consists of the amino acid sequence SEQ ID 125; or
c) the parent CDR6 consists of the amino acid sequence SEQ ID 160 or SEQ ID 170;
i.e. herein referred to as anti-LukGH antibody of one of the embodiments VL-B;

v)

A) the antibody comprises
a) a CDR4 comprising or consisting of the amino acid sequence SEQ ID 176; and
b) a CDR5 comprising or consisting of any of the amino acid sequence SEQ ID 178; and
c) a CDR6 comprising or consisting of the amino acid sequence SEQ ID 180 or SEQ ID 187;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176;
b) the parent CDR5 consists of the amino acid sequence SEQ ID 178;
c) the parent CDR6 consists of the amino acid sequence SEQ ID 180 or SEQ ID 187;
i.e. herein referred to as anti-LukGH antibody of one of the embodiments VL-B;

vi)

A) the antibody comprises
a) a CDR4 comprising or consisting of the amino acid sequence SEQ ID 176 or SEQ ID 200; and
b) a CDR5 comprising or consisting of the amino acid sequence SEQ ID 178 or SEQ ID 201; and
c) a CDR6 comprising or consisting of any of the amino acid sequences SEQ ID 192, SEQ ID 202, or SEQ ID 207;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176 or SEQ ID 200;
b) the parent CDR5 consists of the amino acid sequence SEQ ID 178 or SEQ ID 201;
c) the parent CDR6 consists of the amino acid sequence SEQ ID 192, SEQ ID 202, or SEQ ID 207;
i.e. herein referred to as anti-LukGH antibody of one of the embodiments VL-B;

vii)

A) the antibody comprises
a) a CDR4 comprising or consisting of the amino acid sequence SEQ ID 116; and
b) a CDR5 comprising or consisting of the amino acid sequence SEQ ID 125; and
c) a CDR6 comprising or consisting of any of the amino acid sequences SEQ ID 213, SEQ ID 214, SEQ ID 215, or SEQ ID 216;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 116;
b) the parent CDR5 consists of the amino acid sequence SEQ ID 125;
c) the parent CDR6 consists of the amino acid sequence SEQ ID 213, SEQ ID 214, SEQ ID 215, or SEQ ID 216;
i.e. herein referred to as anti-LukGH antibody of one of the embodiments VL-B;

and viii)

A) the antibody comprises
a) a CDR4 comprising or consisting of the amino acid sequence SEQ ID 176 or SEQ ID 200; and
b) a CDR5 comprising or consisting of the amino acid sequence SEQ ID 178; and
c) a CDR6 comprising or consisting of any of the amino acid sequences SEQ ID 224, SEQ ID 180, SEQ ID 226, or SEQ ID 227;
i.e. herein referred to as anti-LukGH antibody of one of the embodiments VL-A;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176 or SEQ ID 200;
b) the parent CDR5 consists of the amino acid sequence SEQ ID 178;
c) the parent CDR6 consists of the amino acid sequence SEQ ID 224, SEQ ID 180, SEQ ID 226, or SEQ ID 227;
i.e. herein referred to as anti-LukGH antibody of one of the embodiments VL-B.

Specifically, the anti-LukGH antibody comprises a functionally active CDR variant of a parent antibody, wherein the parent antibody is e.g. the anti-LukGH antibody of one of the embodiments VL-A or VL-B above, in particular any of the antibodies listed in Table 2 (any of Groups 1-8), which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

Specifically, the anti-LukGH antibody of group member iv) above, such as including e.g.

iv)

A) the antibody comprising
a) a CDR4 comprising or consisting of the amino acid sequence SEQ ID 159 or SEQ ID 116; and
b) a CDR5 comprising or consisting of the amino acid sequence SEQ ID 125; and
c) a CDR6 comprising or consisting of the amino acid sequence SEQ ID 160 or SEQ ID 170;
i.e. herein referred to as anti-LukGH antibody of embodiment VL-A;

or

B) the antibody which is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 159 or SEQ ID 116;
b) the parent CDR5 consists of the amino acid sequence SEQ ID 125;
c) the parent CDR6 consists of the amino acid sequence SEQ ID 160 or SEQ ID 170;
i.e. herein referred to as anti-LukGH antibody of embodiment VL-B;

is an antibody of embodiment VL-B or a functionally active variant thereof, characterized by any of the following amino acid residues wherein

a) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

b) in VL CDR4 at position 8, the amino acid residue is selected from S, A, D, E, F, G, H, I, K, L, M, N, Q, R, T, V, W, and Y, preferentially I or W;

c) in VL CDR4 at position 9, the amino acid residue is selected from Y, F, R and W, and preferentially R or W;

d) in VL CDR5 at position 1, the amino acid residue is selected from A, G, S, W and Y, and preferentially is G;

e) in VL CDR6 at position 4, the amino acid residue is selected from F, H, M, W and Y;

f) in VL CDR6 at position 5, the amino acid residue is selected from D, A, D, E, F, G, H, I, K, L, M, N, Q, R, S, T, V, W, and Y; and/or

g) in VL CDR6 at position 8, the amino acid residue is selected from F, H, R and W.

Specifically, the anti-LukGH antibody comprises a VL amino acid sequence selected from any of the VL sequences as depicted in FIG. 2, in particular FIG. 2b, or an antibody light chain (LC) amino acid sequence selected from the group consisting of SEQ ID 232, SEQ ID 234, SEQ ID 236, SEQ ID 238, SEQ ID 240, SEQ ID 242, SEQ ID 244, SEQ ID 246, SEQ ID 248, SEQ ID 250, SEQ ID 252, SEQ ID 254, and SEQ ID 256, or a functionally active CDR variant of any of the foregoing, which has an affinity to bind the LukGH complex with a K_{D} of less than 10⁻⁸M, preferably less than 10⁻⁹M.

Specifically, the anti-LukGH antibody or the functionally active variant thereof comprises a VL amino acid sequence selected from any of the VL sequences as depicted in FIG. 2, in particular FIG. 2b, Group 4, or an antibody light chain (LC) amino acid sequence selected from the group consisting of SEQ ID 242, SEQ ID 244, SEQ ID 246, wherein

a) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

b) in VL CDR4 at position 8, the amino acid residue is selected from S, A, D, E, F, G, H, I, K, L, M, N, Q, R, T, V, W, and Y, preferentially I or W;

c) in VL CDR4 at position 9, the amino acid residue is selected from Y, F, R and W, and preferentially R or W;

d) in VL CDR5 at position 1, the amino acid residue is selected from A, G, S, W and Y, and preferentially is G;

e) in VL CDR6 at position 4, the amino acid residue is selected from F, H, M, W and Y;

f) in VL CDR6 at position 5, the amino acid residue is selected from D, A, D, E, F, G, H, I, K, L, M, N, Q, R, S, T, V, W, and Y; and/or

g) in VL CDR6 at position 8, the amino acid residue is selected from F, H, R and W.

Specifically, the anti-LukGH antibody is selected from the group consisting of

a) an antibody comprising
a. the CDR1 sequence SEQ ID 122; and
b. the CDR2 sequence SEQ ID 123; and
c. the CDR3 sequence SEQ ID 114; and
d. the CDR4 sequence SEQ ID 116; and
e. the CDR5 sequence SEQ ID 117; and
f. the CDR6 sequence SEQ ID 119;

b) an antibody comprising
a. the CDR1 sequence SEQ ID 131; and
b. the CDR2 sequence SEQ ID 133; and
c. the CDR3 sequence SEQ ID 135; and
d. the CDR4 sequence SEQ ID 137; and
e. the CDR5 sequence SEQ ID 105; and
f. the CDR6 sequence SEQ ID 138;

c) an antibody comprising
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;

d) an antibody comprising
a. the CDR1 sequence SEQ ID 188; and
b. the CDR2 sequence SEQ ID 189; and
c. the CDR3 sequence SEQ ID 190; and
d. the CDR4 sequence SEQ ID 176; and
e. the CDR5 sequence SEQ ID 178; and
f. the CDR6 sequence SEQ ID 192; and

e) an antibody comprising
a. the CDR1 sequence SEQ ID 198; and
b. the CDR2 sequence SEQ ID 199; and
c. the CDR3 sequence SEQ ID 190; and
d. the CDR4 sequence SEQ ID 200; and
e. the CDR5 sequence SEQ ID 201; and
f. the CDR6 sequence SEQ ID 202;

or a functionally active CDR variant of any of the foregoing, which has an affinity to bind the LukGH complex with a K_{D} of less than 10⁻⁸M, preferably less than 10⁻⁹M.

Specifically, the anti-LukGH antibody is an antibody of group member c) such as characterized by
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;

or a functionally active variant thereof, wherein:

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H;

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y;

k) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

I) in VL CDR4 at position 8, the amino acid residue is selected from S, A, D, E, F, G, H, I, K, L, M, N, Q, R, T, V, W, and Y, preferentially I or W;

m) in VL CDR4 at position 9, the amino acid residue is selected from Y, F, R and W, and preferentially R or W;

n) in VL CDR5 at position 1, the amino acid residue is selected from A, G, S, W and Y, and preferentially is G;

o) in VL CDR6 at position 4, the amino acid residue is selected from F, H, M, W and Y;

p) in VL CDR6 at position 5, the amino acid residue is selected from D, A, D, E, F, G, H, I, K, L, M, N, Q, R, 5, T, V, W, and Y; and/or

q) in VL CDR6 at position 8, the amino acid residue is selected from F, H, R and W.

Specifically, the anti-LukGH antibody comprises a framework including any of the framework regions of the VH and/or VL as listed in Table 2, optionally comprising a Q1E point mutation, if the first amino acid of the VH framework region (VH FR1) is a Q.

Specifically, the anti-LukGH antibody comprises a HC amino acid sequence as depicted in FIG. 2, in particular FIG. 2b.

Specifically, the anti-LukGH antibody is selected from the group consisting of

a) an antibody comprising
a. the HC amino acid sequence SEQ ID 231; and
b. the LC amino acid sequence SEQ ID 232;

b) an antibody comprising
a. the HC amino acid sequence SEQ ID 233; and
b. the LC amino acid sequence SEQ ID 234;

c) an antibody comprising
a. the HC amino acid sequence SEQ ID 235; and
b. the LC amino acid sequence SEQ ID 236;

d) an antibody comprising
a. the HC amino acid sequence SEQ ID 237; and
b. the LC amino acid sequence SEQ ID 238;

e) an antibody comprising
a. the HC amino acid sequence SEQ ID 239; and
b. the LC amino acid sequence SEQ ID 240;

f) an antibody comprising
a. the HC amino acid sequence SEQ ID 241; and
b. the LC amino acid sequence SEQ ID 242;

g) an antibody comprising
a. the HC amino acid sequence SEQ ID 243; and
b. the LC amino acid sequence SEQ ID 244;

h) an antibody comprising
a. the HC amino acid sequence SEQ ID 245; and
b. the LC amino acid sequence SEQ ID 246;

i) an antibody comprising
a. the HC amino acid sequence SEQ ID 247; and
b. the LC amino acid sequence SEQ ID 248;

j) an antibody comprising
a. the HC amino acid sequence SEQ ID 249; and
b. the LC amino acid sequence SEQ ID 250;

k) an antibody comprising
a. the HC amino acid sequence SEQ ID 251; and
b. the LC amino acid sequence SEQ ID 252;

l) an antibody comprising
a. the HC amino acid sequence SEQ ID 253; and
b. the LC amino acid sequence SEQ ID 254; and

m) an antibody comprising
a. the HC amino acid sequence SEQ ID 255; and
b. the LC amino acid sequence SEQ ID 256,

or a functionally active CDR variant of any of the foregoing, which has an affinity to bind the LukGH complex with a K_{D} of less than 10⁻⁸M, preferably less than 10⁻⁹M.

Specifically, the anti-LukGH antibody is an antibody of any of group member f), g) and h) above or a functionally active variant thereof, wherein

group member f): the antibody comprises
a. the HC amino acid sequence SEQ ID 241; and
b. the LC amino acid sequence SEQ ID 242;

group member g): the antibody comprises
a. the HO amino acid sequence SEQ ID 243; and
b. the LC amino acid sequence SEQ ID 244;

group member h): the antibody comprises
a. the HC amino acid sequence SEQ ID 245; and
b. the LC amino acid sequence SEQ ID 246;

and the antibody is an antibody characterized by any of the following amino acid residues:

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H;

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y;

k) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

l) in VL CDR4 at position 8, the amino acid residue is selected from S, A, D, E, F, G, H, I, K, L, M, N, Q, R, T, V, W, and Y, preferentially I or W;

m) in VL CDR4 at position 9, the amino acid residue is selected from Y, F, R and W, and preferentially R or W;

n) in VL CDR5 at position 1, the amino acid residue is selected from A, G, S, W and Y, and preferentially is G;

o) in VL CDR6 at position 4, the amino acid residue is selected from F, H, M, W and Y;

p) in VL CDR6 at position 5, the amino acid residue is selected from D, A, D, E, F, G, H, I, K, L, M, N, Q, R, S, T, V, W, and Y; and/or

q) in VL CDR6 at position 8, the amino acid residue is selected from F, H, R and W

According to a specific aspect, the anti-LukGH antibody has an affinity to bind the LukGH complex with a K_{D} of less than 10⁻⁸M, preferably less than 10⁻⁹M, or less than 10⁻¹⁰M, or less than 10⁻¹¹M, e.g. with an affinity in the picomolar range.

According to a specific aspect, the anti-LukGH antibody has an affinity to bind the individual LukG and/or LukH antigens, which are monomeric in solution, or separated from each other (not complexed in a LukGH complex).

According to a further specific aspect, According to a specific aspect, the anti-LukGH antibody has an affinity to bind the individual LukG and/or LukH antigens, which is lower than the affinity to bind the LukGH complex, preferably with a K_{D} of higher than 10⁻⁷M, preferably higher than 10⁻⁸M. In such case, the binding affinitiy is improved as compared to binding of any of or both of the separated (monomeric) LukG or LukH.

Specifically, the K_{D} difference to preferentially bind the LukGH complex over the individual LukG or LukH antigens is at least 2 logs, preferably at least 3 logs.

According to a specific aspect, the anti-LukGH antibody inhibits the binding of the LukGH complex to phosphocholine or phosphatidylcholine, in particular the phosphatidylcholine of mammalian cell membranes.

Specifically, the anti-LukGH antibody is capable of neutralizing the LukGH complex.

Specifically, the anti-LukGH antibody is cross-reactive between different LukGH variants. Specific antibodies can neutralise the LukGH variants of strain LukGH_TCH1516 (examples AB-31, AB-32-6, AB-32-9, AB-34, AB-34-14, AB-34-6 and AB-34-15), strain MRSA252 (examples AB-29-2, AB-30-3, AB-31, AB-32-6, AB-33, AB-34, AB-34-15) and strain MSHR1132 (examples AB-29-2, AB-30-3, AB-31, AB-32-6, AB-33, AB-34, AB-34-15).

Specifically, the anti-LukGH antibody is cross-neutralizing the LukGH complex and the LukGH complex variants.

Specifically, the anti-LukGH antibody is binding to the LukGH complex derived from the USA300 clone, preferably from the TCH1516 strain, and at least one of the LukGH complex variants.

Specifically, the LukGH complex variants have at least one point mutation in the amino acid sequences of any of the LukG or LukH components, as compared to the LukGH complex derived from the USA300 clone, e.g. a change in one or more of the amino acid residues in the sequence. Even the very different LukGH complex variants derived from MRSA252 and MSHR1132 strains may be cross-specifically bound by the anti-LukGH antibody as described herein, and cross-neutralized.

Specifically, the anti-LukGH antibody is a cross-neutralizing antibody comprising at least one binding site that binds to LukGH from USA300 clone (eg strain TCH_1516) and at least one of the LukGH variants. Specifically the LukGH toxin is selected from the group consisting of genes expressed by the EMRSA16 MRSA252 strain or the MSHR1132 strain.

According to a specific aspect, the anti-LukGH antibody exhibits in vitro neutralization potency in a cell-based assay with an 1050 of less than 100:1 mAb:toxin ratio (mol/mol), preferably less than 50:1, preferably less than 25:1, preferably less than 10:1, more preferably less than 1:1.

According to a further specific aspect, the anti-LukGH antibody neutralizes the targeted LukGH complex in animals, including both, human and non-human animals, and inhibits S. aureus pathogenesis in vivo, preferably any models of pneumonia, bacteremia, sepsis, abscess, skin infection, peritonitis, catheter and prothetic devices related infection and osteomyelitis.

According to a specific aspect, the anti-IGBP antibody is a monoclonal antibody that counteracts Staphylococcus aureus by specifically binding to at least one wild-type immunoglobulin-binding proteins (IGBP) of S. aureus comprising a cross-specific CDR binding site recognizing at least three of the IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity to bind SpA-E with a K_{D} of less than 5×10⁻⁹M, as determined by a standard optical interferometry method for a F(ab)2 or F(ab′)2 fragment.

Specifically, the anti-IGBP antibody is a monoclonal antibody that counteracts Staphylococcus aureus, which comprises a CDR binding site specifically binding to the wild-type SpA-E with a K_{D} of less than 5×10⁻⁹M, as determined by a standard optical interferometry method for a F(ab)2 fragment, which CDR binding site is cross-specific further recognizing at least SpA-A and SpA-D.

Specifically, the CDR binding site further recognizes at least one of the immunoglobulin-binding proteins (IGBP) of S. aureus selected from the group consisting of SpA-B, SpA-C, Sbi-I, and Sbi-II.

Specifically, the CDR binding site further recognizes at least one of SpA-B, SpA-C, Sbi-I, and Sbi-II.

According to a certain aspect, the anti-IGBP antibody has a specificity to recognize at least three of the IGBP domains, preferably at least four, five, or six of the IGBP domains, preferably which recognizes at least three of the IGBP domains each with a K_{D} of less than 5×10⁻⁹M, as determined by a standard optical interferometry method for a F(ab)2 fragment, preferably at least four or five of the IGBP each with a K_{D} of less than 5×10⁻⁹M.

Specifically, the anti-IGBP antibody recognizes at least the SpA-E, SpA-A and SpA-D, each with a K_{D} of less than 5×10⁻⁹M, as determined by a standard optical interferometry method for a F(ab)2 fragment.

According to a specific embodiment, the anti-IGBP antibody recognizes at least SpA-E, SpA-A, and SpA-D.

According to another specific embodiment, the anti-IGBP antibody recognizes at least SpA-E, SpA-A, SpA-B, and SpA-D, SpA-C, Sbi-I, and Sbi-II

According to another specific embodiment, the anti-IGBP antibody recognizes at least SpA-E, SpA-A, SpA-B, SpA-D, and Sbi-I.

According to another specific embodiment, the anti-IGBP antibody recognizes at least SpA-E, SpA-A, SpA-B, SpA-C, SpA-D, and Sbi-I.

According to another specific embodiment, the anti-IGBP antibody recognizes at least SpA-E, SpA-A, SpA-B, SpA-C, SpA-D, Sbi-I, and Sbi-II.

Specifically, the anti-IGBP antibody recognizes at least three of the IGBP domains each with a K_{D} of less than 5×10⁻⁹M, preferably at least four or five of the IGBP each with a K_{D} of less than 5×10⁻⁹M.

Specifically, the anti-IGBP antibody recognizes both, SpA and Sbi, preferably each with a K_{D} of less than 5×10⁻⁹M.

Specifically, the anti-IGBP antibody recognizes the wild-type SpA with at least substantially the same affinity or with substantially higher affinity as compared to the mutant SpA that lacks binding to IgG Fc or VH3, or as compared to the mutant SpA_{KK} or SpA_{KKAA}, preferably wherein the wild-type SpA is any of the SpA-domains comprising the sequence identified by SEQ ID 401 and optionally further comprising the sequence identified by SEQ ID 402, preferably as determined by comparing the affinity to bind the wild-type SpA-D comprising the amino acid sequence SEQ ID 394 and the mutant SpA-D_{KKAA} comprising the amino acid sequence SEQ ID 399.

According to one embodiment, the anti-IGBP antibody is capable of binding the wild-type and the mutant SpA_{KKAA} or SpA_{KK} with at least substantially the same affinity, e.g. wherein the dissociation constant ratio K_{D} (SpA_{KKAA})/K_{D} (SpA) or the ratio K_{D} (SpA_{KK})/K_{D} (SpA), e.g. as determined by the binding to the SpA-D_{KKAA} or SpA-D_{KK} compared to the SpA-D (wild-type) is at least 0.5, or at least 0.75, or about 1 or at least 1.

According to another embodiment, the anti-IGBP antibody is capable of binding the wild-type and the mutant SpA_{KKAA} or SpA_{KK} with substantially higher affinity, e.g. wherein the dissociation constant ratio K_{D} (SpA_{KKAA})/K_{D} (SpA) or the ratio K_{D} (SpA_{KK})/K_{D} (SpA), e.g. as determined by the binding to the SpA-D_{KKAA} or SpA-D_{KK} compared to the SpA-D (wild-type) is at least 2, or at least 3, or at least 4, or at least 5.

The target antigen of the anti-IGBP antibody is understood as any of the S. aureus IgG binding domains of SpA or Sbi, or a specific selection of the domains as further described herein. Specifically, at least SpA-E and at least one or two further of the IGBP domains selected from the group consisting of SpA-A, SpA-B, SpA-C, SpA-D, Sbi-I, and Sbi-II, are recognized with nanomolar or sub-nanomolar affinity.

Such monoclonal antibodies that inhibit the Fc-binding activity of SpA and Sbi are expected to enhance binding of serum IgGs to the surface antigens of S. aureus via their complementary determining regions (CDRs) rather than being inactivated by non-immune binding through their Fc region.

Specifically, the anti-IGBP antibody competes with SpA and optionally Sbi binding to IgG-Fc. Thus, the anti-IGBP antibody specifically is interfering with the IGBP binding to the IgG-Fc of IgG, i.e. inhibiting the binding or reducing the binding of the IGBP to the natural ligand IgG-Fc, thereby reducing the non-immune interaction of the IGBP with serum immunoglobulins. Specifically, the anti-IGBP antibody has a higher affinity to bind the target antigen (i.e. any of the SpA or Sbi, or respective domains) than the non-immune binding of IgG-Fc by the SpA or Sbi, e.g. as determined comparing affinities of the individual IGBP domains. The non-immune IgG-Fc binding by SpA or Sbi is specifically determined by the IgG-Fc binding region which comprises the following consensus sequence:

SEQ ID 401:
QQXAFYXXL

Wherein

X at position 3 is any of N, S, or K

X at position 7 is any of E, Q, or N, and

X at position 8 is any of I or V]

Thus, an anti-IGBP antibody as described herein may bind to the wild-type IGBP at least substantially to the same extent as to the mutant IGBP_{KK} or IGBP_{KKAA}. Specifically, anti-IGBP antibody as described herein may preferentially bind to the wild-type IGBP, e.g. preferentially binding to such consensus sequence of SEQ ID 401 (of the wild-type IGBP, included in each of the SpA and Sbi domains), and bind to the sequence of a mutant IGBP domain only to a less extent.

The IGBP mutant designated IGBP_{KK} (e.g. SpA-A_{KK}, SpA-B_{KK}, SpA-C_{KK}, SpA-D_{KK}, SpA-E_{KK}, Sbi-I_{KK}, SBi-II_{KK}) comprises the following sequence:

SEQ ID 403
KKXAFYXXL

Wherein

X at position 3 is any of N, S, or K

X at position 7 is any of E, Q, or N, and

X at position 8 is any of I or V

The IGBP mutant designated IGBP_{KKAA} (e.g. SpA-A_{KKAA}, SpA-B_{KKAA}, SPA-C_{KKAA}, SpA-D_{KKAA}, SPA-E_{KKAA}) comprises the sequences SEQ ID 403 (see above), and further comprises SEQ ID 404 as follows:

SEQ ID 404
QRNGFIQSLKAAPSXS

Wherein

X at position 15 is any of Q or V.

The respective wild-type consensus sequence comprised in each of the SpA-A, SpA-B, SpA-C, SpA-D, and SpA-E is as follows (SEQ ID 402):

SEQ ID 402
QRNGFIQSLKDDPSXS

Wherein

X at position 15 is any of Q or V.

Specifically, the anti-IGBP antibody is counteracting or neutralizing Staphylococcus aureus by enhanced opsonophagocytosis and killing by phagocytic cells. A specific test for determining this activity of the anti-IGBP antibody is to enumerate live bacteria after incubation with antibody (opsonization) followed by co-incubation with professional phagocytes such as human neutrophil granulocytes. Phagocytes take the up the opsonized pathogen via Fc-receptors which typically results in internalization and intracellular killing of the bacterium.

Specifically, the anti-IGBP antibody is cross-reactive between different SpA and Sbi variants. Specific anti-IGBP antibodies can bind to IGBP variants of at least two strains selected from the group consisting of the strains USA300 TCH1516, MSSA476, JH1, Newman strain, JH9, MW2, Mu3, MRSA252, N315, Mu50, NCTC8325, COL, and USA300_FPR3757. Specific anti-IGBP antibodies can bind the IGBP variants of at least one MSSA strain and at least one MRSA strain. Specific anti-IGBP antibodies can bind the IGBP variants of at least two strains which are MRSA strains.

According to a specific aspect, the anti-IGBP antibody exhibits neutralization potency against the virulence functions of SpA and Sbi, such as Fc and VH3 binding, binding to von Willebrand factor in an in vitro assay with an IC50 of less than 100:1 mAb:protein ratio (mol/mol), preferably less than 50:1, preferably less than 25:1, preferably less than 10:1, more preferably less than 1:1.

According to a further specific aspect, the anti-IGBP antibody binds to S. aureus in animals, including both, human and non-human animals, and inhibits S. aureus pathogenesis in vivo, preferably any models of pneumonia, bacteremia, sepsis, abscess, skin infection, peritonitis, catheter and prothetic devices related infection and osteomyelitis.

Specifically, the anti-IGBP antibody is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence. Preferably, the anti-IGBP antibody is selected from the group consisting of murine, chimeric, humanized or human antibodies, heavy-chain antibodies, Fab, F(ab′)2, Fd, scFv and single-domain antibodies like VH, VHH or VL, preferably a human IgG1 antibody, or a human antibody comprising a IgG-Fc mutation, e.g. to reduce binding of IGBP or SpA to the Fc, such as human IgG3.

Specifically, the anti-IGBP antibody comprises variable regions and/or variable domains, which comprise CDRs and a structure to bind a target antigen through the CDR antigen-binding site, and further comprises constant regions and/or constant domains, e.g. including a (human) framework, e.g. of any of full-length antibodies, heavy-chain antibodies, Fab, F(ab′)2, Fd, scFv and single-domain antibodies like VH, VHH or VL.

Specifically, the anti-IGBP antibody comprises at least an antibody heavy chain variable region (VH), which is characterized by any of the CDR1 to CDR3 sequences as listed in Table 3, and optionally an antibody light chain region (VL), which is characterized by any of the CDR4 to CDR6 sequences as listed in Table 3, which CDR sequences are designated according to the numbering system of Kabat, or functionally active CDR variants of any of the foregoing.

Specifically, the anti-IGBP antibody comprises at least an antibody heavy chain variable region (VH) and an antibody light chain region (VL), which antibody is characterized by any of the CDR1 to CDR3 sequences as listed in Table 3, and optionally further characterized by any of the CDR4 to CDR6 sequences as listed in Table 3, which CDR sequences are designated according to the numbering system of Kabat, or functionally active CDR variants of any of the foregoing.

According to specific examples, the anti-IGBP antibody comprises any of the heavy chain (HC) sequences listed in FIG. 2c. (SEQ ID 408-418), and optionally the light chain (LC) sequence SEQ ID 419.

Specifically, the antibody comprises six CDR sequences, characterized as follows:

VH CDR1:
(SEQ ID 420)
YTFXXXYXH,

wherein

X at position 4 =any of T, R, Q, P, D, E, G, S, A, M;

X at position 5 =any of 5, R, A, E, H, L, G;

X at position 6 =any of Y, L, R, H;

X at position 8 =any of I, M;

VH CDR2:
(SEQ ID 421)
XINPXXXXTXYAQKFQG,

wherein

X at position 1=any of I, W;

X at position 5=any of S, H, N, P, R, M, G;

X at position 6=any of G, V, N, S, L, Y, I, V, F;

X at position 7=any of G, D;

X at position 8=any of S, H, N, R, G;

X at position 10=any of S, H, N;

VH CDR3 is selected from the group consisting of: SEQ ID 259, SEQ ID 262, SEQ ID 265, SEQ ID 280, SEQ ID 292, SEQ ID 307, and SEQ ID 407;

VL CDR1 (CDR4):
(SEQ ID 422)
XASQXXSXXLX,

wherein

X at position 1=any of R, Q;

X at position 5=any of S, D;

X at position 6=any of V, I;

X at position 8=any of S, N;

X at position 9=any of S, Y, N;

X at position 11=any of A, N;

VL CDR2 (CDR5):
(SEQ ID 423)
XASXXXX,

wherein

X at position 1=any of G, A, D;

X at position 4=any of T, S, N;

X at position 5=any of R, L;

X at position 6=any of A, Q, E;

X at position 7=any of T, S;

and

VL CDR3 (CDR6) selected from the group consisting of: SEQ ID 319, SEQ ID 322, SEQ ID 325, SEQ ID 340, SEQ ID 343, SEQ ID 352, and SEQ ID 367.

While the anti-IGBP antibody may be provided as an antibody comprising a binding site determined by CDR sequences of the VH sequence only, e.g. a VH antibody or a heavy chain antibody, according to a specific aspect, the binding site may be further determined by CDR sequences of the antibody light chain variable region (VL), preferably which comprises any of the CDR4 to CDR6 sequences as listed in Table 3, or functionally active CDR variants thereof.

Specifically, the anti-IGBP antibody
a) comprises a VH domain, which is characterized by any of the CDR1 to CDR3 sequence combinations as listed in Table 3, and a VL domain, which is characterized by any of the CDR4 to CDR6 sequence combinations as listed in Table 3;
b) comprises the set of CDR sequences (CDR1-CDR6) of any of the antibodies as listed in Table 3;
c) is any of the antibodies as listed in Table 3; or
d) is a functionally active variant of a parent antibody that is characterized by the sequences of a)-c), preferably wherein
i. the functionally active variant comprises at least one functionally active CDR variant of any of the CDR1-CDR6 of the parent antibody; and/or
ii. the functionally active variant comprises at least one point mutation in the framework region of any of the VH and VL sequences;

and further wherein
iii. the functionally active variant has a specificity to bind the same epitope as the parent antibody; and/or
iv. the functionally active variant is a human, humanized, chimeric or murine and/or affinity matured variant of the parent antibody.

Specifically, the anti-IGBP antibody comprises a functionally active CDR variant of any of the CDR sequences as listed in Table 3, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence.

Specifically, the anti-IGBP antibody is selected from the group consisting of group members i) to vi), wherein

i)

A) the antibody comprises
a) a CDR1 consisting of the amino acid sequence of SEQ ID 269; and
b) a CDR2 consisting of the amino acid sequence of SEQ ID 270; and
c) a CDR3 consisting of the amino acid sequence of SEQ ID 271; and optionally further comprises
d) a CDR4 consisting of the amino acid sequence of SEQ ID 329; and
e) a CDR5 consisting of the amino acid sequence of SEQ ID 330; and
f) a CDR6 consisting of the amino acid sequence of SEQ ID 331;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 69;
b) the parent CDR2 consists of the amino acid sequence SEQ ID 70;
c) the parent CDR3 consists of the amino acid sequence SEQ ID 71;
d) the parent CDR4 consists of the amino acid sequence SEQ ID 329;
e) the parent CDR5 consists of the amino acid sequence SEQ ID 330;
f) the parent CDR6 consists of the amino acid sequence SEQ ID 331;

ii)

A) the antibody comprises
a) a CDR1 consisting of the amino acid sequence of SEQ ID 287; and
b) a CDR2 consisting of the amino acid sequence of SEQ ID 288; and
c) a CDR3 consisting of the amino acid sequence of SEQ ID 289;

and optionally further comprises
d) a CDR4 consisting of the amino acid sequence of SEQ ID 347; and
e) a CDR5 consisting of the amino acid sequence of SEQ ID 348; and
f) a CDR6 consisting of the amino acid sequence of SEQ ID 349;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 287;
b) the parent CDR2 consists of the amino acid sequence SEQ ID 288;
c) the parent CDR3 consists of the amino acid sequence SEQ ID 289;
d) the parent CDR4 consists of the amino acid sequence SEQ ID 347;
e) the parent CDR5 consists of the amino acid sequence SEQ ID 348;
f) the parent CDR6 consists of the amino acid sequence SEQ ID 349;

iii)

A) the antibody comprises
a) a CDR1 consisting of the amino acid sequence of SEQ ID 296; and
b) a CDR2 consisting of the amino acid sequence of SEQ ID 297; and
c) a CDR3 consisting of the amino acid sequence of SEQ ID 298;

and optionally further comprises
d) a CDR4 consisting of the amino acid sequence of SEQ ID 356; and
e) a CDR5 consisting of the amino acid sequence of SEQ ID 357; and
f) a CDR6 consisting of the amino acid sequence of SEQ ID 358;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 296;
b) the parent CDR2 consists of the amino acid sequence SEQ ID 297;
c) the parent CDR3 consists of the amino acid sequence SEQ ID 298;
d) the parent CDR4 consists of the amino acid sequence SEQ ID 356;
e) the parent CDR5 consists of the amino acid sequence SEQ ID 357;
f) the parent CDR6 consists of the amino acid sequence SEQ ID 358;

iv)

A) the antibody comprises
a) a CDR1 consisting of the amino acid sequence of SEQ ID 299; and
b) a CDR2 consisting of the amino acid sequence of SEQ ID 300; and
c) a CDR3 consisting of the amino acid sequence of SEQ ID 301;

and optionally further comprises
d) a CDR4 consisting of the amino acid sequence of SEQ ID 359; and
e) a CDR5 consisting of the amino acid sequence of SEQ ID 360; and
f) a CDR6 consisting of the amino acid sequence of SEQ ID 361;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 299;
b) the parent CDR2 consists of the amino acid sequence SEQ ID 300;
c) the parent CDR3 consists of the amino acid sequence SEQ ID 3;
d) the parent CDR4 consists of the amino acid sequence SEQ ID 359;
e) the parent CDR5 consists of the amino acid sequence SEQ ID 360;
f) the parent CDR6 consists of the amino acid sequence SEQ ID 361;

v)

A) the antibody comprises
a) a CDR1 consisting of the amino acid sequence of SEQ ID 302; and
b) a CDR2 consisting of the amino acid sequence of SEQ ID 303; and
c) a CDR3 consisting of the amino acid sequence of SEQ ID 304;

and optionally further comprises
d) a CDR4 consisting of the amino acid sequence of SEQ ID 362; and
e) a CDR5 consisting of the amino acid sequence of SEQ ID 363; and
f) a CDR6 consisting of the amino acid sequence of SEQ ID 364;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 302;
b) the parent CDR2 consists of the amino acid sequence SEQ ID 303;
c) the parent CDR3 consists of the amino acid sequence SEQ ID 304;
d) the parent CDR4 consists of the amino acid sequence SEQ ID 362;
e) the parent CDR5 consists of the amino acid sequence SEQ ID 363;
f) the parent CDR6 consists of the amino acid sequence SEQ ID 364;

and

vi)

A) the antibody comprises
a) a CDR1 consisting of the amino acid sequence of SEQ ID 314; and
b) a CDR2 consisting of the amino acid sequence of SEQ ID 315; and
c) a CDR3 consisting of the amino acid sequence of SEQ ID 316;

and optionally further comprises
d) a CDR4 consisting of the amino acid sequence of SEQ ID 374; and
e) a CDR5 consisting of the amino acid sequence of SEQ ID 375; and
f) a CDR6 consisting of the amino acid sequence of SEQ ID 376;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 314;
b) the parent CDR2 consists of the amino acid sequence SEQ ID 315;
c) the parent CDR3 consists of the amino acid sequence SEQ ID 316;
d) the parent CDR4 consists of the amino acid sequence SEQ ID 374;
e) the parent CDR5 consists of the amino acid sequence SEQ ID 375;
f) the parent CDR6 consists of the amino acid sequence SEQ ID 376.

According to a specific embodiment, the combination preparation comprises the toxin cross-neutralizing antibody, the anti-LukGH antibody and/or the anti-IGBP antibody, wherein

a) the toxin cross-neutralizing antibody comprises
a. the CDR1 sequence SEQ ID 1; and
b. the CDR2 sequence SEQ ID 2; and
c. the CDR3 sequence SEQ ID 12; and
d. the CDR4 sequence SEQ ID 32; and
e. the CDR5 sequence SEQ ID 33; and
f. the CDR6 sequence SEQ ID 34;

b) the anti-LukGH antibody comprises
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;

and

c) the anti-IGBP antibody comprises
a. the CDR1 sequence SEQ ID 299; and
b. the CDR2 sequence SEQ ID 300; and
c. the CDR3 sequence SEQ ID 301; and
d. the CDR4 sequence SEQ ID 359; and
e. the CDR5 sequence SEQ ID 360; and
f. the CDR6 sequence SEQ ID 361;

or a functionally active CDR variant of any of the foregoing, which has an affinity to bind the target antigen with a K_{D} of less than 10⁻⁸M, preferably less than 5×10⁻⁹M.

Specifically, the combination preparation comprises

a) the toxin cross-neutralizing antibody, which is any of the ASN-1 mAbs as described herein; and

b) the anti-LukGH antibody which is any of the ASN-2 mAbs as described herein.

Such combination preparation has a synergistic effect as proven in the examples section below.

Antibodies comprising the CDR sequences of AB-28 or of its variants AB-28-x, e.g., antibodies of Table 1 are herein called ASN-1. Such mAbs are neutralizing alpha-hemolysin, LukSF, LukED, HIgAB and HIgCB.

LukGH neutralizing antibodies comprising the CDR sequences of AB-29, AB-30, AB-31, AB-32, AB-33, AB-34, AB-35, and AB-36, or of variants of any of the foregoing, are herein referred to as ASN-2 mAbs, e.g., antibodies of Tables 2.1, 2.2, 2.3, 2.4, 2.5, 2.6, 2.7, or 2.8.

The antibodies used in the examples section were in particular the following:

ASN-1:

AB-28: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 3;

VL CDR4: SEQ ID 32;

VL CDR5: SEQ ID 33;

VL CDR6: SEQ ID 34.

AB-28 is specifically characterized by the following HC and LC sequence:

HC: SEQ ID 40,

LC: SEQ ID 52.

AB-28-10: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 12;

VL CDR4: SEQ ID 32;

VL CDR5: SEQ ID 33;

VL CDR6: SEQ ID 34.

AB-28-10 is specifically characterized by the following HC and LC sequences: HC: SEQ ID 48,

LC: SEQ ID 52.

AB-28-7: a mAb characterized by 6 CDR sequences as listed in Table 1.1a, 1.1b, and 1.1c:

VH CDR1: SEQ ID 5;

VH CDR2: SEQ ID 9;

VH CDR3: SEQ ID 3;

VL CDR4: SEQ ID 32;

VL CDR5: SEQ ID 33;

VL CDR6: SEQ ID 34.

AB-28-7 is specifically characterized by the following HC and LC sequences:

HC: SEQ ID 45,

LC: SEQ ID 52.

AB-28-8: a mAb characterized by 6 CDR sequences as listed in Table 1.1a, 1.1b, and 1.1c:

VH CDR1: SEQ ID 5;

VH CDR2: SEQ ID 10;

VH CDR3: SEQ ID 3;

VL CDR4: SEQ ID 32;

VL CDR5: SEQ ID 33;

VL CDR6: SEQ ID 34.

AB-28-8 is specifically characterized by the following HC and LC sequences:

HC: SEQ ID 46,

LC: SEQ ID 52.

AB-28-9: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 12;

VL CDR4: SEQ ID 32;

VL CDR5: SEQ ID 33;

VL CDR6: SEQ ID 34.

AB-28-9 is specifically characterized by the following HC and LC sequences:

HC: SEQ ID 46,

LC: SEQ ID 52.

ASN-2:

AB-30-3: a mAb characterized by 6 CDR sequences as listed in Table 2.2a, 2.2b (Group 2 mAbs):

VH CDR1: SEQ ID 122;

VH CDR2: SEQ ID 123;

VH CDR3: SEQ ID 114;

VL CDR4: SEQ ID 116;

VL CDR5: SEQ ID 117;

VL CDR6: SEQ ID 119.

AB-30-3 is specifically characterized by the following HC and LC sequences:

HC: SEQ ID 235,

LC: SEQ ID 236.

AB-31: a mAb characterized by 6 CDR sequences as listed in Table 2.3a, 2.3b (Group 3 mAbs):

VH CDR1: SEQ ID 131;

VH CDR2: SEQ ID 133;

VH CDR3: SEQ ID 135;

VL CDR4: SEQ ID 137;

VL CDR5: SEQ ID 105;

VL CDR6: SEQ ID 138.

AB-31 is specifically characterized by the following HC and LC sequences:

HC: SEQ ID 239,

LC: SEQ ID 240.

AB-34: a mAb characterized by 6 CDR sequences as listed in Table 2.6a, 2.6b (Group 6 mAbs):

VH CDR1: SEQ ID 188;

VH CDR2: SEQ ID 189;

VH CDR3: SEQ ID 190;

VL CDR4: SEQ ID 176;

VL CDR5: SEQ ID 178;

VL CDR6: SEQ ID 192.

AB-34 is specifically characterized by the following HC and LC sequences:

HC: SEQ ID 249,

LC: SEQ ID 250.

AB-34-6: a mAb characterized by 6 CDR sequences as listed in Table 2.6a, 2.6b (Group 6 mAbs):

VH CDR1: SEQ ID 198;

VH CDR2: SEQ ID 199;

VH CDR3: SEQ ID 190;

VL CDR4: SEQ ID 200;

VL CDR5: SEQ ID 201;

VL CDR6: SEQ ID 202.

AB-34-6 is specifically characterized by the following HC and LC sequences:

HC: SEQ ID 253,

LC: SEQ ID 254.

AB-32-9: a mAb characterized by 6 CDR sequences as listed in Table 2.4a, 2.4b (Group 4 mAbs):

VH CDR1: SEQ ID 167;

VH CDR2: SEQ ID 168;

VH CDR3: SEQ ID 157;

VL CDR4: SEQ ID 159;

VL CDR5: SEQ ID 125;

VL CDR6: SEQ ID 160.

AB-32-9 is specifically characterized by the following HO and LC sequences:

HC: SEQ ID 245,

LC: SEQ ID 246.

According to the examples described herein, any of the mAbs designated AB-28, AB-28-10, AB-28-7, AB-28-8, or AB-28-9, was combined with any of the mAbs designated AB-30-3, AB-31, AB-32-9, AB-34-6, or AB-34.

According to a specific aspect, the combination preparation comprises

a) the toxin cross-neutralizing antibody;

b) the anti-LukGH antibody; and

c) the anti-IGBP antibody.

According to another specific aspect, the combination preparation comprises the toxin cross-neutralizing antibody and the anti-LukGH antibody, without the anti-IGBP antibody.

According to another specific aspect, the combination preparation comprises the toxin cross-neutralizing antibody and the anti-IGBP antibody, without the anti-LukGH antibody.

Specifically, any or each of the toxin cross-neutralizing antibody, the anti-LukGH antibody, and the anti-IGBP antibody is an isolated antibody, in particular a monoclonal antibody.

Specifically, each of the toxin cross-neutralizing antibody, the anti-LukGH antibody, or the anti-IGBP antibody has an affinity to bind the target antigen, with a K_{D} of less than 10⁻⁸M, preferably less than 5×10⁻⁹M, or less than 10⁻⁹M.

The target antigen of the toxin cross-neutralizing antibody is understood as the HIa and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF, or a specific selection as further described herein. Specifically, at least 2, 3 or 4 different toxin molecules, preferably HIa, HIgB, LukF and LukD, are recognized with nanomolar or sub-nanomolar affinity.

A specific embodiment employs the toxin cross-neutralizing antibody recognizing the cytotoxins HIa, LukSF, HIgAB, HIgCB, and LukED.

The target antigen of the anti-LukGH antibody is understood as the LukGH complex. The anti-LukGH antibody is specifically recognizing the epitope formed by assembly of the individual LukG and LukH toxins in solution, thus, an epitope of the LukGH heterodimer. Specifically, the target antigen is recognized with nanomolar or sub-nanomolar affinity, while the affinity to bind any of the individual LukG or LukH is lower than the affinity to bind the LukGH complex, e.g. with a K_{D} of higher than 10⁻⁷M, preferably higher than 10⁻⁶M.

A specific embodiment employs the toxin neutralizing combination recognizing the cytotoxins HIa, LukSF, HIgAB, HIgCB, LukED and LukGH, by the toxin cross-neutralizing antibody recognizing the cytotoxins HIa, LukSF, HIgAB, HIgCB, and LukED; and the anti-LukGH antibody.

The target antigen of the anti-IGBP antibody is understood as any of the S. aureus IgG binding domains of Protein A or Sbi, or a specific selection of the domains as further described herein. Specifically, at least SpA-E and at least two further of the IGBP domains selected from the group consisting of SpA-A, SpA-B, SpA-C, SpA-D, Sbi-I, and Sbi-II, are recognized with nanomolar or sub-nanomolar affinity. Specifically, the antibody is targeting both IgG binding proteins of S. aureus, the SpA and Sbi.

Such monoclonal antibodies that inhibit the Fc-binding activity of SpA and Sbi are expected to enhance binding of serum IgGs to the surface antigens of S. aureus via their complementary determining regions (CDRs) rather than being inactivated by the non-immune binding through their Fc region.

According to a specific embodiment, any or each of the toxin cross-neutralizing antibody, the anti-LukGH antibody, or the anti-IGBP antibody is a full-length monoclonal antibody, an antibody fragment thereof comprising at least one antibody domain incorporating the binding site, or a fusion protein comprising at least one antibody domain incorporating the binding site.

The invention further provides for the medical use of the combination preparation, and the respective method of treatment or method of manufacturing a preparation for medical use.

Specifically, the combination preparation is provided for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

The invention further provides for a pharmaceutical preparation comprising the combination preparation, preferably comprising a parenteral or mucosal formulation, optionally containing a pharmaceutically acceptable carrier or excipient.

Specifically, the pharmaceutical preparation is provided as a mixture of the antibodies in one formulation, or as kit of parts, wherein at least one of the antibodies is provided in a separate formulation.

The invention further provides for a kit for preparing a pharmaceutical preparation, comprising at least the following components in a pharmaceutically acceptable formulation as separate components, e.g. in two or three containments:

a) the toxin cross-neutralizing antibody;

b) the anti-LukGH antibody; and/or

c) the anti-IGBP antibody,

in particular the component a) and at least one of or both of the components b) or c).

Any or each of the components is particularly comprising the respective antibody in the isolated form.

Such kit may be used for preparing a pharmaceutical preparation of the invention, or for medical use, including e.g. the respective method of treatment or method of manufacturing a preparation for medical use.

Specifically, the kit is provided for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

Specifically, the individual antibodies or kit components are administered to the subject concomitantly, in parallel and/or consecutively, or in a mixture.

Specifically, the combination preparation, the pharmaceutical preparation or the kit is provided for protecting against pathogenic S. aureus or against S. aureus infections.

Specifically, the combination preparation, the pharmaceutical preparation or the kit may contain the toxin cross-neutralizing antibody, the anti-LukGH antibody, and/or the OPK antibody, such as the anti-IGBP antibody, as sole active substances, or in combination with other active substances, or a cocktail of active substances, such as a combination or cocktail to administer further antibodies, e.g. further targeting S. aureus, e.g. an OPK antibody or an antibody targeting at least one other toxin. Specifically, a cocktail of antibodies comprises one or more antibodies as described herein in a mixture, and optionally further active substances.

Each individual antibody may be provided by a dose in the same range, such as from 5 to 40 mg/kg for each antibody, e.g. in a 1:1 ratio.

A series of antibodies is herein described as exemplary antibodies as listed in FIGS. 1 and 2, including antibodies of the examples. It is understood that those exemplary antibodies and functionally active variants are included in the subject of the present claims, including, but not limited to, CDR variants, FR variants, murine, chimeric, humanized or human variants, or any antibody domain combination other than a combination composed of the VH and VL or the HC and LC as described herein, e.g. an antibody comprising the same CDR1-6 or VH/VL combination, yet, with different FR sequences.

Herein described are specific functionally active CDR variants of VH or VL sequences or of HC or LC sequences, wherein any of the CDR 1-6 sequences is a functionally active CDR variant of a parent CDR, comprising at least one point mutation in the parent CDR and at least 60% sequence identity, or at least 70%, at least 80%, or at least 90% sequence identity.

In certain aspects, the invention also provides for such variant antibodies, comprising the respective binding sequences, such as the variable sequences and/or the CDR sequences, as derived from any of the exemplary antibodies, which are used as parent antibodies, wherein the binding sequences or the CDR comprises a sequence that has at least 60%, preferably at least 70%, or at least 80%, or at least 90%, or at least 95%, or at least 99% identity to the amino acid sequence as derived from the parent antibodies, and wherein the variant is a functionally active variant.

Any of the exemplary antibodies may be used as parent antibodies to produce functionally active antibody variants of such parent antibodies, wherein the functional activity is determined, if the target antigen is bound with high affinity, e.g. with a K_{D} of less than 10⁻⁸M, preferably less than any of 5×10⁻⁹ M, 4×10⁻⁹ M, 3×10⁻⁹ M, 2×10⁻⁹ M, 10⁻⁹ M, 5×10⁻¹⁰ M, 4×10⁻¹⁰ M, 3×10⁻¹⁰ M, 2×10⁻¹⁰ M, or less than 10⁻¹⁰ M, and/or the binding of the variant antibody to the target antigen competes with the binding by the parent antibody, or the variant antibody binds to the same epitope as the parent antibody.

Exemplary variant antibodies may be mutated to delete a C-terminal lysine, and/or substitute an N-terminal glutamine to glutamate, e.g. to obtain a HC sequence which is characterized by the respective point mutation, herein referred to as Q1EΔK variant.

It is known that recombinantly expressed antibodies are prone to a series of post-translational modifications, among which the pyroglutamate formation, particularly when glutamine is present at the N-terminus of the heavy chain, and cleavage of the C-terminal lysine residue, also from the heavy chain (Liu H, Ponniah G, Zhang H M, Nowak C, Neill A, Gonzalez-Lopez N, Patel R, Cheng G, Kita A Z, Andrien B. In vitro and in vivo modifications of recombinant and human IgG antibodies. MAbs. 2014; 6(5):1145-54). Therefore, for selected antibodies expressed in CHO cells, the N-terminus glutamine is mutated to glutamate, and the C-terminal lysine is removed, to avoid sample heterogeneity, giving Q1EΔK variants

Functionally active variant antibodies may differ in any of the VH or VL sequences, or share the common VH and VL sequences, and comprise modifications in the respective FR. The variant antibody derived from the parent antibody by mutagenesis may be produced by methods well-known in the art.

Exemplary parent antibodies are described in the examples section below and in FIGS. 1 and 2. Specifically, the preparation as described herein may include a functionally active derivative of a parent antibody as listed in FIG. 1 or 2. Variants with one or more modified CDR sequences, and/or with one or more modified FR sequences, such as sequences of FR1, FR2, FR3 or FR4, or a modified constant domain sequence may be engineered.

CDR combinations may be used as listed in FIG. 1 or different CDR combinations, in particular combining CDR sequences of the same group of antibodies, provided, that the antibody is still functionally active.

Specifically, an antibody as described herein comprises the CDR1-6 of any of the antibodies as listed in FIG. 1. However, according to an alternative embodiment, an antibody may comprise different CDR combinations, e.g. wherein an antibody as listed in FIG. 1 (Table 1, or any of the Tables 2, Group 1-8, or Table 3) comprises at least one CDR sequence, such as 1, 2, 3, 4, 5, or 6 CDR sequences of one antibody and at least one further CDR sequence of a different antibody of any of the antibodies as listed in FIG. 1 (Table 1, or any of the Tables 2, Group 1-8, or Table 3), in particular combining CDR sequences of antibodies listed within the same table or Group. According to a specific example, the antibody comprises 1, 2, 3, 4, 5, or 6 CDR sequences, wherein the CDR sequences are CDR combinations of more than 1 antibody, e.g. 2, 3, 4, 5, or 6 different antibodies. For example, the CDR sequences may be combined to preferably comprise 1, 2, or all 3 of CDR1-3 of any of the antibodies as listed in FIGS. 1, and 1, 2, or all 3 of CDR4-6 of the same or any other antibody listed in FIG. 1.

It is herein specifically understood that the CDRs numbered CDR1, 2, and 3 represent the binding region of the VH domain, and CDR4, 5, and 6 represent the binding region of the VL domain.

According to a specific aspect, an antibody as described herein comprises any of the HC and LC amino acid sequence combinations as depicted in FIG. 2, or the binding site formed by such combination of HC and LC amino acid sequences. Alternatively, combinations of the immunoglobulin chains of two different antibodies may be used, provided, that the antibody is still functionally active. For example, the HC sequence of one antibody may be combined with an LC sequence of another antibody, in particular wherein the HC and LC combination is originating from only one of FIG. 2a, FIG. 2b, or 2c. According to further specific embodiments, any of the framework regions as provided in FIG. 1 or 2 may be employed as a framework to any of the CDR sequences and/or VH/VL combinations as described herein.

Any of FIG. 2a, 2b, or 2c show one or more groups of HC sequences with similarities in any of the CDR1, 2, and/or 3, and one or more groups of LC sequences with similarities in any of the CDR4, 5, and/or 6, and supports any HC/LC combination, in particular wherein the HC and LC are of the same group of antibodies, wherein one of the CDR1-3 of one HC, e.g. CDR1 is combined with any other CDR sequence of a second and optionally a third HC, e.g. CDR2 and CDR3 of a second and a third HC, respectively; and wherein one of the CDR4-6 of one LC, e.g. CDR4 is combined with any other CDR sequence of a second and optionally a third LC, e.g. CDR5 and CDR6 of a second and a third LC, respectively.

FIG. 2b shows 8 groups of antibodies (identified in Table 2) characterized by different HC and/or LC sequences with similarities in any of the CDR in each of the groups, and supports any HC/LC combination, in particular a combination of a HC and a LC of the same group.

In particular, the toxin cross-neutralizing antibody may comprise a combination of any of VH/VL of Table 1, or a combination of any of HC and LC of FIG. 2a.

In particular, the anti-LukGH antibody may comprise a combination of any of VH/VL of Table 2 or any of Groups 1-8 of Table 2, or a combination of any of HC and LC of FIG. 2b, or a combination of VH and VL or a combination of HC and LC, each originating from the same Group of any of Groups 1-8.

In particular, the anti-IGBP antibody may comprise a combination of any of VH/VL of Table 3.

Specifically, the functionally active variant differs from a parent antibody, e.g. any of the antibodies as listed in FIG. 1, in at least one point mutation in the amino acid sequence. Specifically, the at least one point mutation is any of an amino acid substitution, deletion and/or insertion of one or more amino acids.

Specifically, the CDR sequence may include at least one point mutation such as to obtain a functionally active CDR variant, e.g. wherein the number of point mutations in each of the CDR amino acid sequences is either 0, 1, 2 or 3.

Specifically, the antibody is derived from such antibodies, employing the respective CDR sequences, or CDR mutants, including functionally active CDR variants, e.g. with 1, 2 or 3 point mutations within one CDR loop, e.g. within a CDR length of 5-18 amino acids, e.g. within a CDR region of 5-15 amino acids or 5-10 amino acids. Alternatively, there may be 1 to 2 point mutations within one CDR loop, e.g. within a CDR length of less than 5 amino acids, to provide for an antibody comprising a functionally active CDR variant. Specific CDR sequences might be short, e.g. the CDR2 or CDR5 sequences. According to a specific embodiment, the functionally active CDR variant comprises 1 or 2 point mutations in any CDR sequence consisting of less than 4 or 5 amino acids.

Specific antibodies are provided as CDR mutated antibodies, e.g. to improve the affinity of an antibody e.g. by affinity maturation, and/or to target the same epitope or epitopes near the epitope that is targeted by a parent antibody (epitope shift).

According to a specific aspect, an antibody as described herein comprises CDR and framework sequences, wherein at least one of the CDR and framework sequences includes human, humanized, chimeric, murine or affinity matured sequences, preferably wherein the framework sequences are of an IgG antibody, e.g. of an IgG1, IgG2, IgG3, or IgG4 subtype, or of an IgA1, IgA2, IgD, IgE, or IgM antibody.

Specific antibodies are provided as framework mutated antibodies, e.g. to improve manufacturability or tolerability of a parent antibody, e.g. to provide an improved (mutated) antibody which has a low immunogenic potential, such as humanized antibodies with mutations in any of the CDR sequences and/or framework sequences as compared to a parent antibody.

Accordingly, any of the antibodies as listed in FIG. 1 or 2 may be used as parent antibodies to engineer improved versions.

It is understood that an antibody as described herein optionally comprises such amino acid sequences of FIG. 1 or 2, with or without the respective signal sequence, or with alternative signal or leader sequences.

According to a specific aspect, each of the sequences of FIG. 1 or 2 may be terminally extended or deleted in the constant region, e.g. a deletion of one or more or the C-terminal amino acids.

Specifically, any of the antibodies described herein is a full-length monoclonal antibody, an antibody fragment thereof comprising at least one antibody domain incorporating the binding site, or a fusion protein comprising at least one antibody domain incorporating the binding site. Preferably, the antibody is selected from the group consisting of murine, chimeric, humanized or human antibodies, heavy-chain antibodies, Fab, Fd, scFv and single-domain antibodies like VH, VHH or VL, preferably a human IgG1 antibody.

The invention further provides for an anti-Staphylococcus aureus antibody preparation comprising one or more antibodies specifically recognizing the S. aureus targets:

a) alpha-toxin (HIa) and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and at least one of b), c), or d), below:

b) any of the LukG or LukH as individual targets, or the LukGH complex; and/or

c) an S. aureus IgG binding domain of SpA or Sbi or an IGBP; and/or

d) any S. aureus surface protein to bind an antibody thereby inducing OPK;

preferably wherein the preparation comprises at least one antibody which is a polyspecific antibody and at least one antibody which is a monospecific antibody.

Thus, the antibody preparation makes use of combining immunotherapeutics recognizing a series of selected targets, e.g. by a combination of monospecific antibodies, or by using at least one polyspecific antibody and optionally further comprising one or more monospecific antibodies.

According to a specific embodiment, the OPK target may be any of the IGBP targets, e.g., a Protein A antibody.

According to another embodiment, the OPK target may be employed as an alternative to targeting the IGBPs.

Specifically, any surface protein that is accessible to bind to an antibody to induce OPK of S. aureus (in particular an antibody with OPK activity) is a suitable target as described herein in combination with the other toxin targets. Thus, according to a specific embodiment, the antibody preparation is specifically targeting any S. aureus surface protein to bind an antibody thereby inducing OPK.

According to a specific embodiment, the surface protein is targeted by an antibody having OPK activity which is combined with a toxin cross-neutralizing antibody and optionally further combined with the anti-LukGH antibody.

According to a specific embodiment, the surface protein is targeted by an antibody having OPK activity which is combined with a toxin cross-neutralizing antibody and optionally further combined with the anti-IGBP antibody.

According to a specific embodiment, the surface protein is targeted by an antibody having OPK activity which is combined with a toxin cross-neutralizing antibody, and further combined with the anti-LukGH antibody and the anti-IGBP antibody.

### FIGURES

FIG. 1:

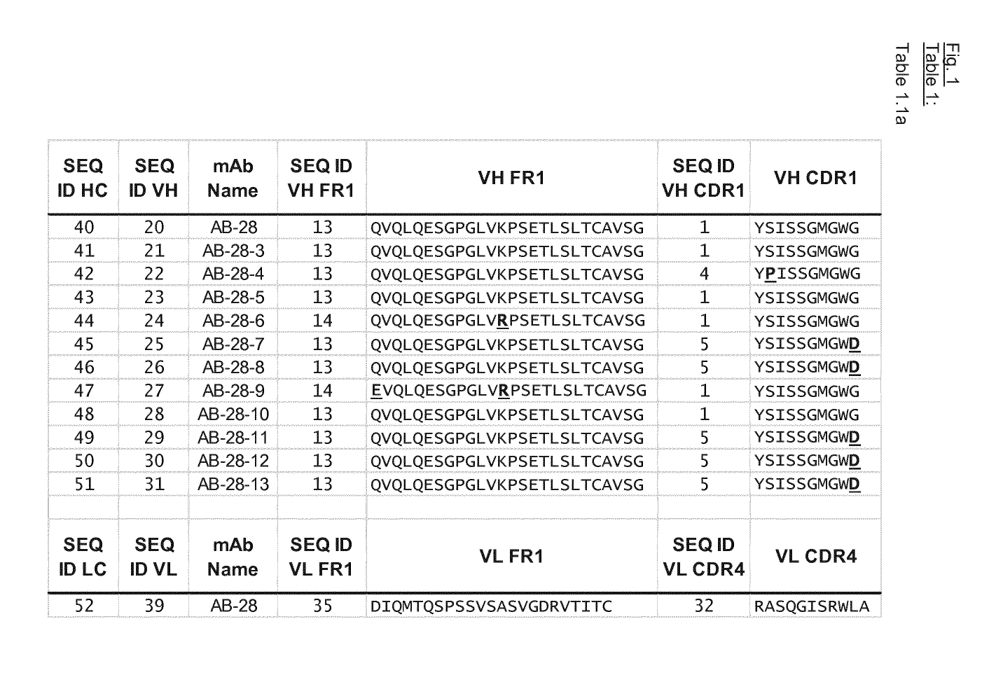

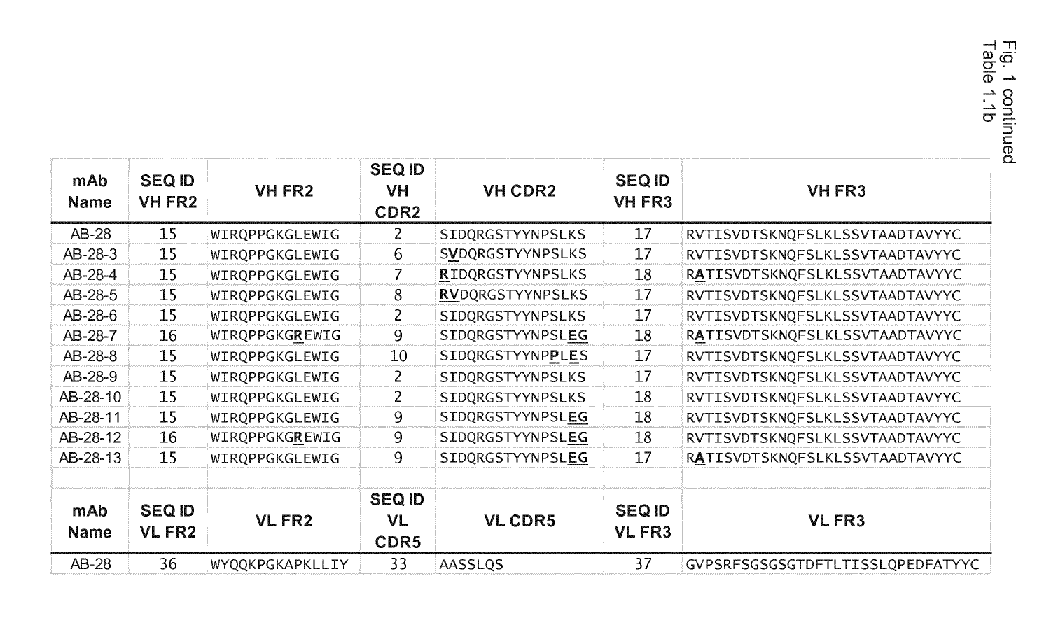

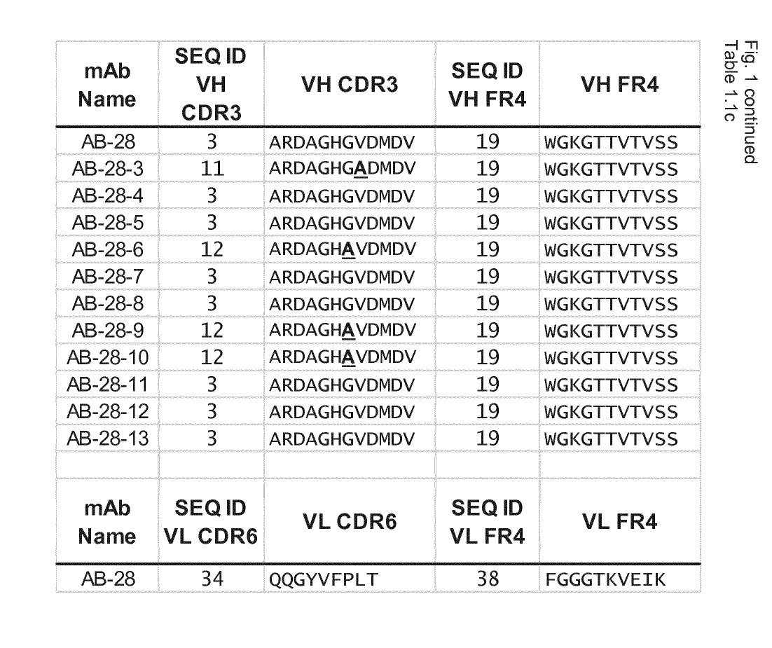

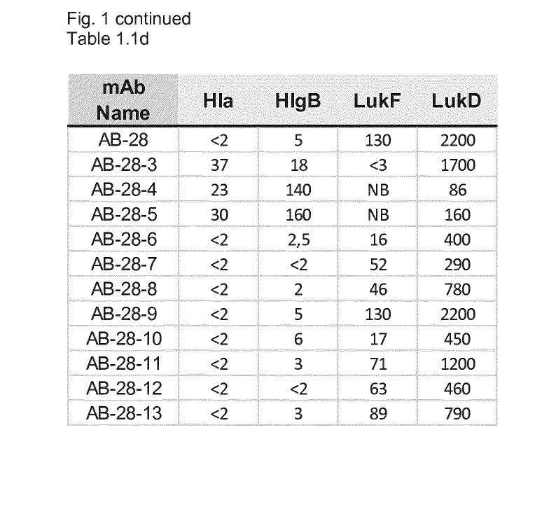

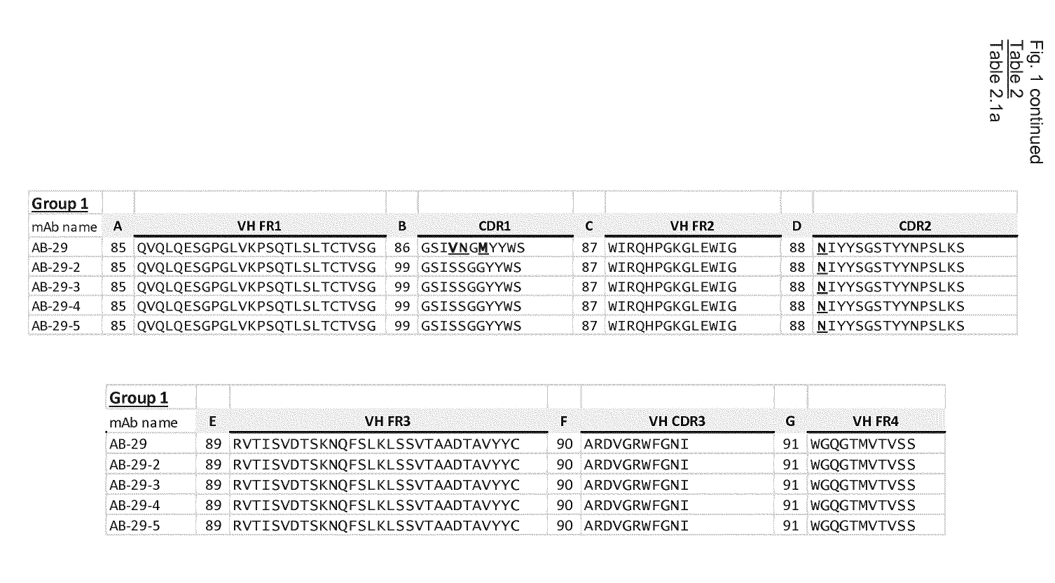

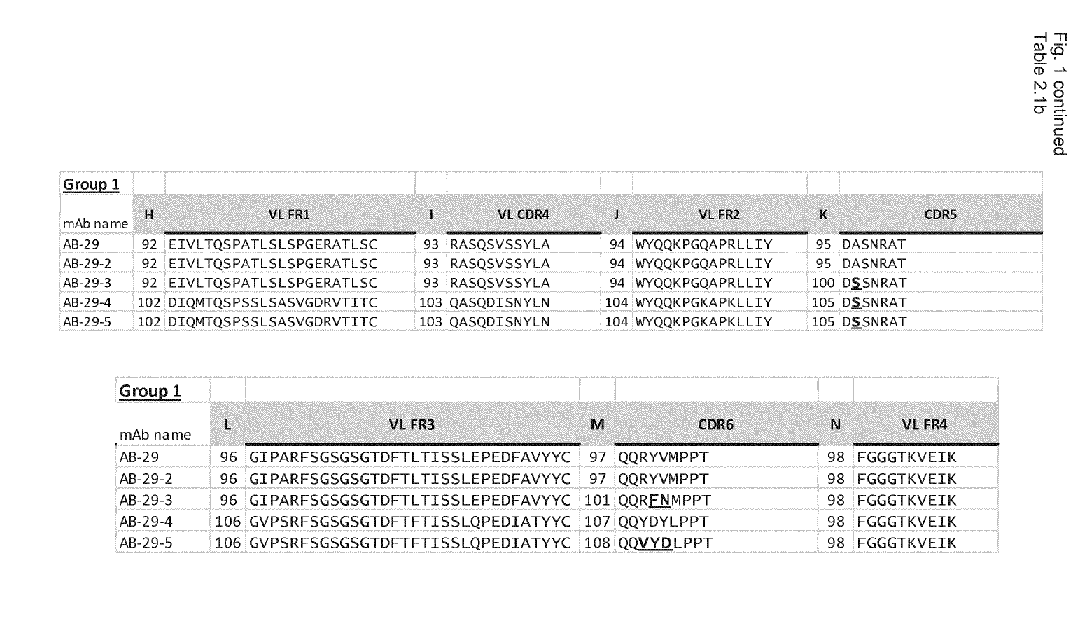

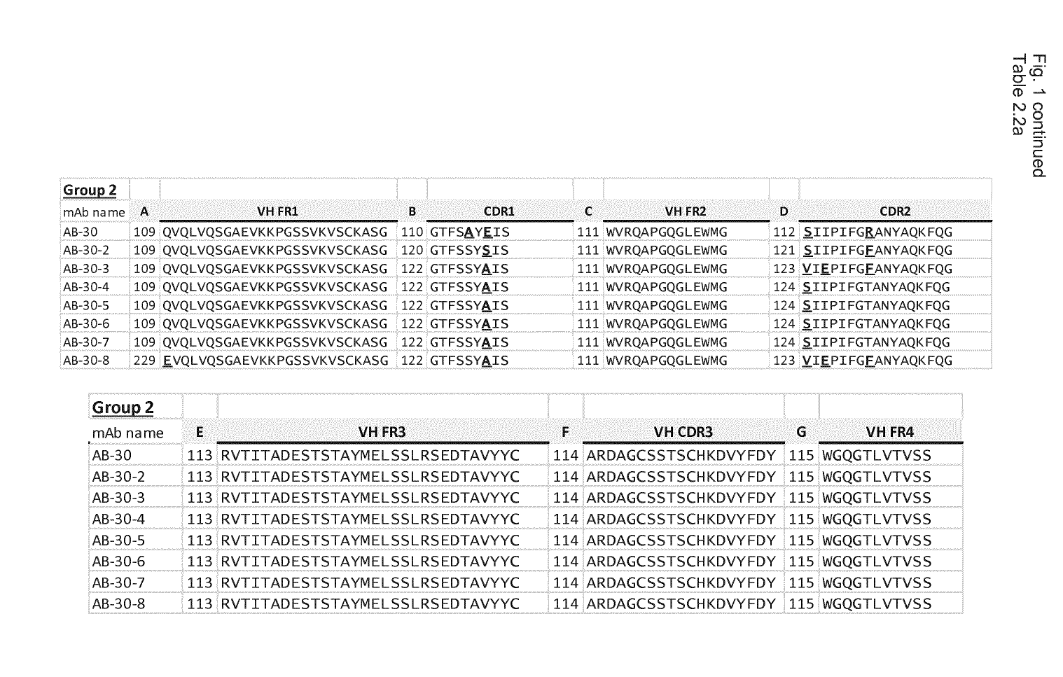

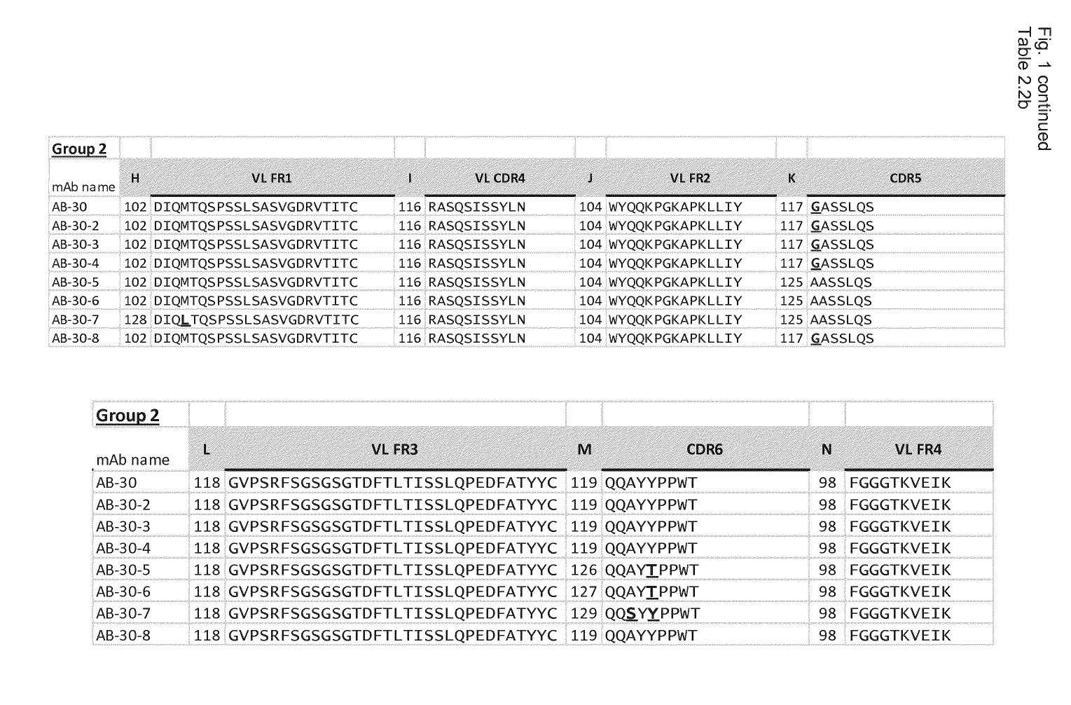

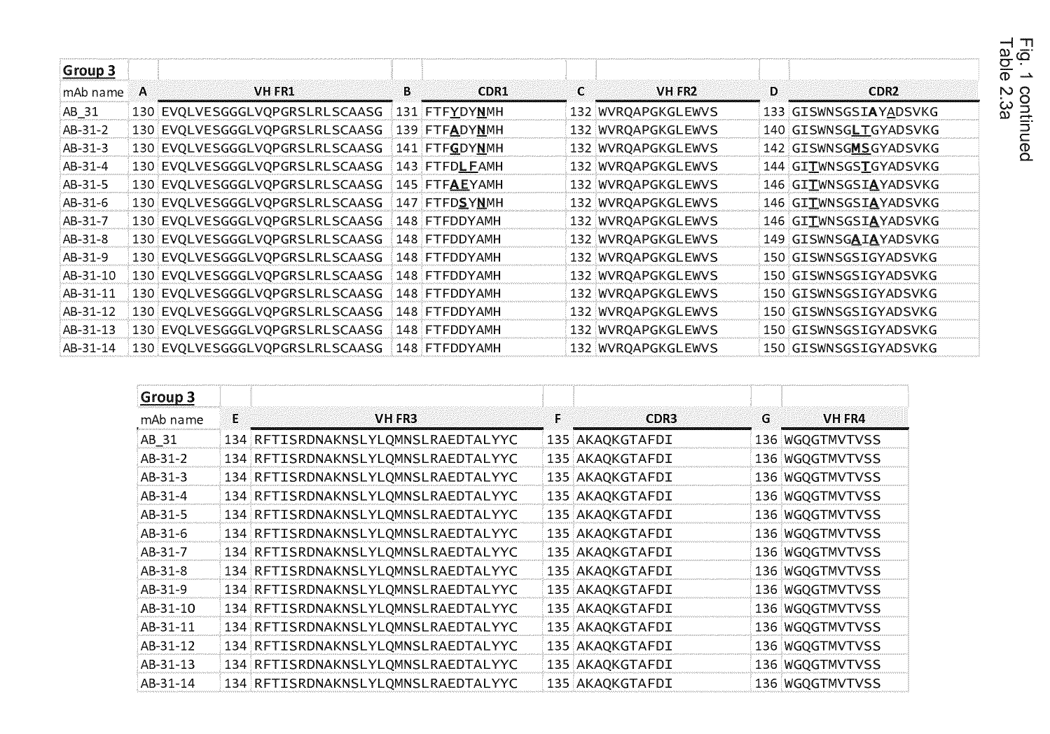

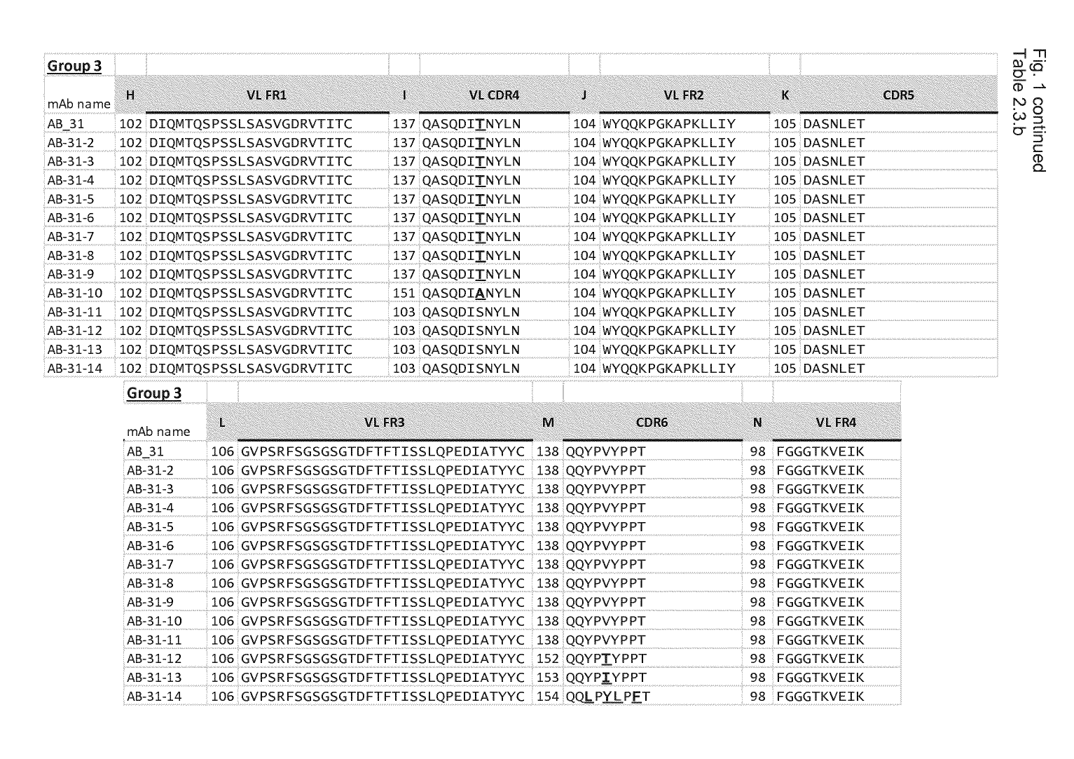

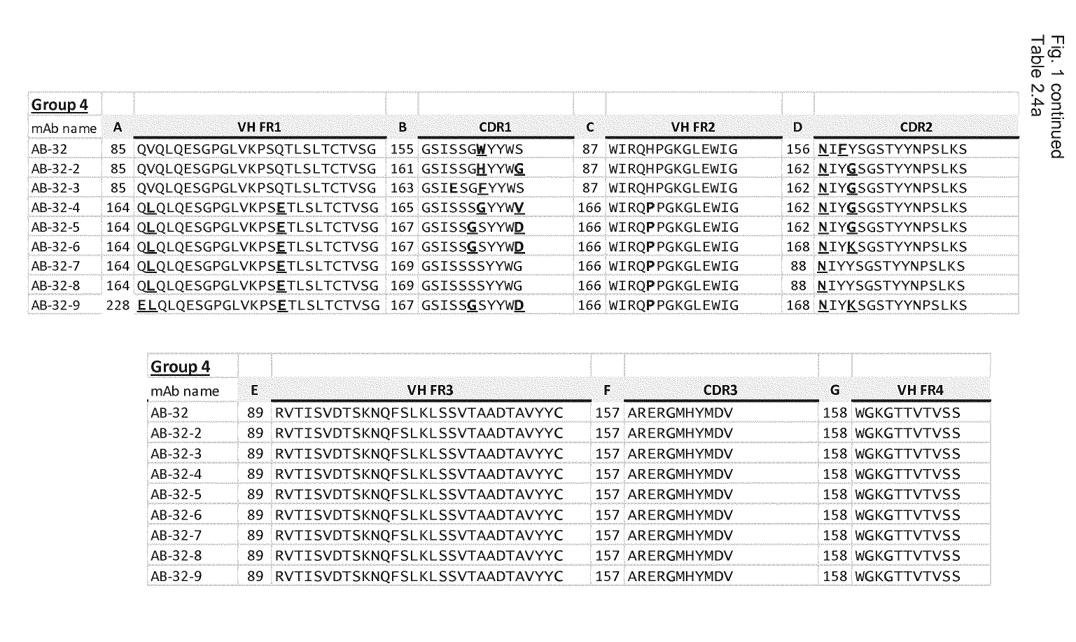

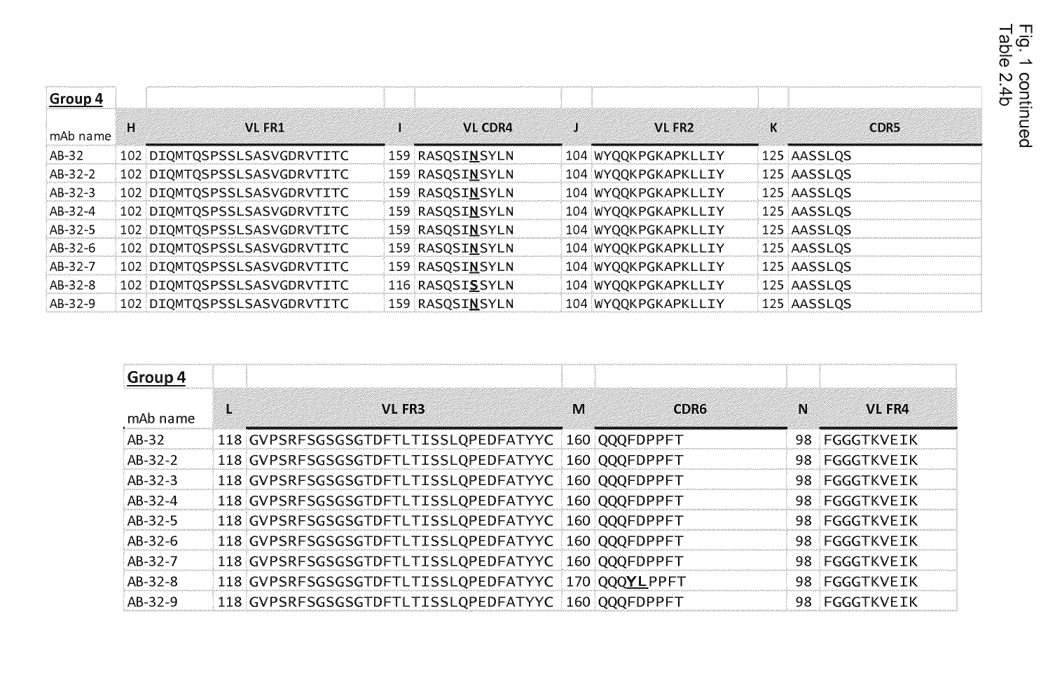

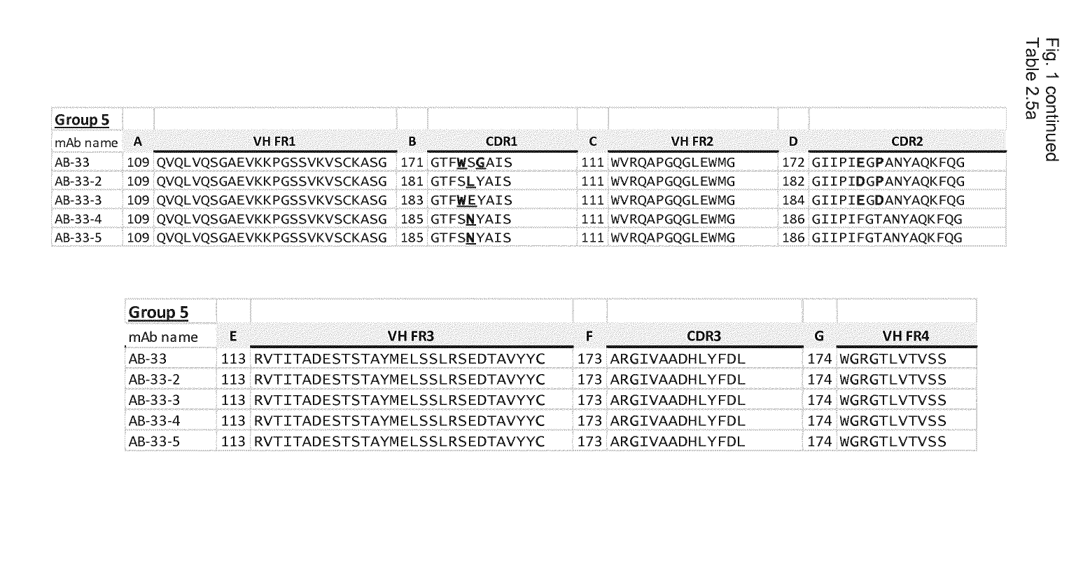

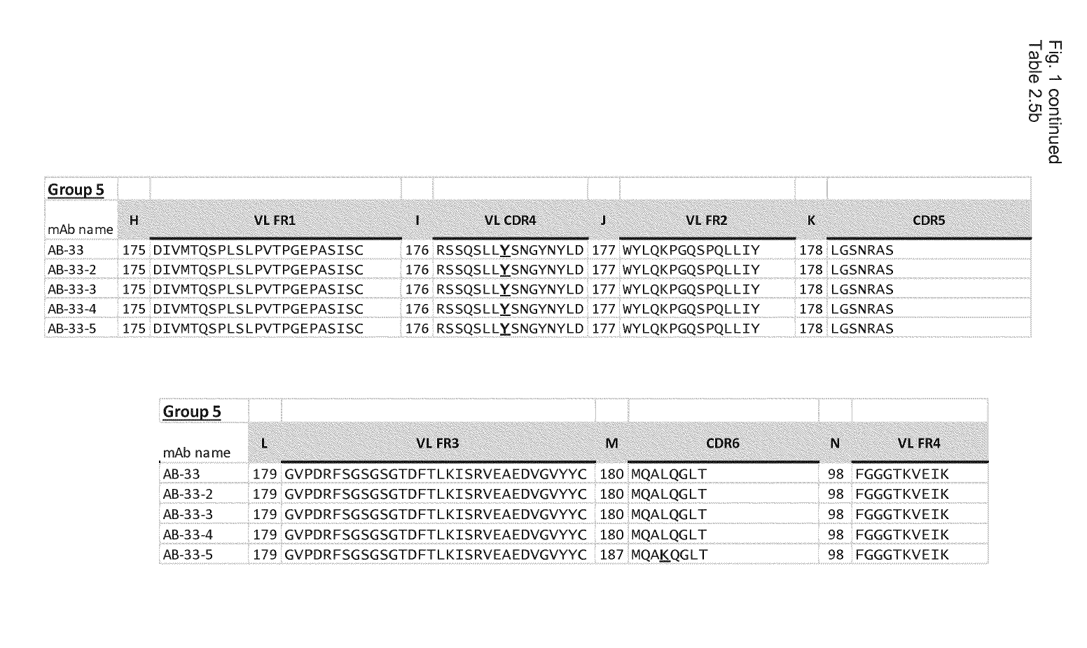

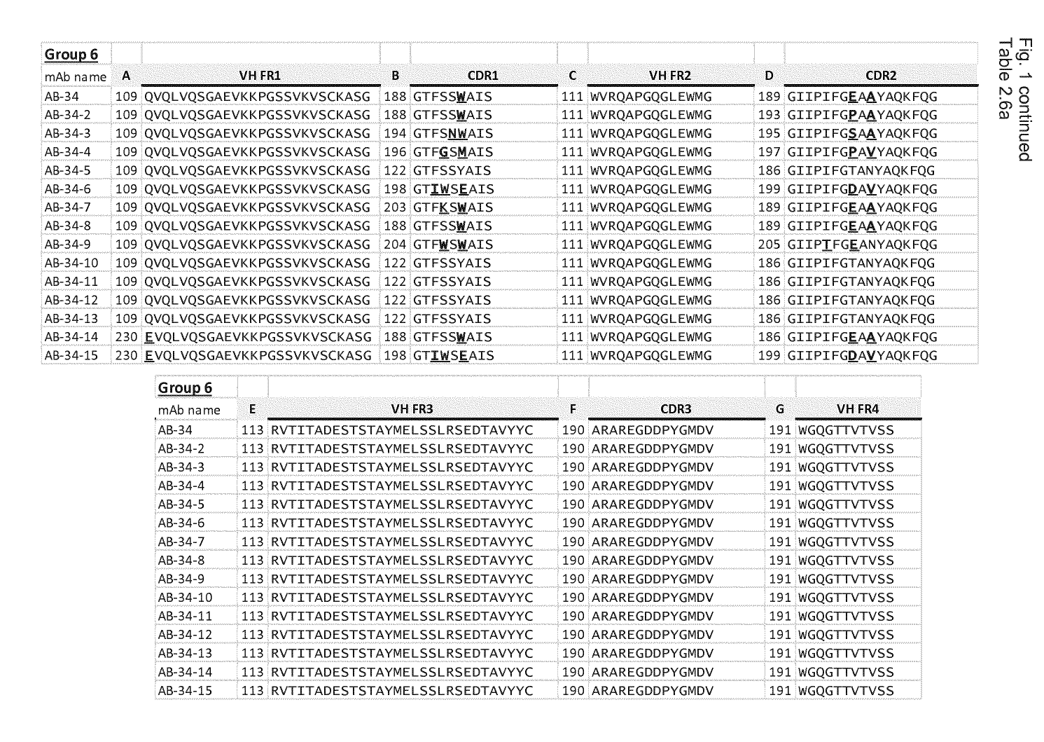

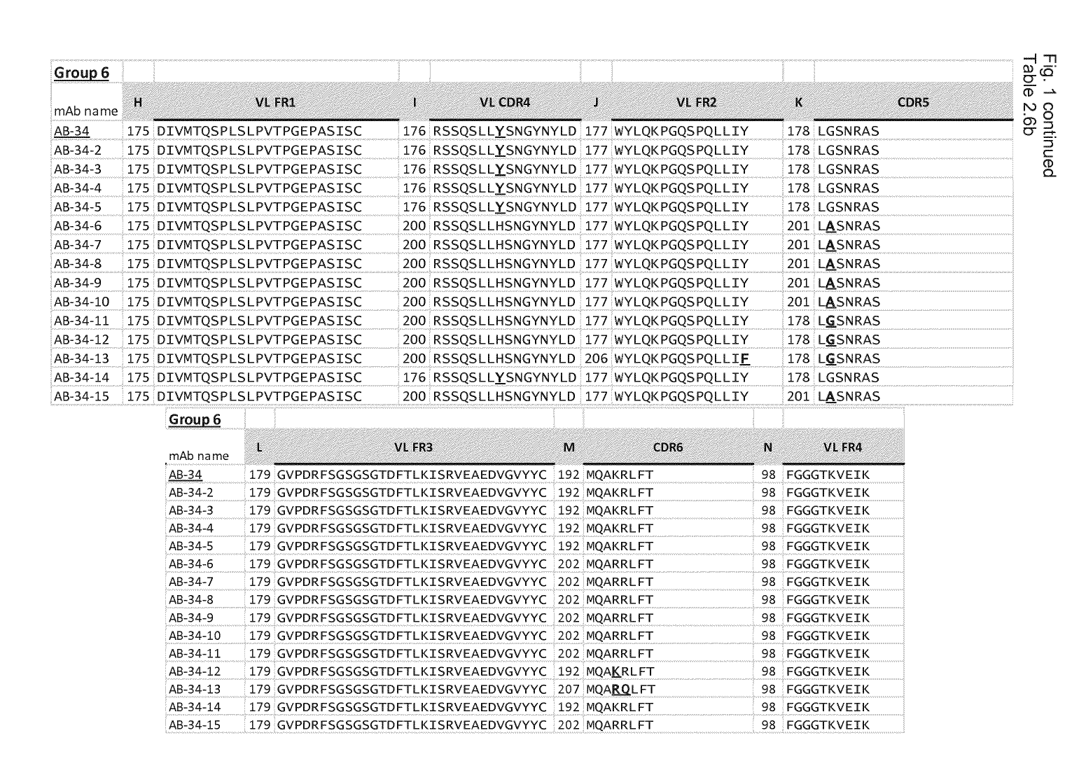

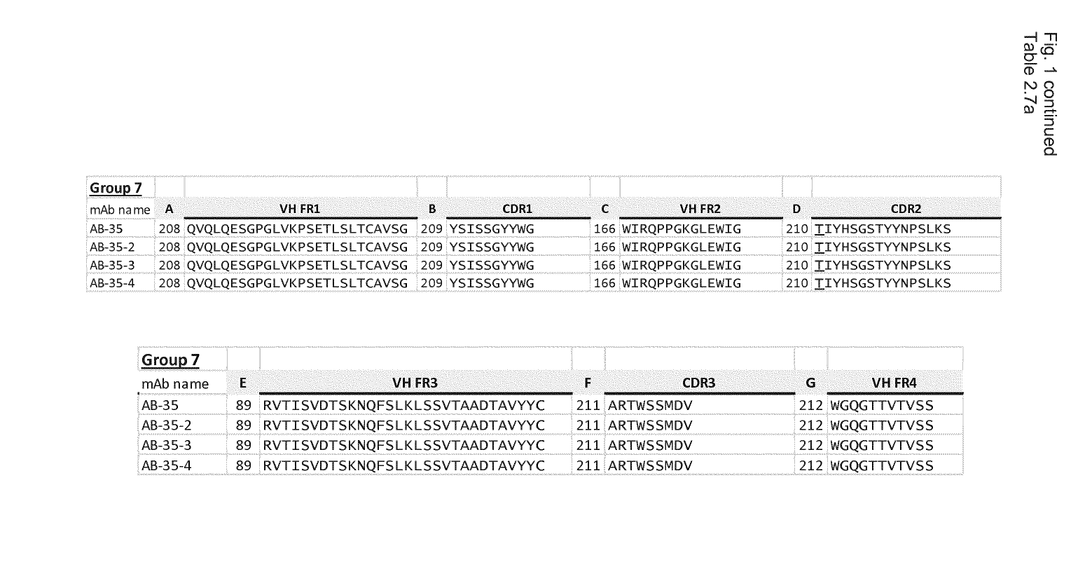

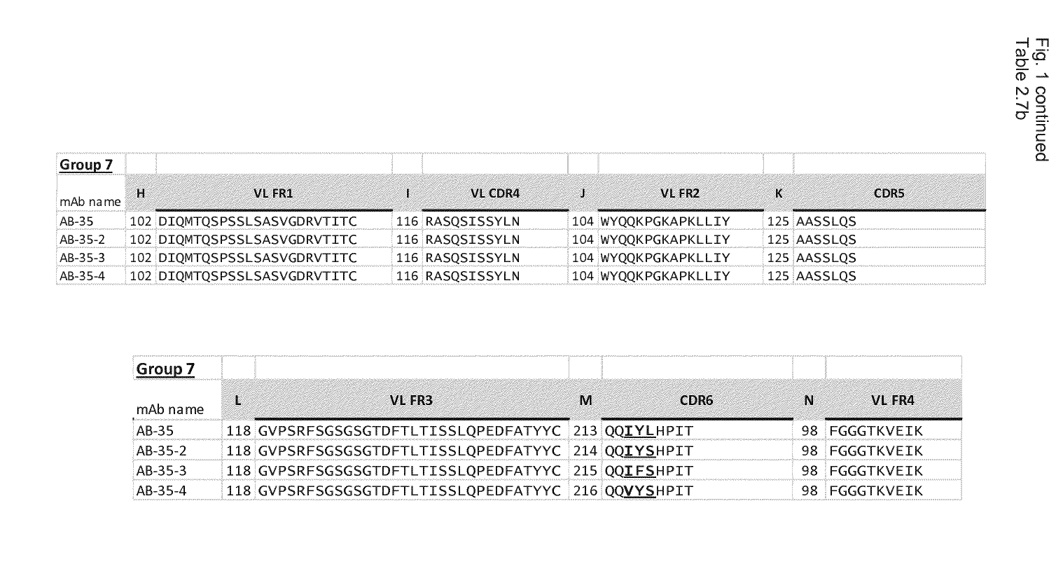

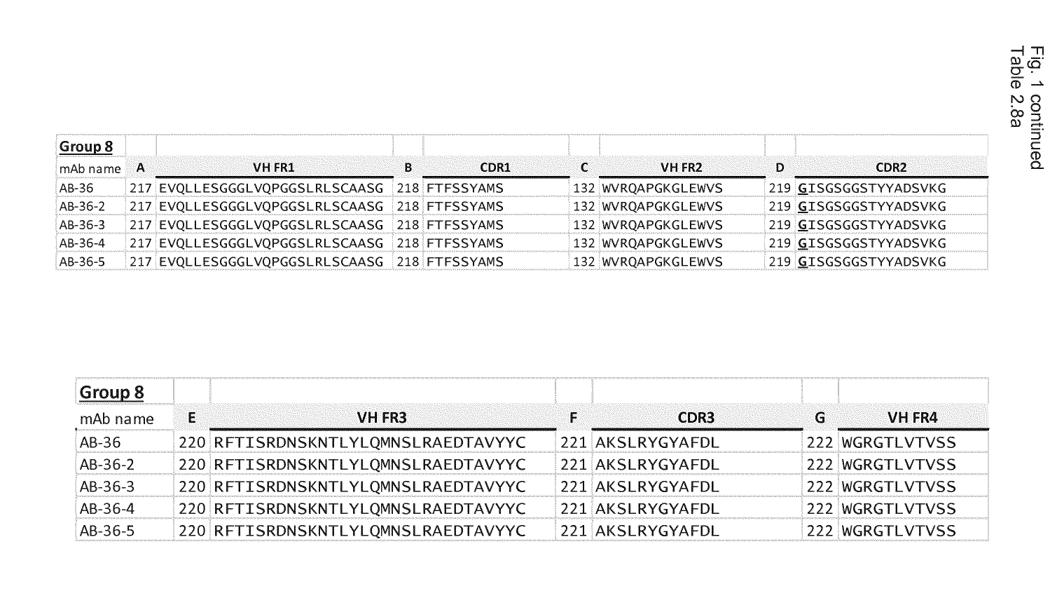

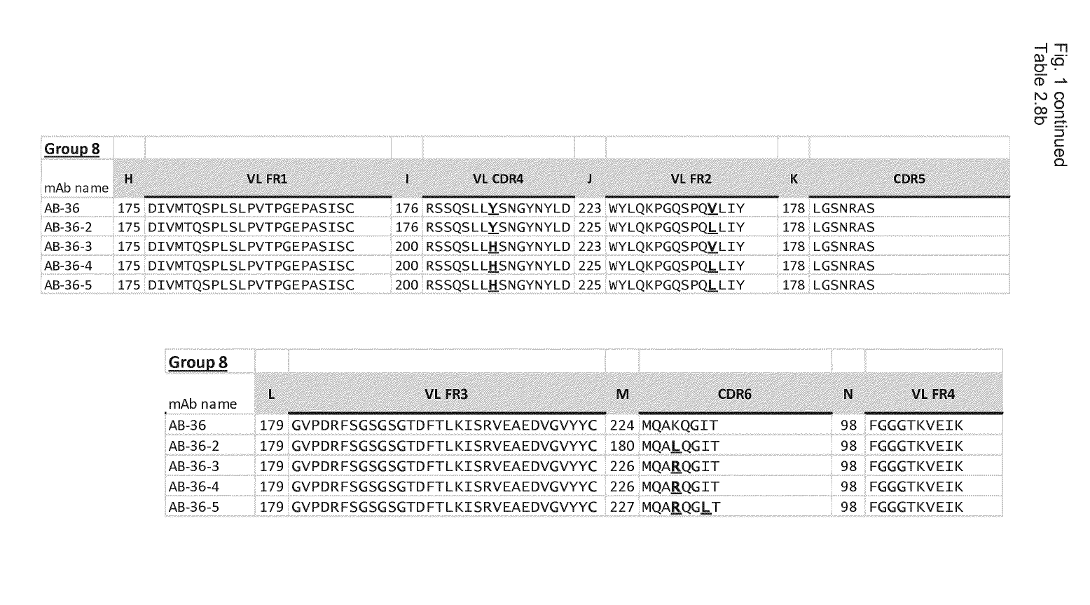

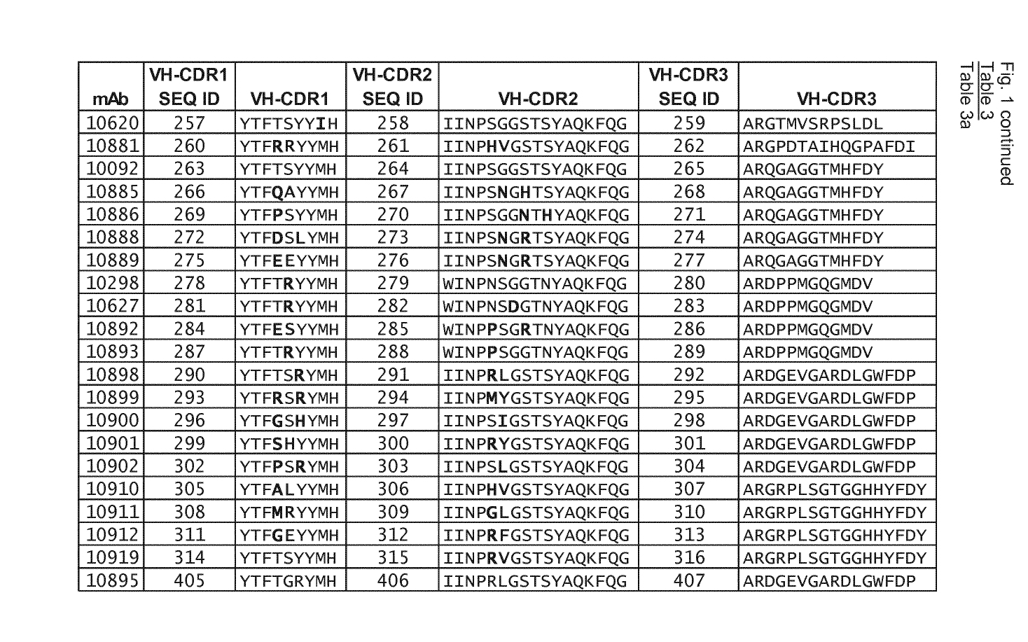

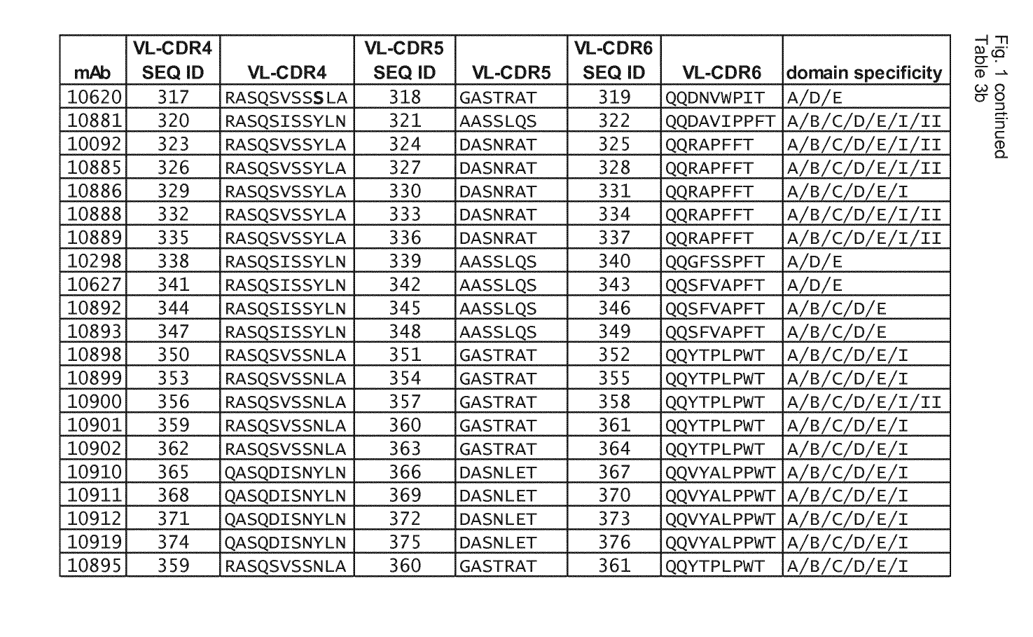

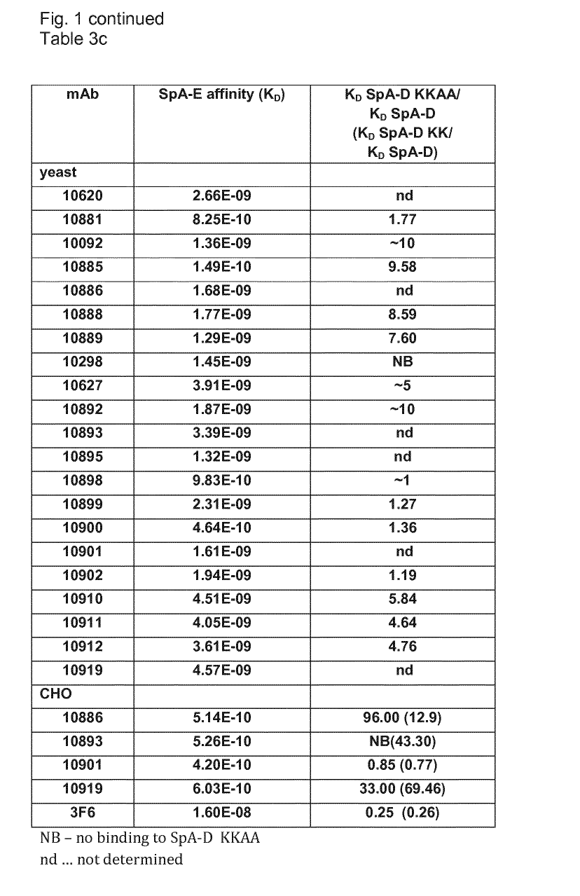

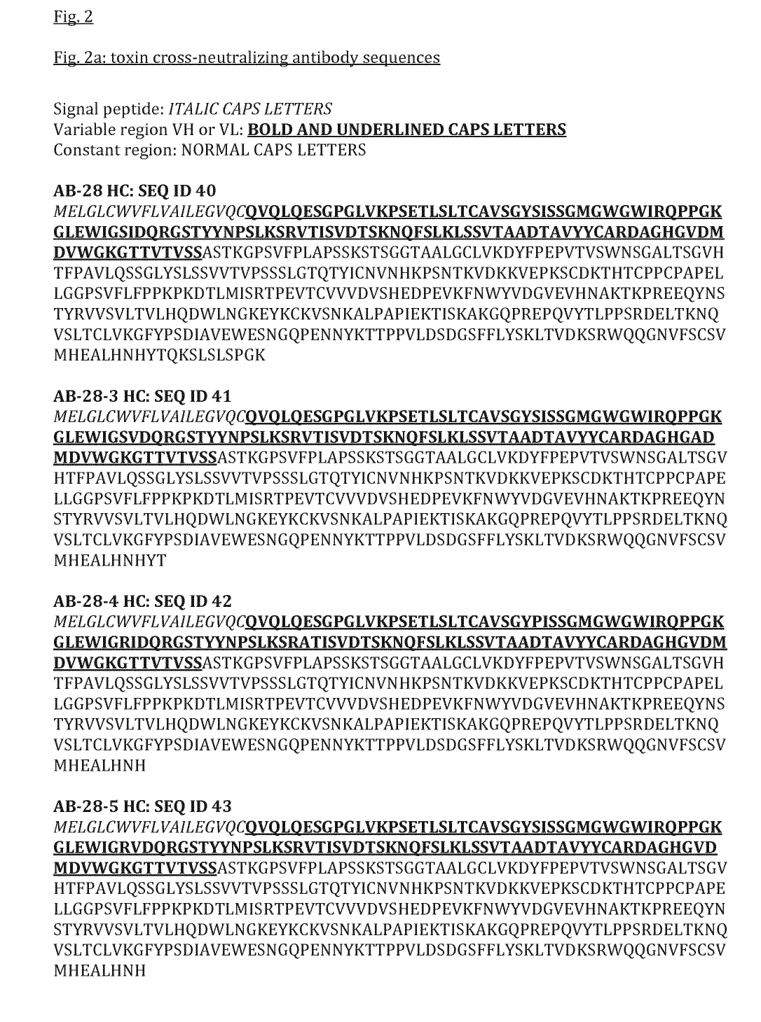

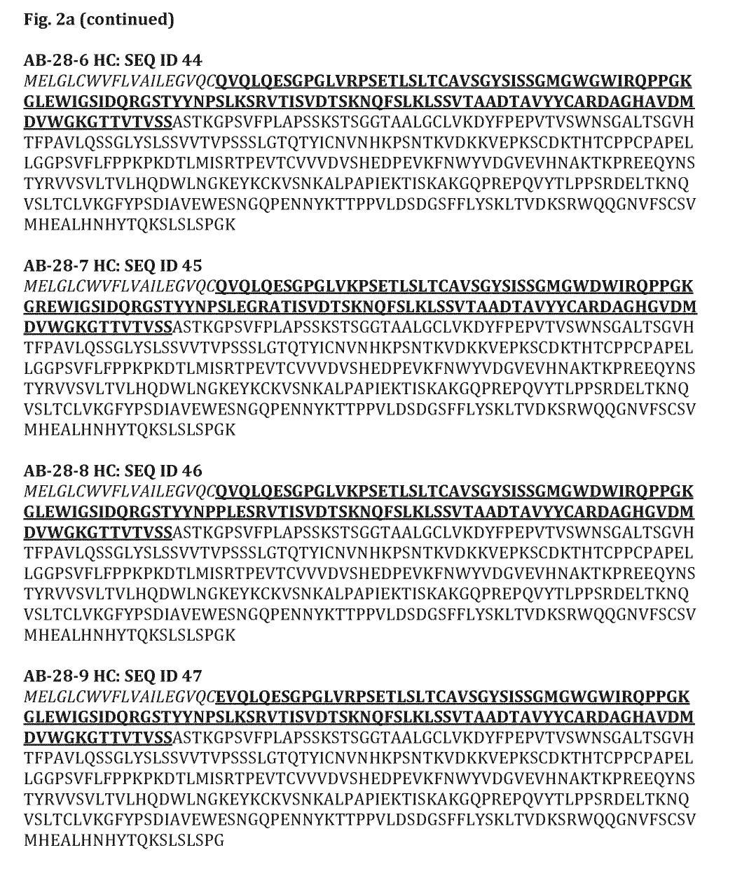

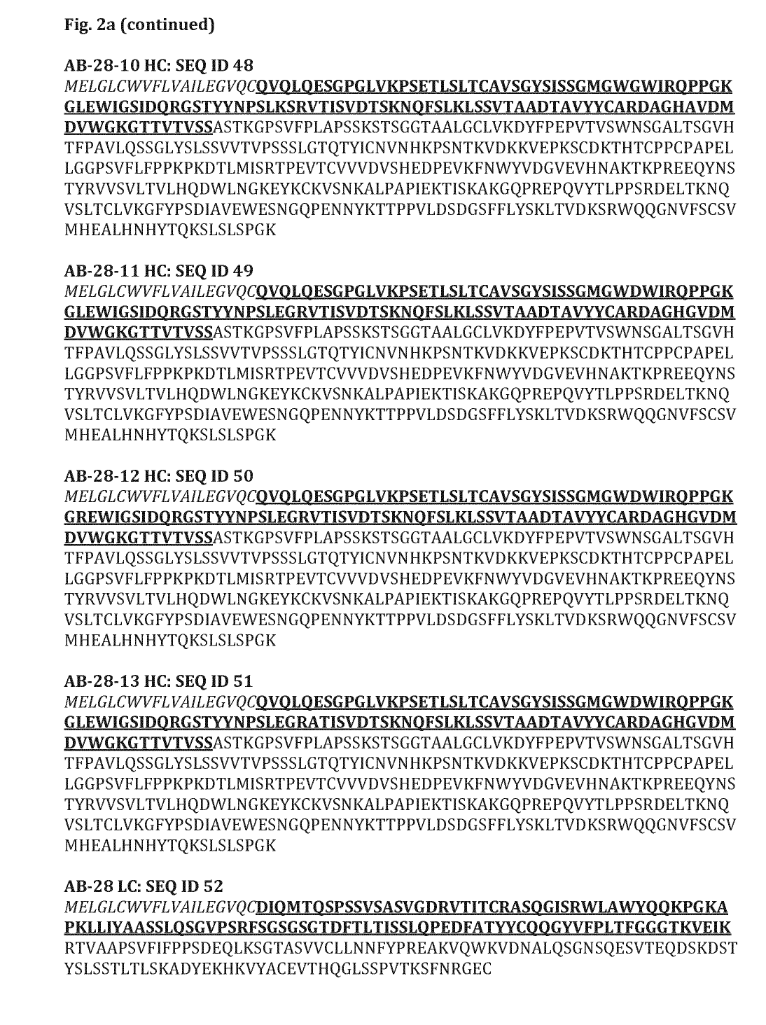

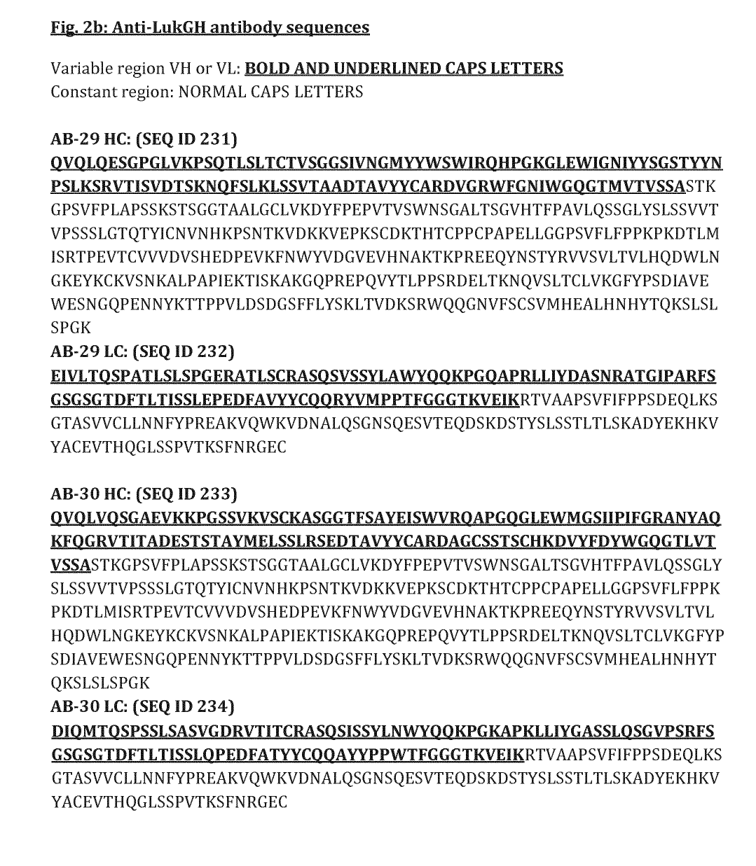

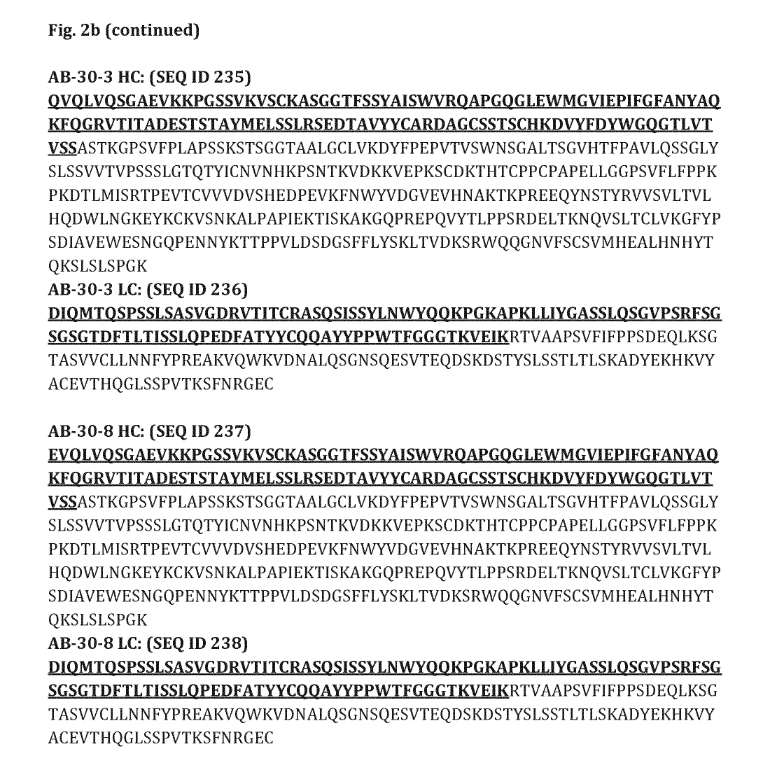

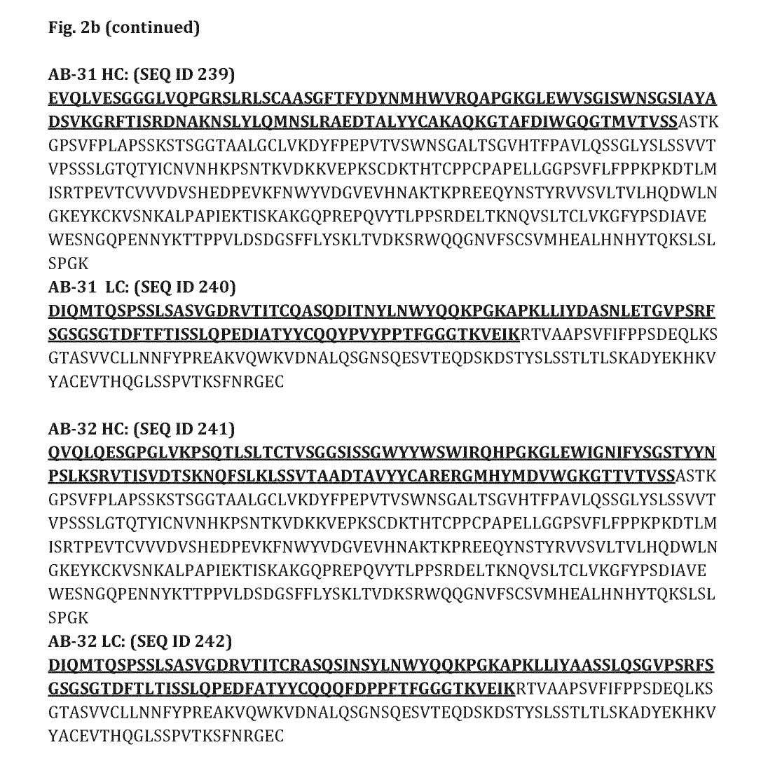

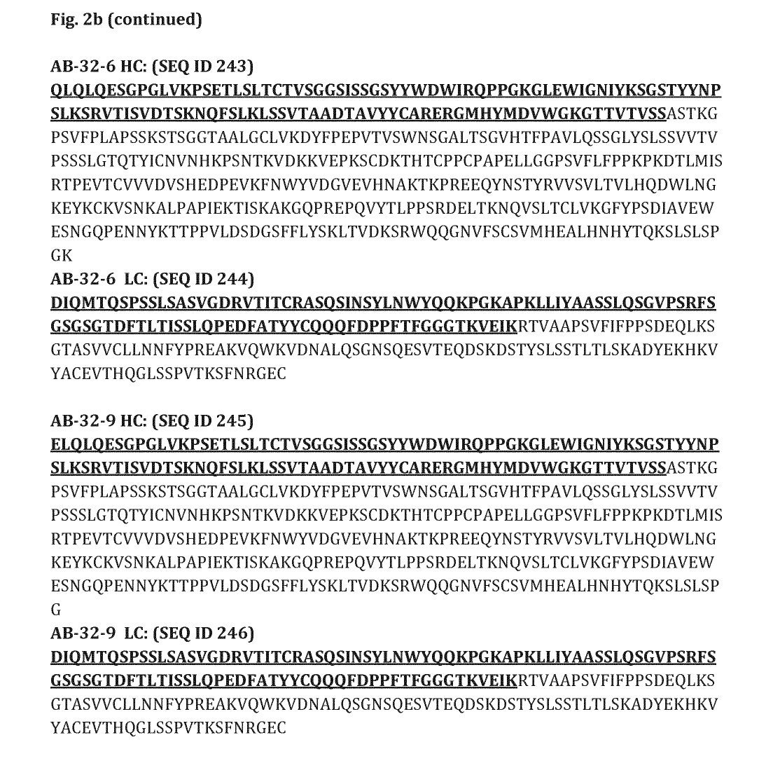

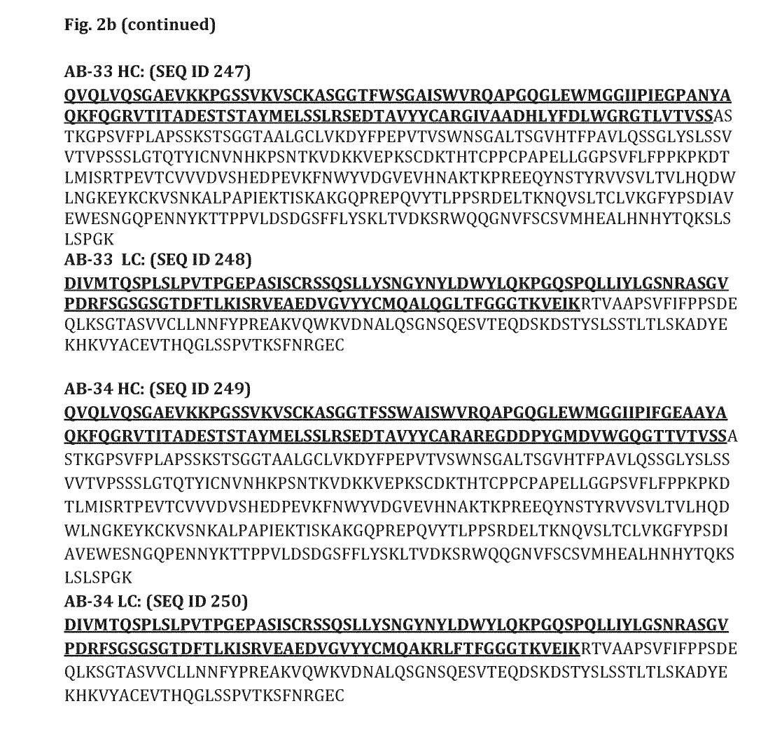

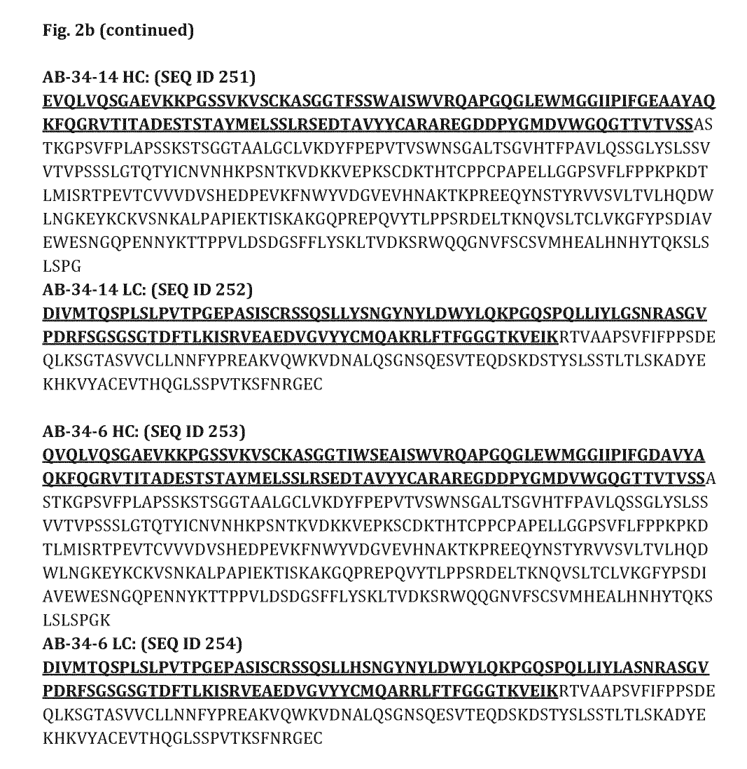

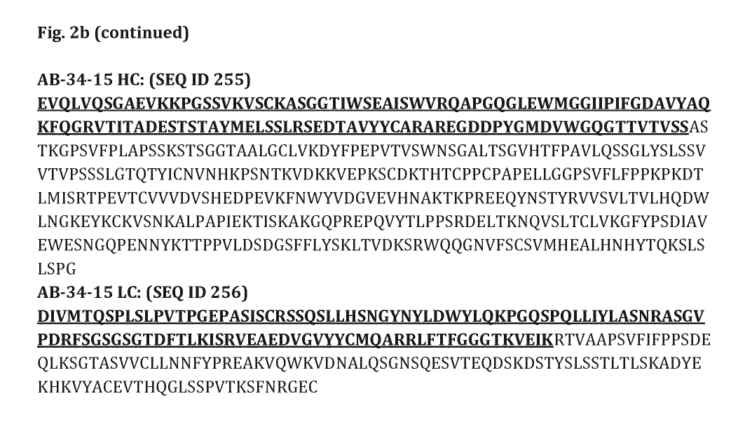

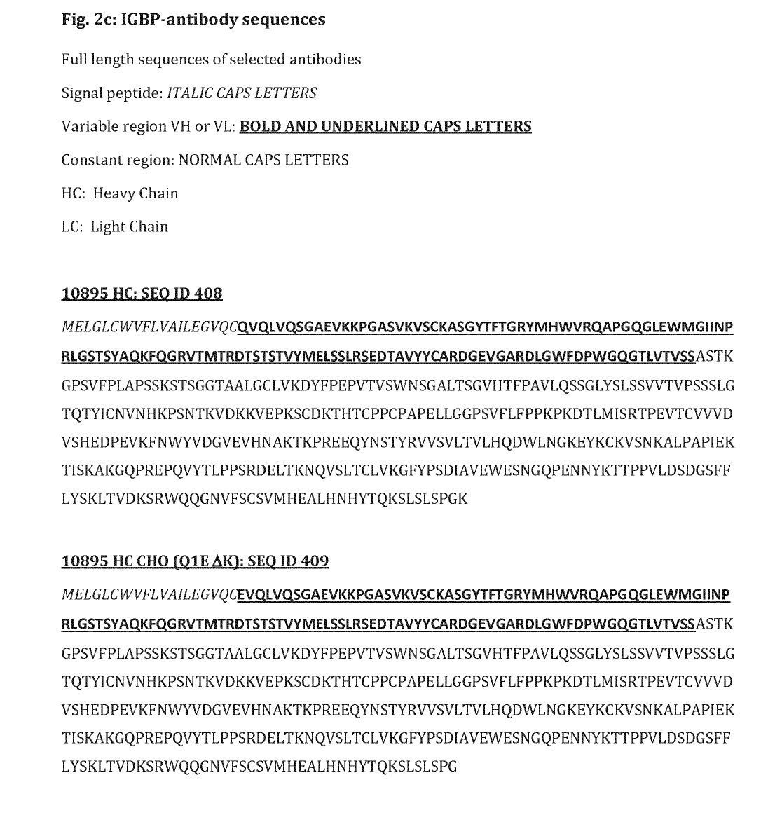

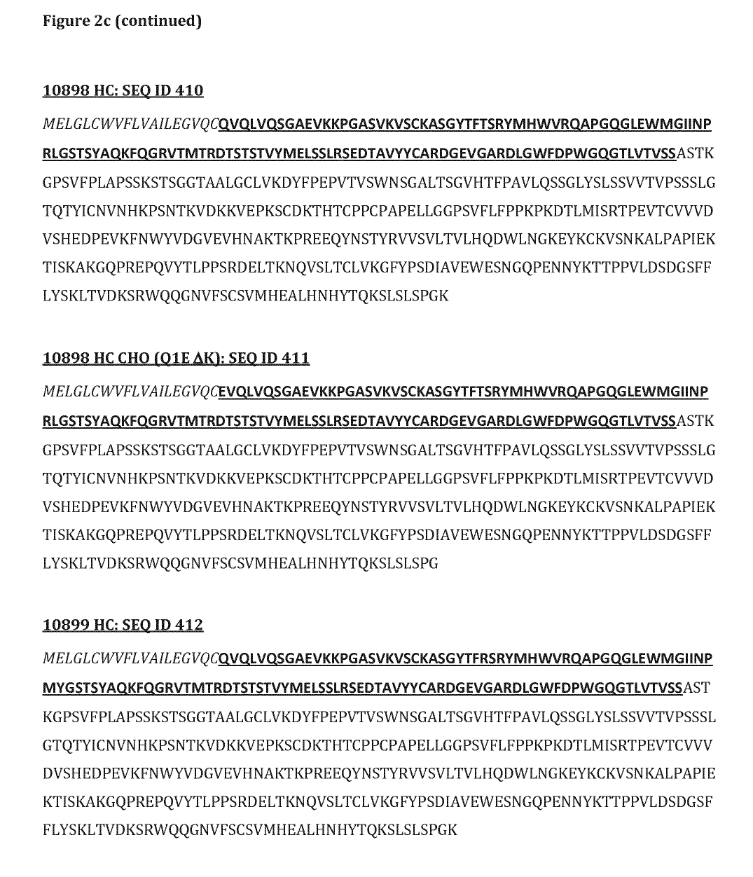

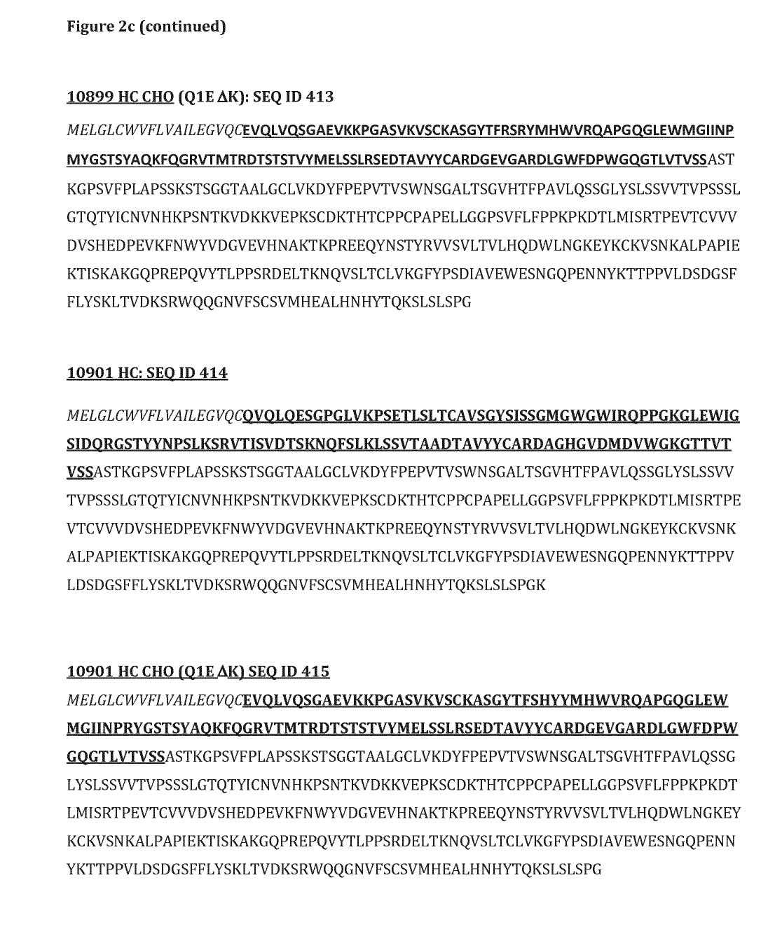

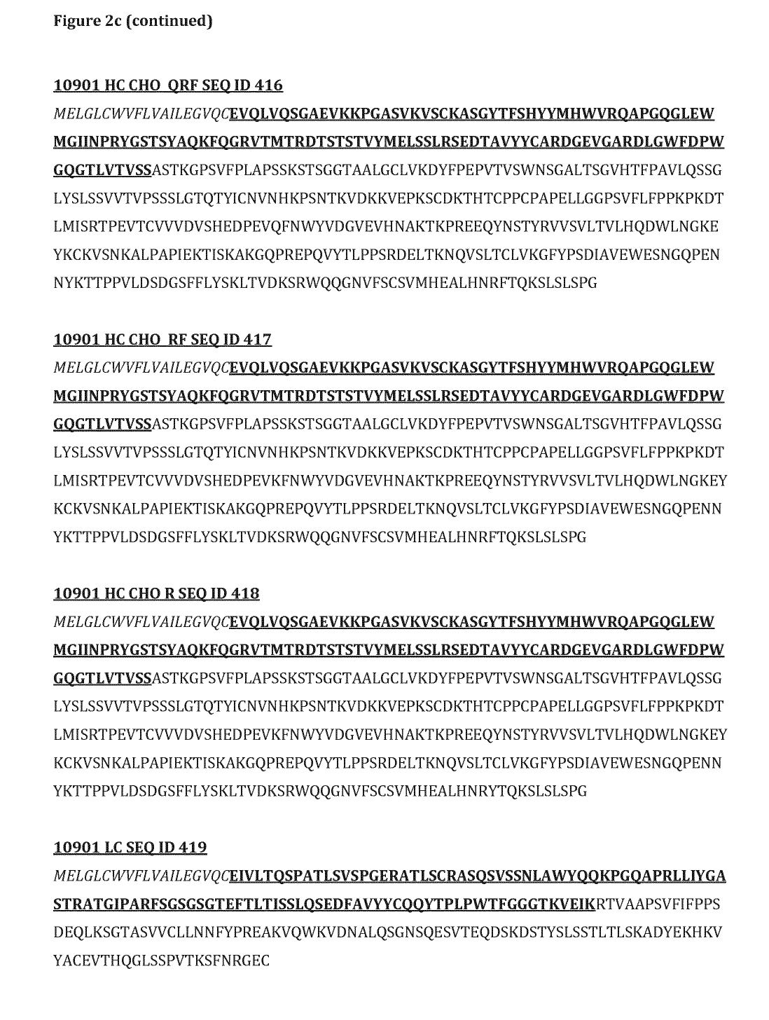

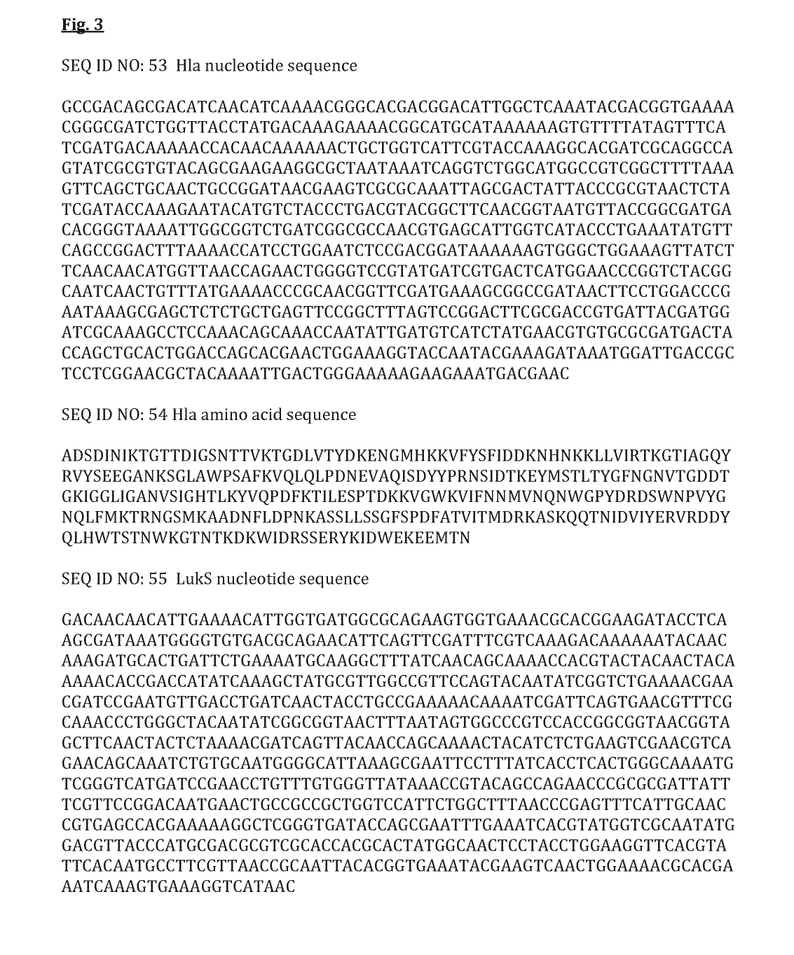

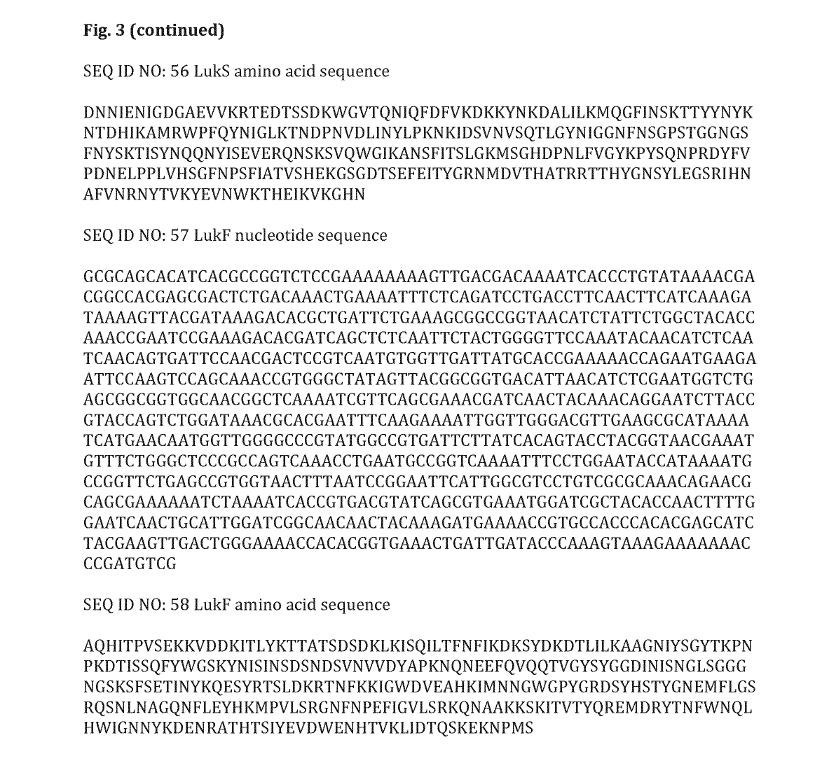

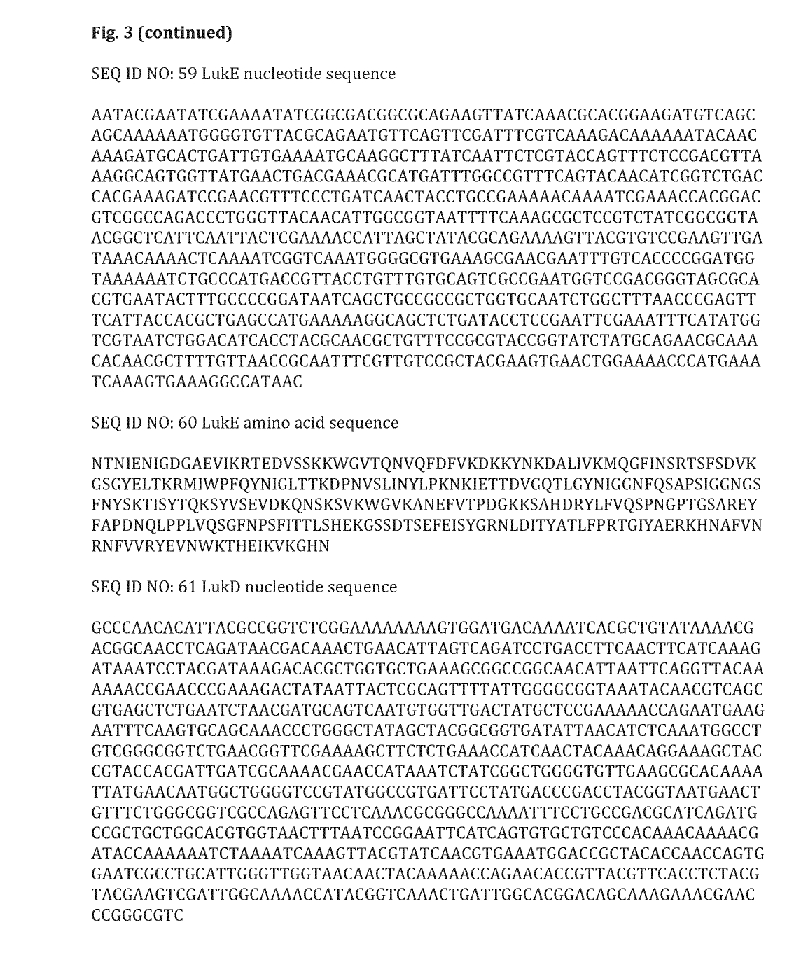

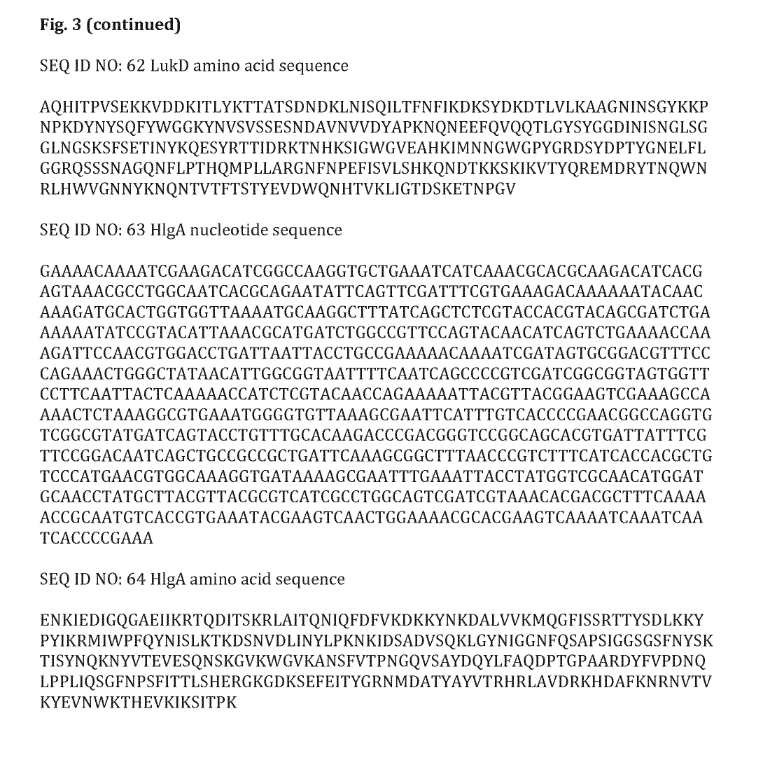

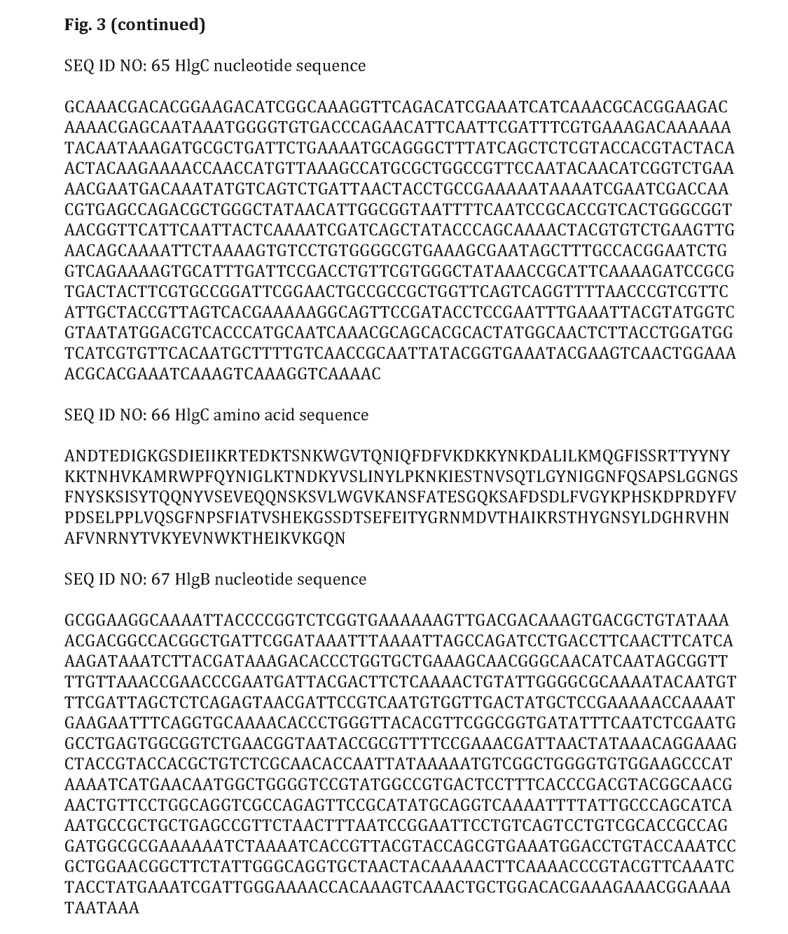

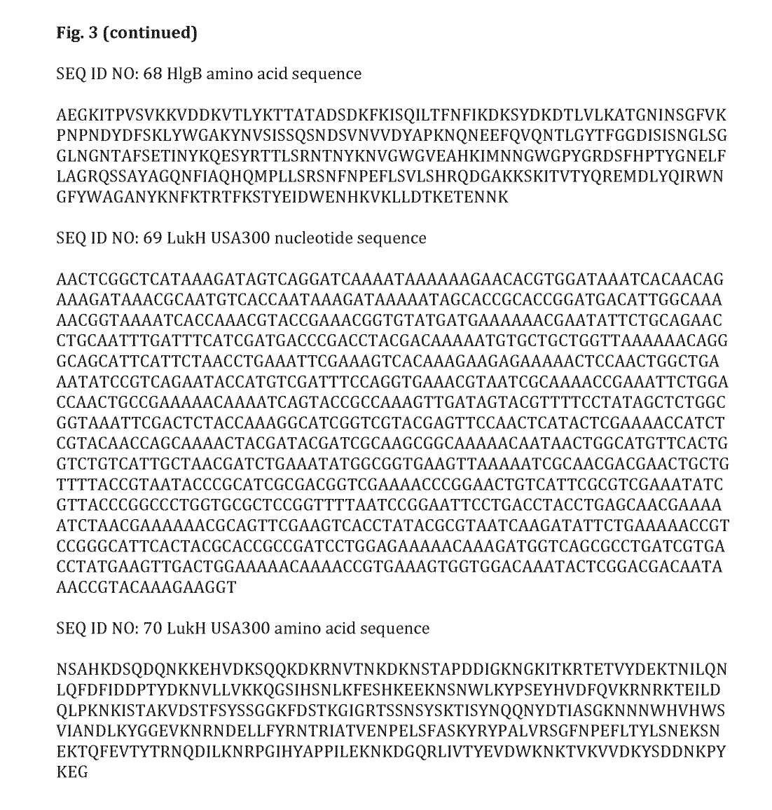

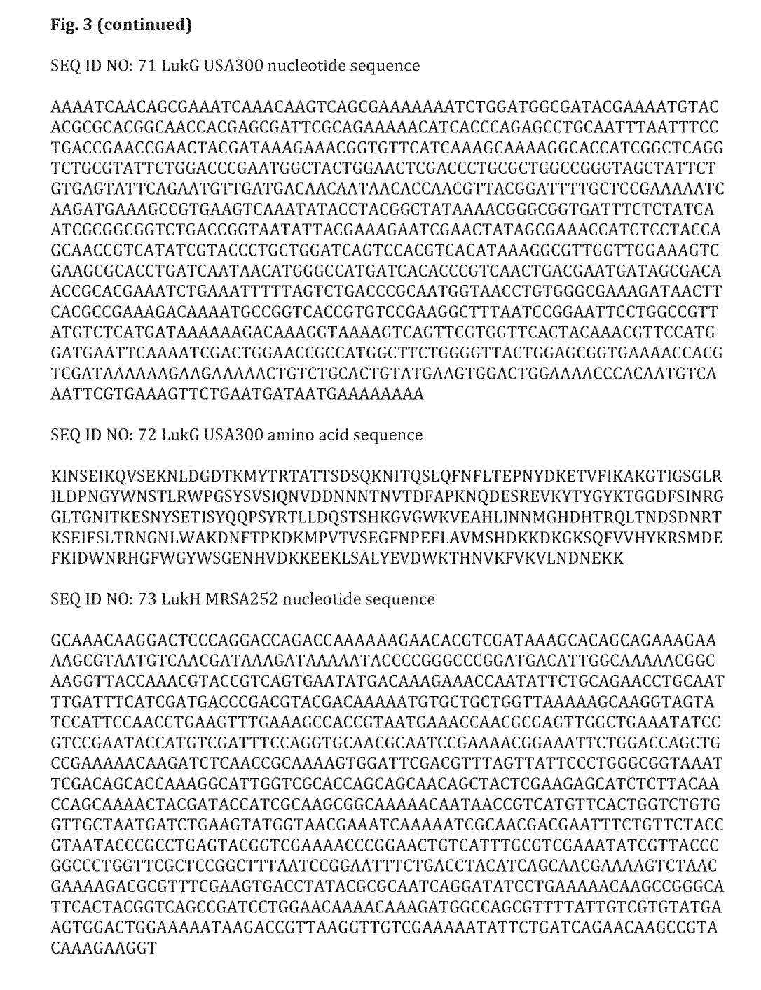

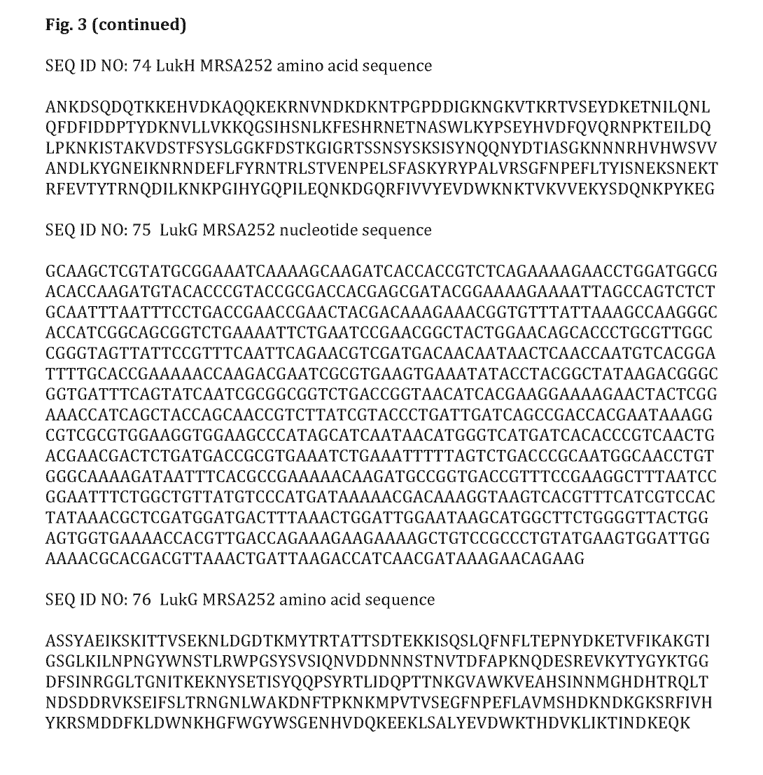

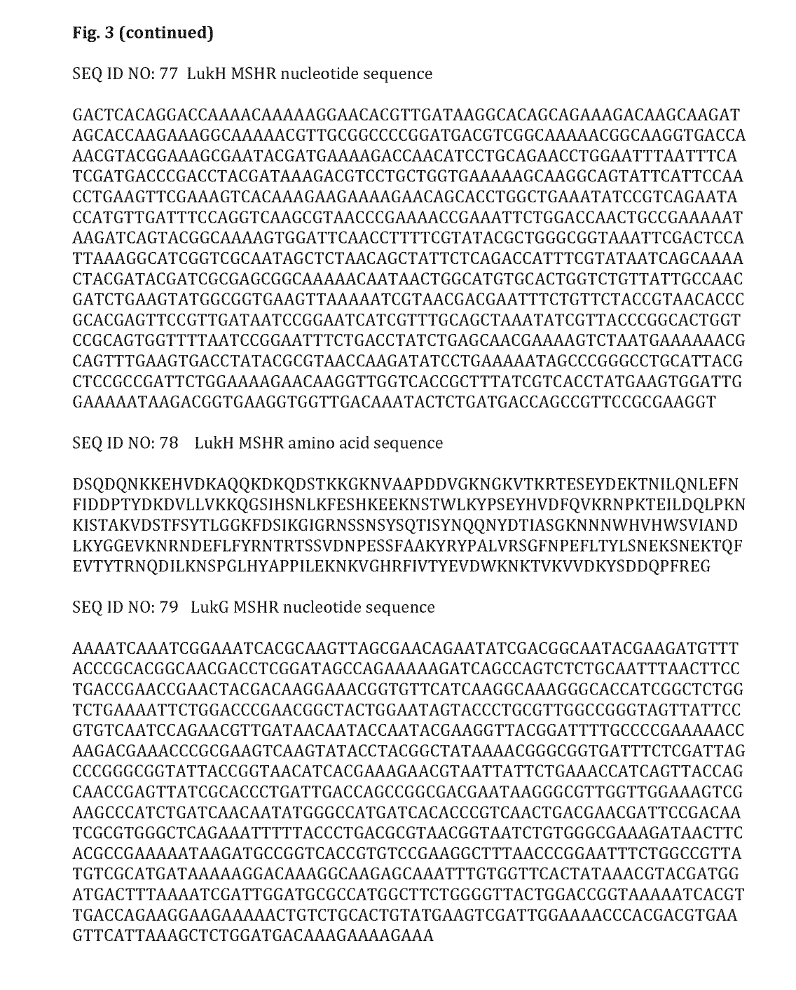

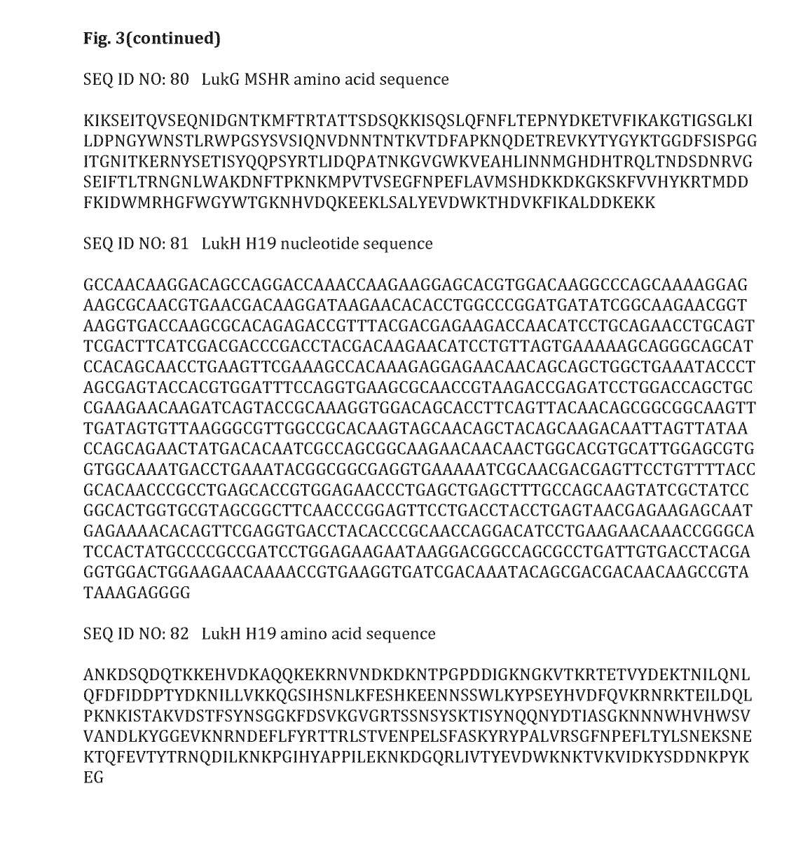

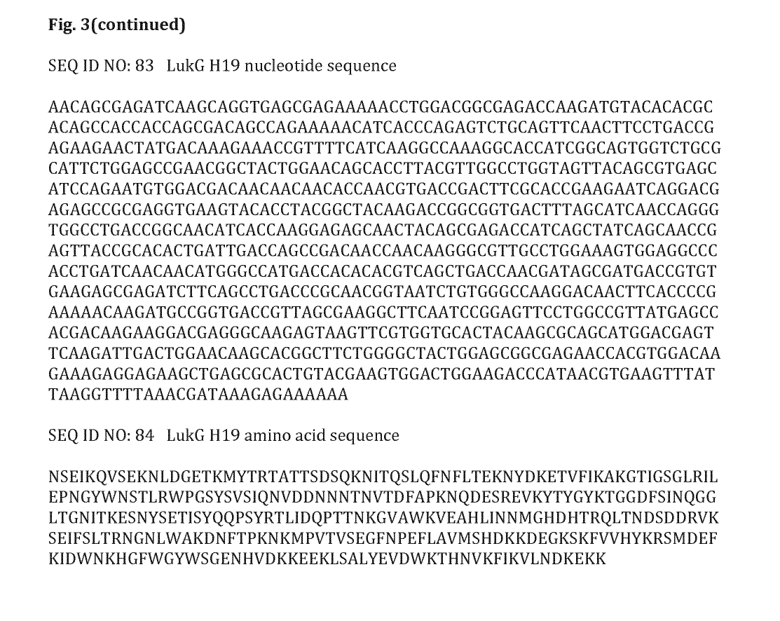

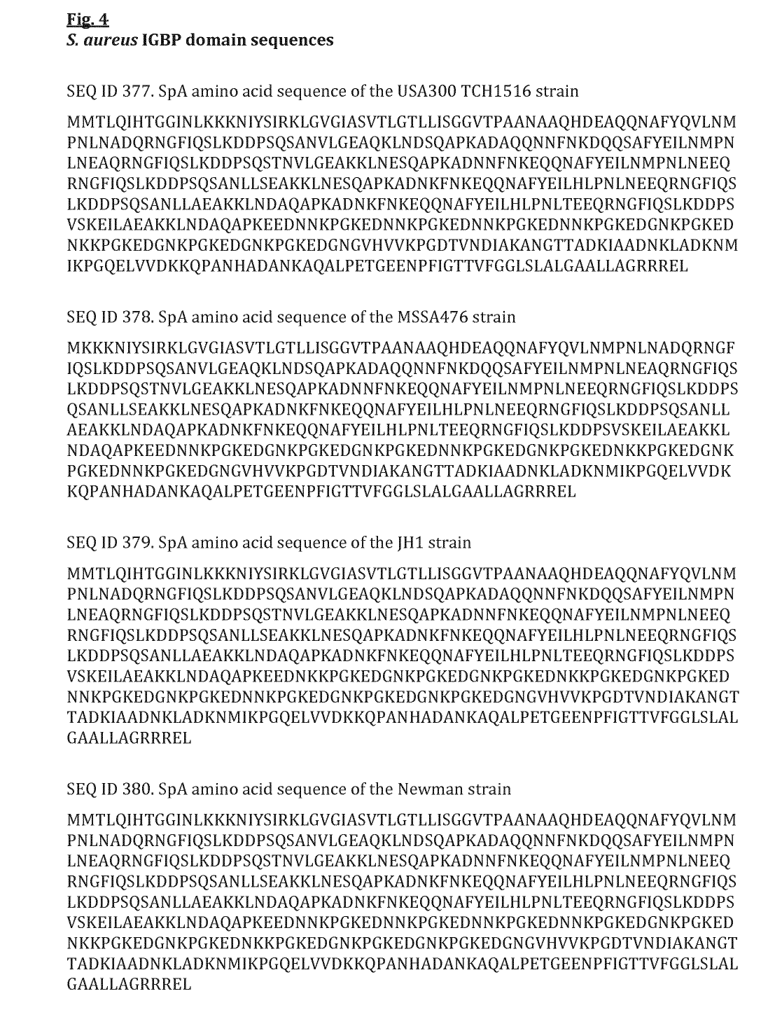

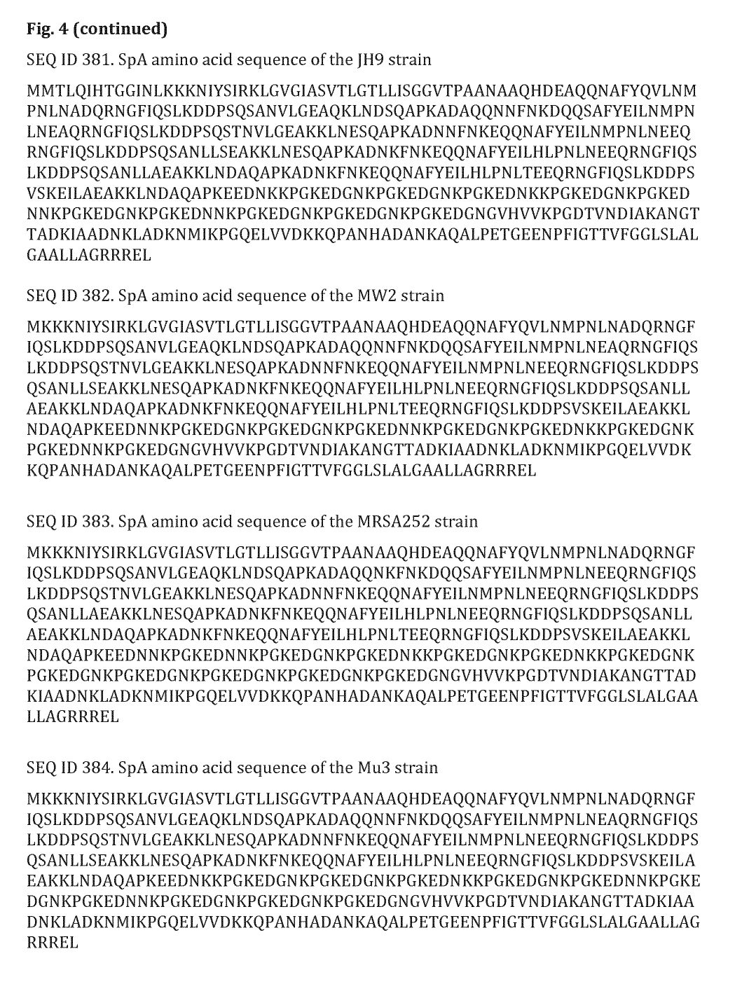

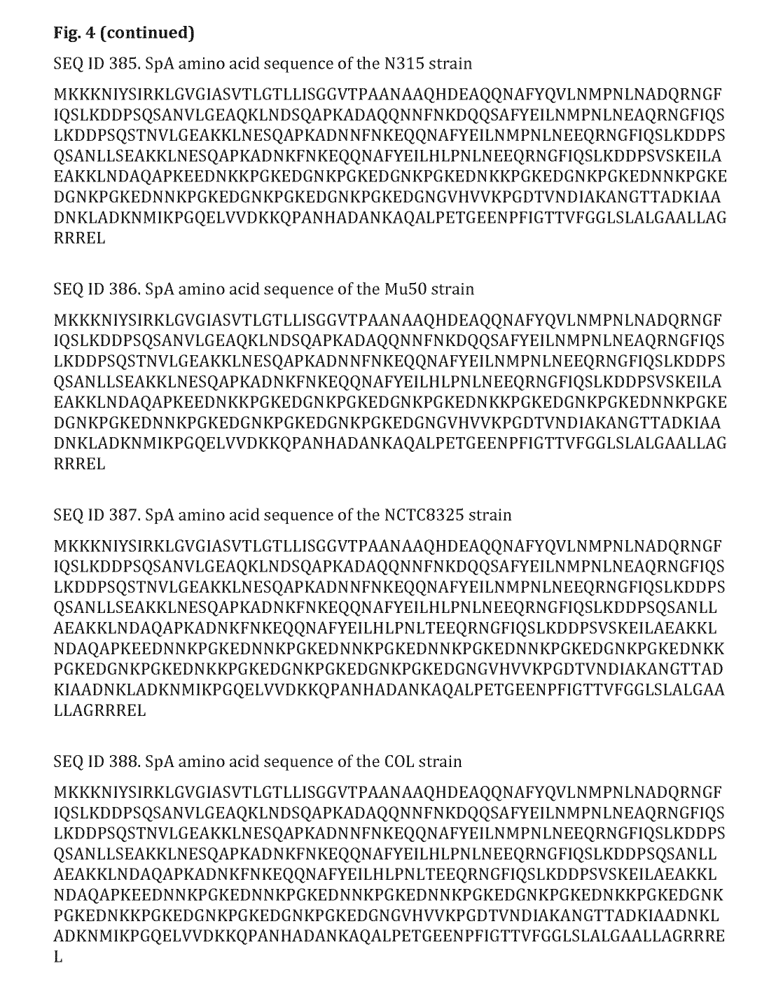

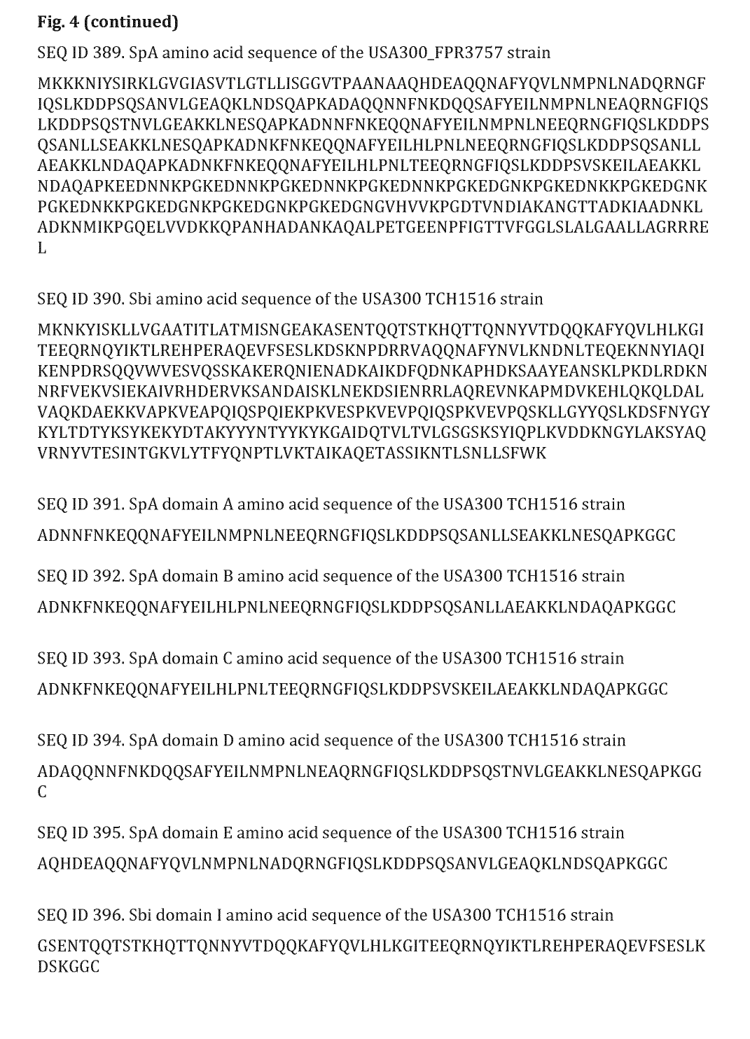

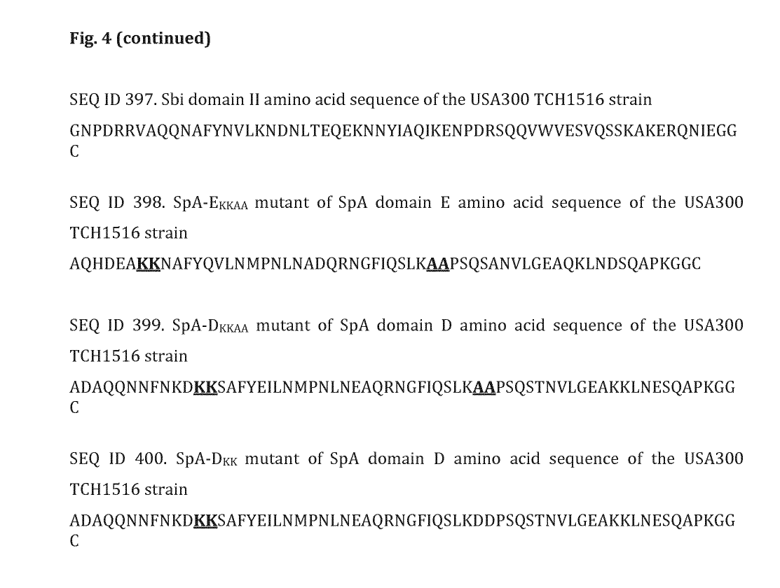

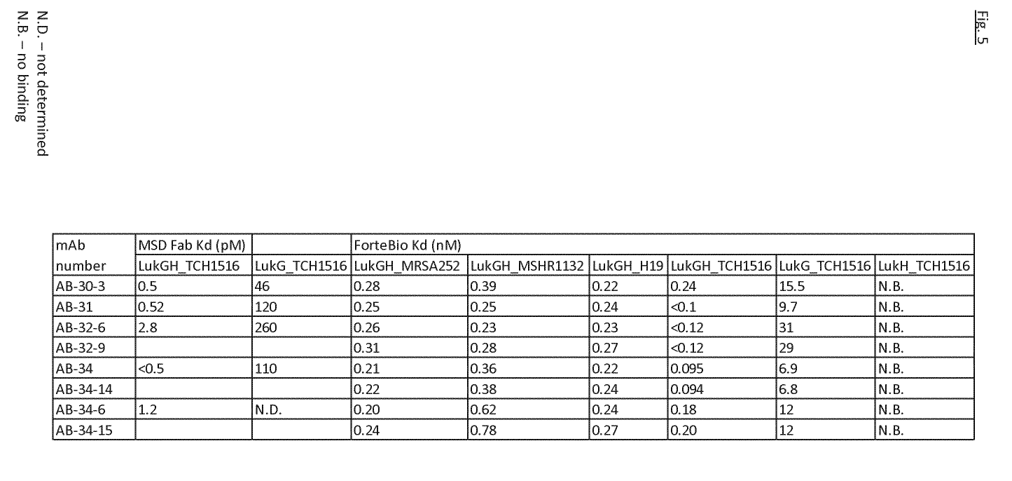

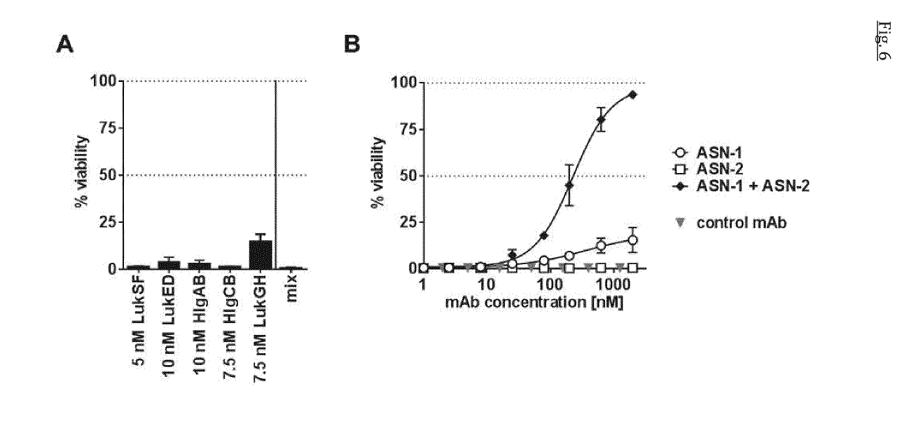

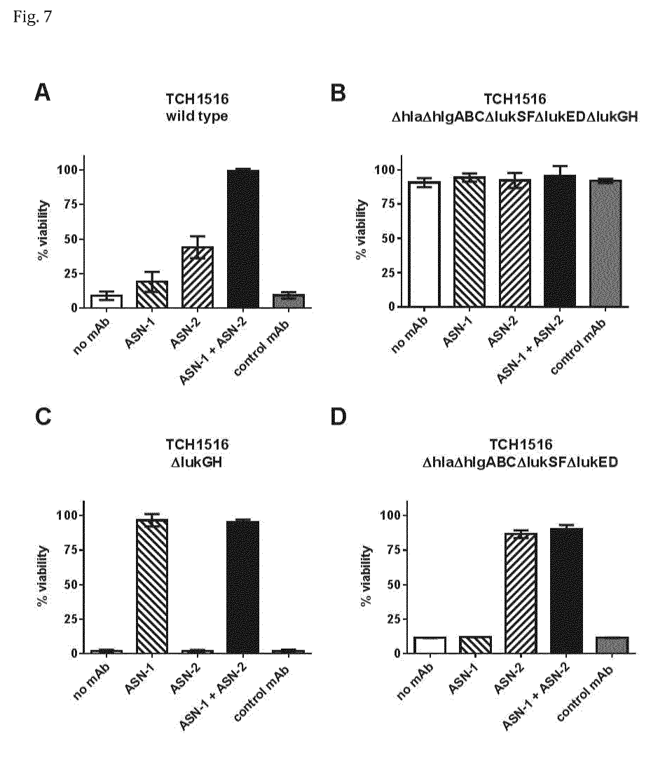

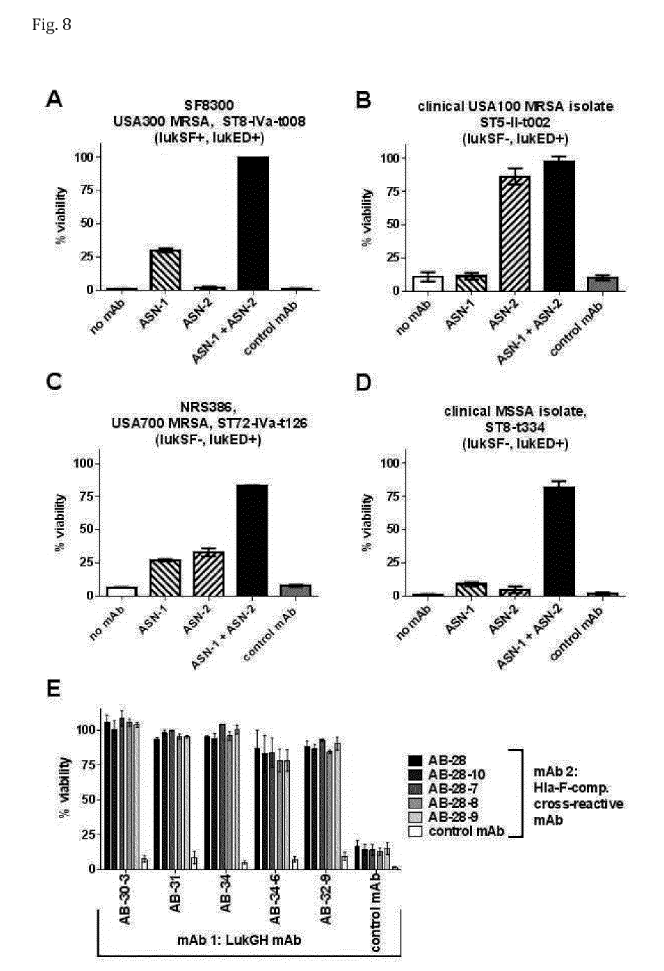

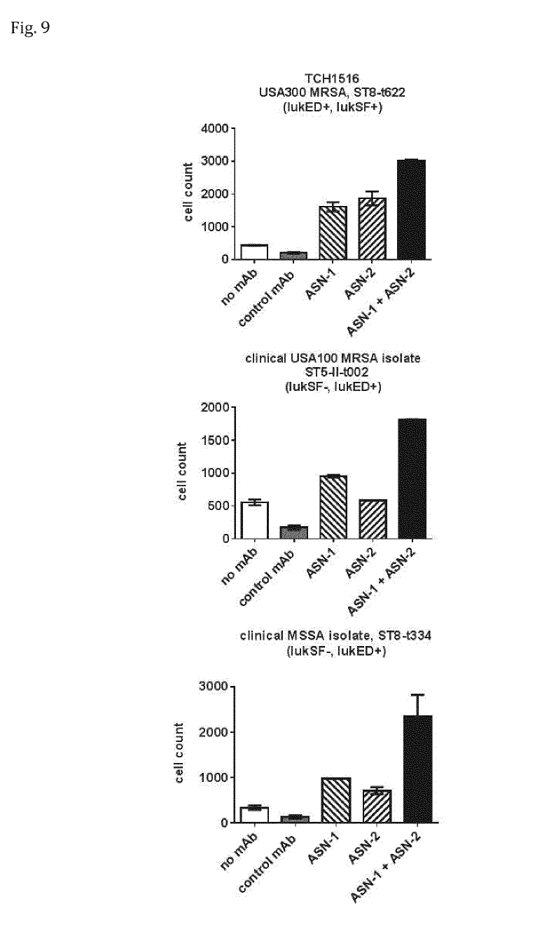

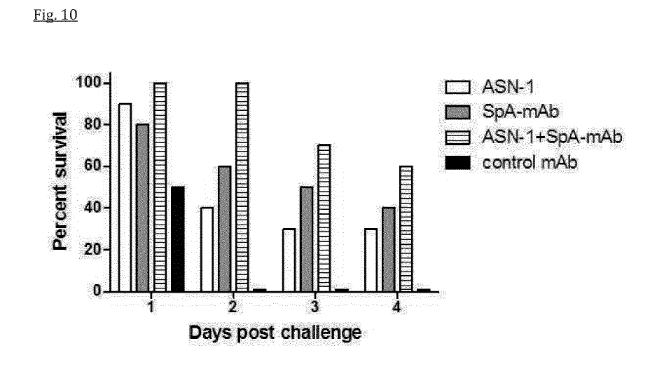

The nomenclature as used herein shall have the following meaning:

VH CDR1=CDR1

VH CDR2=CDR2

VH CDR3=CDR3

VL CDR4=CDR4=VL CDR1

VL CDR5=CDR5=VL CDR2

VL CDR6=CDR6=VL CDR3

Table 1: Amino acid sequences of toxin cross-neutralizing mAbs (Tables 1.1a-c) and Fab K_{D} affinities (Table 1.1d).

Heavy and light chain CDR sequences, FR sequences and full-length sequence information which is the composite sequence of the respective FR and CDR sequences (SEQ ID 1-39), are shown, amino acid changes relative to the parental AB-28 mAb indicated by bold and underlined fonts. Fab K_{D} affinities were measured by MSD method using a Sector Immager 2400 instrument (Meso Scale Discovery). Typically 20 pM of biotinylated antigen was incubated with Fab at various concentations, for 16h at room temperature, and the unbound antigen captured on immobilized IgG. See also for example, Estep et al., “High throughput solution-based measurement of antibody-antigen affinity and epitope binning”, MAbs, Vol. 5(2), pp. 270-278 (2013). Fab K_{D} affinities are indicated in pM for each antibody and for each toxin components.

The antibody designated #AB-28 is used as a parent antibody to produce functionally active CDR variants with one or more modified CDR sequences, and functionally active antibody variants with one or more modified FR sequences, such as sequences of FR1, FR2, FR3 or FR4, or a constant domain sequence, and/or with one or more modified CDR sequences. The variant antibody derived from the parent antibody by mutagenesis are exemplified in Table 1 and designated #AB-28-3, #AB-28-4, #AB-28-5, #AB-28-6, #AB-28-7, #AB-28-8, #AB-28-9, #AB-28-10, #AB-28-11, #AB-28-12, or #AB-28-13. Though these variant antibodies share the common VL sequence of SEQ ID39, it is feasible that also variant VL chains, e.g. with modifications in the respective FR or CDR sequences may be used, which are functionally active.

Table 2: Amino acid sequences of LukGH specific mAbs

Legend: Columns

A . . . SEQ ID VH FR1

B . . . SEQ ID VH CDR1

C . . . SEQ ID VH FR2

D . . . SEQ ID VH CDR2

E . . . SEQ ID VH FR3

F . . . SEQ ID VH CDR3

G . . . SEQ ID VH FR4

H . . . SEQ ID VL FR1

I . . . SEQ ID VL CDR4

J . . . SEQ ID VL FR2

K . . . SEQ ID VL CDR5

L . . . SEQ ID VL FR3

M . . . SEQ ID VL CDR6

N . . . SEQ ID VL FR4

Table 2 is divided in eight parts (for antibodies of Group 1-8): Table 2.1-2.8, each of Tables 2.1-2.8 is divided into Tables a (VH sequences) and b (VL sequences).

Table 2.1a shows the VH FR and CDR sequences of the antibodies of Group 1;

Table 2.1b shows the VL FR and CDR sequences of the antibodies of Group 1;

Table 2.2a shows the VH FR and CDR sequences of the antibodies of Group 2;

Table 2.2b shows the VL FR and CDR sequences of the antibodies of Group 2;

Table 2.3a shows the VH FR and CDR sequences of the antibodies of Group 3;

Table 2.3b shows the VL FR and CDR sequences of the antibodies of Group 3;

Table 2.4a shows the VH FR and CDR sequences of the antibodies of Group 4;

Table 2.4b shows the VL FR and CDR sequences of the antibodies of Group 4;

Table 2.5a shows the VH FR and CDR sequences of the antibodies of Group 5;

Table 2.5b shows the VL FR and CDR sequences of the antibodies of Group 5;

Table 2.6a shows the VH FR and CDR sequences of the antibodies of Group 6;

Table 2.6b shows the VL FR and CDR sequences of the antibodies of Group 6;

Table 2.7a shows the VH FR and CDR sequences of the antibodies of Group 7;

Table 2.7b shows the VL FR and CDR sequences of the antibodies of Group 7;

Table 2.8a shows the VH FR and CDR sequences of the antibodies of Group 8;

Table 2.8b shows the VL FR and CDR sequences of the antibodies of Group 8;

Table 3: Amino acid (CDR) sequences of IGBP specific mAbs

Table 3a: VH CDR sequences

Table 3b: VL CDR sequences

Table 3c: Affinity of selected mAbs to bind SpA-E and SpA wild-type versus SpA mutant:

The affinity was measured as follows. Biotinylated SpA-E, SpA-D and SpA-D_{KKAA} were produced as described in Example 1 and F(ab′)₂ fragments were generated from yeast or CHO derived IgGs by pepsin digestion as described in Example 2. Binding of the mAbs to the SpA domains was measured by interferometry using a ForteBio Octet Red instrument [Pall Life Sciences]; The biotinylated antigen (5 μg/ml) was immobilized on streptavidin sensors, to give a sensor loading of ˜2 nm. The association and dissociation of the antibody F(ab′)₂ fragment (50 nm; 100 nM for the yeast derived material with SpA-E), in solution (PBS, pH 7.2 plus 1% BSA), were measured at 30° C. for 10 min (5 min the yeast derived material with SpA-E) for the association and 5 min (3 min the yeast derived material with SpA-E) for the dissociation phase. The dissociation constants (KD values) were calculated based on the kinetic parameters (kon and koff) determined by fitting simultaneously the association and dissociation phases to a 1:1 binding model using Octet Data Analysis Software version 7. The improved binding to WT versus KKAA mutant SpA-D is expressed as KD ratio. NB indicates no binding to the SpA-D mutant.

For the measurement of selectivity of the anti-SpA mAbs towards WT SpA compared to SpA KKAA, binding of the mAbs to SpA-D (SEQ ID 394) and SpA-D KKAA (SEQ ID 399) was determined using biotinylated antigens as described above. SpA-D KKAA was expressed recombinantly as described for the wild-type domains, purified by anion exchange and size exclusion chromatography and biotinylated as above. In most cases, the anti-SpA mAbs showed decreased binding to the K variant, as opposed to 3F6, which has preference for the SpA-D KKAA.

According to a common protocol, the affinity measurement is performed as follows: Affinity measurements are performed by interferometry using a recombinant IGBP domain as antigen, and the antibody is produced as F(ab′)₂ or F(ab) fragments to determine the affinity of binding the antigen by the CDR binding site. The F(ab′)₂ or F(ab) fragments are expressed by a recombinant host and optionally further purified to avoid contaminating substances which could interfere with the affinity measurement. If an antibody is produced as IgG and further digested by pepsin to obtain the F(ab′)₂ preparation, the F(ab′)₂ preparation is optionally purified to avoid contaminating Fc fragments which could interfere with the affinity measurement.

According to the specific examples, affinity measurements are performed by interferometry using a ForteBio Octet Red instrument [Pall Life Sciences]; the biotinylated antigen was immobilized on streptavidin sensors to give a sensor loading of ˜2 nm. The association and dissociation of the antibody F(ab′)₂ or F(ab) fragments (50-100 and 100-200 nM, respectively), in solution (PBS, pH 7.2 plus 1% BSA), were measured at 30° C. for 3-10 min for the association phase and 3-30 min for the dissociation phase. The dissociation constants (K_{D} values) were calculated based on the kinetic parameters (kon and koff) determined by fitting simultaneously the association and dissociation phases to a 1:1 binding model using Octet Data Analysis Software version 7.

FIG. 2:

FIG. 2a: Amino acid sequence information of toxin cross-neutralizing antibodies: HC of AB-28, AB-28-3, AB-28-4, AB-28-5, AB-28-6, AB-28-7, AB-28-8, AB-28-9, AB-28-10, AB-28-11, AB-28-12, AB-28-13 (SEQ ID 40-51), and LC of AB-28 (SEQ ID 52).

FIG. 2b: HC and LC amino acid sequences of selected LukGH specific mAbs

FIG. 2c: The heavy chain of selected antibodies is listed (SEQ ID 408-418). All antibodies share the light chain 10901 (SEQ ID 419).

10895 HC: SEQ ID 408

10895 HC CHO (Q1E ΔK): SEQ ID 409

10898 HC: SEQ ID 410

10898 HC CHO (Q1E ΔK): SEQ ID 411

10899 HC: SEQ ID 412

10899 HC CHO (Q1E ΔK): SEQ ID 413

10901 HC: SEQ ID 414

10901 HC CHO (Q1E ΔK) SEQ ID 415

10901 HC CHO QRF SEQ ID 416

10901 HC CHO RF SEQ ID 417

10901 HC CHO R SEQ ID 418

10901 LC SEQ ID 419

FIG. 3: S. aureus toxin sequences referred to herein.

SEQ ID 53 HIa nucleotide sequence of the USA300 TCH1516 strain (Genbank, accession number CP000730)

SEQ ID 54: HIa amino acid sequence of the USA300 TCH1516 strain

SEQ ID 55 LukS nucleotide sequence of the USA300 TCH1516 strain

SEQ ID 56: LukS amino acid sequence of the USA300 TCH1516 strain

SEQ ID 57 LukF nucleotide sequence of the USA300 TCH1516 strain

SEQ ID 58: LukF amino acid sequence of the USA300 TCH1516 strain

SEQ ID 59 LukE nucleotide sequence of the USA300 TCH1516 strain

SEQ ID 60: LukE amino acid sequence of the USA300 TCH1516 strain

SEQ ID 61 LukD nucleotide sequence of the USA300 TCH1516 strain

SEQ ID 62: LukD amino acid sequence of the USA300 TCH1516 strain

SEQ ID 63 HIgA nucleotide sequence of the USA300 TCH1516 strain

SEQ ID 64: HIgA amino acid sequence of the USA300 TCH1516 strain

SEQ ID 65 HIgC nucleotide sequence of the USA300 TCH1516 strain

SEQ ID 66: HIgC amino acid sequence of the USA300 TCH1516 strain

SEQ ID 67 HIgB nucleotide sequence of the USA300 TCH1516 strain

SEQ ID 68: HIgB amino acid sequence of the USA300 TCH1516 strain

SEQ ID 69: LukH nucleotide sequence of the USA300 TCH1516 strain

SEQ ID 70: LukH amino acid sequence of the USA300 TCH1516 strain

SEQ ID 71 LukG nucleotide sequence of the USA300 TCH1516 strain

SEQ ID 72: LukG amino acid sequence of the USA300 TCH1516 strain

SEQ ID 73 LukH nucleotide sequence of the MRSA252 strain (Genbank, accession number BX571856)

SEQ ID 74: LukH amino acid sequence of the MRSA252 strain

SEQ ID 75 LukG nucleotide sequence of the MRSA252 strain

SEQ ID 76: LukG amino acid sequence of the MRSA252 strain

SEQ ID 77 LukH nucleotide sequence of the MSHR1132 strain (Genbank, accession number FR821777)

SEQ ID 78: LukH amino acid sequence of the MSHR1132 strain

SEQ ID 79 LukG nucleotide sequence of the MSHR1132 strain

SEQ ID 80: LukG amino acid sequence of the MSHR1132 strain

SEQ ID 81: LukH nucleotide sequence of the H19 strain (Patric, genome ID 72956; Genebank, accession number ACSS01000001 to ACSS01000063);

SEQ ID 82: LukH amino acid sequence of the H19 strain;

SEQ ID 83: LukG nucleotide sequence of the H19 strain;

SEQ ID 84: LukG amino acid sequence of the H19 strain.

FIG. 4: S. aureus IGBP and IGBP domain sequences. The following sequences of IGBP domains may contain a C-terminal GGC tag to facilitate site directed labeling of the antigens. It is understood that the sequences provided herein represent the amino acid sequences of the IGBP domains with or without the GGC tag.

SEQ ID 377. SpA amino acid sequence of the USA300 TCH1516 strain

SEQ ID 378. SpA amino acid sequence of the MSSA476 strain

SEQ ID 379. SpA amino acid sequence of the JH1 strain

SEQ ID 380. SpA amino acid sequence of the Newman strain

SEQ ID 381. SpA amino acid sequence of the JH9 strain

SEQ ID 382. SpA amino acid sequence of the MW2 strain

SEQ ID 383. SpA amino acid sequence of the MRSA252 strain

SEQ ID 384. SpA amino acid sequence of the Mu3 strain

SEQ ID 385. SpA amino acid sequence of the N315 strain

SEQ ID 386. SpA amino acid sequence of the Mu50 strain

SEQ ID 387. SpA amino acid sequence of the NCTC8325 strain

SEQ ID 388. SpA amino acid sequence of the COL strain

SEQ ID 389. SpA amino acid sequence of the USA300_FPR3757 strain

SEQ ID 390. Sbi amino acid sequence of the USA300 TCH1516 strain

SEQ ID 391. SpA domain A amino acid sequence of the USA300 TCH1516 strain

SEQ ID 392. SpA domain B amino acid sequence of the USA300 TCH1516 strain

SEQ ID 393. SpA domain C amino acid sequence of the USA300 TCH1516 strain

SEQ ID 394. SpA domain D amino acid sequence of the USA300 TCH1516 strain

SEQ ID 395. SpA domain E amino acid sequence of the USA300 TCH1516 strain

SEQ ID 396. Sbi domain I amino acid sequence of the USA300 TCH1516 strain

SEQ ID 397. Sbi domain II amino acid sequence of the USA300 TCH1516 strain

SEQ ID 398. SpA-E_{KKAA} mutant of SpA domain E amino acid sequence of the USA300 TCH1516 strain

SEQ ID 399. SpA-D_{KKAA} mutant of SpA domain D amino acid sequence of the USA300 TCH1516 strain

SEQ ID 340. SpA-D_{KK} mutant of SpA domain D amino acid sequence of the USA300 TCH1516 strain

FIG. 5: Binding affinity of selected LukGH mAbs

FIG. 6: Protection of human PMNs from lysis due to recombinant leukocidins in presence of ASN-1 and ASN-2. A: Effect of individual toxins on PMN viability; B:

protective effect of indicated antibodies against a mixture of all these toxins.

FIG. 7: Synergistic and individual effect of ASN-1 and ASN-2 on PMN survival in presence of bacterial culture supernatants derived from S. aureus strain TCH1516 (A) and its isogenic gene deletion mutants lacking genes encoding for HIa, HIgABC, LukSF, LukED and LukGH (B), LukGH (C) and HIa, HIgABC, LukSF and LukED (D).

FIG. 8: Synergistic and individual effect of different HIa-F-component cross-reactive and anti-LukGH mAbs on PMN survival in presence of bacterial culture supernatants derived from diverse S. aureus strains. A: Effect of ASN-1 and ASN-2 in CS neutralization of USA300 MRSA strain SF8300; B: Effect of ASN-1 and ASN-2 in CS neutralization of clinical USA100 MRSA isolate (ST5-114002), C: Effect of ASN-1 and ASN-2 in CS neutralization of USA700 MRSA isolate NRS386 (ST72-IVa-t126); D: Effect of ASN-1 and ASN-2 in CS neutralization of clinical MSSA isolate ST8-t334. E: Synergistic effect of different HIa-F-component cross-reactive mAbs with anti-LukGH mAbs on PMN survival exemplified with a CS derived of clinical MSSA isolate ST8-t334 and HIa-F-component crossreactive mAbs AB-28, AB-28-10, AB-28-7, AB-28-8, AB-28-9 and LukGH mAbs AB-30-3, AB-31, AB-34, AB-34-6 and AB-32-9.

FIG. 9: Synergistic and individual effect of ASN-1 and ASN-2 on PMN survival during infection with live S. aureus. A: USA300 MRSA strain TCH1516 (ST84622); B: Clinical USA100 MRSA isolate (ST5-114002); C: clinical MSSA isolate (ST8-t334).

FIG. 10: Survival of mice treated with anti-S.aureus mAbs in lethal bacteremia/sepsis model.

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VL/VH pair, an antibody comprising a VL/VH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VL/VH pair, an antibody comprising or consisting of a VL/VH domain pair and constant antibody domains, such as heavy-chain antibodies, Fab, F(ab′), (Fab)₂, scFv, Fd, Fv, or a full-length antibody, e.g. of an IgG type (e.g., an IgG1, IgG2, IgG3, or IgG4 sub-type), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin, e.g. human sequences.

The term “antibody” further applies to chimeric antibodies with sequences of origin of different species, such as sequences of murine and human origin.

The term “chimeric” as used with respect to an antibody refers to those anti-bodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized anti-bodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. A human antibody as described herein may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “antibody” specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as anti-bodies originating from animals, e.g. mammalians including human, that comprises genes or sequences from different origin, e.g. murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/ or a fusion protein, in which any domain of the antibody may be fused at any position of one or more other proteins, such as other antibodies, e.g. a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic inter-action, di-sulphide bonding etc. The other substances bound to the antibody may be lipids, carbohydrates, nucleic acids, organic and inorganic molecules or any combination thereof (e.g. PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

The preferred derivatives as described herein are functionally active with regard to the antigen binding, and alike the antibodies that are not derivatized, preferably have a potency to neutralize S. aureus and/or which are protective antibodies.

Antibodies derived from a parent antibody or antibody sequence, such as a parent CDR or FR sequence, are herein particularly understood as mutants or variants obtained by e.g. in silico or recombinant engineering or else by chemical derivatization or synthesis.

Specifically, an antibody derived from an antibody as described herein may comprise at least one or more of the CDR regions or CDR variants thereof, e.g. at least 3 CDRs of the heavy chain variable region and/or at least 3 CDRs of the light chain variable region, with at least one point mutation in at least one of the CDR or FR regions, or in the constant region of the HC or LC, being functionally active, e.g. determined by essentially the same or improved binding characteristics to the target antigen.

It is understood that the term “antibody” also refers to variants of an antibody, including antibodies comprising functionally active CDR variants of a parent CDR sequence, and functionally active variant antibodies of a parent antibody.

The term “variant” shall particularly refer to antibodies, such as mutant anti-bodies or fragments of antibodies, e.g. obtained by mutagenesis methods, in particular to delete, exchange, introduce inserts into a specific antibody amino acid sequence or region or chemically derivatise an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomisation techniques. In some cases positions are chosen randomly, e.g. with either any of the possible amino acids or a selection of preferred amino acids to randomise the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence, e.g. in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant. Further modifications may be made through mutagenesis, e.g. to widen the cross-specificity to target more toxins or toxin components (e.g. different types of toxins or toxin variants originating from different strains), or to target more different antigens (domains of IGBP) than the parent antibody, or to increase its reactivity with one or more of the targets. A specific indicator of functional activity is considered the competitive binding to inhibit binding of any of the toxins or toxin components or heterodimer, such as the LukGH complex or dimer, to the cell membranes, in particular to phosphocholine.

Preferred antibodies as described herein are binding the individual antigens with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or K_{D}). Usually a binder is considered a high affinity binder with a K_{D}<10⁻⁸ M, preferably a K_{D}<10⁻⁹ M, even more preferred is a K_{D}<10⁻¹⁰ M.

Yet, in a particularly preferred embodiment the individual antigen binding affinities are of medium affinity, e.g. with a K_{D} of less than 10⁻⁶ M and up to 10⁻⁸ M, e.g. when binding to at least two antigens.

Medium affinity binders may be provided according to the invention, preferably in conjunction with an affinity maturation process, if necessary.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several log fold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody as described herein exhibits at least a 2 fold increase in affinity of binding, preferably at least a 5, preferably at least 10, preferably at least 50, or preferably at least 100 fold increase. The affinity maturation may be employed in the course of the selection campaigns employing respective libraries of parent molecules, either with antibodies having medium binding affinity to obtain the antibody of the invention having the specific target binding property of a binding affinity K_{D}<10⁻⁸ M. Alternatively, the affinity may be even more increased by affinity maturation of the antibody according to the invention to obtain the high values corresponding to a K_{D} of less than 10⁻⁹ M, preferably less than 10⁻¹⁰ M or even less than 10⁻¹¹ M, most preferred in the picomolar range.

In certain embodiments binding affinity is determined by an affinity ELISA assay. In certain embodiments binding affinity is determined by a BIAcore, ForteBio or MSD assays. In certain embodiments binding affinity is determined by a kinetic method. In certain embodiments binding affinity is determined by an equilibrium/solution method.

The functional activity is preferably determined in an assay for determining the neutralization potency of antibodies against cytolytic toxins, e.g. determined in a standard assay by measuring increased viability or functionality of cells susceptible to the given toxin. Specific tests for determining protection of human PMNs from lysis due to recombinant leukocidins, native cytotoxins present in culture supernatants of S. aureus clinical isolates, or by live S. aureus are described in the examples section.

For example, the functional activity is determined if the antibody exhibits in vitro neutralization potency in a cell-based assay with an IC50 of less than 100:1 mAb:toxin ratio (mol/mol), preferably less than 50:1, preferably less than 25:1, preferably less than 10:1, more preferably less than 1:1. Neutralization is typically expressed by percent of viable cells with and without antibodies. For highly potent antibodies, a preferred way of expressing neutralization potency is the antibody:toxin molar ratio, where lower values correspond to higher potency. Values below 10 define a high functional activity. Optionally, values are in the most stringent assay between 1 and 10.

Typically the functional activity of variants is proven if they exhibit substantially the same functional activity or substantially the same biological activity as the comparable (parent or non-modified) antibody.

The term “substantially the same functional activity” or “substantially the same biological activity” as used herein refers to the activity as indicated by substantially the same activity being at least 20%, at least 50%, at least 75%, at least 90%, e.g. at least 100%, or at least 125%, or at least 150%, or at least 175%, or e.g. up to 200% of the activity as determined for the comparable or parent antibody.

The preferred variants or derivatives as described herein are functionally active with regard to the antigen binding, preferably which have a potency to specifically bind the individual targets, and not significantly binding to other antigens that are not target antigens, e.g. with a K_{D} value difference of at least 2 logs, preferably at least 3 logs. The antigen binding by a functionally active variant is typically not impaired, corresponding to about substantially the same binding affinity as the parent antibody or sequence, or antibody comprising a sequence variant, e.g. with a a K_{D} value difference of less than 2 logs, preferably less than 3 logs, however, with the possibility of even improved affinity, e.g. with a K_{D} value difference of at least 1 log, preferably at least 2 logs.

Functional activity as determined by the specific targeting of the LukGH complex is specifically further characterized by the preferential binding of the LukGH complex over the individual toxins LukG and LukH. Binding of the anti-LukGH antibody as described herein to the heterodimeric or oligomeric LukGH antigen is specifically improved as compared to binding of any of or both of the separated (monomeric) LukG or LukH, e.g. as characterized by a differential affinity or K_{D} of at least 1 or 2 logs difference.

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally-occurring allelic variants, as well as mutants or any other non-naturally occurring (e.g. synthetic or artificial) antibodies, or variants, such as those comprising antigen-binding sequences derived from artificial antibody libraries. As is known in the art, an allelic variant is an alternate form of a (poly)peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a poly-nucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties. The preferred variants as described herein are functionally active with regard to the antigen binding, preferably which have a potency to neutralize S. aureus and/or which are protective antibodies.

An antibody as described herein may or may not exhibit Fc effector function. Though the mode of action is mainly mediated by neutralizing antibodies without Fc effector functions, Fc can recruit complement and aid elimination of the target antigen, such as a toxin, from the circulation via formation of immune complexes.

Specific antibodies may be devoid of an active Fc moiety, thus, either composed of antibody domains that do not contain an Fc part of an antibody or that do not contain an Fcgamma receptor binding site, or comprising antibody domains lacking Fc effector function, e.g. by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis, e.g. mutations in the Fcgamma receptor binding site or by derivatives or agents to interfere with ADCC and/or CDC activity of an antibody format, so to achieve reduction or increase of Fc effector function.

A significant reduction of Fc effector function is typically understood to refer to Fc effector function of less than 10% of the unmodified (wild-type) format, preferably less than 5%, as measured by ADCC and/or CDC activity.

A significant increase of Fc effector function is typically understood to refer to an increase in Fc effector function of at least 10% of the unmodified (wild-type) format, preferably at least 20%, 30%, 40% or 50%, as measured by ADCC and/or CDC activity.

The term “glycoengineered” variants with respect to antibody sequences shall refer to glycosylation variants having modified immunogenic or immunomodulatory (e.g. anti-inflammatory) properties, ADCC and/or CDC, as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC). Removal of N-Glycan at N297, e.g. through mutating N297, e.g. to A, or T299 typically results in aglycosylated antibody formats with reduced ADCC. Specifically, an antibody as described herein may be glycosylated or glycoengineered, or aglycosylated antibodies.

Major differences in antibody glycosylation occur between cell lines, and even minor differences are seen for a given cell line grown under different culture conditions. Expression in bacterial cells typically provides for an aglycosylated antibody. CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180). In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are interposed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

Specifically, the CDR sequences as referred to herein are understood as those amino acid sequences of an antibody as determined according to Kabat nomenclature (see Kabat et al., Sequences of Proteins of Immunological Interest, 5^{th} Ed. Public Health Service, U.S. Department of Health and Human Services. (1991)).

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen. Specifically, the conformational epitope is an epitope which is comprised of a series of amino acid residues which are non-linear in alignment that is that the residues are spaced or grouped in a non-continuous manner along the length of a polypeptide sequence. Such conformational epitope is characterized by a three-dimensional structure with specific structure coordinates as determined by contacting amino acid residues and/or crystallographic analysis, e.g. analysis of a crystal formed by the immune complex of the epitope bound by a specific antibody or Fab fragment.

Herein the term “epitope” shall particularly refer to the single epitope recognized by an antibody, or a series of epitope variants, each recognized by a cross-reactive antibody.

The toxin cross-neutralizing antibody as described herein is a cross-reactive antibody specifically recognizing the rim domain of the toxins, in particular the soluble toxin monomers or toxin components. The rim domain is understood as the domain of the toxin that is juxtaposed to the outer leaflet of the host plasma membrane, which rim domain is involved in cell membrane binding. Thus, the rim region serves as a membrane anchor. The epitope targeted by an antibody as described herein, which is located in the rim region or the rim domain, thus, has the potential of being immunorelevant, i.e. relevant for protection by active or passive immunotherapy.

The anti-LukGH antibody as described herein is specifically recognizing the rim domain of the LukG toxin, in particular the LukG as complexed with the LukH toxin to form the LukGH complex or LukGH heterodimer. The rim domain is understood as the domain of the toxin that is juxtaposed to the outer leaflet of the host plasma membrane, which rim domain is involved in cell membrane binding. Thus, the rim region serves as a membrane anchor. The epitope targeted by the antibody of the invention, which is located in the rim region or the rim domain, thus, has the potential of being immunorelevant, i.e. relevant for protection by active or passive immunotherapy.

The invention specifically employs cross-reactive antibodies, which are obtained by a process to identify neutralizing antibodies with multiple specificities, e.g. by a cross-reactive discovery selection scheme. Accordingly, an antibody library including antibodies showing reactivity with two targets, targets A and B, may first be selected for reactivity with one of the targets, e.g. target A, followed by selection for reactivity with the other target, e.g. target B. Each successive selection round reinforces the reactive strength of the resulting pool towards both targets. Accordingly, this method is particularly useful for identifying antibodies with cross-reactivity directed to the two different targets, and the potential to cross-neutralize a pathogen. The method can be extended to identifying antibodies showing reactivity towards further targets, by including additional rounds of enrichment towards the additional target(s).

Cross-reactive antibodies, in some instances, emerge through screening against single antigens. To increase the likelihood of isolating cross-reactivity clones one would apply multiple selective pressures by processively screening against multiple antigens. Special mAb selection strategies employ the different toxin components or different toxin variants, or different IGBP domains in an alternating fashion. For example, neutralizing anti-HIa mAbs are tested for binding to PVL and PVL like toxins on human neutrophils, which represent the major target for bi-component toxins during S. aureus infection.

The recombinant toxins or IGBP domains produced by recombinant techniques employing the respective sequences as provided in the Figures, or toxins isolated from S. aureus culture supernatants may be used for selecting antibodies from a yeast-based antibody presentation library, as disclosed in, for example, e.g., WO2012/009568; WO2009/036379; WO2010/105256; US2002/0177170; WO2003/074679. Alternatively, antibodies may be selected from, e.g. a yeast-displayed antibody library see, for example: Blaise L, Wehnert A, Steukers M P, van den Beucken T, Hoogenboom H R, Hufton S E. Construction and diversification of yeast cell surface displayed libraries by yeast mating: application to the affinity maturation of Fab antibody fragments. Gene. 2004 Nov. 24; 342(2):211-8; Boder E T, Wittrup K D. Yeast surface display for screening combinatorial polypeptide libraries. Nat Biotechnol. 1997 June; 15(6):553-7; Kuroda K, Ueda M. Cell surface engineering of yeast for applications in white biotechnology. Biotechnol Lett. 2011 January; 33(1):1-9. doi: 10.1007/s10529-010-0403-9. Review; Lauer T M, Agrawal N J, Chennamsetty N, Egodage K, Helk B, Trout B L. Developability index: a rapid in silico tool for the screening of antibody aggregation propensity. J Pharm Sci. 2012 January; 101(1):102-15; Orcutt K. D. and Wittrup K. D. (2010), 207-233 doi: 10.1007/978-3-642-01144-3_15; Rakestraw J A, Aird D, Aha P M, Baynes B M, Lipovsek D. Secretion-and-capture cell-surface display for selection of target-binding proteins. Protein Eng Des Sel. 2011 June; 24(6):525-30; U.S. Pat. No. 6,423,538; U.S. Pat. No. 6,696,251; U.S. Pat. No. 6,699,658; published PCT application publication No. WO2008118476.

In either event cross-reactivity can be further improved by antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

Screening methods for identifying antibodies with the desired neutralizing properties may be inhibition of toxin binding to the target cells, inhibition of formation of dimers or oligomers, inhibition of pore formation, inhibition of cell lysis, inhibition of the induction of cytokines, lymphokines, and any pro-inflammatory signaling, and/or inhibition of in vivo effect on animals (death, hemolysis, overshooting inflammation, organ dysfunction). Reactivity can be assessed based on direct binding to the desired toxins, e.g. using standard assays.

Once cross-neutralizing antibodies with the desired properties have been identified, the dominant epitope or epitopes recognized by the antibodies may be determined. Methods for epitope mapping are well-known in the art and are disclosed, for example, in Epitope Mapping: A Practical Approach, Westwood and Hay, eds., Oxford University Press, 2001.

Antibodies or antibody fragments can be produced by methods well-known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and transfecting a recombinant host cell with the coding sequences for expression, using well known recombinant expression vectors, e.g. the plasmids of the invention or expression cassette(s) comprising the nucleotide sequences encoding the antibody sequences. Recombinant host cells can be prokaryotic and eukaryotic cells, e.g. including animal or human cell lines in cell cultures.

According to a specific aspect, a coding nucleotide sequence may be used for genetic manipulation to humanize the antibody or to improve the affinity, or other characteristics of the antibody. For example, the constant region may be engineered to more nearly resemble human constant regions to avoid immune response, if the anti-body is used in clinical trials and treatments in humans. It may be desirable to genetically manipulate the antibody sequence to obtain greater affinity to the target toxins and greater efficacy against S. aureus. It will be apparent to one of skill in the art that one or more polynucleotide changes can be made to the antibody and still maintain its binding ability to the target antigens.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101 :189-198) describe the production of monoclonal antibodies from separately produced heavy and light chains, using plasmids expressed in separate cultures of E. coli. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

If desired, the polynucleotide sequence encoding any of the exemplified antibodies may be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

Monoclonal antibodies are typically produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

The term “isolated” or “isolation” as used herein with respect to an antibody shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification.

Likewise, the isolated antibody of the invention is specifically non-naturally occurring, e.g. as provided in a combination preparation with another antibody, which combination does not occur in nature (such as a combination with one or more monospecific antibody and/or with a cross-specific antibody which recognizes at least two different targets), or an optimized or affinity-matured variant of a naturally occurring antibody, or an antibody with a framework-region which is engineered to improve the manufacturability of the antibody.

With reference to polypeptides or proteins, such as isolated antibodies of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy. The term “isolated antibodies” as used herein is specifically meant to include recombinant antibodies or monoclonal antibodies obtained from cell culture, such as produced by cultivating recombinant host cells that have been transformed with artificial nucleic acid constructs encoding the antibodies, or those chemically synthesized.

The term “LukGH complex” as used herein shall refer to the dimer or oligomer, including 1:1 dimers or any other ratio of the LukG and the LukH components, preferably a complex comprising at least 1 LukG and at least 1 LukH component, or at least 2, or at least 3, or at least 4 of any of the LukG or LukH components or of both LukG and LukH components. The LukGH dimer is herein understood as a heterodimer of one molecule LukG and one molecule LukH, which assemble in solution, specifically by electrostatic or hydrophilic/hydrophobic interactions. Typically, LukH and LukG form a complex in solution without being in contact with target cells.

The term “IGBP” and “IGBP domains” as used herein is specifically understood as any of the five SpA and the two Sbi domains (SpA-A, SpA-B, SpA-C, SpA-D, SpA-E, Sbi-I, Sbi-II) with a triple helix structure that are able to bind the constant region of IgG (Fc) via conserved residues located on helixes 1 and 2 (Deisenhofer, J. Crystallographic refinement and atomic models of a human Fc fragment and its complex with fragment B of Protein A from S. aureus at 2.9- and 2.8-A resolution, Biochemistry 20, 1981, 2361), with the SpA domains having an additional binding site on helixes 2 and 3, that interacts with the variable region of immunoglobulins with VH3 germline (Graille, M. et al. Crystal structure of a Staphylococcus aureus protein A complexed with the Fab fragment of a human IgM antibody: Structural basis for recognition of B-cell receptors and superantigen activity, Proc. Natl. Acad. Sci. USA 97, 2000, 5399). The avirulent KKAA variants of the IGBP domains show reduced binding to the Fc and VH3 (WO 2014/179744A1, US 2014/0170134).

The term “OPK” is herein understood as opsonophagocytic killing of S. aureus induced by an antibody binding to a surface protein of S. aureus. Among the suitable surface protein targets are e.g. IGBP and respective IGBP domains, Clamping factor A and B (CIfA), IsdB, Fibrinogen Binding Protein A and B, and HarA (reviewed in Oleksiewicz, 2012; Jansen, 2013). Enhancing opsonophagocytic uptake and phagocytic killing of pathogens is a common mode of action of antibodies raised against bacterial pathogens. Against Gram positive organisms it is the main antibacterial mechanism since complement mediated killing is not possible due to the Gram positive cell wall. An antibody with OPK activity typically can promote, mediate, or enhance opsonophagocytic killing of S. aureus. Specifically, such OPK activity is determined in a concentration-dependent and serotype-independent manner.

Standard OPK assays may be used to determine the OPK activity of an antibody. Typically, an antibody is understood as having OPK activity, if ≥50% killing of S. aureus bound by the antibody in an in vitro OPK assay can be shown. A test for OPK activity, is e.g. as follows:

Survival of S. aureus is determined in an in vitro OPK assay using freshly isolated human polymorhonuclear cells (PMNs). S. aureus TCH1516 is grown to mid-logarithmic phase in RPMI supplemented with 1% casamino acids. The culture is then washed with assay buffer (50 g/L human Albumin (Albiomin, Biotest) in RPMI supplemented with 2 mM L-Glutamine and 2 mg/mL Sodium bicarbonate) and diluted to 8.6×10⁴ CFU/ml. Bacteria (20 μl) were pre-opsonized with test and control IgGs at 100 μg/ml concentration for 15 min with agitation at 37° C. Human PMNs, purified from human whole blood by a 2-step Percoll gradient centrifugation (Rouha et al., 2015) are diluted to 1.7×10⁷ cells/ml and seeded in 25 μl volumes into half-area flat bottom 96-well plates. Cells are allowed to sediment for 15 min (37° C., 5% CO₂) before addition of pre-opsonized bacteria to the cell layer in a volume of 75 μl. Bacteria and PMNs are compacted (synchronized) by centrifugation for 8 min at 525×g After 1 hour incubation at 37° C. and 5% CO₂, the reaction is stopped by putting the plate on ice. Content of the wells is re-suspended vigorously in the presence of 1% Saponin to lyse phagocytic cells. After an additional 10 min incubation step on ice to ensure complete cell lysis, samples are serially diluted in water and 100 μl were plated in duplicates on Tryptic-Soy-Agar (TSA) plates and incubated o/n at 37° C. for CFU enumeration on the next day.

The term “neutralizing” or “neutralization” is used herein in the broadest sense and refers to any molecule that counteracts a pathogen or inhibits a pathogen, such as S. aureus from infecting a subject, or to inhibit the pathogen from promoting infections by producing potent virulence factors, or to inhibit the virulence factors from exerting its effect in a subject, irrespective of the mechanism by which neutralization is achieved. Neutralization can be achieved, e.g., by an antibody that inhibits the binding and/or interaction of the S. aureus virulence factor(s) with its binding to molecules on target cells or in solution. In certain embodiments, the antibodies described herein can inhibit the virulence factor activity wherein the in vivo or in vitro effects of the interaction between the virulence factor and the host are reduced or eliminated. In the case of IGBP domains, neutralization may occur by allowing the attack by serum IgG binding to surface antigens of S. aureus without interference by the IGBP of S. aureus. Moreover, the anti-IGBP antibodies described herein counteract the S. aureus by promoting OPK.

The neutralization potency of antibodies against cytolytic toxins is typically determined in a standard assay by measuring increased viability or functionality of cells susceptible to the given toxin. Neutralization can be expressed by percent of viable cells with and without antibodies. For highly potent antibodies, a preferred way of expressing neutralization potency is the antibody:toxin molar ratio, where lower values correspond to higher potency. Values below 10 define high, while values below 1 define very high potency.

The term “cross-neutralizing” as used herein shall refer to neutralizing a number of toxins, e.g. toxins incorporating a cross-reactive epitope recognized by the cross-reactive or polyspecific antibody. The term “cross-neutralizing” shall also refer to variants of the same type of toxin, e.g. originating from different strains of S. aureus.

The term “Staphylococcus aureus” or “S. aureus” or “pathogenic S. aureus” is understood in the following way. Staphylococcus aureus bacteria are normally found on the skin or in the nose of people and animals. The bacteria are generally harmless, unless they enter the body through a cut or other wound. Typically, infections are minor skin problems in healthy people. Historically, infections were treated by broad-spectrum antibiotics, such as methicillin. Now, though, certain strains have emerged that are resistant to methicillin and other beta-lactam antibiotics, such as penicillin and cephalosporins. They are referred to as methicillin-resistant Staphylococcus aureus (also known as multi-drug resistant Staphylococcus aureus, or “MRSA”).

Staphylococcus aureus, an important human pathogen, expresses a multitude of secreted toxins (exotoxins). These can attack various host cell types, including erythrocytes, neutrophil granulocytes and other immune cells, as well as epithelial cells of the lung or skin. A prominent member of S. aureus toxins is alpha hemolysin (HIa), which exerts cytolytic function on lymphocytes, macrophages, lung epithelial cells and pulmonary endothelial cells.

S. aureus infections, including MRSA, generally start as small red bumps that resemble pimples, boils or spider bites. These bumps or blemishes can quickly turn into deep, painful abscesses that require surgical draining. Sometimes the bacteria remain confined to the skin. On occasion, they can burrow deep into the body, causing potentially life-threatening infections in a broad range of human tissue, including skin, soft tissue, bones, joints, surgical wounds, the bloodstream, heart valves, lungs, or other organs. Thus, S. aureus infections can result in disease conditions associated therewith, which are potentially fatal diseases, such as necrotizing fasciitis, endocarditis, sepsis, bacteremia, peritonitis, toxic shock syndrome, and various forms of pneumonia, including necrotizing pneumonia, and toxin production in furunculosis and carbunculosis. MRSA infection is especially troublesome in hospital or nursing home settings where patients are at risk of or prone to open wounds, invasive devices, and weakened immune systems and, thus, are at greater risk for infection than the general public.

Antibodies neutralizing S. aureus toxins are interfering with the pathogens and pathogenic reactions, thus able to limit or prevent infection and/ or to ameliorate a disease condition resulting from such infection, or to inhibit S. aureus pathogenesis, in particular pneumonia, peritonitis, osteomyelitis, bacteremia and sepsis pathogenesis. In this regard “protective antibodies” are understood herein as neutralizing antibodies that are responsible for immunity to an infectious agent observed in active or passive immunity. In particular, protective antibodies as described herein are able to neutralize toxic effects (such as cytolysis, induction of pro-inflammatory cytokine expression by target cells) of secreted virulence factors (exotoxins) and hence interfere with pathogenic potential of S. aureus.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation.

As used herein, the term “specificity” or “specific binding” refers to a binding reaction which is determinative of the cognate ligand of interest in a heterogeneous population of molecules. Thus, under designated conditions (e.g. immunoassay conditions), an antibody specifically binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different (understood as at least 1 log difference), preferably the difference is at least 100 fold (understood as at least 2 logs difference), and more preferred a least 1000 fold (understood as at least 3 logs difference) as compared to another antigen.

The term “specificity” or “specific binding” is also understood to apply to binders which bind to one or more molecules, e.g. cross-specific binders. Preferred cross-specific (also called polyspecific or cross-reactive) binders targeting at least two different antigens or targeting a cross-reactive epitope on at least two different antigens, specifically bind the antigens with substantially similar binding affinity, e.g. with less than 100 fold difference or even less than 10 fold difference.

For example, a cross-specific antibody will be able to bind to the various antigens carrying a cross-reactive epitope. Such binding site of an antibody or and antibody with a specificity to bind at least two different antigens or a cross-reactive epitope of at least two different antigens is also called a polyspecific or cross-specific binding site and antibody, respectively. For example, an antibody may have a polyspecific binding site specifically binding an epitope cross-reactive a number of different antigens with sequence homology within the epitope and/or structural similarities to provide for a conformational epitope of essentially the same structure, e.g.

a) cross-reactive at least the HIa and a bi-component toxin of S. aureus, or

b) cross-reactive at least the LukGH variants of different strains, e.g. of at least two, or at least three different S. aureus strains expressing different LukGH variants, such as LukGH variants of strain LukGH TCH1516 and at least one or both of strain MRSA252 and strain MSHR1132; or

c) cross-reactive at least the SpA-E domain and two further IGBP domains of different type.

The epitope which is recognized by a cross-reactive antibody as described herein prevalent on the different toxins, LukGH variants or IGBP-domains is also called “cross-reactive” epitope.

The immunospecificity of an antibody, its binding capacity and the attendant affinity the antibody exhibits for a cross-reactive binding sequence, are determined by a cross-reactive binding sequence with which the antibody immunoreacts (binds). The cross-reactive binding sequence specificity can be defined, at least in part, by the amino acid residues of the variable region of the heavy chain of the immunoglobulin the antibody and/ or by the light chain variable region amino acid residue sequence.

Use of the term “having the same specificity”, “having the same binding site” or “binding the same epitope” indicates that equivalent antibodies exhibit the same or essentially the same, i.e. similar immunoreaction (binding) characteristics and compete for binding to a pre-selected target binding sequence. The relative specificity of an antibody molecule for a particular target can be relatively determined by competition assays, e.g. as described in Harlow, et al., ANTIBODIES: A LABORATORY MANUAL, Cold Spring Harbor Laboratory Press, Cold Spring Harbor, N.Y., 1988).

In particular, the functional activity of variants is determined by specificity to the target antigen(s), e.g. by binding the same epitope or substantially the same epitope as the respective parent antibody.

Antibodies are said to “bind to the same epitope” or “comprising the same binding site” or have “essentially the same binding” characteristics, if the antibodies cross-compete so that only one antibody can bind to the epitope at a given point of time, i.e. one antibody prevents the binding or modulating effect of the other.

The term “compete” or “cross-compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s). Both competing and cross-competing antibodies are encompassed by the present invention.

Competition herein means a greater relative inhibition than about 30% as determined by competition ELISA analysis or by ForteBio analysis, e.g. as described in the Examples section. It may be desirable to set a higher threshold of relative inhibition as criteria of what is a suitable level of competition in a particular context, e.g., where the competition analysis is used to select or screen for new antibodies designed with the intended function of the binding of additional or other toxins of S. aureus. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

The term “subject” as used herein shall refer to a warm-blooded mammalian, particularly a human being or a non-human animal. MRSA is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a S. aureus infection. The subject may be a patient at risk of a S. aureus infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g. treated for prophylaxis or therapy of S. aureus disease conditions. In particular, the subject is treated, which is either at risk of infection or developing such disease or disease recurrence, or a subject that is suffering from such infection and/ or disease associated with such infection.

Specifically the term “prophylaxis” refers to preventive measures which is intended to encompass prevention of the onset of pathogenesis or prophylactic measures to reduce the risk of pathogenesis.

Specifically, the method for treating, preventing, or delaying a disease condition in a subject as described herein, is by interfering with the pathogenesis of S. aureus as causal agent of the condition, wherein the pathogenesis includes a step of forming a pore on the subject's cellular membrane, e.g. by the specific virulence factors or toxins.

The term “toxin” as used herein shall refer to the alpha-toxin (HIa) and the bi-component toxins of S. aureus. It is specifically understood that the toxins targeted by an antibody as described herein are either the toxins as such, e.g. the soluble monomeric toxins or in the form of the pore forming toxins as expressed by S. aureus, or toxin components, such as the components of the bi-component toxins. Therefore, the term “toxin” as used herein shall refer to both, the toxin or the toxin components bearing the immunorelevant epitope.

The virulence of S. aureus is due to a combination of numerous virulence factors, which include surface-associated proteins that allow the bacterium to adhere to eukaryotic cell membranes, a capsular polysaccharide that protects it from opsonophagocytosis, and several exotoxins. S. aureus causes disease mainly through the production of secreted virulence factors such as hemolysins, enterotoxins and toxic shock syndrome toxin. These secreted virulence factors suppress the immune response by inactivating many immunological mechanisms in the host, and cause tissue destruction and help establish the infection. The latter is accomplished by a group of pore forming toxins, the most prominent of which is HIa, a key virulence factor for S. aureus pneumonia.

S. aureus produces a diverse array of further virulence factors and toxins that enable this bacterium to counteract and withstand attack by different kinds of immune cells, specifically subpopulations of white blood cells that make up the body's primary defense system. The production of these virulence factors and toxins allow S. aureus to maintain an infectious state. Among these virulence factors, S. aureus produces several bi-component leukotoxins, which damage membranes of host defense cells and erythrocytes by the synergistic action of two non-associated proteins or subunits. Among these bi-component toxins, gamma-hemolysin (HIgAB and HIgCB) and the Pantone-Valentine Leukocidin (PVL) are the best characterized.

The toxicity of the leukocidins towards mammalian cells involves the action of two components. The first subunit is named class S component, and the second subunit is named class F component. The S and F subunits act synergistically to form pores on white blood cells including monocytes, macrophages, dendritic cells and neutrophils (collectively known as phagocytes). The gamma hemolysins, especially HIgAB and HIgA-LukD also act on red blood cells and LukED on T cells. The repertoire of bi-component leukotoxins produced by S. aureus is known to include cognate and non-cognate pairs of the F and S components, e.g. gamma-hemolysins, PVL toxins and PVL-like toxins, including HIgAB, HIgCB, LukSF, LukED, LukGH, LukS-HIgB, LukSD, HIgA-LukD, HIgA-LukF, LukG-HIgA, LukEF, LukE-HIgB, HIgC-LukD or HIgC-LukF, which are preferred targets as described herein.

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like). Isolated antibodies as described herein are specifically purified from cell culture or provided as substantially pure proteins.

The term “therapeutically effective amount”, used herein interchangeably with any of the terms “effective amount” or “sufficient amount” of a compound, e.g. an antibody as described herein, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of S. aureus or S. aureus pathogenesis.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

An antibody or combination preparation as described herein may be used prophylactically to inhibit onset of S. aureus infection, or therapeutically to treat S. aureus infection, particularly S. aureus infections such as MRSA that are known to be refractory or in the case of the specific subject, have proven refractory to treatment with other conventional antibiotic therapy.

A therapeutically effective amount of an antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g. for treating acute disease conditions. The combination preparation may contain the respective therapeutically effective amounts of each of the antibodies.

Moreover, a treatment or prevention regime of a subject with a therapeutically effective amount of an antibody as described herein may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

The invention specifically provides pharmaceutical compositions which comprise an antibody or the antibody combination as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody as described herein and one or more therapeutically active agents are formulated. Stable formulations of an antibody are prepared for storage by mixing said antibody having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody as described herein, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, e.g. employing inhalable technology or pulmonary delivery systems, vaginally, parenterally, rectally, or intraocularly.

Exemplary formulations as used for parenteral administration include those suitable for subcutaneous, intramuscular or intravenous injection as, for example, a sterile solution, emulsion or suspension.

In one embodiment, the antibodies or the combination preparation as described herein are the only therapeutically active agents administered to a subject, e.g. as a disease modifying or preventing monotherapy.

In another embodiment, the antibodies or the combination preparation as described herein are combined with further antibodies in a cocktail, e.g. combined in a mixture or kit of parts, to target S. aureus, such that the cocktail contains more therapeutically active agents administered to a subject, e.g. as a disease modifying or preventing combination therapy.

Alternatively, the antibodies or the combination preparation as described herein are administered in combination with one or more other therapeutic or prophylactic agents, including but not limited to standard treatment, e.g. antibiotics, steroid and non-steroid inhibitors of inflammation, and/or other antibody based therapy, e.g. employing anti-bacterial or anti-inflammatory agents.

A combination therapy is particularly employing a standard regimen, e.g. as used for treating MRSA infection. This may include antibiotics, e.g. tygecycline, linezolide, methicillin and/or vancomycin.

In a combination therapy, the antibodies or the combination preparation as described herein may be administered as a mixture, or concomitantly with one or more other therapeutic regimens, e.g. either before, simultaneously or after concomitant therapy.

Another aspect of the present invention provides a kit comprising one or more of the antibodies of the combination preparation as described herein in different containers. The kit may include, in addition to the one or more antibodies, various other therapeutic agents and auxiliary agents and devices to prepare the pharmaceutical formulations ready for use. A kit may also include instructions for use in a therapeutic method. Such instructions can be, for example, provided on a device included in the kit. In another specific embodiment, the kit includes an antibody in the lyophilized form, in combination with pharmaceutically acceptable carrier(s) that can be mixed before use to reconstitute the lyophilisate and to produce an injectable soution for near term administration.

The biological properties of the antibodies or the combination preparation as described herein or the respective pharmaceutical preparations may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example, because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibodies or the combination preparation as described herein and respective pharmaceutical compositions of the present invention may be tested in humans to determine their therapeutic or prophylactic efficacy, toxicity, immunogenicity, pharmacokinetics, and/or other clinical properties.

In some embodiments, a combination of antibodies described herein exhibits a “synergistic” effect, in that the effect (e.g., in vitro and/or in vivo effect described herein) of a combination of antibodies is greater than the additive effect of individual antibodies (and/or a subset of antibodies) included in the combination. In some embodiments, an effect of a combination is 10%, 20%, 30%, 40%, 50%, 60%, 70%, 80%, 90%, 100%, 150%, 200%, or higher, relative to the additive effect of individual antibodies (and/or a subset of antibodies) included in the combination.

The subject matter of the following definitions is considered embodiments of the present invention:

1. An anti-Staphylococcus aureus antibody combination preparation comprising

a) a toxin cross-neutralizing antibody comprising at least one polyspecific binding site that binds to alpha-toxin (HIa) and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and

b) an anti-LukGH antibody comprising at least one binding site that specifically binds to the LukGH complex; and/or

c) an anti-Ig-binding protein (IGBP) antibody comprising at least one CDR binding site recognizing any of the S. aureus IgG binding domains of Protein A or Sbi.

2. The combination preparation of definition 1, wherein the toxin cross-neutralizing antibody has a cross-specificity to bind HIa and at least one of the F-components of the bi-component toxins, preferably at least two or three thereof, preferably wherein the F-components are selected from the group consisting of HIgB, LukF and LukD, or any F-component of the cognate and non-cognate pairs of F and S components of gamma-hemolysins, PVL toxins and PVL-like toxins, preferably HIgAB, HIgCB, LukSF, LukED, LukS-HIgB, LukSD, HIgA-LukD, HIgA-LukF, LukEF, LukE-HIgB, HIgC-LukD or HIgC-LukF.

3. The combination preparation of definition 1 or 2, wherein the toxin cross-neutralizing antibody has a cross-specificity to bind HIa and at least one of HIgAB, HIgCB, LukSF, and LukED.

4. The combination preparation of any of definitions 1 to 3, wherein the toxin cross-neutralizing antibody comprises at least three complementarity determining regions (CDR1 to CDR3) of the antibody heavy chain variable region (VH), wherein

A) the antibody comprises
a) a CDR1 comprising or consisting of the amino acid sequence SEQ ID 1; and
b) a CDR2 comprising or consisting of the amino acid sequence SEQ ID 2; and
c) a CDR3 comprising or consisting of the amino acid sequence SEQ ID 3;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 1;
b) the parent CDR2 consists of the amino acid sequence SEQ ID 2; and
c) the parent CDR3 consists of the amino acid sequence SEQ ID 3.

5. The combination preparation of definition 4, wherein the toxin cross-neutralizing antibody comprises
a) in VH CDR1 at position 5, the amino acid residue selected from the group consisting of S, A, D, E, F, G, H, I, K, L, M, N, Q, R, T V, W and Y, preferentially any of H, R and W;
b) in VH CDR1 at position 7, the amino acid residue selected from the group consisting of M, H, K, Q, R and W, preferentially any of K, R or W;
c) in VH CDR2 at position 3, the amino acid residue is selected from the group consisting of D and R;
d) in VH CDR2 at position 7, the amino acid residue selected from the group consisting of S, A, D, E, F, H, K, M, N, Q, R, T, W and Y, preferentially any of D, H, K, N or Q, and more preferentially is Q;
e) in VH CDR2 at position 9, the amino acid residue selected from the group consisting of Y, F, K, L, Q and R, and preferentially is R;
f) in VH CDR3 at position 5, the amino acid residue selected from the group consisting of G, A, D, F, H, I, M, N, R, S, T, V and Y, preferentially any of D, F, H, I, M, N, R, T, V or Y;
g) in VH CDR3 at position 6, the amino acid residue selected from the group consisting of H, E, Q and S, preferentially any of E or Q;
h) in VH CDR3 at position 7, the amino acid residue selected from the group consisting of G, A, D, E, H, I, M, N, Q, S, T, V and W, and preferentially is W; and/or
i) in VH CDR3 at position 8, the amino acid residue selected from the group consisting of V, A, D, E, G, I, K, L, M, Q, R, S and T, preferentially any of M or R.

6. The combination preparation of definition 4, wherein the toxin cross-neutralizing antibody comprises a functionally active CDR variant which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

7. The combination preparation of any of definitions 4 to 6, wherein the toxin cross-neutralizing antibody comprises at least one functionally active CDR variant which is any of
a) a CDR1 sequence selected from the group consisting of SEQ ID 4, and SEQ ID 5; or
b) a CDR2 sequence selected from the group consisting of SEQ ID 6, SEQ ID 7, SEQ ID 8, SEQ ID 9, and SEQ ID 10; or
c) a CDR3 sequence selected from the group consisting of SEQ ID 11, and SEQ ID 12.

8. The combination preparation of any of definitions 4 to 7, wherein the toxin cross-neutralizing antibody is selected from the group consisting of

a) an antibody comprising
a. the CDR1 sequence SEQ ID 1; and
b. the CDR2 sequence SEQ ID 6; and
c. the CDR3 sequence SEQ ID 11;

b) an antibody comprising
a. the CDR1 sequence SEQ ID 4; and
b. the CDR2 sequence SEQ ID 7; and
c. the CDR3 sequence SEQ ID 3;

c) an antibody comprising
a. the CDR1 sequence SEQ ID 1; and
b. the CDR2 sequence SEQ ID 8; and
c. the CDR3 sequence SEQ ID 3;

d) an antibody comprising
a. the CDR1 sequence SEQ ID 1; and
b. the CDR2 sequence SEQ ID 2; and
c. the CDR3 sequence SEQ ID 12;

e) an antibody comprising
a. the CDR1 sequence SEQ ID 5; and
b. the CDR2 sequence SEQ ID 9; and
c. the CDR3 sequence SEQ ID 3;

and

f) an antibody comprising
a. the CDR1 sequence SEQ ID 5; and
b. the CDR2 sequence SEQ ID 10; and
c. the CDR3 sequence SEQ ID 3;

9. The combination preparation of any of definitions 4 to 7, wherein the toxin cross-neutralizing antibody comprises a VH amino acid sequence selected from the group consisting of SEQ ID 20-31, preferably comprising an antibody heavy chain (HC) amino acid sequence selected from the group consisting of SEQ ID 40-51, or any of the amino acid sequences SEQ ID 40-51 with a deletion of the C-terminal amino acid.

10. The combination preparation of any of definitions 4 to 9, wherein the toxin cross-neutralizing antibody further comprises at least three complementarity determining regions (CDR4 to CDR6) of the antibody light chain variable region (VL), preferably wherein

A) the antibody comprises
a) a CDR4 comprising or consisting of the amino acid sequence SEQ ID 32; and
b) a CDR5 comprising or consisting of the amino acid sequence SEQ ID 33; and
c) a CDR6 comprising or consisting of the amino acid sequence SEQ ID 34;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 32; or
b) the parent CDR5 consists of the amino acid sequence SEQ ID 33; or
c) the parent CDR6 consists of the amino acid sequence SEQ ID 34.

11. The combination preparation of definition 10, wherein the toxin cross-neutralizing antibody comprises
a) in VL CDR4 at position 7, the amino acid residue selected from the group consisting of S, A, E, F, G, K, L, M, N, Q, R, W and Y, preferentially any of L, M, R or W, and more preferentially is R;
b) in VL CDR5 at position 1, the amino acid residue selected from the group consisting of A and G;
c) in VL CDR5 at position 3, the amino acid residue selected from the group consisting of S, A, D, G, H, I, K, L, N, Q, R, T, V and W;
d) in VL CDR5 at position 4, the amino acid residue selected from the group consisting of S, D, E, H, I, K, M, N, Q, R, T and V, preferentially any of K, N, Q and R;
e) in VL CDR6 at position 3, the amino acid residue selected from the group consisting of G, A, D, E, F, H, I, K, L, N, Q, R, S, T, V, W and Y;
f) in VL CDR6 at position 4, the amino acid residue selected from the group consisting of Y, D, F, H, M, R and W;
g) in VL CDR6 at position 5, the amino acid residue selected from the group consisting of V, A, D, E, F, G, H, I, K, L, M, N, Q, R, S, T, and W; and/or
h) in VL CDR6 at position 6, the amino acid residue selected from the group consisting of F and W.

12. The combination preparation of definition 10 or 11, wherein the toxin cross-neutralizing antibody comprises a VL amino acid sequence SEQ ID 39 or an antibody light chain (LC) amino acid SEQ ID 52.

13. The combination preparation of any of definitions 1 to 12, wherein the toxin cross-neutralizing antibody comprises at least one polyspecific binding site that binds to alpha-toxin (HIa) and at least one of the bi-component toxins of S. aureus, which antibody is a functionally active variant antibody of a parent antibody that comprises a polyspecific binding site of the VH amino acid sequence SEQ ID 20, and the VL amino acid sequence SEQ ID 39, which functionally active variant antibody comprises at least one point mutation in any of the framework regions (FR) or constant domains, or complementarity determining regions (CDR1 to CDR6) in any of SEQ ID 20 or SEQ 39, and has an affinity to bind each of the toxins with a K_{D} of less than 10⁻⁸M, preferably less than 10⁻⁹M.

14. The combination preparation of definitions 12, wherein the toxin cross-neutralizing antibody comprises
a) in VH CDR1 at position 5, the amino acid residue selected from the group consisting of S, A, D, E, F, G, H, I, K, L, M, N, Q, R, T V, W and Y, preferentially any of H, R and W;
b) in VH CDR1 at position 7, the amino acid residue selected from the group consisting of M, H, K, Q, R and W, preferentially any of K, R or W;
c) in VH CDR2 at position 3, the amino acid residue selected from the group consisting of D and R;
d) in VH CDR2 at position 7, the amino acid residue selected from the group consisting of S, A, D, E, F, H, K, M, N, Q, R, T, W and Y, preferentially any of D, H, K, N or Q, and more preferentially is Q;
e) in VH CDR2 at position 9, the amino acid residue selected from the group consisting of Y, F, K, L, Q and R, and preferentially is R;
f) in VH CDR3 at position 5, the amino acid residue selected from the group consisting of G, A, D, F, H, I, M, N, R, S, T, V and Y, preferentially any of D, F, H, I, M, N, R, T, V or Y;
g) in VH CDR3 at position 6, the amino acid residue selected from the group consisting of H, E, Q and S, preferentially any of E or Q;
h) in VH CDR3 at position 7, the amino acid residue selected from the group consisting of G, A, D, E, H, I, M, N, O, S, T, V and W, and preferentially is W; and/or
i) in VH CDR3 at position 8, the amino acid residue selected from the group consisting of V, A, D, E, G, I, K, L, M, Q, R, S and T, preferentially any of M or R.

15. The combination preparation of definition 13 or 14, wherein the toxin cross-neutralizing antibody comprises
a) in VL CDR4 at position 7, the amino acid residue selected from the group consisting of S, A, E, F, G, K, L, M, N, Q, R, W and Y, preferentially any of L, M, R or W, and more preferentially is R;
b) in VL CDR5 at position 1, the amino acid residue selected from the group consisting of A and G;
c) in VL CDR5 at position 3, the amino acid residue selected from the group consisting of S, A, D, G, H, I, K, L, N, Q, R, T, V and W;
d) in VL CDR5 at position 4, the amino acid residue selected from the group consisting of S, D, E, H, I, K, M, N, Q, R, T and V, preferentially any of K, N, Q and R;
e) in VL CDR6 at position 3, the amino acid residue selected from the group consisting of G, A, D, E, F, H, I, K, L, N, Q, R, S, T, V, W and Y;
f) in VL CDR6 at position 4, the amino acid residue selected from the group consisting of Y, D, F, H, M, R and W;
g) in VL CDR6 at position 5, the amino acid residue selected from the group consisting of V, A, D, E, F, G, H, I, K, L, M, N, Q, R, S, T, and W; and/or
h) in VL CDR6 at position 6, the amino acid residue selected from the group consisting of F and W.

16. The combination preparation of any of definitions 1 to 15, wherein the anti-LukGH antibody comprises any of the CDR1 to CDR3 sequences as listed in Table 2, or functionally active CDR variants thereof.

17. The combination preparation of definition 16, wherein the anti-LukGH antibody is selected from the group consisting of group members i) to viii), wherein

i)

A) the antibody comprises
a) a CDR1 comprising or consisting of the amino acid sequence SEQ ID 86 or SEQ ID 99; and
b) a CDR2 comprising or consisting of the amino acid sequence SEQ ID 88; and
c) a CDR3 comprising or consisting of the amino acid sequence SEQ ID 90;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 86 or SEQ ID 99; or
b) the parent CDR2 consists of the amino acid sequence SEQ ID 88; or
c) the parent CDR3 consists of the amino acid sequence SEQ ID 90;

ii)

A) the antibody comprises
a) a CDR1 comprising or consisting of any of the amino acid sequences SEQ ID 110, SEQ ID 120, or SEQ ID 122; and
b) a CDR2 comprising or consisting of any of the amino acid sequences SEQ ID 112, SEQ ID 121, SEQ ID 123, or SEQ ID 124; and
c) a CDR3 comprising or consisting of the amino acid sequence SEQ ID 114;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 110, SEQ ID 120, or SEQ ID 122; or
b) the parent CDR2 consists of the amino acid sequence SEQ ID 112, SEQ ID 121, SEQ ID 123, or SEQ ID 124; or
c) the parent CDR3 consists of the amino acid sequence SEQ ID 114;

iii)

A) the antibody comprises
a) a CDR1 comprising or consisting any of the amino acid sequences SEQ ID 131, SEQ ID 139, SEQ ID 141, SEQ ID 143, SEQ ID 145, SEQ ID 147, or SEQ ID 148; and
b) a CDR2 comprising or consisting of any of the amino acid sequences SEQ ID 133, SEQ ID 140, SEQ ID 142, SEQ ID 144, SEQ ID 146, SEQ ID 149, or SEQ ID 150; and
c) a CDR3 comprising or consisting of the amino acid sequence SEQ ID 135;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 131, SEQ ID 139, SEQ ID 141, SEQ ID 143, SEQ ID 145, SEQ ID 147, or SEQ ID 148; or
b) the parent CDR2 consists of the amino acid sequence SEQ ID 133, SEQ ID 140, SEQ ID 142, SEQ ID 144, SEQ ID 146, SEQ ID 149, or SEQ ID 150; or
c) the parent CDR3 consists of the amino acid sequence SEQ ID 135;

iv)

A) the antibody comprises
a) a CDR1 comprising or consisting of any of the amino acid sequences SEQ ID 155, SEQ ID 161, SEQ ID 163, SEQ ID 165, SEQ ID 167, or SEQ ID 169; and
b) a CDR2 comprising or consisting of any of the amino acid sequences SEQ ID 156, SEQ ID 162, SEQ ID 168, or SEQ ID 88; and
c) a CDR3 comprising or consisting of the amino acid sequence SEQ ID 157;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 155, SEQ ID 161, SEQ ID 163, SEQ ID 165, SEQ ID 167, or SEQ ID 169; or
b) the parent CDR2 consists of the amino acid sequence SEQ ID 156, SEQ ID 162, SEQ ID 168, or SEQ ID 88; or
c) the parent CDR3 consists of the amino acid sequence SEQ ID 157;

v)

A) the antibody comprises
a) a CDR1 comprising or consisting of any of the amino acid sequences SEQ ID 171, SEQ ID 181, SEQ ID 183, or SEQ ID 185; and
b) a CDR2 comprising or consisting of any of the amino acid sequences SEQ ID 172, SEQ ID 182, SEQ ID 184, or SEQ ID 186; and
c) a CDR3 comprising or consisting of the amino acid sequence SEQ ID 173;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 171, SEQ ID 181, SEQ ID 183, or SEQ ID 185; or
b) the parent CDR2 consists of the amino acid sequence SEQ ID 172, SEQ ID 182, SEQ ID 184, or SEQ ID 186; or
c) the parent CDR3 consists of the amino acid sequence SEQ ID 173;

vi)

A) the antibody comprises
a) a CDR1 comprising or consisting of any of the amino acid sequences SEQ ID 188, SEQ ID 194, SEQ ID 196, SEQ ID 122, SEQ ID 198, SEQ ID 203, or SEQ ID 204; and
b) a CDR2 comprising or consisting of any of the amino acid sequences SEQ ID 189, SEQ ID 193, SEQ ID 195, SEQ ID 197, SEQ ID 186, SEQ ID 199, or SEQ ID 205; and
c) a CDR3 comprising or consisting of the amino acid sequence SEQ ID 190;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 188, SEQ ID 194, SEQ ID 196, SEQ ID 122, SEQ ID 198, SEQ ID 203, or SEQ ID 204; or
b) the parent CDR2 consists of the amino acid sequence SEQ ID 189, SEQ ID 193, SEQ ID 195, SEQ ID 197, SEQ ID 186, SEQ ID 199, or SEQ ID 205; or
c) the parent CDR3 consists of the amino acid sequence SEQ ID 190;

vii)

A) the antibody comprises
a) a CDR1 comprising or consisting of the amino acid sequence SEQ ID 209; and
b) a CDR2 comprising or consisting of the amino acid sequence SEQ ID 210; and
c) a CDR3 comprising or consisting of the amino acid sequence SEQ ID 211;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 209; or
b) the parent CDR2 consists of the amino acid sequence SEQ ID 210; or
c) the parent CDR3 consists of the amino acid sequence SEQ ID 211;

and viii)

A) the antibody comprises
a) a CDR1 comprising or consisting of the amino acid sequence SEQ ID 218; and
b) a CDR2 comprising or consisting of the amino acid sequence SEQ ID 219; and
c) a CDR3 comprising or consisting of the amino acid sequence SEQ ID 221;

or

B) the antibody is an antibody of A, wherein at least one of the CDR1, CDR2, or CDR3 is a functionally active CDR variant of a parent CDR, comprising at least one point mutation in the parent CDR and at least 60% sequence identity with the parent CDR, wherein
a) the parent CDR1 consists of the amino acid sequence SEQ ID 218; or
b) the parent CDR2 consists of the amino acid sequence SEQ ID 219; or
c) the parent CDR3 consists of the amino acid sequence SEQ ID 221.

18. The combination preparation of definition 17, wherein the anti-LukGH antibody is an antibody of group member iv) or a functionally active variant thereof, wherein

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H; and/or

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y.

19. The combination preparation of any of definitions 16 to 18, wherein the anti-LukGH antibody comprises a functionally active CDR variant which is characterized by at least one of
a) 1, 2, or 3 point mutations in the parent CDR sequence; or
b) 1 or 2 point mutations in any of the four C-terminal or four N-terminal, or four centric amino acid positions of the parent CDR sequence.

20. The combination preparation of any of definitions 16 to 19, wherein the anti-LukGH antibody is selected from the group consisting of

a) an antibody comprising
a. the CDR1 sequence SEQ ID 122; and
b. the CDR2 sequence SEQ ID 123; and
c. the CDR3 sequence SEQ ID 114;

b) an antibody comprising
a. the CDR1 sequence SEQ ID 131; and
b. the CDR2 sequence SEQ ID 133; and
c. the CDR3 sequence SEQ ID 135;

c) an antibody comprising
a. the CDR1 sequence SEQ ID 167; and
b. the CDR2 sequence SEQ ID 168; and
c. the CDR3 sequence SEQ ID 157;

d) an antibody comprising
a. the CDR1 sequence SEQ ID 188; and
b. the CDR2 sequence SEQ ID 189; and
c. the CDR3 sequence SEQ ID 190;

and

e) an antibody comprising
a. the CDR1 sequence SEQ ID 198; and
b. the CDR2 sequence SEQ ID 199; and
c. the CDR3 sequence SEQ ID 190.

21. The combination preparation of any of definitions 16 to 19, wherein the anti-LukGH antibody comprises any of
a) a VH amino acid sequence selected from any of the VH sequences as depicted in FIG. 2;
b) an antibody heavy chain (HC) amino acid sequence selected from the group consisting of SEQ ID 231, SEQ ID 233, SEQ ID 235, SEQ ID 237, SEQ ID 239, SEQ ID 241, SEQ ID 243, SEQ ID 245, SEQ ID 247, SEQ ID 249, SEQ ID 251, SEQ ID 253, and SEQ ID 255; or
c) an antibody heavy chain (HC) amino acid sequence selected from the group consisting of SEQ ID 231, SEQ ID 233, SEQ ID 235, SEQ ID 237, SEQ ID 239, SEQ ID 241, SEQ ID 243, SEQ ID 245, SEQ ID 247, SEQ ID 249, SEQ ID 251, SEQ ID 253, and SEQ ID 255, which is further comprising a deletion of the C-terminal amino acid and/or a Q1E point mutation, if the first amino acid of the VH sequence is a Q.

22. The combination preparation of any of definitions 16 to 21, wherein the anti-LukGH antibody further comprises an antibody light chain variable region (VL), which comprises any of the CDR4 to CDR6 sequences as listed in Table 2, or functionally active CDR variants thereof.

23. The combination preparation of definition 22, wherein the anti-LukGH antibody is selected from the group consisting of group members i) to viii), wherein

i)

A) the antibody comprises
a) a CDR4 comprising or consisting of the amino acid sequence SEQ ID 93 or SEQ ID 103; and
b) a CDR5 comprising or consisting of any of the amino acid sequences SEQ ID 95, SEQ ID 100, or SEQ ID 105; and
c) a CDR6 comprising or consisting of any of the amino acid sequences SEQ ID 97, SEQ ID 101, SEQ ID 107, or SEQ ID 108;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 93 or SEQ ID 103; or
b) the parent CDR5 consists of the amino acid sequence SEQ ID 95, SEQ ID 100, or SEQ ID 105; or
c) the parent CDR6 consists of the amino acid sequence SEQ ID 97, SEQ ID 101, SEQ ID 107, or SEQ ID 108;

ii)

A) the antibody comprises
a) a CDR4 comprising or consisting of the amino acid sequence SEQ ID 116; and
b) a CDR5 comprising or consisting of the amino acid sequence SEQ ID 117 or SEQ ID 125; and
c) a CDR6 comprising or consisting of any of the amino acid sequences SEQ ID 119, SEQ ID 126, SEQ ID 127, or SEQ ID 129;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 116; or
b) the parent CDR5 consists of the amino acid sequence SEQ ID 117 or SEQ ID 125; or
c) the parent CDR6 consists of the amino acid sequence SEQ ID 119, SEQ ID 126, SEQ ID 127, or SEQ ID 129;

iii)

A) the antibody comprises
a) a CDR4 comprising or consisting of any of the amino acid sequences SEQ ID 137, SEQ ID 151, or SEQ ID 103; and
b) a CDR5 comprising or consisting of the amino acid sequence SEQ ID 105; and
c) a CDR6 comprising or consisting of any of the amino acid sequences SEQ ID 138, SEQ ID 152, SEQ ID 153, or SEQ ID 154;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 137, SEQ ID 151, or SEQ ID 103; or
b) the parent CDR5 consists of the amino acid sequence SEQ ID 105; or
c) the parent CDR6 consists of the amino acid sequence SEQ ID 138, SEQ ID 152, SEQ ID 153, or SEQ ID 154;

iv)

A) the antibody comprises
a) a CDR4 comprising or consisting of the amino acid sequence SEQ ID 159 or SEQ ID 116; and
b) a CDR5 comprising or consisting of the amino acid sequence SEQ ID 125; and
c) a CDR6 comprising or consisting of the amino acid sequence SEQ ID 160 or SEQ ID 170;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 159 or SEQ ID 116; or
b) the parent CDR5 consists of the amino acid sequence SEQ ID 125; or
c) the parent CDR6 consists of the amino acid sequence SEQ ID 160 or SEQ ID 170;

v)

A) the antibody comprises
a) a CDR4 comprising or consisting of the amino acid sequence SEQ ID 176; and
b) a CDR5 comprising or consisting of any of the amino acid sequence SEQ ID 178; and
c) a CDR6 comprising or consisting of the amino acid sequence SEQ ID 180 or SEQ ID 187;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176; or
b) the parent CDR5 consists of the amino acid sequence SEQ ID 178; or
c) the parent CDR6 consists of the amino acid sequence SEQ ID 180 or SEQ ID 187;

vi)

A) the antibody comprises
a) a CDR4 comprising or consisting of the amino acid sequence SEQ ID 176 or SEQ ID 200; and
b) a CDR5 comprising or consisting of the amino acid sequence SEQ ID 178 or SEQ ID 201; and
c) a CDR6 comprising or consisting of any of the amino acid sequences SEQ ID 192, SEQ ID 202, or SEQ ID 207;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176 or SEQ ID 200; or
b) the parent CDR5 consists of the amino acid sequence SEQ ID 178 or SEQ ID 201; or
c) the parent CDR6 consists of the amino acid sequence SEQ ID 192, SEQ ID 202, or SEQ ID 207;

vii)

A) the antibody comprises
a) a CDR4 comprising or consisting of the amino acid sequence SEQ ID 116; and
b) a CDR5 comprising or consisting of the amino acid sequence SEQ ID 125; and
c) a CDR6 comprising or consisting of any of the amino acid sequences SEQ ID 213, SEQ ID 214, SEQ ID 215, or SEQ ID 216;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 116; or
b) the parent CDR5 consists of the amino acid sequence SEQ ID 125; or
c) the parent CDR6 consists of the amino acid sequence SEQ ID 213, SEQ ID 214, SEQ ID 215, or SEQ ID 216;

and viii)

A) the antibody comprises
a) a CDR4 comprising or consisting of the amino acid sequence SEQ ID 176 or SEQ ID 200; and
b) a CDR5 comprising or consisting of the amino acid sequence SEQ ID 178; and
c) a CDR6 comprising or consisting of any of the amino acid sequences SEQ ID 224, SEQ ID 180, SEQ ID 226, or SEQ ID 227;

or

B) the antibody is an antibody of A, wherein at least one of the CDR4, CDR5, or CDR6 is a functionally active CDR variant of a parent CDR, comprising at least one point mutation in the parent CDR and at least 60% sequence identity with the parent CDR, wherein
a) the parent CDR4 consists of the amino acid sequence SEQ ID 176 or SEQ ID 200; or
b) the parent CDR5 consists of the amino acid sequence SEQ ID 178; or
c) the parent CDR6 consists of the amino acid sequence SEQ ID 224, SEQ ID 180, SEQ ID 226, or SEQ ID 227.

24. The combination preparation of definition 23, wherein the anti-LukGH antibody is an antibody of group member iv) or a functionally active variant thereof, wherein

a) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

b) in VL CDR4 at position 8, the amino acid residue is selected from S, A, D, E, F, G, H, I, K, L, M, N, Q, R, T, V, W, and Y, preferentially I or W;

c) in VL CDR4 at position 9, the amino acid residue is selected from Y, F, R and W, and preferentially R or W;

d) in VL CDR5 at position 1, the amino acid residue is selected from A, G, S, W and Y, and preferentially is G;

e) in VL CDR6 at position 4, the amino acid residue is selected from F, H, M, W and Y;

f) in VL CDR6 at position 5, the amino acid residue is selected from D, A, D, E, F, G, H, I, K, L, M, N, Q, R, S, T, V, W, and Y; and/or

g) in VL CDR6 at position 8, the amino acid residue is selected from F, H, R and W

25. The combination preparation of definition 22 or 23, wherein the anti-LukGH antibody comprises a VL amino acid sequence selected from any of the VL sequences as depicted in FIG. 2, or an antibody light chain (LC) amino acid sequence selected from the group consisting of SEQ ID 232, SEQ ID 234, SEQ ID 236, SEQ ID 238, SEQ ID 240, SEQ ID 242, SEQ ID 244, SEQ ID 246, SEQ ID 248, SEQ ID 250, SEQ ID 252, SEQ ID 254, and SEQ ID 256, or a functionally active CDR variant of any of the foregoing, which has an affinity to bind the LukGH complex with a K_{D} of less than 10⁻⁸M, preferably less than 10⁻⁹M.

26. The combination preparation of definition 25, wherein the anti-LukGH antibody or the functionally active variant thereof comprises a VL amino acid sequence selected from any of the VL sequences as depicted in FIG. 2, Group 4, or an antibody light chain (LC) amino acid sequence selected from the group consisting of SEQ ID 242, SEQ ID 244, SEQ ID 246, wherein

a) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

b) in VL CDR4 at position 8, the amino acid residue is selected from S, A, D, E, F, G, H, I, K, L, M, N, Q, R, T, V, W, and Y, preferentially I or W;

c) in VL CDR4 at position 9, the amino acid residue is selected from Y, F, R and W, and preferentially R or W;

d) in VL CDR5 at position 1, the amino acid residue is selected from A, G, S, W and Y, and preferentially is G;

e) in VL CDR6 at position 4, the amino acid residue is selected from F, H, M, W and Y;

f) in VL CDR6 at position 5, the amino acid residue is selected from D, A, D, E, F, G, H, I, K, L, M, N, Q, R, S, T, V, W, and Y; and/or

g) in VL CDR6 at position 8, the amino acid residue is selected from F, H, R and W.

27. The combination preparation of any of definitions 16 to 26, wherein the anti-LukGH antibody is selected from the group consisting of

a) an antibody comprising
a. the CDR1 sequence SEQ ID 122; and
b. the CDR2 sequence SEQ ID 123; and
c. the CDR3 sequence SEQ ID 114; and
d. the CDR4 sequence SEQ ID 116; and
e. the CDR5 sequence SEQ ID 117; and
f. the CDR6 sequence SEQ ID 119;

b) an antibody comprising
a. the CDR1 sequence SEQ ID 131; and
b. the CDR2 sequence SEQ ID 133; and
c. the CDR3 sequence SEQ ID 135; and
d. the CDR4 sequence SEQ ID 137; and
e. the CDR5 sequence SEQ ID 105; and
f. the CDR6 sequence SEQ ID 138;

c) an antibody comprising
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;

d) an antibody comprising
a. the CDR1 sequence SEQ ID 188; and
b. the CDR2 sequence SEQ ID 189; and
c. the CDR3 sequence SEQ ID 190; and
d. the CDR4 sequence SEQ ID 176; and
e. the CDR5 sequence SEQ ID 178; and
f. the CDR6 sequence SEQ ID 192;

and

e) an antibody comprising
a. the CDR1 sequence SEQ ID 198; and
b. the CDR2 sequence SEQ ID 199; and
c. the CDR3 sequence SEQ ID 190; and
d. the CDR4 sequence SEQ ID 200; and
e. the CDR5 sequence SEQ ID 201; and
f. the CDR6 sequence SEQ ID 202;

or a functionally active CDR variant of any of the foregoing, which has an affinity to bind the LukGH complex with a K_{D} of less than 10⁻⁸M, preferably less than 10⁻⁹M.

28. The combination preparation of definition 27, wherein the anti-LukGH antibody is an antibody of group member c) or a functionally active variant thereof, wherein:

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H;

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y;

k) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

l) in VL CDR4 at position 8, the amino acid residue is selected from S, A, D, E, F, G, H, I, K, L, M, N, Q, R, T, V, W, and Y, preferentially I or W;

m) in VL CDR4 at position 9, the amino acid residue is selected from Y, F, R and W, and preferentially R or W;

n) in VL CDR5 at position 1, the amino acid residue is selected from A, G, S, W and Y, and preferentially is G;

o) in VL CDR6 at position 4, the amino acid residue is selected from F, H, M, W and Y;

p) in VL CDR6 at position 5, the amino acid residue is selected from D, A, D, E, F, G, H, I, K, L, M, N, Q, R, S, T, V, W, and Y; and/or

q) in VL CDR6 at position 8, the amino acid residue is selected from F, H, R and W

29. The combination preparation of definition 27 or 28, wherein the anti-LukGH antibody comprises a framework including any of the framework regions of the VH and/or VL as listed in Table 2, optionally comprising a Q1E point mutation, if the first amino acid of the VH framework region (VH FR1) is a Q.

30. The combination preparation of any of definitions 27 to 29, wherein the anti-LukGH antibody comprises a HC amino acid sequence as depicted in FIG. 2.

31. The combination preparation of any of definitions 16 to 26, wherein the anti-LukGH antibody is selected from the group consisting of

a) an antibody comprising
a. the HC amino acid sequence SEQ ID 231; and
b. the LC amino acid sequence SEQ ID 232;

b) an antibody comprising
a. the HC amino acid sequence SEQ ID 233; and
b. the LC amino acid sequence SEQ ID 234;

c) an antibody comprising
a. the HC amino acid sequence SEQ ID 235; and
b. the LC amino acid sequence SEQ ID 236;

d) an antibody comprising
a. the HC amino acid sequence SEQ ID 237; and
b. the LC amino acid sequence SEQ ID 238;

e) an antibody comprising
a. the HC amino acid sequence SEQ ID 239; and
b. the LC amino acid sequence SEQ ID 240;

f) an antibody comprising
a. the HC amino acid sequence SEQ ID 241; and
b. the LC amino acid sequence SEQ ID 242;

g) an antibody comprising
a. the HC amino acid sequence SEQ ID 243; and
b. the LC amino acid sequence SEQ ID 244;

h) an antibody comprising
a. the HC amino acid sequence SEQ ID 245; and
b. the LC amino acid sequence SEQ ID 246;

i) an antibody comprising
a. the HC amino acid sequence SEQ ID 247; and
b. the LC amino acid sequence SEQ ID 248;

j) an antibody comprising
a. the HC amino acid sequence SEQ ID 249; and
b. the LC amino acid sequence SEQ ID 250;

k) an antibody comprising
a. the HC amino acid sequence SEQ ID 251; and
b. the LC amino acid sequence SEQ ID 252;

l) an antibody comprising
a. the HC amino acid sequence SEQ ID 253; and
b. the LC amino acid sequence SEQ ID 254;

and

m) an antibody comprising
a. the HC amino acid sequence SEQ ID 255; and
b. the LC amino acid sequence SEQ ID 256,

or a functionally active CDR variant of any of the foregoing, which has an affinity to bind the LukGH complex with a K_{D} of less than 10⁻⁸M, preferably less than 10⁻⁹M.

32. The combination preparation of definition 31, wherein the anti-LukGH antibody is an antibody of any of group member f), g) and h) or a functionally active variant thereof, wherein

a) in VH CDR1 at position 7, the amino acid residue is selected from S, A, D, E, F, G, H, I, K, L, M, N, Q, R, T, V, W, and Y, preferentially any of E, F, H, I, K, L, M, R, V, W or Y, and more preferentially is any of E, F, M, W or Y;

b) in VH CDR2 at position 1, the amino acid residue is selected from N, A, D, E, F, H, L, S, T, V and Y, preferentially any of F, H or Y;

c) in VH CDR2 at position 3, the amino acid residue is selected from Y, H, T and W;

d) in VH CDR2 at position 5, the amino acid residue is selected from S, A, E, F, H, I, K, L, M, N, Q, R, T, V, W and Y, preferentially any of N, R or W, and more preferentially is N or W;

e) in VH CDR2 at position 7, the amino acid residue is selected from S, D, F, H, K, L, M, N, R and W;

f) in VH CDR2 at position 9, the amino acid residue is selected from Y, D, E, F, N, S and W, preferentially D or H, and more preferentially is H;

g) in VH CDR3 at position 4, the amino acid residue is selected from R, A, D, E, F, G, H, I, K, L, M, N, Q, S, T, V and W, preferentially D or H;

h) in VH CDR3 at position 5, the amino acid residue is selected from G, A, F and Y;

i) in VH CDR3 at position 6, the amino acid residue is selected from M, E, F, H and Q, preferentially F or H;

j) in VH CDR3 at position 7, the amino acid residue is selected from H, A, D, E, F, G, I, K, L, M, N, Q, R, S, T, W and Y, preferentially any of E, K, Q, R, W or Y, and more preferentially is W or Y;

k) in VL CDR4 at position 7, the amino acid residue is selected from the group consisting of N, A, D, E, F, G, H, K, L, M, Q, R, S, W and Y, preferentially any of F, L, W, or Y, and more preferentially is L or W;

l) in VL CDR4 at position 8, the amino acid residue is selected from S, A, D, E, F, G, H, I, K, L, M, N, Q, R, T, V, W, and Y, preferentially I or W;

m) in VL CDR4 at position 9, the amino acid residue is selected from Y, F, R and W, and preferentially R or W;

n) in VL CDR5 at position 1, the amino acid residue is selected from A, G, S, W and Y, and preferentially is G;

o) in VL CDR6 at position 4, the amino acid residue is selected from F, H, M, W and Y;

p) in VL CDR6 at position 5, the amino acid residue is selected from D, A, D, E, F, G, H, I, K, L, M, N, Q, R, S, T, V, W, and Y; and/or

q) in VL CDR6 at position 8, the amino acid residue is selected from F, H, R and W

33. The combination preparation of any of definitions 16 to 32, wherein the anti-LukGH antibody has an affinity to bind the LukGH complex with a K_{D} of less than 10⁻⁸M, preferably less than 10⁻⁹M.

34. The combination preparation of definition 33, wherein the anti-LukGH antibody has an affinity to bind the individual LukG and/or LukH antigens which is lower than the affinity to bind the LukGH complex, preferably with a K_{D} of higher than 10⁻⁷M, preferably higher than 10⁻⁶M.

35. The combination preparation of any of definitions 1 to 34, wherein the anti-IGBP antibody comprising a cross-specific CDR binding site recognizing at least three of the IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity to bind SpA-E with a K_{D} of less than 5×10⁻⁹M, as determined by a standard optical interferometry method for a F(ab)2 fragment.

36. The combination preparation of definition 35, wherein the anti-IGBP antibody recognizes at least three of the IGBP domains, preferably at least four, five, or six of the IGBP domains.

37. The combination preparation of definition 35 or 36, wherein the anti-IGBP antibody recognizes at least three of the IGBP domains each with a K_{D} of less than 10⁻⁸M, preferably at least four or five of the IGBP each with a K_{D} of less than 5×10⁻⁹M.

38. The combination preparation of any of definitions 35 to 37, wherein the anti-IGBP antibody recognizes the wild-type SpA with at least substantially the same affinity or with substantially higher affinity as compared to the mutant SpA that lacks binding to IgG Fc or VH3, or as compared to the mutant SpA_{KK} or SpA_{KKAA}, preferably wherein the wild-type SpA is any of the SpA-domains comprising the sequence identified by SEQ ID 401 and optionally further comprising the sequence identified by SEQ ID 402, preferably as determined by comparing the affinity to bind the wild-type SpA-D comprising the amino acid sequence SEQ ID 394 and the mutant SpA-D_{KKAA} comprising the amino acid sequence SEQ ID 399.

39. The combination preparation of any of definitions 35 to 38, wherein the anti-IGBP antibody recognizes both, SpA and Sbi.

40. The combination preparation of any of definitions 35 to 39, wherein the anti-IGBP antibody competes with SpA and optionally Sbi binding to IgG-Fc.

41. The combination preparation of any of definitions 35 to 40, wherein the anti-IGBP antibody is neutralizing Staphylococcus aureus by opsonophagocytosis in human blood or serum.

42. The combination preparation of any of definitions 35 to 41, wherein the anti-IGBP antibody is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence.

42. The combination preparation of any of definitions 35 to 41, wherein the anti-IGBP antibody comprises at least an antibody heavy chain variable region (VH), which is characterized by any of the CDR1 to CDR3 sequences as listed in Table 3, and optionally an antibody light chain region (VL), which is characterized by any of the CDR4 to CDR6 sequences as listed in Table 3, which CDR sequences are designated according to the numbering system of Kabat, or functionally active CDR variants of any of the foregoing.

43. The combination preparation of definition 42, wherein the anti-IGBP antibody
a) comprises a VH domain, which is characterized by any of the CDR1 to CDR3 sequence combinations as listed in Table 3, and a VL domain, which is characterized by any of the CDR4 to CDR6 sequence combinations as listed in Table 3;
b) comprises the set of CDR sequences (CDR1-CDR6) of any of the antibodies as listed in Table 3;
c) is any of the antibodies as listed in Table 3; or
d) is a functionally active variant of a parent antibody that is characterized by the sequences of a)-c),

preferably wherein
i. the functionally active variant comprises at least one functionally active CDR variant of any of the CDR1-CDR6 of the parent antibody; and/or
ii. the functionally active variant comprises at least one point mutation in the framework region of any of the VH and VL sequences;

and further wherein
iii. the functionally active variant has a specificity to bind the same epitope as the parent antibody; and/or
iv. the functionally active variant is a human, humanized, chimeric or murine and/or affinity matured variant of the parent antibody.

44. The combination preparation of definitions 42 or 43, wherein the anti-IGBP antibody comprises a functionally active CDR variant of any of the CDR sequences as listed in Table 3, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence.

45. The combination preparation of any of definitions 42 to 44, wherein the anti-IGBP antibody is selected from the group consisting of group members i) to vi), wherein

i)

A) the antibody comprises
a) a CDR1 consisting of the amino acid sequence of SEQ ID 269; and
b) a CDR2 consisting of the amino acid sequence of SEQ ID 270; and
c) a CDR3 consisting of the amino acid sequence of SEQ ID 271;

and optionally further comprises
d) a CDR4 consisting of the amino acid sequence of SEQ ID 329; and
e) a CDR5 consisting of the amino acid sequence of SEQ ID 330; and
f) a CDR6 consisting of the amino acid sequence of SEQ ID 331;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 69;
b) the parent CDR2 consists of the amino acid sequence SEQ ID 70;
c) the parent CDR3 consists of the amino acid sequence SEQ ID 71;
d) the parent CDR4 consists of the amino acid sequence SEQ ID 329;
e) the parent CDR5 consists of the amino acid sequence SEQ ID 330;
f) the parent CDR6 consists of the amino acid sequence SEQ ID 331;

ii)

A) the antibody comprises
a) a CDR1 consisting of the amino acid sequence of SEQ ID 287; and
b) a CDR2 consisting of the amino acid sequence of SEQ ID 288; and
c) a CDR3 consisting of the amino acid sequence of SEQ ID 289;

and optionally further comprises
d) a CDR4 consisting of the amino acid sequence of SEQ ID 347; and
e) a CDR5 consisting of the amino acid sequence of SEQ ID 348; and
f) a CDR6 consisting of the amino acid sequence of SEQ ID 349;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 287;
b) the parent CDR2 consists of the amino acid sequence SEQ ID 288;
c) the parent CDR3 consists of the amino acid sequence SEQ ID 289;
d) the parent CDR4 consists of the amino acid sequence SEQ ID 347;
e) the parent CDR5 consists of the amino acid sequence SEQ ID 348;
f) the parent CDR6 consists of the amino acid sequence SEQ ID 349;

iii)

A) the antibody comprises
a) a CDR1 consisting of the amino acid sequence of SEQ ID 296; and
b) a CDR2 consisting of the amino acid sequence of SEQ ID 297; and
c) a CDR3 consisting of the amino acid sequence of SEQ ID 298;

and optionally further comprises
d) a CDR4 consisting of the amino acid sequence of SEQ ID 356; and
e) a CDR5 consisting of the amino acid sequence of SEQ ID 357; and
f) a CDR6 consisting of the amino acid sequence of SEQ ID 358;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 296;
b) the parent CDR2 consists of the amino acid sequence SEQ ID 297;
c) the parent CDR3 consists of the amino acid sequence SEQ ID 298;
d) the parent CDR4 consists of the amino acid sequence SEQ ID 356;
e) the parent CDR5 consists of the amino acid sequence SEQ ID 357;
f) the parent CDR6 consists of the amino acid sequence SEQ ID 358;

iv)

A) the antibody comprises
a) a CDR1 consisting of the amino acid sequence of SEQ ID 299; and
b) a CDR2 consisting of the amino acid sequence of SEQ ID 300; and
c) a CDR3 consisting of the amino acid sequence of SEQ ID 301;

and optionally further comprises
d) a CDR4 consisting of the amino acid sequence of SEQ ID 359; and
e) a CDR5 consisting of the amino acid sequence of SEQ ID 360; and
f) a CDR6 consisting of the amino acid sequence of SEQ ID 361;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 299;
b) the parent CDR2 consists of the amino acid sequence SEQ ID 300;
c) the parent CDR3 consists of the amino acid sequence SEQ ID 3;
d) the parent CDR4 consists of the amino acid sequence SEQ ID 359;
e) the parent CDR5 consists of the amino acid sequence SEQ ID 360;
f) the parent CDR6 consists of the amino acid sequence SEQ ID 361;

v)

A) the antibody comprises
a) a CDR1 consisting of the amino acid sequence of SEQ ID 302; and
b) a CDR2 consisting of the amino acid sequence of SEQ ID 303; and
c) a CDR3 consisting of the amino acid sequence of SEQ ID 304;

and optionally further comprises
d) a CDR4 consisting of the amino acid sequence of SEQ ID 362; and
e) a CDR5 consisting of the amino acid sequence of SEQ ID 363; and
f) a CDR6 consisting of the amino acid sequence of SEQ ID 364;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 302;
b) the parent CDR2 consists of the amino acid sequence SEQ ID 303;
c) the parent CDR3 consists of the amino acid sequence SEQ ID 304;
d) the parent CDR4 consists of the amino acid sequence SEQ ID 362;
e) the parent CDR5 consists of the amino acid sequence SEQ ID 363;
f) the parent CDR6 consists of the amino acid sequence SEQ ID 364;

and

vi)

A) the antibody comprises
a) a CDR1 consisting of the amino acid sequence of SEQ ID 314; and
b) a CDR2 consisting of the amino acid sequence of SEQ ID 315; and
c) a CDR3 consisting of the amino acid sequence of SEQ ID 316;

and optionally further comprises
d) a CDR4 consisting of the amino acid sequence of SEQ ID 374; and
e) a CDR5 consisting of the amino acid sequence of SEQ ID 375; and
f) a CDR6 consisting of the amino acid sequence of SEQ ID 376;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 314;
b) the parent CDR2 consists of the amino acid sequence SEQ ID 315;
c) the parent CDR3 consists of the amino acid sequence SEQ ID 316;
d) the parent CDR4 consists of the amino acid sequence SEQ ID 374;
e) the parent CDR5 consists of the amino acid sequence SEQ ID 375;
f) the parent CDR6 consists of the amino acid sequence SEQ ID 376.

46. The combination preparation of any of definitions 1 to 45, wherein

a) the toxin cross-neutralizing antibody comprises
a. the CDR1 sequence SEQ ID 1; and
b. the CDR2 sequence SEQ ID 2; and
c. the CDR3 sequence SEQ ID 12; and
d. the CDR4 sequence SEQ ID 32; and
e. the CDR5 sequence SEQ ID 33; and
f. the CDR6 sequence SEQ ID 34;

b) the anti-LukGH antibody comprises
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;

and

c) the anti-IGBP antibody comprises
a. the CDR1 sequence SEQ ID 299; and
b. the CDR2 sequence SEQ ID 300; and
c. the CDR3 sequence SEQ ID 301; and
d. the CDR4 sequence SEQ ID 359; and
e. the CDR5 sequence SEQ ID 360; and
f. the CDR6 sequence SEQ ID 361;

or a functionally active CDR variant of any of the foregoing, which has an affinity to bind the target antigen with a K_{D} of less than 10⁻⁸M, preferably less than 5×10⁻⁹M.

47. The combination preparation of any of definitions 1 to 46, which comprises

a) the toxin cross-neutralizing antibody;

b) the anti-LukGH antibody; and

c) the anti-IGBP antibody.

48. The combination preparation of any of definitions 1 to 47, wherein each of the toxin cross-neutralizing antibody, the anti-LukGH antibody, or the anti-IGBP antibody has an affinity to bind the target antigen with a K_{D} of less than 10⁻⁸M, preferably less than 5×10⁻⁹M or less than 10⁻⁹M.

49. The combination preparation of any of definitions 1 to 48, wherein each of the toxin cross-neutralizing antibody, the anti-LukGH antibody, or the anti-IGBP antibody is a full-length monoclonal antibody, an antibody fragment thereof comprising at least one antibody domain incorporating the binding site, or a fusion protein comprising at least one antibody domain incorporating the binding site.

50. The combination preparation of any of definitions 1 to 48, for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

51. A pharmaceutical preparation comprising the combination preparation of any of definitions 1 to 48, preferably comprising a parenteral or mucosal formulation, optionally containing a pharmaceutically acceptable carrier or excipient.

52. The pharmaceutical preparation of definition 51, which is provided as a mixture of the antibodies in a formulation, or as kit of parts, wherein at least one of the antibodies is provided in a separate formulation.

53. A kit for preparing a pharmaceutical preparation of definition 51 or 52, comprising at least the following components in a pharmaceutically acceptable formulation:

a) the toxin cross-neutralizing antibody;

b) the anti-LukGH antibody; and/or

c) the anti-IGBP antibody.

54. Use of the kit of definition 53, for preparing a pharmaceutical preparation of definition 51.

55. The kit of definition 53, for use in treating a subject at risk of or suffering from a S. aureus infection comprising administering to the subject an effective amount of the antibody to limit the infection in the subject, to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

56. The kit for use according to definitions 55, wherein the components are administered to the subject concomitantly, in parallel and/or consecutively.

57. An anti-Staphylococcus aureus antibody preparation comprising one or more antibodies specifically recognizing the S. aureus targets:

a) alpha-toxin (HIa) and at least one of the bi-component toxins selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and

b) the LukGH complex; and/or

c) an S. aureus IgG binding domain of Protein A or Sbi or an IGBP; and/or

d) any S. aureus surface protein to bind an antibody thereby inducing OPK, preferably wherein the preparation comprises at least one antibody which is a polyspecific antibody and at least one antibody which is a monospecific antibody.

58. A combination preparation according to any of the preceding definitions, comprising a toxin cross-neutralizing antibody and an anti-LukGH antibody, wherein

a) the toxin cross-neutralizing antibody is any of the mAbs designated ASN-1 as described herein; and

b) the anti-LukGH antibody is any of the mAbs designated ASN-2 as described herein, specifically, wherein

(i) the mAb designated ASN-1 is characterized by the 6 CDR sequences of any of the antibodies listed in Table 1, in particular the CDR sequences of any of the mAbs designated AB-28, AB-28-10, AB-28-7, AB-28-8, or AB-28-9, or a functional variant of any of the foregoing; and

(ii) the mAb designated ASN-2 is characterized by the 6 CDR sequences of any of the antibodies listed in any of the Tables 2.1, 2.2, 2.3, 2.4, 2.5, 2.6, 2.7, or 2.8, in particular the CDR sequences of any of the mAbs designated AB-30-3, AB-31, AB-32-9, AB-34-6, or AB-34, or a functional variant of any of the foregoing,

preferably wherein the functional variant is a functionally active CDR variant of any of the foregoing, which has an affinity to bind the target antigen with a K_{D} of less than 10⁻⁸M, preferably less than 5×10⁻⁹M.

The foregoing description will be more fully understood with reference to the following examples. Such examples are, however, merely representative of methods of practicing one or more embodiments of the present invention and should not be read as limiting the scope of invention.

### EXAMPLES

### Example 1

### Synergistic Protective Effect of Combinations of Antibodies Neutralizing Mixtures of Recombinant S. aureus Leukocidins

The synergistic effect of a toxin cross-reactive mAb ASN-1 (Rouha, 2015) has been shown with a series of antibodies comprising the CDR sequences of AB-28 or of its variants AB-28-x. Antibodies comprising the CDR sequences of AB-28 or of its variants AB-28-x, e.g., antibodies of Table 1 are herein called ASN-1. Such mAbs are neutralizing alpha-hemolysin, LukSF, LukED, HIgAB and HIgCB.

The ASN-1 mAb was tested alone or in combination with a LukGH neutralizing antibody, ASN-2.

LukGH neutralizing antibodies comprising the CDR sequences of AB-29, AB-30, AB-31, AB-32, AB-33, AB-34, AB-35, and AB-36, or of variants of any of the foregoing, are herein referred to as ASN-2 mAbs, e.g., antibodies of Tables 2.1, 2.2, 2.3, 2.4, 2.5, 2.6, 2.7, or 2.8.

The examples were performed with a series of antibodies comprising the CDR sequences of AB-30, AB-31, AB-32, AB-34, or of variants of any of the foregoing, in particular AB-30-3, AB-31, AB-34, AB34-6, AB32-9. The antibodies used in the examples section were in particular the following:

ASN-1:

AB-28: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 3;

VL CDR4: SEQ ID 32;

VL CDR5: SEQ ID 33;

VL CDR6: SEQ ID 34.

AB-28 is specifically characterized by the following HC and LC sequence:

HC: SEQ ID 40,

LC: SEQ ID 52.

AB-28-10: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 12;

VL CDR4: SEQ ID 32;

VL CDR5: SEQ ID 33;

VL CDR6: SEQ ID 34.

AB-28-10 is specifically characterized by the following HC and LC sequences:

HC: SEQ ID 48,

LC: SEQ ID 52.

AB-28-7: a mAb characterized by 6 CDR sequences as listed in Table 1.1a, 1.1b, and 1.1c:

VH CDR1: SEQ ID 5;

VH CDR2: SEQ ID 9;

VH CDR3: SEQ ID 3;

VL CDR4: SEQ ID 32;

VL CDR5: SEQ ID 33;

VL CDR6: SEQ ID 34.

AB-28-7 is specifically characterized by the following HC and LC sequences:

HC: SEQ ID 45,

LC: SEQ ID 52.

AB-28-8: a mAb characterized by 6 CDR sequences as listed in Table 1.1a, 1.1b, and 1.1c:

VH CDR1: SEQ ID 5;

VH CDR2: SEQ ID 10;

VH CDR3: SEQ ID 3;

VL CDR4: SEQ ID 32;

VL CDR5: SEQ ID 33;

VL CDR6: SEQ ID 34.

AB-28-8 is specifically characterized by the following HC and LC sequences: HC: SEQ ID 46,

LC: SEQ ID 52.

AB-28-9: a mAb characterized by 6 CDR sequences as listed in Table 1.1a, 1.1b, and 1.1c:

VH CDR1: SEQ ID 1;

VH CDR2: SEQ ID 2;

VH CDR3: SEQ ID 12;

VL CDR4: SEQ ID 32;

VL CDR5: SEQ ID 33;

VL CDR6: SEQ ID 34.

AB-28-9 is specifically characterized by the following HC and LC sequences:

HC: SEQ ID 46,

LC: SEQ ID 52.

ASN-2:

AB-30-3: a mAb characterized by 6 CDR sequences as listed in Table 2.2a, 2.2b (Group 2 mAbs):

VH CDR1: SEQ ID 122;

VH CDR2: SEQ ID 123;

VH CDR3: SEQ ID 114;

VL CDR4: SEQ ID 116;

VL CDR5: SEQ ID 117;

VL CDR6: SEQ ID 119.

AB-30-3 is specifically characterized by the following HC and LC sequences:

HC: SEQ ID 235,

LC: SEQ ID 236.

AB-31: a mAb characterized by 6 CDR sequences as listed in Table 2.3a, 2.3b (Group 3 mAbs):

VH CDR1: SEQ ID 131;

VH CDR2: SEQ ID 133;

VH CDR3: SEQ ID 135;

VL CDR4: SEQ ID 137;

VL CDR5: SEQ ID 105;

VL CDR6: SEQ ID 138.

AB-31 is specifically characterized by the following HC and LC sequences:

HC: SEQ ID 239,

LC: SEQ ID 240.

AB-34: a mAb characterized by 6 CDR sequences as listed in Table 2.6a, 2.6b (Group 6 mAbs):

VH CDR1: SEQ ID 188;

VH CDR2: SEQ ID 189;

VH CDR3: SEQ ID 190;

VL CDR4: SEQ ID 176;

VL CDR5: SEQ ID 178;

VL CDR6: SEQ ID 192.

AB-34 is specifically characterized by the following HC and LC sequences:

HC: SEQ ID 249,

LC: SEQ ID 250.

AB-34-6: a mAb characterized by 6 CDR sequences as listed in Table 2.6a, 2.6b (Group 6 mAbs):

VH CDR1: SEQ ID 198;

VH CDR2: SEQ ID 199;

VH CDR3: SEQ ID 190;

VL CDR4: SEQ ID 200;

VL CDR5: SEQ ID 201;

VL CDR6: SEQ ID 202.

AB-34-6 is specifically characterized by the following HC and LC sequences: HC: SEQ ID 253,

LC: SEQ ID 254.

AB-32-9: a mAb characterized by 6 CDR sequences as listed in Table 2.4a, 2.4b (Group 4 mAbs):

VH CDR1: SEQ ID 167;

VH CDR2: SEQ ID 168;

VH CDR3: SEQ ID 157;

VL CDR4: SEQ ID 159;

VL CDR5: SEQ ID 125;

VL CDR6: SEQ ID 160.

AB-32-9 is specifically characterized by the following HC and LC sequences:

HC: SEQ ID 245,

LC: SEQ ID 246.

According to the first example, the synergistic effect was determined in a PMN viability assay using a mixture of recombinant toxins. The five recombinant bi-component toxins of S. aureus were expressed in E. coli cells as described previously (Rouha, 2015; Badarau, 2015). LukSF, LukED, HIgAB, and HIgCB sequences were derived from the genome of the TCH1516 strains (GenBank Accession number CP000730.1). While LukSF, LukED, HIgAB and HIgCB are highly conserved among different S. aureus isolates, several different LukGH sequence variants exist of which 4 of the most distantly related ones were expressed in E.coli (TCH1516, MSHR1132, MRSA252 and H19). All 4 LukGH variants were included in this assay at 1.875 nM each, equaling 7.5 nM total LukGH concentration. Target cell viability was measured in an ATP viability assay using human polymorphonuclear leukocytes (PMNs). A three-fold serial dilution of the antibodies ASN-1, ASN-2, or ASN-1 in combination with ASN-2, and a negative control IgG1 against an irrelevant antigen was prepared in RPMI with 10% FBS and L-Glutamine in 96-well, white half area luminescence plates (Costar Corning). The final antibody concentration was ranging from 2000 nM to 0.1 nM for single antibodies, and from 4000 nM to 0.2 nM with ASN-1+ASN-2 combination or in case of the control mAb. Toxins were mixed and incubated with antibodies for 30 minutes at RT with shaking at 200 rpm in a benchtop shaker (New Brunswick Innova 40R). Individual toxins were used at the concentration that allowed >80% cell lysis: LukSF 5 nM, LukED 7.5 nM, HIgAB 7.5 nM, HIgCB at 7.5 nM and LukGH (total) 7.5 nM. Following the 30 minutes incubation time, the PMN suspension was added with a final concentration of 2.5×10⁴ cells per well and the plates were incubated for 4 hours at 37° C., 5% CO₂, without shaking. The plates were allowed to cool down to RT for 10 minutes, followed by measurement of cell viability using the CellTiter-Glo® Luminescent Cell Viability Assay (Promega) in a Synergy HT Plate Reader.

The individual toxins caused more than 80% reduction in cell viability on their own, the mixture of all five toxins resulted in ˜99% cells lysis (FIG. 6A). ASN-2 that neutralizes LukGH, one of the five cytotoxins, alone did not show protection from lysis similarly to the negative control mAb. ASN-1 that neutralizes four of the five toxins protected approximately 15% of the PMNs, those that were not killed by the LukGH toxins. The combination of ASN-1 and ASN-2 was highly potent to preserve PMN viability (˜95%) (FIG. 6B).

### Example 2

### Protecting Human PMNs from S. aureus Culture Supernatant Mediated Killing by Combination of Toxin Neutralizing Antibodies

The number of toxins encoded by the genome different S. aureus strains ranges from three to five, lukSF and lukED genes are not present in all strains. S. aureus secretes the cytolytic leukocidins into the culture supernatant where they typically reach highest levels during the stationary growth phase.

According to this example, any of the mAbs designated AB-28, AB-28-10, AB-28-7, AB-28-8, or AB-28-9, was combined with any of the mAbs designated AB-30-3, AB-31, AB-32-9, AB-34-6, or AB-34.

To test the individual and combined inhibitory capacity of the anti-toxin mAbs against the secreted exotoxins, bacterial culture supernatants (CS) were prepared in RPMI supplemented with 1% of casamino acids (Amresco). Bacteria were grown from a single colony to stationary phase in 20 ml medium at 37° C. shaking at 200 rpm. CS were harvested by culture centrifugation at 5000×g, followed by filter sterilization of the supernatant using 0.1 μm pore size syringe filters (Millipore).

Antibody mediated toxin-neutralization was measured using serial CS dilutions and fixed antibody concentrations with freshly purified human PMNs in RPMI supplemented with 10% FBS and L-Glutamine in 96-well plates (Costar Corning). The final antibody concentration in these assays was 1000 nM for single antibodies, and 1000 nM ASN-1+1000 nM ASN-2 for mAb combinations. The control mAb was used at 2000 nM concentration.

After a 30 min pre-incubation of mAbs and CS, 25,000 PMNs were added per well following incubation for 4 hours at 37° C., 5% CO₂, without shaking. PMN viability was assessed using a CellTiter-Glo® Luminescent Cell Viability Assay (Promega) according to the manufacturer's instructions.

FIGS. 7 and 8 depict the results obtained at the 8× CS dilution, at which CS caused >80% cell death in absence of neutralizing mAb for all strains (—except for the CS of the isogenic mutant lacking all 6 toxin genes which did not affect PMN viability.

When using the CS prepared from the sequenced TCH1516 strain (CA-MRSA USA300) which carries all five leukocidin genes, >90% reduction in PMN viability was observed (FIG. 7A). The single ASN-1 and ASN-2 antibodies could only partially inhibit toxicity but upon combination PMN viability could be fully maintained. The culture supernatant of the isogenic TCH1516 mutant lacking all the toxins targeted by ASN-1 and ASN-2 did not cause PMN killing in these experiments (<10% reduction in viability) (FIG. 7B). Removal of lukGH allowed ASN-1 to be fully protective (FIG. 7C), while deletion of the lukSF, lukED and hIgABC genes rendered ASN-1 dispensable for protection of PMNs (FIG. 7D).

CS samples from several S. aureus prototype strains and different clinical isolates obtained from endotracheal aspirate (ETA) of ventilated patients (Stulik, 2014) were tested in similar assays. Greatly different patterns were observed with such strains when single antibodies were used. Results are shown in FIG. 8. Single antibodies showed partial protection with some CSs (FIG. 8A, B, C), but no effect with others (FIG. 8D). However, the combination of the two mAbs was always highly effective in maintaining PMN viability (FIG. 8A-D). It should be noted, that in contrast to the experiment using a recombinant toxin mixture with clearly defined amounts of the individual proteins, the absolute and the relative amount of toxins in bacterial culture supernatants varies in different S. aureus isolates based on genetics and environmental influence. Not all toxins targeted by the antibodies are necessarily present in this in vitro model. This explains why the neutralizing effect of the single antibodies in absence of the complementing mAb can vary significantly among different strains.

Importantly, the pan-neutralizing capacity of a mAb cocktail targeting all leukocidins is not restricted to any specific ASN-1 and ASN-2 combination. Also antibody sequence variants (other than exemplified herein) with comparable binding patterns can be combined. An example is shown in FIG. 8E, using the CS of the clinical MSSA isolate ST8t334. Neither the HIa-LukF-HIgB-LukD cross-neutralizing mAbs, nor the anti-LukGH mAbs alone could prevent PMN lysis mediated by the concerted activity of all secreted toxins, but the combination of the two antibody types conferred protection in all possible permutations.

### Example 3

### Combining Toxin-neutralizing Antibodies to Prevent Killing of Human PMNs by Toxins Produced by Live S. ureus Cells

It has been reported that S. aureus is able to up-regulate the expression of leukocidins when encountering human PMNs (Malachowa, 2011). It was therefore important to test, whether the toxin-neutralizing antibodies would be also able to counteract the effect of bacterial toxins produced in situ in response to human phagocytes and not only after pre-incubation with preformed toxins in culture supernatant. For this purpose, PMNs were infected with live bacteria and measured their survival based on flow cytometric detection of live cells.

Purified PMNs from human heparinized whole blood were diluted to 4×10⁶ cells/mL in RPMI supplemented with 10% FBS and L-Glutamine. S. aureus strains were grown to mid-logarithmic phase in RPMI supplemented with 1% of casamino acids at 37° C. with shaking at 200 rpm (New Brunswick Innova 40R), washed once with 1× DPBS (Life Technologies) and re-suspended in RPMI supplemented with 10% FBS and L-Glutamine at a concentration of 2×10⁸ CFU/mL. The mAbs—ASN-1, ASN-2, ASN-1 in combination with ASN-2, and a negative control IgG against an irrelevant antigen—were added to 96-well plates with a final concentration for each single antibody of 2000 nM, for combinations of 2000 nM ASN-1+2000 nM ASN-2. The control mAb was used at 4000 nM concentration. The bacterial suspensions were added with a final concentration of 1×10⁸ CFU/mL, followed by the addition of the PMNs to the antibody-bacteria mixture. The PMN concentration was 1×10⁶ cells per mL, corresponding to a multiplicity of infection (MOI) of 100 (1×10⁶ PMNs/mL:1×10⁸ CFU/mL). Plates were incubated for 2 hours at 37° C. with 5% CO₂, without shaking. After the 2 hours incubation time, the plates were centrifuged for 7 min at 1027×g and the supernatant was discarded. PMNs were re-suspended in Hank's balanced salt solution (HBSS, Life Technologies) supplemented with 0.01% of sodium azide. Live PMN counts were enumerated by flow cytometry using an iCyt Eclipse Flow Cytometer. Data were analyzed with FCS Express software (Flow Research Edition).

Similar to the assays performed with sterile filtrated culture supernatants, ASN-1 or ASN-2 alone were partially effective to inhibit PMN killing by the different S. aureus strains (examples shown in FIG. 9), proving that the antibodies were able to neutralize toxins produced in situ. Although the effect of the single antibodies differs among the different strains, the highest cell number after infection could be recovered in presence of both antibodies, proving their additive effects.

### Example 4

### Beneficial Effect of Antibody Combinations to Protect Mice from Lethal S. Aureus Infection

Almost all S. aureus leukocidins are species specific and do not lyse murine immune cells (except LukED at higher concentrations) (Spaan, 2014). Therefore the neutralizing-effect against cytotoxicity of ASN-2 cannot be evaluated in mice and the ASN-1 protective effect is mainly associated with alpha-hemolysin neutralization. Nonetheless, the potential beneficial effect of toxin neutralization by ASN-1 and disarming the SpA and Sbi virulence functions can be investigated. As anti-IGBP antibody, ASN-3 was used (the example was performed with a series of antibodies comprising the CDR sequences of the antibodies listed in Table 3, thus, such antibodies of Table 3 are herein called ASN-3).

For these experiments S. aureus strain TCH1516 (BAA-1717™, from ATCC) was grown to mid-log phase (OD₆₀₀ of 0.5) in tryptic soy broth and diluted for intravenous injection (100 μl with 2×10⁷ cfu challenge dose per animal). In all experiments, female 6-8 week old BALB/cJRj mice were used. 2×10⁷ cfu challenge dose per animal was applied intravenously. Passive immunization with antibodies was performed by intraperitoneal injection of mAbs diluted in 500 μl in PBS 24 h prior to the lethal, intravenous challenge by bacteria. 100 μg of each mAb was administered per animal in two independent experiments with 5 mice/group (total 10 mice/mAb treatment). Control groups received 200 μg isotype-matched (IgG1) irrelevant mAb. Survival-statistics were calculated against control mAb based on log-rank (Mantel-Cox) test using GraphPad Prism (Version 5.04).

In this highly stringent model, all control mice died within 48 hours after bacterial challenge. ASN-1 or SpA mAb treated mice exhibited partial protection (40 and 60%, respectively), while all animals treated with the combination of ASN-1 and SpA mAbs were alive at 48 hours post-challenge (FIG. 10). The increased protection achieved with the combination of ASN-1 with ASN-3 was detected throughout the entire experiments.

### REFERENCES

Lowy F D. 1998. S. aureus infections. N. Engl. J. Med. 339:520-32.

Rigby K M, DeLeo F R. 2012. Neutrophils in innate host defense against Staphylococcus aureus infections. Semin Immunopathol. 34:237-259.

Vandenesch F, Lina G, Henry T. 2012. Staphylococcus aureus hemolysins, bi-component leukocidins, and cytolytic peptides: a redundant arsenal of membrane-damaging virulence factors? Front Cell Infect Microbiol. 2:12.

Spaan, A. N., Surewaard, B. G., Nijland, R. & van Strijp, J. A. 2013. Neutrophils versus Staphylococcus aureus: a biological tug of war. Annu. Rev. Microbiol. 67, 629-650.

Alonzo F 3rd, Torres V J. 2013. Bacterial survival amidst an immune onslaught: the contribution of the Staphylococcus aureus leukotoxins. PLoS Pathog. 9:e1003143.

Alonzo F, Torres V J. 2014. The Bicomponent Pore-Forming Leucocidins of Staphylococcus aureus. Microbiology and Molecular Biology Reviews 78:199-230.

Spaan A N, Henry T, van Rooijen W J, Perret M, Badiou C, Aerts P C, Kemmink J, de Haas C J, van Kessel K P, Vandenesch F, Lina G, van Strijp J A. 2013. The staphylococcal toxin panton-valentine leukocidin targets human c5a receptors. Cell Host Microbe. 13:584-94.

Spaan A N, Vrieling M, Wallet P, Badiou C, Reyes-Robles T, Ohneck E A, Benito Y, de Haas C J, Day C J, Jennings M P, Lina G, Vandenesch F, van Kessel K P, Torres V J, van Strijp J A, Henry T. 2014. The staphylococcal toxins γ-haemolysin AB and CB differentially target phagocytes by employing specific chemokine receptors. Nat Commun. 5:5438.

Dumont A L, Yoong P, Day C J, Alonzo F 3rd, McDonald W H, Jennings M P, Torres V J. 2013. Staphylococcus aureus LukAB cytotoxin kills human neutrophils by targeting the CD11b subunit of the integrin Mac-1. Proc Natl Acad Sci USA 110:10794-9.

Reyes-Robles T, Alonzo F 3rd, Kozhaya L, Lacy D B, Unutmaz D, Torres V J. 2013. Staphylococcus aureus leukotoxin ED targets the chemokine receptors CXCR1 and CXCR2 to kill leukocytes and promote infection. Cell Host Microbe. 14:453-9.

Oleksiewicz M B, Nagy G, Nagy E. 2012. Anti-bacterial monoclonal antibodies: back to the future? Arch Biochem Biophys. 526:124-31.

Jansen K U, Girgenti D Q, Scully I L, Anderson A S. 2013. Vaccine review: “Staphyloccocus aureus vaccines: Problems and prospects”. Vaccine. 31:2723-30.

Dryla A, Prustomersky S, Gelbmann D, Hanner M, Bettinger E, Kocsis B, Kustos T, Henics T, Meinke A, Nagy E. 2005. Comparison of antibody repertoires against Staphylococcus aureus in healthy individuals and acutely infected patients. Clin. Diagn. Lab. Immunol. 12: 387-98.

Adhikari R P, Ajao A O, Aman M J, Karauzum H, Sarwar J, Lydecker A D, Johnson J K, Nguyen C, Chen W H, Roghmann M C. 2012. Lower Antibody Levels to Staphylococcus aureus Exotoxins Are Associated With Sepsis in Hospitalized Adults With Invasive S. aureus Infections. J Infect Dis. 206:915-23.

Fritz S A, Tiemann K M, Hogan P G, Epplin E K, Rodriguez M, Al-Zubeidi D N, Bubeck Wardenburg J, Hunstad D A. 2013. A serologic correlate of protective immunity against community-onset Staphylococcus aureus infection. Clin. Infect. Dis. 56:1554-61.

Rouha H, Badarau A, Visram Z C, Battles M B, Prinz B, Magyarics Z, Nagy G, Mirkina I, Stulik L, Zerbs M, Jagerhofer M, Maierhofer B, Teubenbacher A, Dolezilkova I, Gross K, Banerjee S, Zauner G, Malafa S, Zmajkovic J, Maier S, Mabry R, Krauland E, Wittrup K D, Gerngross T U, Nagy E. 2015. Five birds, one stone: Neutralization of alpha-hemolysin and four bi-component leukocidins of Staphylococcus aureus with a single human monoclonal antibody. MAbs. 7:243-54.

DuMont A L, Yoong P, Liu X, Day C J, Chumbler N M, James D B, Alonzo F 3^{rd}, Bode N J, Lacy D B, Jennings M P, Torres V J. 2014. Identification of a crucial residue required for Staphylococcus aureus LukAB cytotoxicity and receptor recognition. Infect. Immun. 82:1268-76.

Badarau A, Rouha H, Malafa S, Logan D T, Hakansson M, Stulik L, Dolezilkova I, Teubenbacher A, Gross K, Maierhofer B, Weber A, Jagerhofer M, Hoffman D, Nagy E. 2015. Structure-function analysis of heterodimer formation, oligomerization and receptor binding of the Staphylococcus aureus bi-component toxin LukGH. J Biol Chem. 290:142-56.

Falugi F, Kim H K, Missiakas D M, Schneewind O. 2013. Role of Protein A in the evasion of host adaptive immune responses by Staphylococcus aureus. MBio. 4:e00575-13. doi: 10.1128/mBio.00575-13.

Smith E, Visai L, Kerrigan S, Speziale P, Foster T. 2011. The Sbi protein is a multifunctional immune evasion factor of Staphylococcus aureus. Infect Immun 79: 3801-9.

Stulik L, Malafa S, Hudcova J, Henics B Z, Craven D, Sonnevand A M, Nagy E. 2014. α-Hemolysin Activity of Methicillin-Susceptible Staphylococcus aureus Predicts Ventilator-associated Pneumonia. Am J Respir Crit Care Med 190:1139-48.

Malachowa N, Whitney A R, Kobayashi S D, Sturdevant D E, Kennedy A D, Braughton K R, Shabb D W, Diep B A, Chambers H F, Otto M, DeLeo F R. 2011. Global changes in Staphylococcus aureus gene expression in human blood. PLoS One. 6:e18617.

1-17. (canceled)

18. An anti-Staphylococcus aureus antibody combination preparation comprising
a) a toxin cross-neutralizing antibody comprising at least one polyspecific binding site that binds to alpha-toxin (HIa) and at least one bi-component toxin selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and
b) an anti-LukGH antibody; and/or
c) an OPK antibody which recognizes a S. aureus surface protein thereby inducing OPK.

19. the combination preparation of claim 18, wherein the OPK antibody comprises an anti-Ig-binding protein (IGBP) antibody comprising at least one CDR binding site that recognizes any of the S. aureus IgG binding domains of Protein A or Sbi.

20. The combination preparation of claim 18, wherein the toxin cross-neutralizing antibody has a cross-specificity to bind HIa and at least one F-component and/or at least one S-component of the bi-component toxin.

21. The combination preparation of claim 18, wherein the toxin cross-neutralizing antibody comprises three complementarity determining regions (CDR1 to CDR3) of an antibody heavy chain variable region (VH) and three complementarity determining regions (CDR4 to CDR6) of an antibody light chain variable region (VL), wherein
A) the antibody comprises
a) a CDR1 comprising the amino acid sequence SEQ ID 1; and
b) a CDR2 comprising the amino acid sequence SEQ ID 2; and
c) a CDR3 comprising the amino acid sequence SEQ ID 3;
or
B) the antibody comprises the antibody of A), wherein at least one of CDR1, CDR2, and CDR3 is a functionally active CDR variant of a CDR in A) (parent CDR), comprising at least one point mutation and at least 60% sequence identity with the parent CDR.

22. The combination preparation of claim 21, wherein
A) the antibody comprises
a) a CDR4 comprising the amino acid sequence SEQ ID 32; and
b) a CDR5 comprising the amino acid sequence SEQ ID 33; and
c) a CDR6 comprising the amino acid sequence SEQ ID 34;
or
B) the antibody comprises the antibody of A, wherein at least one of CDR4, CDR5, and CDR6 is a functionally active CDR variant of a CDR in A) (parent CDR), comprising at least one point mutation and at least 60% sequence identity with the parent CDR.

23. The combination preparation of claim 18, wherein the anti-LukGH antibody comprises at least one binding site that specifically binds to a LukGH complex.

24. The combination preparation of claim 18, wherein the anti-LukGH antibody comprises an antibody heavy chain variable region (VH) comprising the CDR1, CDR2, and CDR3 sequences of any antibody sequence listed in Table 2, or a functionally active CDR variant thereof, and an antibody light chain variable region (VL) comprising the CDR4, CDR5, and CDR6 sequences of any antibody sequence listed in Table 2, or a functionally active CDR variant thereof.

25. The combination preparation of claim 24, wherein the anti-LukGH antibody is selected from the group consisting of i) to viii):
i)
A1) an antibody comprising
a) a CDR1 comprising the amino acid sequence SEQ ID 86 or SEQ ID 99; and
b) a CDR2 comprising the amino acid sequence SEQ ID 88; and
c) a CDR3 comprising the amino acid sequence SEQ ID 90; or
B1) an antibody comprising the antibody of A1, wherein at least one of CDR1, CDR2, or CDR3 is a functionally active CDR variant of a parent CDR, comprising at least one point mutation and at least 60% sequence identity with the parent CDR;
ii)
A2) an antibody comprising
a) a CDR1 comprising any of the amino acid sequences SEQ ID 110, SEQ ID 120, or SEQ ID 122; and
b) a CDR2 comprising any of the amino acid sequences SEQ ID 112, SEQ ID 121, SEQ ID 123, or SEQ ID 124; and
c) a CDR3 comprising the amino acid sequence SEQ ID 114; or
B2) an antibody comprising the antibody of A2, wherein at least one of CDR1, CDR2, or CDR3 is a functionally active CDR variant of a parent CDR, comprising at least one point mutation and at least 60% sequence identity with the parent CDR;
iii)
A3) an antibody comprising
a) a CDR1 comprising any of the amino acid sequences SEQ ID 131, SEQ ID 139, SEQ ID 141, SEQ ID 143, SEQ ID 145, SEQ ID 147, or SEQ ID 148; and
b) a CDR2 comprising any of the amino acid sequences SEQ ID 133, SEQ ID 140, SEQ ID 142, SEQ ID 144, SEQ ID 146, SEQ ID 149, or SEQ ID 150; and
c) a CDR3 comprising the amino acid sequence SEQ ID 135; or
B3) an antibody comprising the antibody of A3, wherein at least one of CDR1, CDR2, or CDR3 is a functionally active CDR variant of a parent CDR, comprising at least one point mutation and at least 60% sequence identity with the parent CDR;
iv)
A4) an antibody comprising
a) a CDR1 comprising any of the amino acid sequences SEQ ID 155, SEQ ID 161, SEQ ID 163, SEQ ID 165, SEQ ID 167, or SEQ ID 169; and
b) a CDR2 comprising any of the amino acid sequences SEQ ID 156, SEQ ID 162, SEQ ID 168, or SEQ ID 88; and
c) a CDR3 comprising the amino acid sequence SEQ ID 157; or
B4) an antibody comprising the antibody of A4, wherein at least one of CDR1, CDR2, or CDR3 is a functionally active CDR variant of a parent CDR, comprising at least one point mutation and at least 60% sequence identity with the parent CDR;
v)
A5) an antibody comprising
a) a CDR1 comprising any of the amino acid sequences SEQ ID 171, SEQ ID 181, SEQ ID 183, or SEQ ID 185; and
b) a CDR2 comprising any of the amino acid sequences SEQ ID 172, SEQ ID 182, SEQ ID 184, or SEQ ID 186; and
c) a CDR3 comprising the amino acid sequence SEQ ID 173; or
B5) an antibody comprising the antibody of A5, wherein at least one of CDR1, CDR2, or CDR3 is a functionally active CDR variant of a parent CDR, comprising at least one point mutation and at least 60% sequence identity with the parent CDR;
vi)
A6) an antibody comprising
a) a CDR1 comprising any of the amino acid sequences SEQ ID 188, SEQ ID 194, SEQ ID 196, SEQ ID 122, SEQ ID 198, SEQ ID 203, or SEQ ID 204; and
b) a CDR2 comprising any of the amino acid sequences SEQ ID 189, SEQ ID 193, SEQ ID 195, SEQ ID 197, SEQ ID 186, SEQ ID 199, or SEQ ID 205; and
c) a CDR3 comprising the amino acid sequence SEQ ID 190; or
B6) an antibody comprising the antibody of A6, wherein at least one of CDR1, CDR2, or CDR3 is a functionally active CDR variant of a parent CDR, comprising at least one point mutation and at least 60% sequence identity with the parent CDR;
vii)
A7) an antibody comprising
a) a CDR1 comprising the amino acid sequence SEQ ID 209; and
b) a CDR2 comprising the amino acid sequence SEQ ID 210; and
c) a CDR3 comprising the amino acid sequence SEQ ID 211; or
B7) an antibody comprising the antibody of A7, wherein at least one of CDR1, CDR2, or CDR3 is a functionally active CDR variant of a parent CDR, comprising at least one point mutation and at least 60% sequence identity with the parent CDR; and
viii)
A8) an antibody comprising
a) a CDR1 comprising the amino acid sequence SEQ ID 218; and
b) a CDR2 comprising the amino acid sequence SEQ ID 219; and
c) a CDR3 comprising the amino acid sequence SEQ ID 221; or
B8) an antibody comprising the antibody of A8, wherein at least one of CDR1, CDR2, or CDR3 is a functionally active CDR variant of a parent CDR, comprising at least one point mutation and at least 60% sequence identity with the parent CDR.

26. The combination preparation of claim 24, wherein the anti-LukGH antibody comprises:
i)
A1) an antibody comprising
a) a CDR4 comprising the amino acid sequence SEQ ID 93 or SEQ ID 103; and
b) a CDR5 comprising any of the amino acid sequences SEQ ID 95, SEQ ID 100, or SEQ ID 105; and
c) a CDR6 comprising any of the amino acid sequences SEQ ID 97, SEQ ID 101, SEQ ID 107, or SEQ ID 108; or
B1) an antibody comprising the antibody of Al, wherein at least one of CDR4, CDR5, or CDR6 is a functionally active CDR variant of a parent CDR, comprising at least one point mutation and at least 60% sequence identity with the parent CDR;
ii)
A2) an antibody comprising
a) a CDR4 comprising the amino acid sequence SEQ ID 116; and
b) a CDR5 comprising the amino acid sequence SEQ ID 117 or SEQ ID 125; and
c) a CDR6 comprising any of the amino acid sequences SEQ ID 119, SEQ ID 126, SEQ ID 127, or SEQ ID 129; or
B2) an antibody comprising the antibody of A2, wherein at least one of CDR4, CDR5, or CDR6 is a functionally active CDR variant of a parent CDR, comprising at least one point mutation and at least 60% sequence identity with the parent CDR;
iii)
A3) an antibody comprising
a) a CDR4 comprising any of the amino acid sequences SEQ ID 137, SEQ ID 151, or SEQ ID 103; and
b) a CDR5 comprising the amino acid sequence SEQ ID 105; and
c) a CDR6 comprising any of the amino acid sequences SEQ ID 138, SEQ ID 152, SEQ ID 153, or SEQ ID 154; or
B3) an antibody comprising the antibody of A3, wherein at least one of CDR4, CDR5, or CDR6 is a functionally active CDR variant of a parent CDR, comprising at least one point mutation and at least 60% sequence identity with the parent CDR;
iv)
A4) an antibody comprising
a) a CDR4 comprising the amino acid sequence SEQ ID 159 or SEQ ID 116; and
b) a CDR5 comprising the amino acid sequence SEQ ID 125; and
c) a CDR6 comprising the amino acid sequence SEQ ID 160 or SEQ ID 170; or
B4) an antibody comprising the antibody of A4, wherein at least one of CDR4, CDR5, or CDR6 is a functionally active CDR variant of a parent CDR, comprising at least one point mutation and at least 60% sequence identity with the parent CDR;
v)
A5) an antibody comprising
a) a CDR4 comprising the amino acid sequence SEQ ID 176; and
b) a CDR5 comprising any of the amino acid sequence SEQ ID 178; and
c) a CDR6 comprising the amino acid sequence SEQ ID 180 or SEQ ID 187; or
B5) an antibody comprising the antibody of A5, wherein at least one of CDR4, CDR5, or CDR6 is a functionally active CDR variant of a parent CDR, comprising at least one point mutation and at least 60% sequence identity with the parent CDR;
vi)
A6) an antibody comprising
a) a CDR4 comprising the amino acid sequence SEQ ID 176 or SEQ ID 200; and
b) a CDR5 comprising the amino acid sequence SEQ ID 178 or SEQ ID 201; and
c) a CDR6 comprising any of the amino acid sequences SEQ ID 192, SEQ ID 202, or SEQ ID 207; or
B6) an antibody comprising the antibody of A6, wherein at least one of CDR4, CDR5, or CDR6 is a functionally active CDR variant of a parent CDR, comprising at least one point mutation and at least 60% sequence identity with the parent CDR;
vii)
A7) an antibody comprising
a) a CDR4 comprising the amino acid sequence SEQ ID 116; and
b) a CDR5 comprising the amino acid sequence SEQ ID 125; and
c) a CDR6 comprising any of the amino acid sequences SEQ ID 213, SEQ ID 214, SEQ ID 215, or SEQ ID 216; or
B7) an antibody comprising the antibody of A7, wherein at least one of CDR4, CDR5, or CDR6 is a functionally active CDR variant of a parent CDR, comprising at least one point mutation and at least 60% sequence identity with the parent CDR; and
viii)
A8) an antibody comprising
a) a CDR4 comprising the amino acid sequence SEQ ID 176 or SEQ ID 200; and
b) a CDR5 comprising the amino acid sequence SEQ ID 178; and
c) a CDR6 comprising any of the amino acid sequences SEQ ID 224, SEQ ID 180, SEQ ID 226, or SEQ ID 227; or
B8) an antibody comprising the antibody of A8, wherein at least one of CDR4, CDR5, or CDR6 is a functionally active CDR variant of a parent CDR, comprising at least one point mutation and at least 60% sequence identity with the parent CDR.

27. The combination preparation of claim 24, wherein the anti-LukGH antibody is selected from the group consisting of
A. an antibody comprising
a. the CDR1 sequence SEQ ID 122; and
b. the CDR2 sequence SEQ ID 123; and
c. the CDR3 sequence SEQ ID 114; and
d. the CDR4 sequence SEQ ID 116; and
e. the CDR5 sequence SEQ ID 117; and
f. the CDR6 sequence SEQ ID 119;
B. an antibody comprising
a. the CDR1 sequence SEQ ID 131; and
b. the CDR2 sequence SEQ ID 133; and
c. the CDR3 sequence SEQ ID 135; and
d. the CDR4 sequence SEQ ID 137; and
e. the CDR5 sequence SEQ ID 105; and
f. the CDR6 sequence SEQ ID 138;
C. an antibody comprising
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160;
D. an antibody comprising
a. the CDR1 sequence SEQ ID 188; and
b. the CDR2 sequence SEQ ID 189; and
c. the CDR3 sequence SEQ ID 190; and
d. the CDR4 sequence SEQ ID 176; and
e. the CDR5 sequence SEQ ID 178; and
f. the CDR6 sequence SEQ ID 192;
and
E. an antibody comprising
a. the CDR1 sequence SEQ ID 198; and
b. the CDR2 sequence SEQ ID 199; and
c. the CDR3 sequence SEQ ID 190; and
d. the CDR4 sequence SEQ ID 200; and
e. the CDR5 sequence SEQ ID 201; and
f. the CDR6 sequence SEQ ID 202;
or a functionally active CDR variant of any of A) through E), wherein the functionally active CDR variant has an affinity for binding the LukGH complex with a K_{D} of less than 10⁻⁸M.

28. The combination preparation of claim 18, wherein the OPK antibody is an anti Ig-binding protein (anti-IGBP) antibody comprising a cross-specific CDR binding site that recognizes at least three IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity for binding SpA-E with a K_{D} of less than 5×10⁻⁹M, as determined by a standard optical interferometry method for a F(ab)2 fragment.

29. The combination preparation of claim 28, wherein the anti-IGBP antibody recognizes a wild-type SpA with at least substantially the same affinity or with substantially higher affinity as compared to a mutant SpA_{KKAA}.

30. The combination preparation of claim 28, wherein the anti-IGBP antibody comprises an antibody heavy chain variable region (VH), wherein the VH comprises a CDR1, CDR2, and CDR3 sequence of any of the antibody sequences listed in Table 3, and an antibody light chain region (VL), wherein the VL comprises a CDR4, CDR5, and CDR6 sequence of any of the antibody sequences listed in Table 3, wherein the CDR sequences are designated according to the numbering system of Kabat, or a functionally active CDR variant thereof.

31. The combination preparation of claim 28, wherein the anti-IGBP antibody is selected from the group consisting of i) to vi), wherein the antibody comprises:
i)
A1) an antibody comprising
a) a CDR1 consisting of the amino acid sequence of SEQ ID 269; and
b) a CDR2 consisting of the amino acid sequence of SEQ ID 270; and
c) a CDR3 consisting of the amino acid sequence of SEQ ID 271;
and optionally further comprises
d) a CDR4 consisting of the amino acid sequence of SEQ ID 329; and
e) a CDR5 consisting of the amino acid sequence of SEQ ID 330; and
f) a CDR6 consisting of the amino acid sequence of SEQ ID 331; or
B1) an antibody comprising the antibody of Al, wherein at least one of CDR1-CDR6 is a functionally active CDR variant of a parent CDR, comprising at least one point mutation and at least 60% sequence identity with the parent CDR;
ii)
A2) an antibody comprising
a) a CDR1 consisting of the amino acid sequence of SEQ ID 287; and
b) a CDR2 consisting of the amino acid sequence of SEQ ID 288; and
c) a CDR3 consisting of the amino acid sequence of SEQ ID 289;
and optionally further comprising
d) a CDR4 consisting of the amino acid sequence of SEQ ID 347; and
e) a CDR5 consisting of the amino acid sequence of SEQ ID 348; and
f) a CDR6 consisting of the amino acid sequence of SEQ ID 349; or
B2) an antibody comprising the antibody of A2, wherein at least one of CDR1-CDR6 is a functionally active CDR variant of a parent CDR, comprising at least one point mutation and at least 60% sequence identity with the parent CDR;
iii)
A3) an antibody comprising
a) a CDR1 consisting of the amino acid sequence of SEQ ID 296; and
b) a CDR2 consisting of the amino acid sequence of SEQ ID 297; and
c) a CDR3 consisting of the amino acid sequence of SEQ ID 298;
and optionally further comprising
d) a CDR4 consisting of the amino acid sequence of SEQ ID 356; and
e) a CDR5 consisting of the amino acid sequence of SEQ ID 357; and
f) a CDR6 consisting of the amino acid sequence of SEQ ID 358; or
B3) an antibody comprising the antibody of A3, wherein at least one of CDR1-CDR6 is a functionally active CDR variant of a parent CDR, comprising at least one point mutation and at least 60% sequence identity with the parent CDR;
iv)
A4) an antibody comprising
a) a CDR1 consisting of the amino acid sequence of SEQ ID 299; and
b) a CDR2 consisting of the amino acid sequence of SEQ ID 300; and
c) a CDR3 consisting of the amino acid sequence of SEQ ID 301;
and optionally further comprising
d) a CDR4 consisting of the amino acid sequence of SEQ ID 359; and
e) a CDR5 consisting of the amino acid sequence of SEQ ID 360; and
f) a CDR6 consisting of the amino acid sequence of SEQ ID 361; or
B4) an antibody comprising the antibody of A4, wherein at least one of CDR1-6 is a functionally active CDR variant of a parent CDR, comprising at least one point mutation and at least 60% sequence identity with the parent CDR;
v)
A5) an antibody comprising
a) a CDR1 consisting of the amino acid sequence of SEQ ID 302; and
b) a CDR2 consisting of the amino acid sequence of SEQ ID 303; and
c) a CDR3 consisting of the amino acid sequence of SEQ ID 304;
and optionally further comprising
d) a CDR4 consisting of the amino acid sequence of SEQ ID 362; and
e) a CDR5 consisting of the amino acid sequence of SEQ ID 363; and
f) a CDR6 consisting of the amino acid sequence of SEQ ID 364; or
B5) an antibody comprising the antibody of A5, wherein at least one of CDR1-CDR6 is a functionally active CDR variant of a parent CDR, comprising at least one point mutation and at least 60% sequence identity with the parent CDR; and
vi)
A6) an antibody comprising
a) a CDR1 consisting of the amino acid sequence of SEQ ID 314; and
b) a CDR2 consisting of the amino acid sequence of SEQ ID 315; and
c) a CDR3 consisting of the amino acid sequence of SEQ ID 316;
and optionally further comprising
d) a CDR4 consisting of the amino acid sequence of SEQ ID 374; and
e) a CDR5 consisting of the amino acid sequence of SEQ ID 375; and
f) a CDR6 consisting of the amino acid sequence of SEQ ID 376; or
B6) an antibody comprising the antibody of A6, wherein at least one of CDR1-CDR6 is a functionally active CDR variant of a parent CDR, comprising at least one point mutation and at least 60% sequence identity with the parent CDR.

32. The combination preparation of claim 18, wherein
a) the toxin cross-neutralizing antibody comprises
a. the CDR1 sequence SEQ ID 1; and
b. the CDR2 sequence SEQ ID 2; and
c. the CDR3 sequence SEQ ID 12; and
d. the CDR4 sequence SEQ ID 32; and
e. the CDR5 sequence SEQ ID 33; and
f. the CDR6 sequence SEQ ID 34;
b) the anti-LukGH antibody comprises
a. the CDR1 sequence SEQ ID 167; and
b. the CDR2 sequence SEQ ID 168; and
c. the CDR3 sequence SEQ ID 157; and
d. the CDR4 sequence SEQ ID 159; and
e. the CDR5 sequence SEQ ID 125; and
f. the CDR6 sequence SEQ ID 160; and
c) the anti-IGBP antibody comprises
a. the CDR1 sequence SEQ ID 299; and
b. the CDR2 sequence SEQ ID 300; and
c. the CDR3 sequence SEQ ID 301; and
d. the CDR4 sequence SEQ ID 359; and
e. the CDR5 sequence SEQ ID 360; and
f. the CDR6 sequence SEQ ID 361;
or a functionally active CDR variant of any of the foregoing, wherein the functionally active CDR variant has an affinity for binding the target antigen with a K_{D} of less than 10⁻⁸M.

33. The combination preparation of claim 18, wherein each of the toxin cross-neutralizing antibody, the anti-LukGH antibody, and the anti-IGBP antibody is a full-length monoclonal antibody, an antibody fragment thereof comprising at least one antibody domain incorporating the binding site, or a fusion protein comprising at least one antibody domain incorporating the binding site.

34. A method of treating a subject at risk of or suffering from a S. aureus infection comprising administering to the subject an effective amount of an antibody to limit the infection in the subject, to ameliorate a disease condition resulting from said infection or to inhibit S. aureus disease pathogenesis, wherein the antibody is comprised in an anti-Staphylococcus aureus antibody combination preparation comprising
a) a toxin cross-neutralizing antibody comprising at least one polyspecific binding site that binds to alpha-toxin (HIa) and at least one bi-component toxin selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and
b) an anti-LukGH antibody; and/or
c) an OPK antibody which recognizes a S. aureus surface protein thereby inducing OPK.

35. An anti-Staphylococcus aureus antibody preparation comprising one or more antibodies specifically recognizing one or more S. aureus targets, the targets comprising:
a) alpha-toxin (HIa) and at least one bi-component toxin selected from the group consisting of HIgAB, HIgCB, LukSF, LukED, LukS-HIgB, LukSD, HIgA-LukD, HIgA-LukF, LukEF, LukE-HIgB, HIgC-LukD and HIgC-LukF; and
b) the LukGH complex; and/or
c) an S. aureus IgG binding domain of Protein A or Sbi; and/or
d) an S. aureus surface protein inducing OPK when bound by the antibody.

36. The combination preparation of claim 18, wherein the combination preparation comprises the toxin cross-neutralizing antibody and the anti-LukGH antibody, wherein
a) the toxin cross-neutralizing antibody is a mAb designated ASN-1; and
b) the anti-LukGH antibody is a mAb designated ASN-2, wherein
(i) the mAb designated ASN-1 comprises 6 CDR sequences of any of the antibody sequences listed in Table 1, or a functional variant thereof; and
(ii) the mAb designated ASN-2 comprising 6 CDR sequences of any of the antibody sequences listed in any of the Tables 2.1, 2.2, 2.3, 2.4, 2.5, 2.6, 2.7, or 2.8, or a functional variant thereof;
wherein any functional variant is a functionally active CDR variant of any of the foregoing, and wherein any functional variant has an affinity for binding the target antigen with a K_{D} of less than 10⁻⁸M.


SEQUENCE LISTING (423 sequences)

SEQ ID NO: 1 - protein, 10 aa, Artificial Sequence
  partial antibody sequence
YSISSGMGWG

SEQ ID NO: 2 - protein, 16 aa, Artificial Sequence
  partial antibody sequence
SIDQRGSTYYNPSLKS

SEQ ID NO: 3 - protein, 12 aa, Artificial Sequence
  partial antibody sequence
ARDAGHGVDMDV

SEQ ID NO: 4 - protein, 10 aa, Artificial Sequence
  partial antibody sequence
YPISSGMGWG

SEQ ID NO: 5 - protein, 10 aa, Artificial Sequence
  partial antibody sequence
YSISSGMGWD

SEQ ID NO: 6 - protein, 16 aa, Artificial Sequence
  partial antibody sequence
SVDQRGSTYYNPSLKS

SEQ ID NO: 7 - protein, 16 aa, Artificial Sequence
  partial antibody sequence
RIDQRGSTYYNPSLKS

SEQ ID NO: 8 - protein, 16 aa, Artificial Sequence
  partial antibody sequence
RVDQRGSTYYNPSLKS

SEQ ID NO: 9 - protein, 16 aa, Artificial Sequence
  partial antibody sequence
SIDQRGSTYYNPSLEG

SEQ ID NO: 10 - protein, 16 aa, Artificial Sequence
  partial antibody sequence
SIDQRGSTYYNPPLES

SEQ ID NO: 11 - protein, 12 aa, Artificial Sequence
  partial antibody sequence
ARDAGHGADMDV

SEQ ID NO: 12 - protein, 12 aa, Artificial Sequence
  partial antibody sequence
ARDAGHAVDMDV

SEQ ID NO: 13 - protein, 26 aa, Artificial Sequence
  partial antibody sequence
QVQLQESGPGLVKPSETLSLTCAVSG

SEQ ID NO: 14 - protein, 26 aa, Artificial Sequence
  partial antibody sequence
QVQLQESGPGLVRPSETLSLTCAVSG

SEQ ID NO: 15 - protein, 14 aa, Artificial Sequence
  partial antibody sequence
WIRQPPGKGLEWIG

SEQ ID NO: 16 - protein, 14 aa, Artificial Sequence
  partial antibody sequence
WIRQPPGKGREWIG

SEQ ID NO: 17 - protein, 30 aa, Artificial Sequence
  partial antibody sequence
RVTISVDTSKNQFSLKLSSVTAADTAVYYC

SEQ ID NO: 18 - protein, 30 aa, Artificial Sequence
  partial antibody sequence
RATISVDTSKNQFSLKLSSVTAADTAVYYC

SEQ ID NO: 19 - protein, 11 aa, Artificial Sequence
  partial antibody sequence
WGKGTTVTVSS

SEQ ID NO: 20 - protein, 119 aa, Artificial Sequence
  VH-Sequence
QVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHGVDMDVWGKGTTVTVSS

SEQ ID NO: 21 - protein, 119 aa, Artificial Sequence
  VH-Sequence
QVQLQESGPGLVKPSETLSLTCAVSGYSISSGMGWGWIRQPPGKGLEWIGSVDQRGSTYYNPSLKSRVTISVDTSKNQFSLKLSSVTAADTAVYYCARDAGHGADMDVWGKGTTVTVSS

SEQ ID NO: 22 - protein, 119 aa, Artificial Sequence
  VH-Sequence
QVQLQESGPGLVKPSETLSLTCAVSGYPISSGMGWGWIRQPPGKGLEWIGRIDQRGSTYYNPSLKSRATISVDTSKNQFSLKLSSVTAADTAVYYCARDAGHGVDMDVWGKGTTVTVSS

SEQ ID NO: 23 - protein, 119 aa, Artificial Sequence
  VH-Sequence
QVQLQESGPGLVKPSETLSLTCAVSGYSISSGMGWGWIRQPPGKGLEWIGRVDQRGSTYYNPSLKSRVTISVDTSKNQFSLKLSSVTAADTAVYYCARDAGHGVDMDVWGKGTTVTVSS

SEQ ID NO: 24 - protein, 119 aa, Artificial Sequence
  VH-Sequence
QVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 25 - protein, 119 aa, Artificial Sequence
  VH-Sequence
QVQLQESGPGLVKPSETLSLTCAVSGYSISSGMGWDWIRQPPGKGREWIGSIDQRGSTYYNPSLEGRATISVDTSKNQFSLKLSSVTAADTAVYYCARDAGHGVDMDVWGKGTTVTVSS

SEQ ID NO: 26 - protein, 119 aa, Artificial Sequence
  VH-Sequence
QVQLQESGPGLVKPSETLSLTCAVSGYSISSGMGWDWIRQPPGKGLEWIGSIDQRGSTYYNPPLESRVTISVDTSKNQFSLKLSSVTAADTAVYYCARDAGHGVDMDVWGKGTTVTVSS

SEQ ID NO: 27 - protein, 119 aa, Artificial Sequence
  VH-Sequence
EVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 28 - protein, 119 aa, Artificial Sequence
  VH-Sequence
QVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSS

SEQ ID NO: 29 - protein, 119 aa, Artificial Sequence
  VH-Sequence
QVQLQESGPGLVKPSETLSLTCAVSGYSISSGMGWDWIRQPPGKGLEWIGSIDQRGSTYYNPSLEGRVTISVDTSKNQFSLKLSSVTAADTAVYYCARDAGHGVDMDVWGKGTTVTVSS

SEQ ID NO: 30 - protein, 119 aa, Artificial Sequence
  VH-Sequence
QVQLQESGPGLVKPSETLSLTCAVSGYSISSGMGWDWIRQPPGKGREWIGSIDQRGSTYYNPSLEGRVTISVDTSKNQFSLKLSSVTAADTAVYYCARDAGHGVDMDVWGKGTTVTVSS

SEQ ID NO: 31 - protein, 119 aa, Artificial Sequence
  VH-Sequence
QVQLQESGPGLVKPSETLSLTCAVSGYSISSGMGWDWIRQPPGKGLEWIGSIDQRGSTYYNPSLEGRATISVDTSKNQFSLKLSSVTAADTAVYYCARDAGHGVDMDVWGKGTTVTVSS

SEQ ID NO: 32 - protein, 11 aa, Artificial Sequence
  partial antibody sequence
RASQGISRWLA

SEQ ID NO: 33 - protein, 7 aa, Artificial Sequence
  partial antibody sequence
AASSLQS

SEQ ID NO: 34 - protein, 9 aa, Artificial Sequence
  partial antibody sequence
QQGYVFPLT

SEQ ID NO: 35 - protein, 23 aa, Artificial Sequence
  partial antibody sequence
DIQMTQSPSSVSASVGDRVTITC

SEQ ID NO: 36 - protein, 15 aa, Artificial Sequence
  partial antibody sequence
WYQQKPGKAPKLLIY

SEQ ID NO: 37 - protein, 32 aa, Artificial Sequence
  partial antibody sequence
GVPSRFSGSGSGTDFTLTISSLQPEDFATYYC

SEQ ID NO: 38 - protein, 10 aa, Artificial Sequence
  partial antibody sequence
FGGGTKVEIK

SEQ ID NO: 39 - protein, 107 aa, Artificial Sequence
  VL-Sequence
DIQMTQSPSSVSASVGDRVTITCRASQGISRWLAWYQQKPGKAPKLLIYAASSLQSGVPSRFSGSGSGTDFTLTISSLQPEDFATYYCQQGYVFPLTFGGGTKVEIK

SEQ ID NO: 40 - protein, 468 aa, Artificial Sequence
  HC-Sequence
MELGLCWVFLVAILEGVQCQVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 458 aa, Artificial Sequence
  HC-Sequence
MELGLCWVFLVAILEGVQCQVQLQESGPGLVKPSETLSLTCAVSGYSISSGMGWGWIRQPPGKGLEWIGSVDQRGSTYYNPSLKSRVTISVDTSKNQFSLKLSSVTAADTAVYYCARDAGHGADMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYT

SEQ ID NO: 42 - protein, 456 aa, Artificial Sequence
  HC-Sequence
MELGLCWVFLVAILEGVQCQVQLQESGPGLVKPSETLSLTCAVSGYPISSGMGWGWIRQPPGKGLEWIGRIDQRGSTYYNPSLKSRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNH

SEQ ID NO: 43 - protein, 456 aa, Artificial Sequence
  HC-Sequence
MELGLCWVFLVAILEGVQCQVQLQESGPGLVKPSETLSLTCAVSGYSISSGMGWGWIRQPPGKGLEWIGRVDQRGSTYYNPSLKSRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNH

SEQ ID NO: 44 - protein, 468 aa, Artificial Sequence
  HC-Sequence
MELGLCWVFLVAILEGVQCQVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 468 aa, Artificial Sequence
  HC-Sequence
MELGLCWVFLVAILEGVQCQVQLQESGPGLVKPSETLSLTCAVSGYSISSGMGWDWIRQPPGKGREWIGSIDQRGSTYYNPSLEGRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 468 aa, Artificial Sequence
  HC-Sequence
MELGLCWVFLVAILEGVQCQVQLQESGPGLVKPSETLSLTCAVSGYSISSGMGWDWIRQPPGKGLEWIGSIDQRGSTYYNPPLESRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 467 aa, Artificial Sequence
  HC-Sequence
MELGLCWVFLVAILEGVQCEVQLQESGPGLVRPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 468 aa, Artificial Sequence
  HC-Sequence
MELGLCWVFLVAILEGVQCQVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHAVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 468 aa, Artificial Sequence
  HC-Sequence
MELGLCWVFLVAILEGVQCQVQLQESGPGLVKPSETLSLTCAVSGYSISSGMGWDWIRQPPGKGLEWIGSIDQRGSTYYNPSLEGRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 468 aa, Artificial Sequence
  HC-Sequence
MELGLCWVFLVAILEGVQCQVQLQESGPGLVKPSETLSLTCAVSGYSISSGMGWDWIRQPPGKGREWIGSIDQRGSTYYNPSLEGRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 468 aa, Artificial Sequence
  HC-Sequence
MELGLCWVFLVAILEGVQCQVQLQESGPGLVKPSETLSLTCAVSGYSISSGMGWDWIRQPPGKGLEWIGSIDQRGSTYYNPSLEGRATISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 233 aa, Artificial Sequence
  LC-Sequence
MELGLCWVFLVAILEGVQCDIQMTQSPSSVSASVGDRVTITCRASQGISRWLAWYQQKPGKAPKLLIYAASSLQSGVPSRFSGSGSGTDFTLTISSLQPEDFATYYCQQGYVFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 879 bp, Staphylococcus aureus
gccgacagcgacatcaacatcaaaacgggcacgacggacattggctcaaatacgacggtgaaaacgggcgatctggttacctatgacaaagaaaacggcatgcataaaaaagtgttttatagtttcatcgatgacaaaaaccacaacaaaaaactgctggtcattcgtaccaaaggcacgatcgcaggccagtatcgcgtgtacagcgaagaaggcgctaataaatcaggtctggcatggccgtcggcttttaaagttcagctgcaactgccggataacgaagtcgcgcaaattagcgactattacccgcgtaactctatcgataccaaagaatacatgtctaccctgacgtacggcttcaacggtaatgttaccggcgatgacacgggtaaaattggcggtctgatcggcgccaacgtgagcattggtcataccctgaaatatgttcagccggactttaaaaccatcctggaatctccgacggataaaaaagtgggctggaaagttatcttcaacaacatggttaaccagaactggggtccgtatgatcgtgactcatggaacccggtctacggcaatcaactgtttatgaaaacccgcaacggttcgatgaaagcggccgataacttcctggacccgaataaagcgagctctctgctgagttccggctttagtccggacttcgcgaccgtgattacgatggatcgcaaagcctccaaacagcaaaccaatattgatgtcatctatgaacgtgtgcgcgatgactaccagctgcactggaccagcacgaactggaaaggtaccaatacgaaagataaatggattgaccgctcctcggaacgctacaaaattgactgggaaaaagaagaaatgacgaac

SEQ ID NO: 54 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 55 - DNA, 852 bp, Staphylococcus aureus
gacaacaacattgaaaacattggtgatggcgcagaagtggtgaaacgcacggaagatacctcaagcgataaatggggtgtgacgcagaacattcagttcgatttcgtcaaagacaaaaaatacaacaaagatgcactgattctgaaaatgcaaggctttatcaacagcaaaaccacgtactacaactacaaaaacaccgaccatatcaaagctatgcgttggccgttccagtacaatatcggtctgaaaacgaacgatccgaatgttgacctgatcaactacctgccgaaaaacaaaatcgattcagtgaacgtttcgcaaaccctgggctacaatatcggcggtaactttaatagtggcccgtccaccggcggtaacggtagcttcaactactctaaaacgatcagttacaaccagcaaaactacatctctgaagtcgaacgtcagaacagcaaatctgtgcaatggggcattaaagcgaattcctttatcacctcactgggcaaaatgtcgggtcatgatccgaacctgtttgtgggttataaaccgtacagccagaacccgcgcgattatttcgttccggacaatgaactgccgccgctggtccattctggctttaacccgagtttcattgcaaccgtgagccacgaaaaaggctcgggtgataccagcgaatttgaaatcacgtatggtcgcaatatggacgttacccatgcgacgcgtcgcaccacgcactatggcaactcctacctggaaggttcacgtattcacaatgccttcgttaaccgcaattacacggtgaaatacgaagtcaactggaaaacgcacgaaatcaaagtgaaaggtcataac

SEQ ID NO: 56 - protein, 284 aa, Staphylococcus aureus
DNNIENIGDGAEVVKRTEDTSSDKWGVTQNIQFDFVKDKKYNKDALILKMQGFINSKTTYYNYKNTDHIKAMRWPFQYNIGLKTNDPNVDLINYLPKNKIDSVNVSQTLGYNIGGNFNSGPSTGGNGSFNYSKTISYNQQNYISEVERQNSKSVQWGIKANSFITSLGKMSGHDPNLFVGYKPYSQNPRDYFVPDNELPPLVHSGFNPSFIATVSHEKGSGDTSEFEITYGRNMDVTHATRRTTHYGNSYLEGSRIHNAFVNRNYTVKYEVNWKTHEIKVKGHN

SEQ ID NO: 57 - DNA, 903 bp, Staphylococcus aureus
gcgcagcacatcacgccggtctccgaaaaaaaagttgacgacaaaatcaccctgtataaaacgacggccacgagcgactctgacaaactgaaaatttctcagatcctgaccttcaacttcatcaaagataaaagttacgataaagacacgctgattctgaaagcggccggtaacatctattctggctacaccaaaccgaatccgaaagacacgatcagctctcaattctactggggttccaaatacaacatctcaatcaacagtgattccaacgactccgtcaatgtggttgattatgcaccgaaaaaccagaatgaagaattccaagtccagcaaaccgtgggctatagttacggcggtgacattaacatctcgaatggtctgagcggcggtggcaacggctcaaaatcgttcagcgaaacgatcaactacaaacaggaatcttaccgtaccagtctggataaacgcacgaatttcaagaaaattggttgggacgttgaagcgcataaaatcatgaacaatggttggggcccgtatggccgtgattcttatcacagtacctacggtaacgaaatgtttctgggctcccgccagtcaaacctgaatgccggtcaaaatttcctggaataccataaaatgccggttctgagccgtggtaactttaatccggaattcattggcgtcctgtcgcgcaaacagaacgcagcgaaaaaatctaaaatcaccgtgacgtatcagcgtgaaatggatcgctacaccaacttttggaatcaactgcattggatcggcaacaactacaaagatgaaaaccgtgccacccacacgagcatctacgaagttgactgggaaaaccacacggtgaaactgattgatacccaaagtaaagaaaaaaacccgatgtcg

SEQ ID NO: 58 - protein, 301 aa, Staphylococcus aureus
AQHITPVSEKKVDDKITLYKTTATSDSDKLKISQILTFNFIKDKSYDKDTLILKAAGNIYSGYTKPNPKDTISSQFYWGSKYNISINSDSNDSVNVVDYAPKNQNEEFQVQQTVGYSYGGDINISNGLSGGGNGSKSFSETINYKQESYRTSLDKRTNFKKIGWDVEAHKIMNNGWGPYGRDSYHSTYGNEMFLGSRQSNLNAGQNFLEYHKMPVLSRGNFNPEFIGVLSRKQNAAKKSKITVTYQREMDRYTNFWNQLHWIGNNYKDENRATHTSIYEVDWENHTVKLIDTQSKEKNPMS

SEQ ID NO: 59 - DNA, 849 bp, Staphylococcus aureus
aatacgaatatcgaaaatatcggcgacggcgcagaagttatcaaacgcacggaagatgtcagcagcaaaaaatggggtgttacgcagaatgttcagttcgatttcgtcaaagacaaaaaatacaacaaagatgcactgattgtgaaaatgcaaggctttatcaattctcgtaccagtttctccgacgttaaaggcagtggttatgaactgacgaaacgcatgatttggccgtttcagtacaacatcggtctgaccacgaaagatccgaacgtttccctgatcaactacctgccgaaaaacaaaatcgaaaccacggacgtcggccagaccctgggttacaacattggcggtaattttcaaagcgctccgtctatcggcggtaacggctcattcaattactcgaaaaccattagctatacgcagaaaagttacgtgtccgaagttgataaacaaaactcaaaatcggtcaaatggggcgtgaaagcgaacgaatttgtcaccccggatggtaaaaaatctgcccatgaccgttacctgtttgtgcagtcgccgaatggtccgacgggtagcgcacgtgaatactttgccccggataatcagctgccgccgctggtgcaatctggctttaacccgagtttcattaccacgctgagccatgaaaaaggcagctctgatacctccgaattcgaaatttcatatggtcgtaatctggacatcacctacgcaacgctgtttccgcgtaccggtatctatgcagaacgcaaacacaacgcttttgttaaccgcaatttcgttgtccgctacgaagtgaactggaaaacccatgaaatcaaagtgaaaggccataac

SEQ ID NO: 60 - protein, 283 aa, Staphylococcus aureus
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 61 - DNA, 903 bp, Staphylococcus aureus
gcccaacacattacgccggtctcggaaaaaaaagtggatgacaaaatcacgctgtataaaacgacggcaacctcagataacgacaaactgaacattagtcagatcctgaccttcaacttcatcaaagataaatcctacgataaagacacgctggtgctgaaagcggccggcaacattaattcaggttacaaaaaaccgaacccgaaagactataattactcgcagttttattggggcggtaaatacaacgtcagcgtgagctctgaatctaacgatgcagtcaatgtggttgactatgctccgaaaaaccagaatgaagaatttcaagtgcagcaaaccctgggctatagctacggcggtgatattaacatctcaaatggcctgtcgggcggtctgaacggttcgaaaagcttctctgaaaccatcaactacaaacaggaaagctaccgtaccacgattgatcgcaaaacgaaccataaatctatcggctggggtgttgaagcgcacaaaattatgaacaatggctggggtccgtatggccgtgattcctatgacccgacctacggtaatgaactgtttctgggcggtcgccagagttcctcaaacgcgggccaaaatttcctgccgacgcatcagatgccgctgctggcacgtggtaactttaatccggaattcatcagtgtgctgtcccacaaacaaaacgataccaaaaaatctaaaatcaaagttacgtatcaacgtgaaatggaccgctacaccaaccagtggaatcgcctgcattgggttggtaacaactacaaaaaccagaacaccgttacgttcacctctacgtacgaagtcgattggcaaaaccatacggtcaaactgattggcacggacagcaaagaaacgaacccgggcgtc

SEQ ID NO: 62 - protein, 301 aa, Staphylococcus aureus
AQHITPVSEKKVDDKITLYKTTATSDNDKLNISQILTFNFIKDKSYDKDTLVLKAAGNINSGYKKPNPKDYNYSQFYWGGKYNVSVSSESNDAVNVVDYAPKNQNEEFQVQQTLGYSYGGDINISNGLSGGLNGSKSFSETINYKQESYRTTIDRKTNHKSIGWGVEAHKIMNNGWGPYGRDSYDPTYGNELFLGGRQSSSNAGQNFLPTHQMPLLARGNFNPEFISVLSHKQNDTKKSKIKVTYQREMDRYTNQWNRLHWVGNNYKNQNTVTFTSTYEVDWQNHTVKLIGTDSKETNPGV

SEQ ID NO: 63 - DNA, 840 bp, Staphylococcus aureus
gaaaacaaaatcgaagacatcggccaaggtgctgaaatcatcaaacgcacgcaagacatcacgagtaaacgcctggcaatcacgcagaatattcagttcgatttcgtgaaagacaaaaaatacaacaaagatgcactggtggttaaaatgcaaggctttatcagctctcgtaccacgtacagcgatctgaaaaaatatccgtacattaaacgcatgatctggccgttccagtacaacatcagtctgaaaaccaaagattccaacgtggacctgattaattacctgccgaaaaacaaaatcgatagtgcggacgtttcccagaaactgggctataacattggcggtaattttcaatcagccccgtcgatcggcggtagtggttccttcaattactcaaaaaccatctcgtacaaccagaaaaattacgttacggaagtcgaaagccaaaactctaaaggcgtgaaatggggtgttaaagcgaattcatttgtcaccccgaacggccaggtgtcggcgtatgatcagtacctgtttgcacaagacccgacgggtccggcagcacgtgattatttcgttccggacaatcagctgccgccgctgattcaaagcggctttaacccgtctttcatcaccacgctgtcccatgaacgtggcaaaggtgataaaagcgaatttgaaattacctatggtcgcaacatggatgcaacctatgcttacgttacgcgtcatcgcctggcagtcgatcgtaaacacgacgctttcaaaaaccgcaatgtcaccgtgaaatacgaagtcaactggaaaacgcacgaagtcaaaatcaaatcaatcaccccgaaa

SEQ ID NO: 64 - protein, 280 aa, Staphylococcus aureus
ENKIEDIGQGAEIIKRTQDITSKRLAITQNIQFDFVKDKKYNKDALVVKMQGFISSRTTYSDLKKYPYIKRMIWPFQYNISLKTKDSNVDLINYLPKNKIDSADVSQKLGYNIGGNFQSAPSIGGSGSFNYSKTISYNQKNYVTEVESQNSKGVKWGVKANSFVTPNGQVSAYDQYLFAQDPTGPAARDYFVPDNQLPPLIQSGFNPSFITTLSHERGKGDKSEFEITYGRNMDATYAYVTRHRLAVDRKHDAFKNRNVTVKYEVNWKTHEVKIKSITPK

SEQ ID NO: 65 - DNA, 858 bp, Staphylococcus aureus
gcaaacgacacggaagacatcggcaaaggttcagacatcgaaatcatcaaacgcacggaagacaaaacgagcaataaatggggtgtgacccagaacattcaattcgatttcgtgaaagacaaaaaatacaataaagatgcgctgattctgaaaatgcagggctttatcagctctcgtaccacgtactacaactacaagaaaaccaaccatgttaaagccatgcgctggccgttccaatacaacatcggtctgaaaacgaatgacaaatatgtcagtctgattaactacctgccgaaaaataaaatcgaatcgaccaacgtgagccagacgctgggctataacattggcggtaattttcaatccgcaccgtcactgggcggtaacggttcattcaattactcaaaatcgatcagctatacccagcaaaactacgtgtctgaagttgaacagcaaaattctaaaagtgtcctgtggggcgtgaaagcgaatagctttgccacggaatctggtcagaaaagtgcatttgattccgacctgttcgtgggctataaaccgcattcaaaagatccgcgtgactacttcgtgccggattcggaactgccgccgctggttcagtcaggttttaacccgtcgttcattgctaccgttagtcacgaaaaaggcagttccgatacctccgaatttgaaattacgtatggtcgtaatatggacgtcacccatgcaatcaaacgcagcacgcactatggcaactcttacctggatggtcatcgtgttcacaatgcttttgtcaaccgcaattatacggtgaaatacgaagtcaactggaaaacgcacgaaatcaaagtcaaaggtcaaaac

SEQ ID NO: 66 - protein, 286 aa, Staphylococcus aureus
ANDTEDIGKGSDIEIIKRTEDKTSNKWGVTQNIQFDFVKDKKYNKDALILKMQGFISSRTTYYNYKKTNHVKAMRWPFQYNIGLKTNDKYVSLINYLPKNKIESTNVSQTLGYNIGGNFQSAPSLGGNGSFNYSKSISYTQQNYVSEVEQQNSKSVLWGVKANSFATESGQKSAFDSDLFVGYKPHSKDPRDYFVPDSELPPLVQSGFNPSFIATVSHEKGSSDTSEFEITYGRNMDVTHAIKRSTHYGNSYLDGHRVHNAFVNRNYTVKYEVNWKTHEIKVKGQN

SEQ ID NO: 67 - DNA, 900 bp, Staphylococcus aureus
gcggaaggcaaaattaccccggtctcggtgaaaaaagttgacgacaaagtgacgctgtataaaacgacggccacggctgattcggataaatttaaaattagccagatcctgaccttcaacttcatcaaagataaatcttacgataaagacaccctggtgctgaaagcaacgggcaacatcaatagcggttttgttaaaccgaacccgaatgattacgacttctcaaaactgtattggggcgcaaaatacaatgtttcgattagctctcagagtaacgattccgtcaatgtggttgactatgctccgaaaaaccaaaatgaagaatttcaggtgcaaaacaccctgggttacacgttcggcggtgatatttcaatctcgaatggcctgagtggcggtctgaacggtaataccgcgttttccgaaacgattaactataaacaggaaagctaccgtaccacgctgtctcgcaacaccaattataaaaatgtcggctggggtgtggaagcccataaaatcatgaacaatggctggggtccgtatggccgtgactcctttcacccgacgtacggcaacgaactgttcctggcaggtcgccagagttccgcatatgcaggtcaaaattttattgcccagcatcaaatgccgctgctgagccgttctaactttaatccggaattcctgtcagtcctgtcgcaccgccaggatggcgcgaaaaaatctaaaatcaccgttacgtaccagcgtgaaatggacctgtaccaaatccgctggaacggcttctattgggcaggtgctaactacaaaaacttcaaaacccgtacgttcaaatctacctatgaaatcgattgggaaaaccacaaagtcaaactgctggacacgaaagaaacggaaaataataaa

SEQ ID NO: 68 - protein, 300 aa, Staphylococcus aureus
AEGKITPVSVKKVDDKVTLYKTTATADSDKFKISQILTFNFIKDKSYDKDTLVLKATGNINSGFVKPNPNDYDFSKLYWGAKYNVSISSQSNDSVNVVDYAPKNQNEEFQVQNTLGYTFGGDISISNGLSGGLNGNTAFSETINYKQESYRTTLSRNTNYKNVGWGVEAHKIMNNGWGPYGRDSFHPTYGNELFLAGRQSSAYAGQNFIAQHQMPLLSRSNFNPEFLSVLSHRQDGAKKSKITVTYQREMDLYQIRWNGFYWAGANYKNFKTRTFKSTYEIDWENHKVKLLDTKETENNK

SEQ ID NO: 69 - DNA, 972 bp, Staphylococcus aureus
aactcggctcataaagatagtcaggatcaaaataaaaaagaacacgtggataaatcacaacagaaagataaacgcaatgtcaccaataaagataaaaatagcaccgcaccggatgacattggcaaaaacggtaaaatcaccaaacgtaccgaaacggtgtatgatgaaaaaacgaatattctgcagaacctgcaatttgatttcatcgatgacccgacctacgacaaaaatgtgctgctggttaaaaaacagggcagcattcattctaacctgaaattcgaaagtcacaaagaagagaaaaactccaactggctgaaatatccgtcagaataccatgtcgatttccaggtgaaacgtaatcgcaaaaccgaaattctggaccaactgccgaaaaacaaaatcagtaccgccaaagttgatagtacgttttcctatagctctggcggtaaattcgactctaccaaaggcatcggtcgtacgagttccaactcatactcgaaaaccatctcgtacaaccagcaaaactacgatacgatcgcaagcggcaaaaacaataactggcatgttcactggtctgtcattgctaacgatctgaaatatggcggtgaagttaaaaatcgcaacgacgaactgctgttttaccgtaatacccgcatcgcgacggtcgaaaacccggaactgtcattcgcgtcgaaatatcgttacccggccctggtgcgctccggttttaatccggaattcctgacctacctgagcaacgaaaaatctaacgaaaaaacgcagttcgaagtcacctatacgcgtaatcaagatattctgaaaaaccgtccgggcattcactacgcaccgccgatcctggagaaaaacaaagatggtcagcgcctgatcgtgacctatgaagttgactggaaaaacaaaaccgtgaaagtggtggacaaatactcggacgacaataaaccgtacaaagaaggt

SEQ ID NO: 70 - protein, 324 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 71 - DNA, 927 bp, Staphylococcus aureus
aaaatcaacagcgaaatcaaacaagtcagcgaaaaaaatctggatggcgatacgaaaatgtacacgcgcacggcaaccacgagcgattcgcagaaaaacatcacccagagcctgcaatttaatttcctgaccgaaccgaactacgataaagaaacggtgttcatcaaagcaaaaggcaccatcggctcaggtctgcgtattctggacccgaatggctactggaactcgaccctgcgctggccgggtagctattctgtgagtattcagaatgttgatgacaacaataacaccaacgttacggattttgctccgaaaaatcaagatgaaagccgtgaagtcaaatatacctacggctataaaacgggcggtgatttctctatcaatcgcggcggtctgaccggtaatattacgaaagaatcgaactatagcgaaaccatctcctaccagcaaccgtcatatcgtaccctgctggatcagtccacgtcacataaaggcgttggttggaaagtcgaagcgcacctgatcaataacatgggccatgatcacacccgtcaactgacgaatgatagcgacaaccgcacgaaatctgaaatttttagtctgacccgcaatggtaacctgtgggcgaaagataacttcacgccgaaagacaaaatgccggtcaccgtgtccgaaggctttaatccggaattcctggccgttatgtctcatgataaaaaagacaaaggtaaaagtcagttcgtggttcactacaaacgttccatggatgaattcaaaatcgactggaaccgccatggcttctggggttactggagcggtgaaaaccacgtcgataaaaaagaagaaaaactgtctgcactgtatgaagtggactggaaaacccacaatgtcaaattcgtgaaagttctgaatgataatgaaaaaaaa

SEQ ID NO: 72 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 73 - DNA, 966 bp, Staphylococcus aureus
gcaaacaaggactcccaggaccagaccaaaaaagaacacgtcgataaagcacagcagaaagaaaagcgtaatgtcaacgataaagataaaaataccccgggcccggatgacattggcaaaaacggcaaggttaccaaacgtaccgtcagtgaatatgacaaagaaaccaatattctgcagaacctgcaatttgatttcatcgatgacccgacgtacgacaaaaatgtgctgctggttaaaaagcaaggtagtatccattccaacctgaagtttgaaagccaccgtaatgaaaccaacgcgagttggctgaaatatccgtccgaataccatgtcgatttccaggtgcaacgcaatccgaaaacggaaattctggaccagctgccgaaaaacaagatctcaaccgcaaaagtggattcgacgtttagttattccctgggcggtaaattcgacagcaccaaaggcattggtcgcaccagcagcaacagctactcgaagagcatctcttacaaccagcaaaactacgataccatcgcaagcggcaaaaacaataaccgtcatgttcactggtctgtggttgctaatgatctgaagtatggtaacgaaatcaaaaatcgcaacgacgaatttctgttctaccgtaatacccgcctgagtacggtcgaaaacccggaactgtcatttgcgtcgaaatatcgttacccggccctggttcgctccggctttaatccggaatttctgacctacatcagcaacgaaaagtctaacgaaaagacgcgtttcgaagtgacctatacgcgcaatcaggatatcctgaaaaacaagccgggcattcactacggtcagccgatcctggaacaaaacaaagatggccagcgttttattgtcgtgtatgaagtggactggaaaaataagaccgttaaggttgtcgaaaaatattctgatcagaacaagccgtacaaagaaggt

SEQ ID NO: 74 - protein, 322 aa, Staphylococcus aureus
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 75 - DNA, 945 bp, Staphylococcus aureus
gcaagctcgtatgcggaaatcaaaagcaagatcaccaccgtctcagaaaagaacctggatggcgacaccaagatgtacacccgtaccgcgaccacgagcgatacggaaaagaaaattagccagtctctgcaatttaatttcctgaccgaaccgaactacgacaaagaaacggtgtttattaaagccaagggcaccatcggcagcggtctgaaaattctgaatccgaacggctactggaacagcaccctgcgttggccgggtagttattccgtttcaattcagaacgtcgatgacaacaataactcaaccaatgtcacggattttgcaccgaaaaaccaagacgaatcgcgtgaagtgaaatatacctacggctataagacgggcggtgatttcagtatcaatcgcggcggtctgaccggtaacatcacgaaggaaaagaactactcggaaaccatcagctaccagcaaccgtcttatcgtaccctgattgatcagccgaccacgaataaaggcgtcgcgtggaaggtggaagcccatagcatcaataacatgggtcatgatcacacccgtcaactgacgaacgactctgatgaccgcgtgaaatctgaaatttttagtctgacccgcaatggcaacctgtgggcaaaagataatttcacgccgaaaaacaagatgccggtgaccgtttccgaaggctttaatccggaatttctggctgttatgtcccatgataaaaacgacaaaggtaagtcacgtttcatcgtccactataaacgctcgatggatgactttaaactggattggaataagcatggcttctggggttactggagtggtgaaaaccacgttgaccagaaagaagaaaagctgtccgccctgtatgaagtggattggaaaacgcacgacgttaaactgattaagaccatcaacgataaagaacagaag

SEQ ID NO: 76 - protein, 315 aa, Staphylococcus aureus
ASSYAEIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 77 - DNA, 954 bp, Staphylococcus aureus
gactcacaggaccaaaacaaaaaggaacacgttgataaggcacagcagaaagacaagcaagatagcaccaagaaaggcaaaaacgttgcggccccggatgacgtcggcaaaaacggcaaggtgaccaaacgtacggaaagcgaatacgatgaaaagaccaacatcctgcagaacctggaatttaatttcatcgatgacccgacctacgataaagacgtcctgctggtgaaaaagcaaggcagtattcattccaacctgaagttcgaaagtcacaaagaagaaaagaacagcacctggctgaaatatccgtcagaataccatgttgatttccaggtcaagcgtaacccgaaaaccgaaattctggaccaactgccgaaaaataagatcagtacggcaaaagtggattcaaccttttcgtatacgctgggcggtaaattcgactccattaaaggcatcggtcgcaatagctctaacagctattctcagaccatttcgtataatcagcaaaactacgatacgatcgcgagcggcaaaaacaataactggcatgtgcactggtctgttattgccaacgatctgaagtatggcggtgaagttaaaaatcgtaacgacgaatttctgttctaccgtaacacccgcacgagttccgttgataatccggaatcatcgtttgcagctaaatatcgttacccggcactggtccgcagtggttttaatccggaatttctgacctatctgagcaacgaaaagtctaatgaaaaaacgcagtttgaagtgacctatacgcgtaaccaagatatcctgaaaaatagcccgggcctgcattacgctccgccgattctggaaaagaacaaggttggtcaccgctttatcgtcacctatgaagtggattggaaaaataagacggtgaaggtggttgacaaatactctgatgaccagccgttccgcgaaggt

SEQ ID NO: 78 - protein, 318 aa, Staphylococcus aureus
DSQDQNKKEHVDKAQQKDKQDSTKKGKNVAAPDDVGKNGKVTKRTESEYDEKTNILQNLEFNFIDDPTYDKDVLLVKKQGSIHSNLKFESHKEEKNSTWLKYPSEYHVDFQVKRNPKTEILDQLPKNKISTAKVDSTFSYTLGGKFDSIKGIGRNSSNSYSQTISYNQQNYDTIASGKNNNWHVHWSVIANDLKYGGEVKNRNDEFLFYRNTRTSSVDNPESSFAAKYRYPALVRSGFNPEFLTYLSNEKSNEKTQFEVTYTRNQDILKNSPGLHYAPPILEKNKVGHRFIVTYEVDWKNKTVKVVDKYSDDQPFREG

SEQ ID NO: 79 - DNA, 927 bp, Staphylococcus aureus
aaaatcaaatcggaaatcacgcaagttagcgaacagaatatcgacggcaatacgaagatgtttacccgcacggcaacgacctcggatagccagaaaaagatcagccagtctctgcaatttaacttcctgaccgaaccgaactacgacaaggaaacggtgttcatcaaggcaaagggcaccatcggctctggtctgaaaattctggacccgaacggctactggaatagtaccctgcgttggccgggtagttattccgtgtcaatccagaacgttgataacaataccaatacgaaggttacggattttgccccgaaaaaccaagacgaaacccgcgaagtcaagtatacctacggctataaaacgggcggtgatttctcgattagcccgggcggtattaccggtaacatcacgaaagaacgtaattattctgaaaccatcagttaccagcaaccgagttatcgcaccctgattgaccagccggcgacgaataagggcgttggttggaaagtcgaagcccatctgatcaacaatatgggccatgatcacacccgtcaactgacgaacgattccgacaatcgcgtgggctcagaaatttttaccctgacgcgtaacggtaatctgtgggcgaaagataacttcacgccgaaaaataagatgccggtcaccgtgtccgaaggctttaacccggaatttctggccgttatgtcgcatgataaaaaggacaaaggcaagagcaaatttgtggttcactataaacgtacgatggatgactttaaaatcgattggatgcgccatggcttctggggttactggaccggtaaaaatcacgttgaccagaaggaagaaaaactgtctgcactgtatgaagtcgattggaaaacccacgacgtgaagttcattaaagctctggatgacaaagaaaagaaa

SEQ ID NO: 80 - protein, 309 aa, Staphylococcus aureus
KIKSEITQVSEQNIDGNTKMFTRTATTSDSQKKISQSLQFNFLTEPNYDKETVFIKAKGTIGSGLKILDPNGYWNSTLRWPGSYSVSIQNVDNNTNTKVTDFAPKNQDETREVKYTYGYKTGGDFSISPGGITGNITKERNYSETISYQQPSYRTLIDQPATNKGVGWKVEAHLINNMGHDHTRQLTNDSDNRVGSEIFTLTRNGNLWAKDNFTPKNKMPVTVSEGFNPEFLAVMSHDKKDKGKSKFVVHYKRTMDDFKIDWMRHGFWGYWTGKNHVDQKEEKLSALYEVDWKTHDVKFIKALDDKEKK

SEQ ID NO: 81 - DNA, 966 bp, Staphylococcus aureus
gccaacaaggacagccaggaccaaaccaagaaggagcacgtggacaaggcccagcaaaaggagaagcgcaacgtgaacgacaaggataagaacacacctggcccggatgatatcggcaagaacggtaaggtgaccaagcgcacagagaccgtttacgacgagaagaccaacatcctgcagaacctgcagttcgacttcatcgacgacccgacctacgacaagaacatcctgttagtgaaaaagcagggcagcatccacagcaacctgaagttcgaaagccacaaagaggagaacaacagcagctggctgaaataccctagcgagtaccacgtggatttccaggtgaagcgcaaccgtaagaccgagatcctggaccagctgccgaagaacaagatcagtaccgcaaaggtggacagcaccttcagttacaacagcggcggcaagtttgatagtgttaagggcgttggccgcacaagtagcaacagctacagcaagacaattagttataaccagcagaactatgacacaatcgccagcggcaagaacaacaactggcacgtgcattggagcgtggtggcaaatgacctgaaatacggcggcgaggtgaaaaatcgcaacgacgagttcctgttttaccgcacaacccgcctgagcaccgtggagaaccctgagctgagctttgccagcaagtatcgctatccggcactggtgcgtagcggcttcaacccggagttcctgacctacctgagtaacgagaagagcaatgagaaaacacagttcgaggtgacctacacccgcaaccaggacatcctgaagaacaaaccgggcatccactatgccccgccgatcctggagaagaataaggacggccagcgcctgattgtgacctacgaggtggactggaagaacaaaaccgtgaaggtgatcgacaaatacagcgacgacaacaagccgtataaagagggg

SEQ ID NO: 82 - protein, 322 aa, Staphylococcus aureus
ANKDSQDQTKKEHVDKAQQKEKRNVNDKDKNTPGPDDIGKNGKVTKRTETVYDEKTNILQNLQFDFIDDPTYDKNILLVKKQGSIHSNLKFESHKEENNSSWLKYPSEYHVDFQVKRNRKTEILDQLPKNKISTAKVDSTFSYNSGGKFDSVKGVGRTSSNSYSKTISYNQQNYDTIASGKNNNWHVHWSVVANDLKYGGEVKNRNDEFLFYRTTRLSTVENPELSFASKYRYPALVRSGFNPEFLTYLSNEKSNEKTQFEVTYTRNQDILKNKPGIHYAPPILEKNKDGQRLIVTYEVDWKNKTVKVIDKYSDDNKPYKEG

SEQ ID NO: 83 - DNA, 921 bp, Staphylococcus aureus
aacagcgagatcaagcaggtgagcgagaaaaacctggacggcgagaccaagatgtacacacgcacagccaccaccagcgacagccagaaaaacatcacccagagtctgcagttcaacttcctgaccgagaagaactatgacaaagaaaccgttttcatcaaggccaaaggcaccatcggcagtggtctgcgcattctggagccgaacggctactggaacagcaccttacgttggcctggtagttacagcgtgagcatccagaatgtggacgacaacaacaacaccaacgtgaccgacttcgcaccgaagaatcaggacgagagccgcgaggtgaagtacacctacggctacaagaccggcggtgactttagcatcaaccagggtggcctgaccggcaacatcaccaaggagagcaactacagcgagaccatcagctatcagcaaccgagttaccgcacactgattgaccagccgacaaccaacaagggcgttgcctggaaagtggaggcccacctgatcaacaacatgggccatgaccacacacgtcagctgaccaacgatagcgatgaccgtgtgaagagcgagatcttcagcctgacccgcaacggtaatctgtgggccaaggacaacttcaccccgaaaaacaagatgccggtgaccgttagcgaaggcttcaatccggagttcctggccgttatgagccacgacaagaaggacgagggcaagagtaagttcgtggtgcactacaagcgcagcatggacgagttcaagattgactggaacaagcacggcttctggggctactggagcggcgagaaccacgtggacaagaaagaggagaagctgagcgcactgtacgaagtggactggaagacccataacgtgaagtttattaaggttttaaacgataaagagaaaaaa

SEQ ID NO: 84 - protein, 307 aa, Staphylococcus aureus
NSEIKQVSEKNLDGETKMYTRTATTSDSQKNITQSLQFNFLTEKNYDKETVFIKAKGTIGSGLRILEPNGYWNSTLRWPGSYSVSIQNVDDNNNTNVTDFAPKNQDESREVKYTYGYKTGGDFSINQGGLTGNITKESNYSETISYQQPSYRTLIDQPTTNKGVAWKVEAHLINNMGHDHTRQLTNDSDDRVKSEIFSLTRNGNLWAKDNFTPKNKMPVTVSEGFNPEFLAVMSHDKKDEGKSKFVVHYKRSMDEFKIDWNKHGFWGYWSGENHVDKKEEKLSALYEVDWKTHNVKFIKVLNDKEKK

SEQ ID NO: 85 - protein, 26 aa, Artificial Sequence
  Framework sequence
QVQLQESGPGLVKPSQTLSLTCTVSG

SEQ ID NO: 86 - protein, 11 aa, Artificial Sequence
  CDR sequence
GSIVNGMYYWS

SEQ ID NO: 87 - protein, 14 aa, Artificial Sequence
  Framework sequence
WIRQHPGKGLEWIG

SEQ ID NO: 88 - protein, 16 aa, Artificial Sequence
  CDR sequence
NIYYSGSTYYNPSLKS

SEQ ID NO: 89 - protein, 30 aa, Artificial Sequence; identical to SEQ ID NO: 17
  Framework sequence

SEQ ID NO: 90 - protein, 11 aa, Artificial Sequence
  CDR sequence
ARDVGRWFGNI

SEQ ID NO: 91 - protein, 11 aa, Artificial Sequence
  Framework sequence
WGQGTMVTVSS

SEQ ID NO: 92 - protein, 23 aa, Artificial Sequence
  Framework sequence
EIVLTQSPATLSLSPGERATLSC

SEQ ID NO: 93 - protein, 11 aa, Artificial Sequence
  CDR sequence
RASQSVSSYLA

SEQ ID NO: 94 - protein, 15 aa, Artificial Sequence
  Framework sequence
WYQQKPGQAPRLLIY

SEQ ID NO: 95 - protein, 7 aa, Artificial Sequence
  CDR sequence
DASNRAT

SEQ ID NO: 96 - protein, 32 aa, Artificial Sequence
  Framework sequence
GIPARFSGSGSGTDFTLTISSLEPEDFAVYYC

SEQ ID NO: 97 - protein, 9 aa, Artificial Sequence
  CDR sequence
QQRYVMPPT

SEQ ID NO: 98 - protein, 10 aa, Artificial Sequence
  Framework sequence
FGGGTKVEIK

SEQ ID NO: 99 - protein, 11 aa, Artificial Sequence
  CDR sequence
GSISSGGYYWS

SEQ ID NO: 100 - protein, 7 aa, Artificial Sequence
  CDR sequence
DSSNRAT

SEQ ID NO: 101 - protein, 9 aa, Artificial Sequence
  CDR sequence
QQRFNMPPT

SEQ ID NO: 102 - protein, 23 aa, Artificial Sequence
  Framework sequence
DIQMTQSPSSLSASVGDRVTITC

SEQ ID NO: 103 - protein, 11 aa, Artificial Sequence
  CDR sequence
QASQDISNYLN

SEQ ID NO: 104 - protein, 15 aa, Artificial Sequence
  Framework sequence
WYQQKPGKAPKLLIY

SEQ ID NO: 105 - protein, 7 aa, Artificial Sequence
  CDR sequence
DSSNRAT

SEQ ID NO: 106 - protein, 32 aa, Artificial Sequence
  Framework sequence
GVPSRFSGSGSGTDFTFTISSLQPEDIATYYC

SEQ ID NO: 107 - protein, 9 aa, Artificial Sequence
  CDR sequence
QQYDYLPPT

SEQ ID NO: 108 - protein, 9 aa, Artificial Sequence
  CDR sequence
QQVYDLPPT

SEQ ID NO: 109 - protein, 26 aa, Artificial Sequence
  Framework sequence
QVQLVQSGAEVKKPGSSVKVSCKASG

SEQ ID NO: 110 - protein, 9 aa, Artificial Sequence
  CDR sequence
GTFSAYEIS

SEQ ID NO: 111 - protein, 14 aa, Artificial Sequence
  Framework sequence
WVRQAPGQGLEWMG

SEQ ID NO: 112 - protein, 17 aa, Artificial Sequence
  CDR sequence
SIIPIFGRANYAQKFQG

SEQ ID NO: 113 - protein, 30 aa, Artificial Sequence
  Framework sequence
RVTITADESTSTAYMELSSLRSEDTAVYYC

SEQ ID NO: 114 - protein, 19 aa, Artificial Sequence
  CDR sequence
ARDAGCSSTSCHKDVYFDY

SEQ ID NO: 115 - protein, 11 aa, Artificial Sequence
  Framework sequence
WGQGTLVTVSS

SEQ ID NO: 116 - protein, 11 aa, Artificial Sequence
  CDR sequence
RASQSISSYLN

SEQ ID NO: 117 - protein, 7 aa, Artificial Sequence
  CDR sequence
GASSLQS

SEQ ID NO: 118 - protein, 32 aa, Artificial Sequence; identical to SEQ ID NO: 37
  Framework sequence

SEQ ID NO: 119 - protein, 9 aa, Artificial Sequence
  CDR sequence
QQAYYPPWT

SEQ ID NO: 120 - protein, 9 aa, Artificial Sequence
  CDR sequence
GTFSSYSIS

SEQ ID NO: 121 - protein, 17 aa, Artificial Sequence
  CDR sequence
SIIPIFGFANYAQKFQG

SEQ ID NO: 122 - protein, 9 aa, Artificial Sequence
  CDR sequence
GTFSSYAIS

SEQ ID NO: 123 - protein, 17 aa, Artificial Sequence
  CDR sequence
VIEPIFGFANYAQKFQG

SEQ ID NO: 124 - protein, 17 aa, Artificial Sequence
  CDR sequence
SIIPIFGTANYAQKFQG

SEQ ID NO: 125 - protein, 7 aa, Artificial Sequence
  CDR sequence
AASSLQS

SEQ ID NO: 126 - protein, 9 aa, Artificial Sequence
  CDR sequence
QQAYTPPWT

SEQ ID NO: 127 - protein, 9 aa, Artificial Sequence
  CDR sequence
QQAYTPPWT

SEQ ID NO: 128 - protein, 23 aa, Artificial Sequence
  Framework sequence
DIQLTQSPSSLSASVGDRVTITC

SEQ ID NO: 129 - protein, 9 aa, Artificial Sequence
  CDR sequence
QQSYYPPWT

SEQ ID NO: 130 - protein, 26 aa, Artificial Sequence
  Framework sequence
EVQLVESGGGLVQPGRSLRLSCAASG

SEQ ID NO: 131 - protein, 9 aa, Artificial Sequence
  CDR sequence
FTFYDYNMH

SEQ ID NO: 132 - protein, 14 aa, Artificial Sequence
  Framework sequence
WVRQAPGKGLEWVS

SEQ ID NO: 133 - protein, 17 aa, Artificial Sequence
  CDR sequence
GISWNSGSIAYADSVKG

SEQ ID NO: 134 - protein, 30 aa, Artificial Sequence
  Framework sequence
RFTISRDNAKNSLYLQMNSLRAEDTALYYC

SEQ ID NO: 135 - protein, 11 aa, Artificial Sequence
  CDR sequence
AKAQKGTAFDI

SEQ ID NO: 136 - protein, 11 aa, Artificial Sequence
  Framework sequence
WGQGTMVTVSS

SEQ ID NO: 137 - protein, 11 aa, Artificial Sequence
  CDR sequence
QASQDITNYLN

SEQ ID NO: 138 - protein, 9 aa, Artificial Sequence
  CDR sequence
QQYPVYPPT

SEQ ID NO: 139 - protein, 9 aa, Artificial Sequence
  CDR sequence
FTFADYNMH

SEQ ID NO: 140 - protein, 17 aa, Artificial Sequence
  CDR sequence
GISWNSGLTGYADSVKG

SEQ ID NO: 141 - protein, 9 aa, Artificial Sequence
  CDR sequence
FTFGDYNMH

SEQ ID NO: 142 - protein, 17 aa, Artificial Sequence
  CDR sequence
GISWNSGMSGYADSVKG

SEQ ID NO: 143 - protein, 9 aa, Artificial Sequence
  CDR sequence
FTFDLFAMH

SEQ ID NO: 144 - protein, 17 aa, Artificial Sequence
  CDR sequence
GITWNSGSTGYADSVKG

SEQ ID NO: 145 - protein, 9 aa, Artificial Sequence
  CDR sequence
FTFAEYAMH

SEQ ID NO: 146 - protein, 17 aa, Artificial Sequence
  CDR sequence
GITWNSGSIAYADSVKG

SEQ ID NO: 147 - protein, 9 aa, Artificial Sequence
  CDR sequence
FTFDSYNMH

SEQ ID NO: 148 - protein, 9 aa, Artificial Sequence
  CDR sequence
FTFDDYAMH

SEQ ID NO: 149 - protein, 17 aa, Artificial Sequence
  CDR sequence
GISWNSGAIAYADSVKG

SEQ ID NO: 150 - protein, 17 aa, Artificial Sequence
  CDR sequence
GISWNSGSIGYADSVKG

SEQ ID NO: 151 - protein, 11 aa, Artificial Sequence
  CDR sequence
QASQDIANYLN

SEQ ID NO: 152 - protein, 9 aa, Artificial Sequence
  CDR sequence
QQYPTYPPT

SEQ ID NO: 153 - protein, 9 aa, Artificial Sequence
  CDR sequence
QQYPIYPPT

SEQ ID NO: 154 - protein, 9 aa, Artificial Sequence
  CDR sequence
QQLPYLPFT

SEQ ID NO: 155 - protein, 11 aa, Artificial Sequence
  CDR sequence
GSISSGWYYWS

SEQ ID NO: 156 - protein, 16 aa, Artificial Sequence
  CDR sequence
NIFYSGSTYYNPSLKS

SEQ ID NO: 157 - protein, 11 aa, Artificial Sequence
  CDR sequence
ARERGMHYMDV

SEQ ID NO: 158 - protein, 11 aa, Artificial Sequence
  Framework sequence
WGKGTTVTVSS

SEQ ID NO: 159 - protein, 11 aa, Artificial Sequence
  CDR sequence
RASQSINSYLN

SEQ ID NO: 160 - protein, 9 aa, Artificial Sequence
  CDR sequence
QQQFDPPFT

SEQ ID NO: 161 - protein, 11 aa, Artificial Sequence
  CDR sequence
GSISSGHYYWG

SEQ ID NO: 162 - protein, 16 aa, Artificial Sequence
  CDR sequence
NIYGSGSTYYNPSLKS

SEQ ID NO: 163 - protein, 11 aa, Artificial Sequence
  CDR sequence
GSIESGFYYWS

SEQ ID NO: 164 - protein, 26 aa, Artificial Sequence
  Framework sequence
QLQLQESGPGLVKPSETLSLTCTVSG

SEQ ID NO: 165 - protein, 11 aa, Artificial Sequence
  CDR sequence
GSISSSGYYWV

SEQ ID NO: 166 - protein, 14 aa, Artificial Sequence
  Framework sequence
WIRQPPGKGLEWIG

SEQ ID NO: 167 - protein, 11 aa, Artificial Sequence
  CDR sequence
GSISSGSYYWD

SEQ ID NO: 168 - protein, 16 aa, Artificial Sequence
  CDR sequence
NIYKSGSTYYNPSLKS

SEQ ID NO: 169 - protein, 11 aa, Artificial Sequence
  CDR sequence
GSISSSSYYWG

SEQ ID NO: 170 - protein, 9 aa, Artificial Sequence
  CDR sequence
QQQYLPPFT

SEQ ID NO: 171 - protein, 9 aa, Artificial Sequence
  CDR sequence
GTFWSGAIS

SEQ ID NO: 172 - protein, 17 aa, Artificial Sequence
  CDR sequence
GIIPIEGPANYAQKFQG

SEQ ID NO: 173 - protein, 14 aa, Artificial Sequence
  CDR sequence
ARGIVAADHLYFDL

SEQ ID NO: 174 - protein, 11 aa, Artificial Sequence
  Framework sequence
WGRGTLVTVSS

SEQ ID NO: 175 - protein, 23 aa, Artificial Sequence
  Framework sequence
DIVMTQSPLSLPVTPGEPASISC

SEQ ID NO: 176 - protein, 16 aa, Artificial Sequence
  CDR sequence
RSSQSLLYSNGYNYLD

SEQ ID NO: 177 - protein, 15 aa, Artificial Sequence
  Framework sequence
WYLQKPGQSPQLLIY

SEQ ID NO: 178 - protein, 7 aa, Artificial Sequence
  CDR sequence
LGSNRAS

SEQ ID NO: 179 - protein, 32 aa, Artificial Sequence
  Framework sequence
GVPDRFSGSGSGTDFTLKISRVEAEDVGVYYC

SEQ ID NO: 180 - protein, 8 aa, Artificial Sequence
  CDR sequence
MQALQGLT

SEQ ID NO: 181 - protein, 9 aa, Artificial Sequence
  CDR sequence
GTFSLYAIS

SEQ ID NO: 182 - protein, 17 aa, Artificial Sequence
  CDR sequence
GIIPIDGPANYAQKFQG

SEQ ID NO: 183 - protein, 9 aa, Artificial Sequence
  CDR sequence
GTFWEYAIS

SEQ ID NO: 184 - protein, 17 aa, Artificial Sequence
  CDR sequence
GIIPIEGDANYAQKFQG

SEQ ID NO: 185 - protein, 9 aa, Artificial Sequence
  CDR sequence
GTFSNYAIS

SEQ ID NO: 186 - protein, 17 aa, Artificial Sequence
  CDR sequence
GIIPIFGTANYAQKFQG

SEQ ID NO: 187 - protein, 8 aa, Artificial Sequence
  CDR sequence
MQAKQGLT

SEQ ID NO: 188 - protein, 9 aa, Artificial Sequence
  CDR sequence
GTFSSWAIS

SEQ ID NO: 189 - protein, 17 aa, Artificial Sequence
  CDR sequence
GIIPIFGEAAYAQKFQG

SEQ ID NO: 190 - protein, 14 aa, Artificial Sequence
  CDR sequence
ARAREGDDPYGMDV

SEQ ID NO: 191 - protein, 11 aa, Artificial Sequence
  Framework sequence
WGQGTTVTVSS

SEQ ID NO: 192 - protein, 8 aa, Artificial Sequence
  CDR sequence
MQAKRLFT

SEQ ID NO: 193 - protein, 17 aa, Artificial Sequence
  CDR sequence
GIIPIFGPAAYAQKFQG

SEQ ID NO: 194 - protein, 9 aa, Artificial Sequence
  CDR sequence
GTFSNWAIS

SEQ ID NO: 195 - protein, 17 aa, Artificial Sequence
  CDR sequence
GIIPIFGSAAYAQKFQG

SEQ ID NO: 196 - protein, 9 aa, Artificial Sequence
  CDR sequence
GTFGSMAIS

SEQ ID NO: 197 - protein, 17 aa, Artificial Sequence
  CDR sequence
GIIPIFGPAVYAQKFQG

SEQ ID NO: 198 - protein, 9 aa, Artificial Sequence
  CDR sequence
GTIWSEAIS

SEQ ID NO: 199 - protein, 17 aa, Artificial Sequence
  CDR sequence
GIIPIFGDAVYAQKFQG

SEQ ID NO: 200 - protein, 16 aa, Artificial Sequence
  CDR sequence
RSSQSLLHSNGYNYLD

SEQ ID NO: 201 - protein, 7 aa, Artificial Sequence
  CDR sequence
LASNRAS

SEQ ID NO: 202 - protein, 8 aa, Artificial Sequence
  CDR sequence
MQARRLFT

SEQ ID NO: 203 - protein, 9 aa, Artificial Sequence
  CDR sequence
GTFKSWAIS

SEQ ID NO: 204 - protein, 9 aa, Artificial Sequence
  CDR sequence
GTFWSWAIS

SEQ ID NO: 205 - protein, 17 aa, Artificial Sequence
  CDR sequence
GIIPTFGEANYAQKFQG

SEQ ID NO: 206 - protein, 15 aa, Artificial Sequence
  Framework sequence
WYLQKPGQSPQLLIF

SEQ ID NO: 207 - protein, 8 aa, Artificial Sequence
  CDR sequence
MQARQLFT

SEQ ID NO: 208 - protein, 26 aa, Artificial Sequence
  Framework sequence
QVQLQESGPGLVKPSETLSLTCAVSG

SEQ ID NO: 209 - protein, 10 aa, Artificial Sequence
  CDR sequence
YSISSGYYWG

SEQ ID NO: 210 - protein, 16 aa, Artificial Sequence
  CDR sequence
TIYHSGSTYYNPSLKS

SEQ ID NO: 211 - protein, 9 aa, Artificial Sequence
  CDR sequence
ARTWSSMDV

SEQ ID NO: 212 - protein, 11 aa, Artificial Sequence
  Framework sequence
WGQGTTVTVSS

SEQ ID NO: 213 - protein, 9 aa, Artificial Sequence
  CDR sequence
QQIYLHPIT

SEQ ID NO: 214 - protein, 9 aa, Artificial Sequence
  CDR sequence
QQIYSHPIT

SEQ ID NO: 215 - protein, 9 aa, Artificial Sequence
  CDR sequence
QQIFSHPIT

SEQ ID NO: 216 - protein, 9 aa, Artificial Sequence
  CDR sequence
QQVYSHPIT

SEQ ID NO: 217 - protein, 26 aa, Artificial Sequence
  Framework sequence
EVQLLESGGGLVQPGGSLRLSCAASG

SEQ ID NO: 218 - protein, 9 aa, Artificial Sequence
  CDR sequence
FTFSSYAMS

SEQ ID NO: 219 - protein, 17 aa, Artificial Sequence
  CDR sequence
GISGSGGSTYYADSVKG

SEQ ID NO: 220 - protein, 30 aa, Artificial Sequence
  Framework sequence
RFTISRDNSKNTLYLQMNSLRAEDTAVYYC

SEQ ID NO: 221 - protein, 12 aa, Artificial Sequence
  CDR sequence
AKSLRYGYAFDL

SEQ ID NO: 222 - protein, 11 aa, Artificial Sequence
  Framework sequence
WGRGTLVTVSS

SEQ ID NO: 223 - protein, 15 aa, Artificial Sequence
  Framework sequence
WYLQKPGQSPQVLIY

SEQ ID NO: 224 - protein, 8 aa, Artificial Sequence
  CDR sequence
MQAKQGIT

SEQ ID NO: 225 - protein, 15 aa, Artificial Sequence
  Framework sequence
WYLQKPGQSPQLLIY

SEQ ID NO: 226 - protein, 8 aa, Artificial Sequence
  CDR sequence
MQARQGIT

SEQ ID NO: 227 - protein, 8 aa, Artificial Sequence
  CDR sequence
MQARQGLT

SEQ ID NO: 228 - protein, 26 aa, Artificial Sequence
  Framework sequence
ELQLQESGPGLVKPSETLSLTCTVSG

SEQ ID NO: 229 - protein, 26 aa, Artificial Sequence
  Framework sequence
EVQLVQSGAEVKKPGSSVKVSCKASG

SEQ ID NO: 230 - protein, 26 aa, Artificial Sequence
  Framework sequence
EVQLVQSGAEVKKPGSSVKVSCKASG

SEQ ID NO: 231 - protein, 449 aa, Artificial Sequence
  Heavy chain sequence
QVQLQESGPGLVKPSQTLSLTCTVSGGSIVNGMYYWSWIRQHPGKGLEWIGNIYYSGSTYYNPSLKSRVTISVDTSKNQFSLKLSSVTAADTAVYYCARDVGRWFGNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 214 aa, Artificial Sequence
  Light chain sequence
EIVLTQSPATLSLSPGERATLSCRASQSVSSYLAWYQQKPGQAPRLLIYDASNRATGIPARFSGSGSGTDFTLTISSLEPEDFAVYYCQQRYVMPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 456 aa, Artificial Sequence
  Heavy chain sequence
QVQLVQSGAEVKKPGSSVKVSCKASGGTFSAYEISWVRQAPGQGLEWMGSIIPIFGRANYAQKFQGRVTITADESTSTAYMELSSLRSEDTAVYYCARDAGCSSTSCHKDVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 214 aa, Artificial Sequence
  Light chain sequence
DIQMTQSPSSLSASVGDRVTITCRASQSISSYLNWYQQKPGKAPKLLIYGASSLQSGVPSRFSGSGSGTDFTLTISSLQPEDFATYYCQQAYYPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 456 aa, Artificial Sequence
  Heavy chain sequence
QVQLVQSGAEVKKPGSSVKVSCKASGGTFSSYAISWVRQAPGQGLEWMGVIEPIFGFANYAQKFQGRVTITADESTSTAYMELSSLRSEDTAVYYCARDAGCSSTSCHKDVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 214 aa, Artificial Sequence; identical to SEQ ID NO: 234
  Light chain sequence

SEQ ID NO: 237 - protein, 456 aa, Artificial Sequence
  Heavy chain sequence
EVQLVQSGAEVKKPGSSVKVSCKASGGTFSSYAISWVRQAPGQGLEWMGVIEPIFGFANYAQKFQGRVTITADESTSTAYMELSSLRSEDTAVYYCARDAGCSSTSCHKDVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 214 aa, Artificial Sequence; identical to SEQ ID NO: 234
  Light chain sequence

SEQ ID NO: 239 - protein, 448 aa, Artificial Sequence
  Heavy chain sequence
EVQLVESGGGLVQPGRSLRLSCAASGFTFYDYNMHWVRQAPGKGLEWVSGISWNSGSIAYADSVKGRFTISRDNAKNSLYLQMNSLRAEDTALYYCAKAQKGTAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 214 aa, Artificial Sequence
  Light chain sequence
DIQMTQSPSSLSASVGDRVTITCQASQDITNYLNWYQQKPGKAPKLLIYDASNLETGVPSRFSGSGSGTDFTFTISSLQPEDIATYYCQQYPVYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 449 aa, Artificial Sequence
  Heavy chain sequence
QVQLQESGPGLVKPSQTLSLTCTVSGGSISSGWYYWSWIRQHPGKGLEWIGNIFYSGSTYYNPSLKSRVTISVDTSKNQFSLKLSSVTAADTAVYYCARERGMHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 214 aa, Artificial Sequence
  Light chain sequence
DIQMTQSPSSLSASVGDRVTITCRASQSINSYLNWYQQKPGKAPKLLIYAASSLQSGVPSRFSGSGSGTDFTLTISSLQPEDFATYYCQQQFDPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 449 aa, Artificial Sequence
  Heavy chain sequence
QLQLQESGPGLVKPSETLSLTCTVSGGSISSGSYYWDWIRQPPGKGLEWIGNIYKSGSTYYNPSLKSRVTISVDTSKNQFSLKLSSVTAADTAVYYCARERGMHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, Artificial Sequence; identical to SEQ ID NO: 242
  Light chain sequence

SEQ ID NO: 245 - protein, 448 aa, Artificial Sequence
  Heavy chain sequence
ELQLQESGPGLVKPSETLSLTCTVSGGSISSGSYYWDWIRQPPGKGLEWIGNIYKSGSTYYNPSLKSRVTISVDTSKNQFSLKLSSVTAADTAVYYCARERGMHYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 246 - protein, 214 aa, Artificial Sequence; identical to SEQ ID NO: 242
  Light chain sequence

SEQ ID NO: 247 - protein, 451 aa, Artificial Sequence
  Heavy chain sequence
QVQLVQSGAEVKKPGSSVKVSCKASGGTFWSGAISWVRQAPGQGLEWMGGIIPIEGPANYAQKFQGRVTITADESTSTAYMELSSLRSEDTAVYYCARGIVAADHLYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 218 aa, Artificial Sequence
  Light chain sequence
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQALQGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 451 aa, Artificial Sequence
  Heavy chain sequence
QVQLVQSGAEVKKPGSSVKVSCKASGGTFSSWAISWVRQAPGQGLEWMGGIIPIFGEAAYAQKFQGRVTITADESTSTAYMELSSLRSEDTAVYYCARAREGDDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 218 aa, Artificial Sequence
  Light chain sequence
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQAKRLFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 450 aa, Artificial Sequence
  Heavy chain sequence
EVQLVQSGAEVKKPGSSVKVSCKASGGTFSSWAISWVRQAPGQGLEWMGGIIPIFGEAAYAQKFQGRVTITADESTSTAYMELSSLRSEDTAVYYCARAREGDDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 252 - protein, 218 aa, Artificial Sequence; identical to SEQ ID NO: 250
  Light chain sequence

SEQ ID NO: 253 - protein, 451 aa, Artificial Sequence
  Heavy chain sequence
QVQLVQSGAEVKKPGSSVKVSCKASGGTIWSEAISWVRQAPGQGLEWMGGIIPIFGDAVYAQKFQGRVTITADESTSTAYMELSSLRSEDTAVYYCARAREGDDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 218 aa, Artificial Sequence
  Light chain sequence
DIVMTQSPLSLPVTPGEPASISCRSSQSLLHSNGYNYLDWYLQKPGQSPQLLIYLASNRASGVPDRFSGSGSGTDFTLKISRVEAEDVGVYYCMQARRLFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 450 aa, Artificial Sequence
  Heavy chain sequence
EVQLVQSGAEVKKPGSSVKVSCKASGGTIWSEAISWVRQAPGQGLEWMGGIIPIFGDAVYAQKFQGRVTITADESTSTAYMELSSLRSEDTAVYYCARAREGDDPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 256 - protein, 218 aa, Artificial Sequence; identical to SEQ ID NO: 254
  Light chain sequence

SEQ ID NO: 257 - protein, 9 aa, Artificial Sequence
  VH-CDR1 Sequence
YTFTSYYIH

SEQ ID NO: 258 - protein, 17 aa, Artificial Sequence
  VH-CDR2 Sequence
IINPSGGSTSYAQKFQG

SEQ ID NO: 259 - protein, 13 aa, Artificial Sequence
  VH-CDR3 Sequence
ARGTMVSRPSLDL

SEQ ID NO: 260 - protein, 9 aa, Artificial Sequence
  VH-CDR1 Sequence
YTFRRYYMH

SEQ ID NO: 261 - protein, 17 aa, Artificial Sequence
  VH-CDR2 Sequence
IINPHVGSTSYAQKFQG

SEQ ID NO: 262 - protein, 16 aa, Artificial Sequence
  VH-CDR3 Sequence
ARGPDTAIHQGPAFDI

SEQ ID NO: 263 - protein, 9 aa, Artificial Sequence
  VH-CDR1 Sequence
YTFTSYYMH

SEQ ID NO: 264 - protein, 17 aa, Artificial Sequence
  VH-CDR2 Sequence
IINPSGGSTSYAQKFQG

SEQ ID NO: 265 - protein, 13 aa, Artificial Sequence
  VH-CDR3 Sequence
ARQGAGGTMHFDY

SEQ ID NO: 266 - protein, 9 aa, Artificial Sequence
  VH-CDR1 Sequence
YTFQAYYMH

SEQ ID NO: 267 - protein, 17 aa, Artificial Sequence
  VH-CDR2 Sequence
IINPSNGHTSYAQKFQG

SEQ ID NO: 268 - protein, 13 aa, Artificial Sequence
  VH-CDR3 Sequence
ARQGAGGTMHFDY

SEQ ID NO: 269 - protein, 9 aa, Artificial Sequence
  VH-CDR1 Sequence
YTFPSYYMH

SEQ ID NO: 270 - protein, 17 aa, Artificial Sequence
  VH-CDR2 Sequence
IINPSGGNTHYAQKFQG

SEQ ID NO: 271 - protein, 13 aa, Artificial Sequence
  VH-CDR3 Sequence
ARQGAGGTMHFDY

SEQ ID NO: 272 - protein, 9 aa, Artificial Sequence
  VH-CDR1 Sequence
YTFDSLYMH

SEQ ID NO: 273 - protein, 17 aa, Artificial Sequence
  VH-CDR2 Sequence
IINPSNGRTSYAQKFQG

SEQ ID NO: 274 - protein, 13 aa, Artificial Sequence
  VH-CDR3 Sequence
ARQGAGGTMHFDY

SEQ ID NO: 275 - protein, 9 aa, Artificial Sequence
  VH-CDR1 Sequence
YTFEEYYMH

SEQ ID NO: 276 - protein, 17 aa, Artificial Sequence
  VH-CDR2 Sequence
IINPSNGRTSYAQKFQG

SEQ ID NO: 277 - protein, 13 aa, Artificial Sequence
  VH-CDR3 Sequence
ARQGAGGTMHFDY

SEQ ID NO: 278 - protein, 9 aa, Artificial Sequence
  VH-CDR1 Sequence
YTFTRYYMH

SEQ ID NO: 279 - protein, 17 aa, Artificial Sequence
  VH-CDR2 Sequence
WINPNSGGTNYAQKFQG

SEQ ID NO: 280 - protein, 12 aa, Artificial Sequence
  VH-CDR3 Sequence
ARDPPMGQGMDV

SEQ ID NO: 281 - protein, 9 aa, Artificial Sequence
  VH-CDR1 Sequence
YTFTRYYMH

SEQ ID NO: 282 - protein, 17 aa, Artificial Sequence
  VH-CDR2 Sequence
WINPNSDGTNYAQKFQG

SEQ ID NO: 283 - protein, 12 aa, Artificial Sequence
  VH-CDR3 Sequence
ARDPPMGQGMDV

SEQ ID NO: 284 - protein, 9 aa, Artificial Sequence
  VH-CDR1 Sequence
YTFESYYMH

SEQ ID NO: 285 - protein, 17 aa, Artificial Sequence
  VH-CDR2 Sequence
WINPPSGRTNYAQKFQG

SEQ ID NO: 286 - protein, 12 aa, Artificial Sequence
  VH-CDR3 Sequence
ARDPPMGQGMDV

SEQ ID NO: 287 - protein, 9 aa, Artificial Sequence
  VH-CDR1 Sequence
YTFTRYYMH

SEQ ID NO: 288 - protein, 17 aa, Artificial Sequence
  VH-CDR2 Sequence
WINPPSGGTNYAQKFQG

SEQ ID NO: 289 - protein, 12 aa, Artificial Sequence
  VH-CDR3 Sequence
ARDPPMGQGMDV

SEQ ID NO: 290 - protein, 9 aa, Artificial Sequence
  VH-CDR1 Sequence
YTFTSRYMH

SEQ ID NO: 291 - protein, 17 aa, Artificial Sequence
  VH-CDR2 Sequence
IINPRLGSTSYAQKFQG

SEQ ID NO: 292 - protein, 16 aa, Artificial Sequence
  VH-CDR3 Sequence
ARDGEVGARDLGWFDP

SEQ ID NO: 293 - protein, 9 aa, Artificial Sequence
  VH-CDR1 Sequence
YTFRSRYMH

SEQ ID NO: 294 - protein, 17 aa, Artificial Sequence
  VH-CDR2 Sequence
IINPMYGSTSYAQKFQG

SEQ ID NO: 295 - protein, 16 aa, Artificial Sequence
  VH-CDR3 Sequence
ARDGEVGARDLGWFDP

SEQ ID NO: 296 - protein, 9 aa, Artificial Sequence
  VH-CDR1 Sequence
YTFGSHYMH

SEQ ID NO: 297 - protein, 17 aa, Artificial Sequence
  VH-CDR2 Sequence
IINPSIGSTSYAQKFQG

SEQ ID NO: 298 - protein, 16 aa, Artificial Sequence
  VH-CDR3 Sequence
ARDGEVGARDLGWFDP

SEQ ID NO: 299 - protein, 9 aa, Artificial Sequence
  VH-CDR1 Sequence
YTFSHYYMH

SEQ ID NO: 300 - protein, 17 aa, Artificial Sequence
  VH-CDR2 Sequence
IINPRYGSTSYAQKFQG

SEQ ID NO: 301 - protein, 16 aa, Artificial Sequence
  VH-CDR3 Sequence
ARDGEVGARDLGWFDP

SEQ ID NO: 302 - protein, 9 aa, Artificial Sequence
  VH-CDR1 Sequence
YTFPSRYMH

SEQ ID NO: 303 - protein, 17 aa, Artificial Sequence
  VH-CDR2 Sequence
IINPSLGSTSYAQKFQG

SEQ ID NO: 304 - protein, 16 aa, Artificial Sequence
  VH-CDR3 Sequence
ARDGEVGARDLGWFDP

SEQ ID NO: 305 - protein, 9 aa, Artificial Sequence
  VH-CDR1 Sequence
YTFALYYMH

SEQ ID NO: 306 - protein, 17 aa, Artificial Sequence
  VH-CDR2 Sequence
IINPHVGSTSYAQKFQG

SEQ ID NO: 307 - protein, 17 aa, Artificial Sequence
  VH-CDR3 Sequence
ARGRPLSGTGGHHYFDY

SEQ ID NO: 308 - protein, 9 aa, Artificial Sequence
  VH-CDR1 Sequence
YTFMRYYMH

SEQ ID NO: 309 - protein, 17 aa, Artificial Sequence
  VH-CDR2 Sequence
IINPGLGSTSYAQKFQG

SEQ ID NO: 310 - protein, 17 aa, Artificial Sequence
  VH-CDR3 Sequence
ARGRPLSGTGGHHYFDY

SEQ ID NO: 311 - protein, 9 aa, Artificial Sequence
  VH-CDR1 Sequence
YTFGEYYMH

SEQ ID NO: 312 - protein, 17 aa, Artificial Sequence
  VH-CDR2 Sequence
IINPRFGSTSYAQKFQG

SEQ ID NO: 313 - protein, 17 aa, Artificial Sequence
  VH-CDR3 Sequence
ARGRPLSGTGGHHYFDY

SEQ ID NO: 314 - protein, 9 aa, Artificial Sequence
  VH-CDR1 Sequence
YTFTSYYMH

SEQ ID NO: 315 - protein, 17 aa, Artificial Sequence
  VH-CDR2 Sequence
IINPRVGSTSYAQKFQG

SEQ ID NO: 316 - protein, 17 aa, Artificial Sequence
  VH-CDR3 Sequence
ARGRPLSGTGGHHYFDY

SEQ ID NO: 317 - protein, 11 aa, Artificial Sequence
  VL-CDR4 Sequence
RASQSVSSSLA

SEQ ID NO: 318 - protein, 7 aa, Artificial Sequence
  VL-CDR5 Sequence
GASTRAT

SEQ ID NO: 319 - protein, 9 aa, Artificial Sequence
  VL-CDR6 Sequence
QQDNVWPIT

SEQ ID NO: 320 - protein, 11 aa, Artificial Sequence
  VL-CDR4 Sequence
RASQSISSYLN

SEQ ID NO: 321 - protein, 7 aa, Artificial Sequence
  VL-CDR5 Sequence
AASSLQS

SEQ ID NO: 322 - protein, 10 aa, Artificial Sequence
  VL-CDR6 Sequence
QQDAVIPPFT

SEQ ID NO: 323 - protein, 11 aa, Artificial Sequence
  VL-CDR4 Sequence
RASQSVSSYLA

SEQ ID NO: 324 - protein, 7 aa, Artificial Sequence
  VL-CDR5 Sequence
DASNRAT

SEQ ID NO: 325 - protein, 8 aa, Artificial Sequence
  VL-CDR6 Sequence
QQRAPFFT

SEQ ID NO: 326 - protein, 11 aa, Artificial Sequence
  VL-CDR4 Sequence
RASQSVSSYLA

SEQ ID NO: 327 - protein, 7 aa, Artificial Sequence
  VL-CDR5 Sequence
DASNRAT

SEQ ID NO: 328 - protein, 8 aa, Artificial Sequence
  VL-CDR6 Sequence
QQRAPFFT

SEQ ID NO: 329 - protein, 11 aa, Artificial Sequence
  VL-CDR4 Sequence
RASQSVSSYLA

SEQ ID NO: 330 - protein, 7 aa, Artificial Sequence
  VL-CDR5 Sequence
DASNRAT

SEQ ID NO: 331 - protein, 8 aa, Artificial Sequence
  VL-CDR6 Sequence
QQRAPFFT

SEQ ID NO: 332 - protein, 11 aa, Artificial Sequence
  VL-CDR4 Sequence
RASQSVSSYLA

SEQ ID NO: 333 - protein, 7 aa, Artificial Sequence
  VL-CDR5 Sequence
DASNRAT

SEQ ID NO: 334 - protein, 8 aa, Artificial Sequence
  VL-CDR6 Sequence
QQRAPFFT

SEQ ID NO: 335 - protein, 11 aa, Artificial Sequence
  VL-CDR4 Sequence
RASQSVSSYLA

SEQ ID NO: 336 - protein, 7 aa, Artificial Sequence
  VL-CDR5 Sequence
DASNRAT

SEQ ID NO: 337 - protein, 8 aa, Artificial Sequence
  VL-CDR6 Sequence
QQRAPFFT

SEQ ID NO: 338 - protein, 11 aa, Artificial Sequence
  VL-CDR4 Sequence
RASQSISSYLN

SEQ ID NO: 339 - protein, 7 aa, Artificial Sequence
  VL-CDR5 Sequence
AASSLQS

SEQ ID NO: 340 - protein, 9 aa, Artificial Sequence
  VL-CDR6 Sequence
QQGFSSPFT

SEQ ID NO: 341 - protein, 11 aa, Artificial Sequence
  VL-CDR4 Sequence
RASQSISSYLN

SEQ ID NO: 342 - protein, 7 aa, Artificial Sequence
  VL-CDR5 Sequence
AASSLQS

SEQ ID NO: 343 - protein, 9 aa, Artificial Sequence
  VL-CDR6 Sequence
QQSFVAPFT

SEQ ID NO: 344 - protein, 11 aa, Artificial Sequence
  VL-CDR4 Sequence
RASQSISSYLN

SEQ ID NO: 345 - protein, 7 aa, Artificial Sequence
  VL-CDR5 Sequence
AASSLQS

SEQ ID NO: 346 - protein, 9 aa, Artificial Sequence
  VL-CDR6 Sequence
QQSFVAPFT

SEQ ID NO: 347 - protein, 11 aa, Artificial Sequence
  VL-CDR4 Sequence
RASQSISSYLN

SEQ ID NO: 348 - protein, 7 aa, Artificial Sequence
  VL-CDR5 Sequence
AASSLQS

SEQ ID NO: 349 - protein, 9 aa, Artificial Sequence
  VL-CDR6 Sequence
QQSFVAPFT

SEQ ID NO: 350 - protein, 11 aa, Artificial Sequence
  VL-CDR4 Sequence
RASQSVSSNLA

SEQ ID NO: 351 - protein, 7 aa, Artificial Sequence
  VL-CDR5 Sequence
GASTRAT

SEQ ID NO: 352 - protein, 9 aa, Artificial Sequence
  VL-CDR6 Sequence
QQYTPLPWT

SEQ ID NO: 353 - protein, 11 aa, Artificial Sequence
  VL-CDR4 Sequence
RASQSVSSNLA

SEQ ID NO: 354 - protein, 7 aa, Artificial Sequence
  VL-CDR5 Sequence
GASTRAT

SEQ ID NO: 355 - protein, 9 aa, Artificial Sequence
  VL-CDR6 Sequence
QQYTPLPWT

SEQ ID NO: 356 - protein, 11 aa, Artificial Sequence
  VL-CDR4 Sequence
RASQSVSSNLA

SEQ ID NO: 357 - protein, 7 aa, Artificial Sequence
  VL-CDR5 Sequence
GASTRAT

SEQ ID NO: 358 - protein, 9 aa, Artificial Sequence
  VL-CDR6 Sequence
QQYTPLPWT

SEQ ID NO: 359 - protein, 11 aa, Artificial Sequence
  VL-CDR4 Sequence
RASQSVSSNLA

SEQ ID NO: 360 - protein, 7 aa, Artificial Sequence
  VL-CDR5 Sequence
GASTRAT

SEQ ID NO: 361 - protein, 9 aa, Artificial Sequence
  VL-CDR6 Sequence
QQYTPLPWT

SEQ ID NO: 362 - protein, 11 aa, Artificial Sequence
  VL-CDR4 Sequence
RASQSVSSNLA

SEQ ID NO: 363 - protein, 7 aa, Artificial Sequence
  VL-CDR5 Sequence
GASTRAT

SEQ ID NO: 364 - protein, 9 aa, Artificial Sequence
  VL-CDR6 Sequence
QQYTPLPWT

SEQ ID NO: 365 - protein, 11 aa, Artificial Sequence
  VL-CDR4 Sequence
QASQDISNYLN

SEQ ID NO: 366 - protein, 7 aa, Artificial Sequence
  VL-CDR5 Sequence
DASNLET

SEQ ID NO: 367 - protein, 10 aa, Artificial Sequence
  VL-CDR6 Sequence
QQVYALPPWT

SEQ ID NO: 368 - protein, 11 aa, Artificial Sequence
  VL-CDR4 Sequence
QASQDISNYLN

SEQ ID NO: 369 - protein, 7 aa, Artificial Sequence
  VL-CDR5 Sequence
DASNLET

SEQ ID NO: 370 - protein, 10 aa, Artificial Sequence
  VL-CDR6 Sequence
QQVYALPPWT

SEQ ID NO: 371 - protein, 11 aa, Artificial Sequence
  VL-CDR4 Sequence
QASQDISNYLN

SEQ ID NO: 372 - protein, 7 aa, Artificial Sequence
  VL-CDR5 Sequence
DASNLET

SEQ ID NO: 373 - protein, 10 aa, Artificial Sequence
  VL-CDR6 Sequence
QQVYALPPWT

SEQ ID NO: 374 - protein, 11 aa, Artificial Sequence
  VL-CDR4 Sequence
QASQDISNYLN

SEQ ID NO: 375 - protein, 7 aa, Artificial Sequence
  VL-CDR5 Sequence
DASNLET

SEQ ID NO: 376 - protein, 10 aa, Artificial Sequence
  VL-CDR6 Sequence
QQVYALPPWT

SEQ ID NO: 377 - protein, 504 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 378 - protein, 492 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNNKPGKEDGNKPGKEDNKKPGKEDGNKPGKEDNNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 379 - protein, 520 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNKKPGKEDGNKPGKEDGNKPGKEDNKKPGKEDGNKPGKEDNNKPGKEDGNKPGKEDNNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 380 - protein, 520 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 381 - protein, 520 aa, Staphylococcus aureus; identical to SEQ ID NO: 379

SEQ ID NO: 382 - protein, 492 aa, Staphylococcus aureus; identical to SEQ ID NO: 378

SEQ ID NO: 383 - protein, 516 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDGNKPGKEDNKKPGKEDGNKPGKEDNKKPGKEDGNKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 384 - protein, 450 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSVSKEILAEAKKLNDAQAPKEEDNKKPGKEDGNKPGKEDGNKPGKEDNKKPGKEDGNKPGKEDNNKPGKEDGNKPGKEDNNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 385 - protein, 450 aa, Staphylococcus aureus; identical to SEQ ID NO: 384

SEQ ID NO: 386 - protein, 450 aa, Staphylococcus aureus; identical to SEQ ID NO: 384

SEQ ID NO: 387 - protein, 516 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 388 - protein, 508 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 389 - protein, 508 aa, Staphylococcus aureus; identical to SEQ ID NO: 388

SEQ ID NO: 390 - protein, 436 aa, Staphylococcus aureus [sequence omitted]

SEQ ID NO: 391 - protein, 61 aa, Staphylococcus aureus
ADNNFNKEQQNAFYEILNMPNLNEEQRNGFIQSLKDDPSQSANLLSEAKKLNESQAPKGGC

SEQ ID NO: 392 - protein, 61 aa, Staphylococcus aureus
ADNKFNKEQQNAFYEILHLPNLNEEQRNGFIQSLKDDPSQSANLLAEAKKLNDAQAPKGGC

SEQ ID NO: 393 - protein, 61 aa, Staphylococcus aureus
ADNKFNKEQQNAFYEILHLPNLTEEQRNGFIQSLKDDPSVSKEILAEAKKLNDAQAPKGGC

SEQ ID NO: 394 - protein, 64 aa, Staphylococcus aureus
ADAQQNNFNKDQQSAFYEILNMPNLNEAQRNGFIQSLKDDPSQSTNVLGEAKKLNESQAPKGGC

SEQ ID NO: 395 - protein, 59 aa, Staphylococcus aureus
AQHDEAQQNAFYQVLNMPNLNADQRNGFIQSLKDDPSQSANVLGEAQKLNDSQAPKGGC

SEQ ID NO: 396 - protein, 70 aa, Staphylococcus aureus
GSENTQQTSTKHQTTQNNYVTDQQKAFYQVLHLKGITEEQRNQYIKTLREHPERAQEVFSESLKDSKGGC

SEQ ID NO: 397 - protein, 64 aa, Staphylococcus aureus
GNPDRRVAQQNAFYNVLKNDNLTEQEKNNYIAQIKENPDRSQQVWVESVQSSKAKERQNIEGGC

SEQ ID NO: 398 - protein, 59 aa, Staphylococcus aureus
AQHDEAKKNAFYQVLNMPNLNADQRNGFIQSLKAAPSQSANVLGEAQKLNDSQAPKGGC

SEQ ID NO: 399 - protein, 64 aa, Artificial Sequence
  SpA-DKKAA mutant
ADAQQNNFNKDKKSAFYEILNMPNLNEAQRNGFIQSLKAAPSQSTNVLGEAKKLNESQAPKGGC

SEQ ID NO: 400 - protein, 64 aa, Artificial Sequence
  SpA-DKK mutant
ADAQQNNFNKDKKSAFYEILNMPNLNEAQRNGFIQSLKDDPSQSTNVLGEAKKLNESQAPKGGC

SEQ ID NO: 401 - protein, 9 aa, Artificial Sequence
  SpA consensus sequence
  MUTAGEN 3: wherein X is any of N, S or K
  MUTAGEN 7: wherein X is any of E, Q or N
  MUTAGEN 8: wherein X is any of I or V
QQXAFYXXL

SEQ ID NO: 402 - protein, 16 aa, Artificial Sequence
  SpA consensus sequence
  MUTAGEN 15: wherein X is any of Q or V
QRNGFIQSLKDDPSXS

SEQ ID NO: 403 - protein, 9 aa, Artificial Sequence
  IGBP mutant consensus sequence
  MUTAGEN 3: wherein X is any of N, S or K
  MUTAGEN 7: wherein X is any of E, Q or N
  MUTAGEN 8: wherein X is any of I or V
KKXAFYXXL

SEQ ID NO: 404 - protein, 16 aa, Artificial Sequence
  IGBP mutant consensus sequence
  MUTAGEN 15: wherein X is any of Q or V
QRNGFIQSLKAAPSXS

SEQ ID NO: 405 - protein, 9 aa, Artificial Sequence
  VH-CDR1
YTFTGRYMH

SEQ ID NO: 406 - protein, 17 aa, Artificial Sequence
  VH-CDR2
IINPRLGSTSYAQKFQG

SEQ ID NO: 407 - protein, 16 aa, Artificial Sequence
  VH-CDR3
ARDGEVGARDLGWFDP

SEQ ID NO: 408 - protein, 472 aa, Artificial Sequence
  Heavy Chain
MELGLCWVFLVAILEGVQCQVQLVQSGAEVKKPGASVKVSCKASGYTFTGRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 471 aa, Artificial Sequence
  Heavy Chain
MELGLCWVFLVAILEGVQCEVQLVQSGAEVKKPGASVKVSCKASGYTFTGRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 410 - protein, 472 aa, Artificial Sequence
  Heavy Chain
MELGLCWVFLVAILEGVQCQVQLVQSGAEVKKPGASVKVSCKASGYTFTSRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - protein, 471 aa, Artificial Sequence
  Heavy Chain
MELGLCWVFLVAILEGVQCEVQLVQSGAEVKKPGASVKVSCKASGYTFTSRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 412 - protein, 472 aa, Artificial Sequence
  Heavy Chain
MELGLCWVFLVAILEGVQCQVQLVQSGAEVKKPGASVKVSCKASGYTFRSRYMHWVRQAPGQGLEWMGIINPMYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 413 - protein, 471 aa, Artificial Sequence
  Heavy Chain
MELGLCWVFLVAILEGVQCEVQLVQSGAEVKKPGASVKVSCKASGYTFRSRYMHWVRQAPGQGLEWMGIINPMYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 414 - protein, 468 aa, Artificial Sequence; identical to SEQ ID NO: 40
  Heavy Chain

SEQ ID NO: 415 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 416 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 417 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 418 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 419 - protein, 233 aa, Artificial Sequence
  Light Chain
MELGLCWVFLVAILEGVQCEIVLTQSPATLSVSPGERATLSCRASQSVSSNLAWYQQKPGQAPRLLIYGASTRATGIPARFSGSGSGTEFTLTISSLQSEDFAVYYCQQYTPLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 9 aa, Artificial Sequence
  VH CDR1
  MUTAGEN 4: wherein X is any of T, R, Q, P, D, E, G, S, A, M
  MUTAGEN 5: wherein X is any of S, R, A, E, H, L, G
  MUTAGEN 6: wherein X is any of Y, L, R, H
  MUTAGEN 8: wherein X is any of I, M
YTFXXXYXH

SEQ ID NO: 421 - protein, 17 aa, Artificial Sequence
  MUTAGEN 1: wherein X is any of I, W
  VH CDR2
  MUTAGEN 5: wherein X is any of S, H, N, P, R, M, G
  MUTAGEN 6: wherein X is any of G, V, N, S, L, Y, I, V, F
  MUTAGEN 7: wherein X is any of G, D
  MUTAGEN 8: wherein X is any of S, H, N, R, G
  MUTAGEN 10: wherein X is any of S, H, N
XINPXXXXTXYAQKFQG

SEQ ID NO: 422 - protein, 11 aa, Artificial Sequence
  MUTAGEN 1: wherein X is any of R, Q
  VL CDR1
  MUTAGEN 5: wherein X is any of S, D
  MUTAGEN 6: wherein X is any of V, I
  MUTAGEN 8: wherein X is any of S, N
  MUTAGEN 9: wherein X is any of S, Y, N
  MUTAGEN 11: wherein X is any of A, N
XASQXXSXXLX

SEQ ID NO: 423 - protein, 7 aa, Artificial Sequence
  MUTAGEN 1: wherein X is any of G, A, D
  VL CDR2
  MUTAGEN 4: wherein X is any T, S, N
  MUTAGEN 5: wherein X is any R, L
  MUTAGEN 6: wherein X is any A, Q, E
  MUTAGEN 7: wherein X is any T, S
XASXXXX